(19) United States
(12) Patent Application Publication
(10) Pub. No.: US 2026/0263558 A1
(43) Pub. Date: Sep. 10, 2026

(54) PREFERENTIALLY EXPRESSED ANTIGEN IN MELANOMA (PRAME) T CELL RECEPTORS AND METHODS OF USE THEREOF

(71) Applicant: Regeneron Pharmaceuticals, Inc., Tarrytown, NY (US)

(72) Inventors: Natalie Bowerman, Stamford, CT (US); Johanna Hansen, Greenwich, CT (US)

(21) Appl. No.: 19/567,614

(22) Filed: Mar. 16, 2026

Related U.S. Application Data
(62) Division of application No. 17/793,952, filed on Jan. 22, 2021, now Pat. No. 12,636,344.
(60) Provisional application No. 62/965,231, filed on Jan. 24, 2020.

(51) Int. Cl. A61K 38/17; A61K 35/17; A61K 39/00; C07K 14/705; C07K 16/32; C12N 5/0783; C12N 15/62; C12N 15/86

(52) U.S. Cl. CPC A61K 38/1774; A61K 35/17; A61K 39/001189; C07K 14/7051; C07K 16/32; C12N 5/0636; C12N 15/625; C12N 15/86; A61K 2039/5156; C12N 2740/10043

(57) ABSTRACT

The present invention provides isolated T cell receptors (TCRs) that specifically bind to an HLA-displayed cancer testis antigen preferentially expressed antigen in melanoma (PRAME) peptide, as well as therapeutic and diagnostic methods of using those isolated TCRs.

### RELATED APPLICATIONS

This application is a divisional of U.S. patent application Ser. No. 17/793,952, filed on Jul. 20, 2022, which is a 35 U.S.C. § 371 national stage filing of International Application No. PCT/US2021/014490, filed on Jan. 22, 2021, which in turn claims the benefit of priority to U.S. Provisional Application No. 62/965,231, filed on Jan. 24, 2020. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Mar. 16, 2026, is named 118003_00503_SL.txt and is 1,329,659 bytes in size.

### BACKGROUND

T cell receptors (TCRs) are membrane bound heterodimers comprising an α and β chain resembling an immunoglobulin variable (V) and constant (C) region. The TCR α chain includes a covalently linked V-α chain and C-α chain, whereas the β chain includes a V-β chain covalently linked to a C-β chain. The V-α and V-β chains form a pocket or cleft that can bind an antigen in the context of a major histocompatibility complex (MHC) (known in humans as an HLA complex). (Davis Ann. Rev. of Immunology 3: 537 (1985); Fundamental Immunology 3rd Ed., W. Paul Ed. New York (1993)).

TCRs are primary effectors of the immune system that have unique advantages as a platform for developing therapeutics. While antibody therapeutics are limited to recognition of pathogens in the blood and extracellular spaces, or to protein targets on the cell surface, T cell receptors can recognize antigens displayed with MHC molecules on the surface of cells, including antigens derived from intracellular proteins. Depending on the subtype of T cells that recognize displayed antigen and become activated, TCRs can participate in controlling various immune responses. For instance, T cells are involved in regulation of the humoral immune response through induction of differentiation of B cells into antibody producing cells. In addition, activated T cells act to initiate cell-mediated immune responses. Thus, TCRs can recognize additional targets not available to antibodies. In addition, TCRs have been reported to mediate cell killing, increase B cell proliferation, and impact the development and severity of various disorders including cancer, allergies, viral infections and autoimmune disorders.

In view of the function of TCRs, antigen-specific TCRs have been evaluated for use in immunotherapy for their ability to redirect T cells to tumors expressing the antigen. TCRs will bind to a small peptide, only 8-12 amino acids in length, which are bound on the surface of a target cell by the Major Histocompatibility Complex (MHC). TCRs can therefore recognize intracellular antigens derived from cancer or viral proteins because these antigens are processed and displayed as peptides in the context of the surface MHC. Hence, TCRs can recognize additional internal cell targets not available to antibodies or therapies that cannot penetrate the cell.

However, the challenge of the industry is to engineer TCRs that lack immunogenicity when administered to a patient and have fine specificity to the particular peptide antigen of interest, without cross-reacting to other peptides on MHC or similar epitopes found in the natural protein repertoire.

Preferentially expressed antigen in melanoma, or PRAME, is a well-known cancer-testis antigen (CTA) encoded on the X chromosome. It was first identified as a tumor antigen that could be recognized by HLA-A*24 restricted cytotoxic T lymphocytes in metastatic cutaneous melanoma (Ikeda H., et al. (1997) Immunity 6:199-208). PRAME has been shown to act as a repressor of retinoic acid receptor and, thus, may confer a growth advantage to cancer cells via this mechanism (see, e.g., Epping M. T., et al. (2005) Cell 122:835-847).

PRAME is abundantly re-expressed by many tumors of different histological types, including melanoma, renal cell cancer, non-small cell lung cancer (NSCLC), neuroblastoma, breast cancer, multiple myeloma, acute leukemia, chronic myeloid leukemia, multiple sarcoma subtypes, and primary and metastatic uveal melanoma but, in normal healthy adult tissues, PRAME expression is restricted to the testes.

There is an unmet need in the art for new targeting agents based on T cell receptors that specifically bind to PRAME antigens, as well as methods for producing and using such agents in therapeutic and diagnostic settings.

### SUMMARY

The present invention provides T cell receptors (TCRs) that were generated against a PRAME peptide antigen in the context of MHC (HLA-A2). The unique TCR sequences identified have shown specific binding to a small peptide PRAME presented in the groove of an HLA molecule.

Accordingly, in one aspect, the present invention provides a T cell receptor (TCR) (e.g., an isolated TCR or a TCR expressed on an isolated cell) that binds specifically to an HLA-A2 presented cancer testis antigen preferentially expressed antigen in melanoma (PRAME) peptide comprising the amino acid sequence of RLDQLLRHV (SEQ ID NO:929) (PRAME 312-320) wherein the TCR comprises an alpha chain variable domain comprising a complementary determining region (CDR) 3, wherein the CDR3 comprises the amino acid sequence of any one of the alpha chain variable domain CDR3 amino acid sequences set forth in Table 3.

In another aspect, the present invention provides a T cell receptor (TCR) (e.g., an isolated TCR or a TCR expressed on an isolated cell) that binds specifically to an HLA-A2 presented cancer testis antigen preferentially expressed antigen in melanoma (PRAME) peptide comprising the amino acid sequence of RLDQLLRHV (SEQ ID NO:929) (PRAME 312-320), wherein the TCR comprises a beta chain variable domain comprising a complementary determining region (CDR) 3, wherein the CDR3 comprises the amino acid sequence of any one of the beta chain variable domain CDR3 amino acid sequences set forth in Table 3.

In some embodiments, the alpha chain variable domain further comprises a CDR1 and a CDR2, wherein the CDR1 comprises any one of the alpha chain variable domain CDR1 amino acid sequences set forth in Table 3 and the CDR2 independently comprises any one of the alpha chain variable domain CDR2 amino acid sequences set forth in Table 3.

In some embodiments, the beta chain variable domain further comprises a CDR1 and a CDR2, wherein the CDR1 comprises any one of the beta chain variable CDR1 amino acid sequences set forth in Table 3 and the CDR2 independently comprises any one of the beta chain variable domain CDR2 amino acid sequences set forth in Table 3.

The TCR may include at least one TCR alpha chain variable domain and/or at least one beta chain variable domain; or the TCR may include a TCR alpha chain variable domain and a TCR beta chain variable domain.\

In some embodiments, the TCR comprises alpha chain variable domain CDR1, CDR2 and CDR3 contained within any one of the alpha chain variable domain sequences listed in Table 5; and beta chain variable domain CDR1, CDR2 and CDR3 contained within any one of the beta chain variable domain sequences listed in Table 5.

In some embodiments, the TCR comprises an alpha chain variable domain having an amino acid sequence that has at least 85% amino acid identity to the entire amino acid sequence of any one of the amino acid sequences of the alpha chain variable domain amino acid sequences listed in Table 5.

In some embodiments, the TCR comprises a beta chain variable domain having an amino acid sequence that has at least 85% amino acid identity to the entire amino acid sequence of any one of the amino acid sequences of the beta chain variable domain amino acid sequences listed in Table 5.

In some embodiments, the TCR comprises (a) an alpha chain variable domain having an amino acid sequence that has at least 85% amino acid identity to the entire amino acid sequence of any one of the amino acid sequences of the alpha chain variable domain amino acid sequences listed in Table 5; and (b) a beta chain variable domain having an amino acid sequence that has at least 85% amino acid identity to the entire amino acid sequence of any one of the amino acid sequences of the beta chain variable domain amino acid sequences listed in Table 5.

In some embodiments, the TCR comprises (a) an alpha chain variable domain CDR1 domain having an amino acid sequence selected from the group consisting of SEQ ID NOs: 1, 7, 13, 19, 25, 31, 37, 43, 49, 55, 61, 67, 73, 79, 85, 91, 97, and 103; (b) an alpha chain variable domain CDR2 domain having an amino acid sequence selected from the group consisting of SEQ ID NOs: 2, 8, 14, 20, 26, 32, 38, 44, 50, 56, 62, 68, 74, 80, 86, 92, 98, and 104; (c) an alpha chain variable domain CDR3 domain having an amino acid sequence selected from the group consisting of SEQ ID NOs: 3, 9, 15, 21, 27, 33, 39, 45, 51, 57, 63, 69, 75, 81, 87, 93, 99, and 105; (d) a beta chain variable domain CDR1 having an amino acid sequence selected from the group consisting of SEQ ID NOs: 4, 10, 16, 22, 28, 34, 40, 46, 52, 58, 64, 70, 76, 82, 88, 94, 100, and 106; (e) a beta chain variable domain CDR2 having an amino acid sequence selected from the group consisting of SEQ ID NOs: 5, 11, 17, 23, 29, 35, 41, 47, 53, 59, 65, 71, 77, 83, 89, 95, 101, and 107; and (f) a beta chain variable domain CDR3 having an amino acid sequence selected from the group consisting of SEQ ID NOs: 6, 12, 18, 24, 30, 36, 42, 48, 54, 60, 66, 72, 78, 84, 90, 96, 102, and 108.

In some embodiments, the TCR comprises an alpha chain variable domain/beta chain variable domain amino acid sequence pair selected from the group consisting of SEQ ID NOs: 217/219, 229/231, 237/239, 241/243, and 285/287.

In some embodiments, the TCR comprises an alpha chain variable domain/beta chain variable domain amino acid sequence pair selected from the group consisting of SEQ ID NOs: 217/219, 221/223, 225/227, 229/231, 233/235, 237/239, 241/243, 245/247, 249/251, 253/255, 257/259, 261/263, 265/267, 269/271, 273/275, 277/279, 281/283, and 285/287.

The present invention also provides a TCR (e.g., an isolated TCR or a TCR expressed on an isolated cell) that complete for binding to any one or more of the TCRs of the invention.

In one aspect, the present invention provides a T cell receptor (TCR) (e.g., an isolated TCR or a TCR expressed on an isolated cell) that binds specifically to an HLA-A2 presented cancer testis antigen preferentially expressed antigen in melanoma (PRAME) peptide comprising the amino acid sequence of SLLQHLIGL (SEQ ID NO:930) (PRAME 425-433), wherein the TCR comprises an alpha chain variable domain comprising a complementary determining region (CDR) 3, wherein the CDR3 comprises the amino acid sequence of any one of the alpha chain variable domain CDR3 amino acid sequences set forth in Table 6.

In another aspect, the present invention provides a T cell receptor (TCR) (e.g., an isolated TCR or a TCR expressed on an isolated cell) that binds specifically to an HLA-A2 presented preferentially expressed antigen in melanoma (PRAME) peptide comprising the amino acid sequence of SLLQHLIGL (SEQ ID NO:930) (PRAME 425-433), wherein the TCR comprises a beta chain variable domain comprising a complementary determining region (CDR) 3, wherein the CDR3 comprises the amino acid sequence of any one of the beta chain variable domain CDR3 amino acid sequences set forth in Table 6.

In some embodiments, the alpha chain variable domain further comprises a CDR1 and a CDR2, wherein the CDR1 comprises any one of the alpha chain variable domain CDR1 amino acid sequences set forth in Table 6 and the CDR2 independently comprises any one of the alpha chain variable domain CDR2 amino acid sequences set forth in Table 6.

In some embodiments, the beta chain variable domain further comprises a CDR1 and a CDR2, wherein the CDR1 comprises any one of the beta chain variable CDR1 amino acid sequences set forth in Table 6 and the CDR2 independently comprises any one of the beta chain variable domain CDR2 amino acid sequences set forth in Table 6.

The TCR may include at least one TCR alpha chain variable domain and/or at least one beta chain variable domain; or the TCR may include a TCR alpha chain variable domain and a TCR beta chain variable domain.

In some embodiments, the TCR comprises alpha chain variable domain CDR1, CDR2 and CDR3 contained within any one of the alpha chain variable domain sequences listed in Table 8; and beta chain variable domain CDR1, CDR2 and CDR3 contained within any one of the beta chain variable domain sequences listed in Table 8.

In some embodiments, the TCR comprises an alpha chain variable domain having an amino acid sequence that has at least 85% amino acid identity to the entire amino acid sequence of any one of the amino acid sequences of the alpha chain variable domain amino acid sequences listed in Table 8.

In some embodiments, the TCR comprises a beta chain variable domain having an amino acid sequence that has at least 85% amino acid identity to the entire amino acid sequence of any one of the amino acid sequences of the beta chain variable domain amino acid sequences listed in Table 8.

In some embodiments, the TCR comprises (a) an alpha chain variable domain having an amino acid sequence that has at least 85% amino acid identity to the entire amino acid sequence of any one of the amino acid sequences of the alpha chain variable domain amino acid sequences listed in Table 8; and (b) a beta chain variable domain having an amino acid sequence that has at least 85% amino acid identity to the entire amino acid sequence of any one of the amino acid sequences of the beta chain variable domain amino acid sequences listed in Table 8.

In some embodiments, the TCR comprises (a) an alpha chain variable domain CDR1 domain having an amino acid sequence selected from the group consisting of SEQ ID NOs: 289, 295, 301, 307, 313, 319, 325, 331, 337, 343, 349, 355, 361, 367, 373, 379, 385, 391, 397, 403, 409, 415, 421, 427, 433, 439, 445, 451, 457, 463, 469, 475, 481, 487, 493, 499, 505, 511, 517, and 523; (b) an alpha chain variable domain CDR2 domain having an amino acid sequence selected from the group consisting of SEQ ID NOs: 290, 296, 302, 308, 314, 320, 326, 332, 338, 344, 350, 356, 362, 368, 374, 380, 386, 392, 398, 404, 410, 416, 422, 428, 434, 440, 446, 452, 458, 464, 470, 476, 482, 488, 494, 500, 506, 512, 518, and 524; (c) an alpha chain variable domain CDR3 domain having an amino acid sequence selected from the group consisting of SEQ ID NOs: 291, 297, 303, 309, 315, 321, 327, 333, 339, 345, 351, 357, 363, 369, 375, 381, 387, 393, 399, 405, 411, 417, 423, 429, 435, 441, 447, 453, 459, 465, 471, 477, 483, 489, 495, 501, 507, 513, 519, and 525; (d) a beta chain variable domain CDR1 having an amino acid sequence selected from the group consisting of SEQ ID NOs: 292, 298, 304, 310, 316, 322, 328, 334, 340, 346, 352, 358, 364, 370, 376, 382, 388, 394, 400, 406, 412, 418, 424, 430, 436, 442, 448, 454, 460, 466, 472, 478, 484, 490, 496, 502, 508, 514, 520, and 526; (e) a beta chain variable domain CDR2 having an amino acid sequence selected from the group consisting of SEQ ID NOs: 293, 299, 305, 311, 317, 323, 329, 335, 341, 347, 353, 359, 365, 371, 377, 383, 389, 395, 401, 407, 413, 419, 425, 431, 437, 443, 449, 455, 461, 467, 473, 479, 485, 491, 497, 503, 509, 515, 521, and 527; and (f) a beta chain variable domain CDR3 having an amino acid sequence selected from the group consisting of SEQ ID NOs: 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, and 528.

In some embodiments, the TCR comprises an alpha chain variable domain/beta chain variable domain amino acid sequence pair selected from the group consisting of SEQ ID NOs: 825/827, 845/847, 853/855, 857/859, 865/867, 873/875, 885/887, 893/805, 897/899, 901/903, 913/915, and 925/927.

In some embodiments, the TCR comprises an alpha chain variable domain/beta chain variable domain amino acid sequence pair selected from the group consisting of SEQ ID NOs: 769/771, 773/775, 777/779, 781/783, 785/787, 789/791, 793/795, 797/799, 801/803, 805/807, 809/811, 813/815, 817/819, 821/823, 825/827, 829/831, 833/835, 837/839, 841/843, 845/847, 849/851, 853/855, 857/859, 861/863, 865/867, 869/871, 873/875, 877/879, 881/883, 885/887, 889/891, 893/805, 897/899, 901/903, 905/907, 909/911, 913/915, 917/919, 921/923, and 925/927.

The present invention also provides a TCR (e.g., an isolated TCR or a TCR expressed on an isolated cell) that complete for binding to any one or more of the TCRs of the invention.

In some embodiments, the TCRs of the invention further comprise a detectable moiety.

The present invention further provides pharmaceutical compositions comprising any of the TCRs of the invention, and a pharmaceutically acceptable carrier or diluent; as well as isolated cells presenting any of the TCRs of the invention.

In one aspect, the present invention provides isolated polynucleotide molecules comprising a polynucleotide sequence that encodes an alpha chain variable domain of any of the TCRs of the invention.

In another aspect, the present invention provides isolated polynucleotide molecules comprising a polynucleotide sequence that encodes a beta chain variable domain of any of the TCRs of the invention.

The present invention also provides vectors comprising the polynucleotide molecule of the invention; cells expressing the vectors of the invention.

In one aspect, the present invention provides a method of treating a subject having a PRAME-associated disease or disorder. The methods includes administering to the subject a therapeutically effective amount of a TCR (e.g., an isolated TCR or a TCR expressed on an isolated cell), pharmaceutical composition, or a plurality of the cells of the invention, thereby treating the subject.

In some embodiments, the PRAME-associated disease or disorder is PRAME-associated cancer.

In some embodiments, the PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

In some embodiments of the invention, a TCR (e.g., an isolated TCR or a TCR expressed on an isolated cell), pharmaceutical composition, or a plurality of the cells of the invention is administered to the subject in combination with a second therapeutic agent.

The TCR, the pharmaceutical composition, or the plurality of cells may be administered subcutaneously, intravenously, intradermally, intraperitoneally, orally, intramuscularly or intracranially to the subject.

In one aspect, the present invention provides an isolated nucleic acid molecule encoding a T cell receptor (TCR), wherein the TCR binds specifically to an HLA-A2 presented cancer testis antigen preferentially expressed antigen in melanoma (PRAME) peptide comprising the amino acid sequence of RLDQLLRHV (SEQ ID NO:929) (PRAME 312-320), wherein the TCR comprises an alpha chain variable domain comprising a complementary determining region (CDR) 3, wherein the CDR3 comprises the amino acid sequence of any one of the alpha chain variable domain CDR3 amino acid sequences set forth in Table 5.

In another aspect, the present invention provides an isolated nucleic acid molecule encoding a T cell receptor (TCR), wherein the TCR binds specifically to an HLA-A2 presented cancer testis antigen preferentially expressed antigen in melanoma (PRAME) peptide comprising the amino acid sequence of RLDQLLRHV (SEQ ID NO:929) (PRAME 312-320), wherein the TCR comprises a beta chain variable domain comprising a complementary determining region (CDR) 3, wherein the CDR3 comprises the amino acid sequence of any one of the beta chain variable domain CDR3 amino acid sequences set forth in Table 5.

In some embodiments, the isolated nucleic acid molecule encodes at least one TCR alpha chain variable domain and/or at least one beta chain variable domain.

In some embodiments, the TCR comprises alpha chain variable domain complementary determining regions (CDR) 1, CDR2, and CDR3 contained within any one of the alpha chain variable domain sequences listed in Table 5; and beta chain variable domain CDR1, CDR2 and CDR3 contained within any one of the beta chain variable domain sequences listed in Table 5.

In some embodiments, the TCR (e.g., isolated TCR or TCR expressed on an isolated cell) comprises alpha chain variable domain having an amino acid sequence that has at least 85% amino acid identity to the entire amino acid sequence of any one of the amino acid sequences of the alpha chain variable domain amino acid sequences listed in Table 5.

In some embodiments, the TCR comprises beta chain variable domain having an amino acid sequence that has at least 85% amino acid identity to the entire amino acid sequence of any one of the amino acid sequences of the beta chain variable domain amino acid sequences listed in Table 5.

In some embodiments, the TCR comprises (a) an alpha chain variable domain having an amino acid sequence that has at least 85% amino acid identity to the entire amino acid sequence of any one of the amino acid sequences of the alpha chain variable domain amino acid sequences listed in Table 5; and (b) a beta chain variable domain having an amino acid sequence that has at least 85% amino acid identity to the entire amino acid sequence of any one of the amino acid sequences of the beta chain variable domain amino acid sequences listed in Table 5.

In some embodiments, the isolated antigen-binding protein comprises (a) an alpha chain variable domain CDR1 domain having an amino acid sequence selected from the group consisting of SEQ ID NOs: 1, 7, 13, 19, 25, 31, 37, 43, 49, 55, 61, 67, 73, 79, 85, 91, 97, and 103; (b) an alpha chain variable domain CDR2 domain having an amino acid sequence selected from the group consisting of SEQ ID NOs: 2, 8, 14, 20, 26, 32, 38, 44, 50, 56, 62, 68, 74, 80, 86, 92, 98, and 104; (c) an alpha chain variable domain CDR3 domain having an amino acid sequence selected from the group consisting of SEQ ID NOs: 3, 9, 15, 21, 27, 33, 39, 45, 51, 57, 63, 69, 75, 81, 87, 93, 99, and 105; (d) a beta chain variable domain CDR1 having an amino acid sequence selected from the group consisting of SEQ ID NOs: 4, 10, 16, 22, 28, 34, 40, 46, 52, 58, 64, 70, 76, 82, 88, 94, 100, and 106; (e) a beta chain variable domain CDR2 having an amino acid sequence selected from the group consisting of SEQ ID NOs: 5, 11, 17, 23, 29, 35, 41, 47, 53, 59, 65, 71, 77, 83, 89, 95, 101, and 107; and (f) a beta chain variable domain CDR3 having an amino acid sequence selected from the group consisting of SEQ ID NOs: 6, 12, 18, 24, 30, 36, 42, 48, 54, 60, 66, 72, 78, 84, 90, 96, 102, and 108.

In some embodiments, the TCR comprises an alpha chain variable domain/beta chain variable domain amino acid sequence pair selected from the group consisting of SEQ ID NOs: 217/219, 229/231, 237/239, 241/243, and 285/287.

In some embodiments, the TCR comprises an alpha chain variable domain/beta chain variable domain amino acid sequence pair selected from the group consisting of SEQ ID NOs: 217/219, 221/223, 225/227, 229/231, 233/235, 237/239, 241/243, 245/247, 249/251, 253/255, 257/259, 261/263, 265/267, 269/271, 273/275, 277/279, 281/283, and 285/287.

In some embodiments, the isolated antigen-binding protein comprises (a) an alpha chain variable domain CDR1 encoded by a nucleic acid sequence selected from the group consisting of SEQ ID NOs: 109, 115, 121, 127, 133, 139, 145, 151, 157, 163, 169, 175, 181, 187, 193, 199, 205, and 211; (b) an alpha chain variable domain CDR2 encoded by a nucleic acid sequence selected from the group consisting of SEQ ID NOs: 110, 116, 122, 128, 134, 140, 146, 152, 158, 164, 170, 176, 182, 188, 194, 200, 206, and 212; (c) an alpha chain variable domain CDR3 encoded by a nucleic acid sequence selected from the group consisting of SEQ ID NOs: 111, 117, 123, 129, 135, 141, 147, 153, 159, 165, 171, 177, 183, 189, 195, 201, 207, and 213; (d) a beta chain variable domain CDR1 encoded by a nucleic acid sequence selected from the group consisting of SEQ ID NOs: 112, 118, 124, 130, 136, 142, 148, 154, 160, 166, 172, 178, 184, 190, 196, 202, 208, and 214; (e) a beta chain variable domain CDR2 encoded by a nucleic acid sequence selected from the group consisting of SEQ ID NOs: 113, 119, 125, 131, 137, 143, 149, 155, 161, 167, 173, 179, 185, 191, 197, 203, 209, and 215; and (f) a beta chain variable domain CDR3 encoded by a nucleic acid sequence selected from the group consisting of SEQ ID NOs: 114, 120, 126, 132, 138, 144, 150, 156, 162, 168, 174, 180, 186, 192, 198, 204, 210, and 216.

In some embodiments, the TCR comprises an alpha chain variable domain/beta chain variable domain nucleic acid sequence pair selected from the group consisting of SEQ ID NOs: 218/220, 222/224, 226/228, 230/232, 234/236, 238/240, 242/244, 246/248, 250/252, 254/256, 258/260, 262/264, 266/268, 270/272, 274/276, 278/280, 282/284, and 286/288.

The present invention also provides vectors comprising an isolated nucleic acid molecule of the invention and isolated cells comprising a vector of the invention.

In one aspect, the present invention provides isolated polynucleotide molecules comprising a polynucleotide sequence that encodes an alpha chain variable domain of any of the TCRs of the invention.

In another aspect, the present invention provides isolated polynucleotide molecules comprising a polynucleotide sequence that encodes a beta chain variable domain of any of the TCRs of the invention.

The present invention also provides vectors comprising the polynucleotide molecule of the invention; cells expressing the vectors of the invention.

In one aspect, the present invention provides a method of treating a subject having a PRAME-associated disease or disorder. The methods includes administering to the subject a therapeutically effective amount of a TCR (e.g., an isolated TCR or a TCR expressed on an isolated cell), pharmaceutical composition, or a plurality of the cells of the invention, thereby treating the subject.

In some embodiments, the PRAME-associated disease or disorder is PRAME-associated cancer.

In some embodiments, the PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

In some embodiments of the invention, a TCR (e.g., an isolated TCR or a TCR expressed on an isolated cell), pharmaceutical composition, or a plurality of the cells of the invention is administered to the subject in combination with a second therapeutic agent.

The TCR, the pharmaceutical composition, or the plurality of cells may be administered subcutaneously, intravenously, intradermally, intraperitoneally, orally, intramuscularly or intracranially to the subject.

In one aspect, the present invention provides an isolated nucleic acid molecule encoding a T cell receptor (TCR), wherein the TCR binds specifically to an HLA-A2 presented cancer testis antigen preferentially expressed antigen in melanoma (PRAME) peptide comprising the amino acid sequence of SLLQHLIGL (SEQ ID NO:930) (PRAME 425-433), wherein the TCR comprises an alpha chain variable domain comprising a complementary determining region (CDR) 3, wherein the CDR3 comprises the amino acid sequence of any one of the alpha chain variable domain CDR3 amino acid sequences set forth in Table 8.

In another aspect, the present invention provides an isolated nucleic acid molecule encoding a T cell receptor (TCR), wherein the TCR binds specifically to an HLA-A2 presented cancer testis antigen preferentially expressed antigen in melanoma (PRAME) peptide comprising the amino acid sequence of SLLQHLIGL (SEQ ID NO:930) (PRAME 425-433), wherein the TCR comprises a beta chain variable domain comprising a complementary determining region (CDR) 3, wherein the CDR3 comprises the amino acid sequence of any one of the beta chain variable domain CDR3 amino acid sequences set forth in Table 8.

In some embodiments, the isolated nucleic acid molecule encodes at least one TCR alpha chain variable domain and/or at least one beta chain variable domain.

In some embodiments, the TCR comprises alpha chain variable domain complementary determining regions (CDR) 1, CDR2, and CDR3 contained within any one of the alpha chain variable domain sequences listed in Table 8; and beta chain variable domain CDR1, CDR2 and CDR3 contained within any one of the beta chain variable domain sequences listed in Table 8.

In some embodiments, the TCR (e.g., isolated TCR or TCR expressed on an isolated cell) comprises alpha chain variable domain having an amino acid sequence that has at least 85% amino acid identity to the entire amino acid sequence of any one of the amino acid sequences of the alpha chain variable domain amino acid sequences listed in Table 8.

In some embodiments, the TCR comprises beta chain variable domain having an amino acid sequence that has at least 85% amino acid identity to the entire amino acid sequence of any one of the amino acid sequences of the beta chain variable domain amino acid sequences listed in Table 8.

In some embodiments, the TCR comprises (a) an alpha chain variable domain having an amino acid sequence that has at least 85% amino acid identity to the entire amino acid sequence of any one of the amino acid sequences of the alpha chain variable domain amino acid sequences listed in Table 8; and (b) a beta chain variable domain having an amino acid sequence that has at least 85% amino acid identity to the entire amino acid sequence of any one of the amino acid sequences of the beta chain variable domain amino acid sequences listed in Table 8.

In some embodiments, the isolated antigen-binding protein comprises (a) an alpha chain variable domain CDR1 domain having an amino acid sequence selected from the group consisting of SEQ ID NOs: 289, 295, 301, 307, 313, 319, 325, 331, 337, 343, 349, 355, 361, 367, 373, 379, 385, 391, 397, 403, 409, 415, 421, 427, 433, 439, 445, 451, 457, 463, 469, 475, 481, 487, 493, 499, 505, 511, 517, and 523; (b) an alpha chain variable domain CDR2 domain having an amino acid sequence selected from the group consisting of SEQ ID NOs: 290, 296, 302, 308, 314, 320, 326, 332, 338, 344, 350, 356, 362, 368, 374, 380, 386, 392, 398, 404, 410, 416, 422, 428, 434, 440, 446, 452, 458, 464, 470, 476, 482, 488, 494, 500, 506, 512, 518, and 524; (c) an alpha chain variable domain CDR3 domain having an amino acid sequence selected from the group consisting of SEQ ID NOs: 291, 297, 303, 309, 315, 321, 327, 333, 339, 345, 351, 357, 363, 369, 375, 381, 387, 393, 399, 405, 411, 417, 423, 429, 435, 441, 447, 453, 459, 465, 471, 477, 483, 489, 495, 501, 507, 513, 519, and 525; (d) a beta chain variable domain CDR1 having an amino acid sequence selected from the group consisting of SEQ ID NOs: 292, 298, 304, 310, 316, 322, 328, 334, 340, 346, 352, 358, 364, 370, 376, 382, 388, 394, 400, 406, 412, 418, 424, 430, 436, 442, 448, 454, 460, 466, 472, 478, 484, 490, 496, 502, 508, 514, 520, and 526; (e) a beta chain variable domain CDR2 having an amino acid sequence selected from the group consisting of SEQ ID NOs: 293, 299, 305, 311, 317, 323, 329, 335, 341, 347, 353, 359, 365, 371, 377, 383, 389, 395, 401, 407, 413, 419, 425, 431, 437, 443, 449, 455, 461, 467, 473, 479, 485, 491, 497, 503, 509, 515, 521, and 527; and (f) a beta chain variable domain CDR3 having an amino acid sequence selected from the group consisting of SEQ ID NOs: 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, and 528.

In some embodiments, the TCR comprises an alpha chain variable domain/beta chain variable domain amino acid sequence pair selected from the group consisting of SEQ ID NOs: 825/827, 845/847, 853/855, 857/859, 865/867, 873/875, 885/887, 893/805, 897/899, 901/903, 913/915, and 925/927.

In some embodiments, the TCR comprises an alpha chain variable domain/beta chain variable domain amino acid sequence pair selected from the group consisting of SEQ ID NOs: 769/771, 773/775, 777/779, 781/783, 785/787, 789/791, 793/795, 797/799, 801/803, 805/807, 809/811, 813/815, 817/819, 821/823, 825/827, 829/831, 833/835, 837/839, 841/843, 845/847, 849/851, 853/855, 857/859, 861/863, 865/867, 869/871, 873/875, 877/879, 881/883, 885/887, 889/891, 893/805, 897/899, 901/903, 905/907, 909/911, 913/915, 917/919, 921/923, and 925/927.

In some embodiments, the isolated antigen-binding protein comprises (a) an alpha chain variable domain CDR1 encoded by a nucleic acid sequence selected from the group consisting of SEQ ID NOs: 529, 535, 541, 547, 553, 559, 565, 571, 577, 583, 589, 595, 601, 607, 613, 619, 625, 631, 637, 643, 649, 655, 661, 667, 673, 679, 685, 691, 697, 703, 709, 715, 721, 727, 733, 739, 745, 751, 757, and 763; (b) an alpha chain variable domain CDR2 encoded by a nucleic acid sequence selected from the group consisting of SEQ ID NOs: 530, 536, 542, 548, 554, 560, 566, 572, 578, 584, 590, 596, 602, 608, 614, 620, 626, 632, 638, 644, 650, 656, 662, 668, 674, 680, 686, 692, 698, 704, 710, 716, 722, 728, 734, 740, 746, 752, 758, and 764; (c) an alpha chain variable domain CDR3 encoded by a nucleic acid sequence selected from the group consisting of SEQ ID NOs: 531, 537, 543, 549, 555, 561, 567, 573, 579, 585, 591, 597, 603, 609, 615, 621, 627, 633, 639, 645, 651, 657, 663, 669, 675, 681, 687, 693, 699, 705, 711, 717, 723, 729, 735, 741, 747, 753, 759, and 765; (d) a beta chain variable domain CDR1 encoded by a nucleic acid sequence selected from the group consisting of SEQ ID NOs: 532, 538, 544, 550, 556, 562, 568, 574, 580, 586, 592, 598, 604, 610, 616, 622, 628, 634, 640, 646, 652, 658, 664, 670, 676, 682, 688, 694, 700, 706, 712, 718, 724, 730, 736, 742, 748, 754, 760, and 766; (e) a beta chain variable domain CDR2 encoded by a nucleic acid sequence selected from the group consisting of SEQ ID NOs: 533, 539, 545, 551, 557, 563, 569, 575, 581, 587, 593, 599, 605, 611, 617, 623, 629, 635, 641, 647, 653, 659, 665, 671, 677, 683, 689, 695, 701, 707, 713, 719, 725, 731, 737, 743, 749, 755, 761, and 767; and (f) a beta chain variable domain CDR3 encoded by a nucleic acid sequence selected from the group consisting of SEQ ID NOs: 534, 540, 546, 552, 558, 564, 570, 576, 582, 588, 594, 600, 606, 612, 618, 624, 630, 636, 642, 648, 654, 660, 666, 672, 678, 684, 690, 696, 702, 708, 714, 720, 726, 732, 738, 744, 750, 756, 762, and 768.

In some embodiments, the TCR comprises an alpha chain variable domain/beta chain variable domain nucleic acid sequence pair selected from the group consisting of SEQ ID NOs: 770/772, 774/776, 778/780, 782/784, 786/788, 790/792, 794/796, 798/800, 802/804, 806/808, 810/812, 814/816, 818/820, 822/824, 826/828, 830/832, 834/836, 838/840, 842/844, 846/848, 850/852, 854/856, 858/860, 862/864, 866/868, 870/872, 874/876, 878/880, 882/884, 886/888, 890/892, 894/896, 898/900, 902/904, 906/908, 910/912, 914/916, 918/920, 922/924, and 926/928.

The present invention also provides vectors comprising an isolated nucleic acid molecule of the invention and isolated cells comprising a vector of the invention.

In one aspect, the present invention provides a method of treating a subject having a PRAME-associated disease or disorder, comprising administering to the subject a plurality of the cells comprising a vector of the invention, thereby treating the subject.

In some embodiments, the PRAME-associated disease or disorder is PRAME-associated cancer.

In some embodiments, the PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

In some embodiments, a plurality of cells is administered to the subject in combination with a second therapeutic agent.

The present invention is further illustrated by the following detailed description.

### DETAILED DESCRIPTION

The present invention provides T cell receptors (TCRs) that were generated against a PRAME peptide antigen in the context of MHC (HLA-A2). The unique TCR sequences identified have shown specific binding to the small peptide PRAME presented in the groove of an HLA molecule.

### I. Definitions

In order that the present invention may be more readily understood, certain terms are first defined. In addition, it should be noted that whenever a value or range of values of a parameter are recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

The term “comprising” or “comprises” is used herein in reference to compositions, methods, and respective component(s) thereof, that are essential to the disclosure, yet open to the inclusion of unspecified elements, whether essential or not.

The term “consisting of” refers to compositions, methods, and respective components thereof as described herein, which are exclusive of any element not recited in that description of the embodiment.

The term “T cell receptor” (TCR), as used herein, refers to an immunoglobulin superfamily member having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having a and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Kabat numbering at the N-terminus), and one constant region (e.g., TCR constant domain α or Cα and typically amino acids 117 to 259 based on Kabat, TCR constant domain β or Cβ, typically amino acids 117 to 295 based on Kabat) adjacent to the cell membrane. Also, like immunoglobulins, the variable domains contain complementary determining regions (CDRs) separated by framework regions (FRs). In certain embodiments, a TCR is found on the surface of T cells (or T lymphocytes) and associates with the CD3 complex. The source of a TCR of the present disclosure may be from various animal species, such as a human, mouse, rat, rabbit or other mammal. In preferred embodiments, the source of a TCR of the present invention is a mouse genetically engineered to produce TCRs comprising human alpha and beta chains (see, e.g., PCT Publication No. WO 2016/164492, the entire contents of which are incorporated herein by reference).

The term “variable region” (variable region of an alpha chain (Vα), variable region of a beta chain (Vβ)) as used herein denotes each of the alpha and beta chains which is involved directly in binding the TCR to the antigen.

The “constant region” of the alpha chain and of the beta chain are not involved directly in binding of a TCR to an antigen, but exhibit various effector functions.

The term “antigen” as used herein is meant any substance that causes the immune system to produce antibodies or specific cell-mediated immune responses against it. A disease-associated antigen is any substance that is associated with any disease that causes the immune system to produce antibodies or a specific-cell mediated response against it.

The term “PRAME” or “preferentially expressed antigen in melanoma” refers to the well-known cancer-testis antigen (CTA) that is re-expressed in numerous cancer types.

The nucleotide sequence of PRAME is known and may be found in, for example, GenBank Accession Nos. NM_001291715.2 (SEQ ID NO: 931), NM_001291716.2 (SEQ ID NO: 932), NM_001291717.2 (SEQ ID NO: 933), NM_001291719.2 (SEQ ID NO: 934), NM_001318126.1 (SEQ ID NO: 935), NM_001318127.1 (SEQ ID NO: 936), NM_006115.5 (SEQ ID NO: 937), NM_206956.3 (SEQ ID NO: 938), NM_206955.2 (SEQ ID NO: 939), NM_206954.3 (SEQ ID NO: 940), and NM_206953.2 (SEQ ID NO: 941). The amino acid sequence of full-length PRAME is known and may be found in, for example, GenBank as Accession Nos. NP_001278646.1 (SEQ ID NO: 942), NP_006106.1 (SEQ ID NO: 943), NP_996837.1 (SEQ ID NO: 944), NP_996836.1 (SEQ ID NO: 945), NP_996839.1 (SEQ ID NO: 946), NP_996838.1 (SEQ ID NO: 947), NP_001278644.1 (SEQ ID NO: 948), NP_001305055.1 (SEQ ID NO: 949), NP_001305056.1 (SEQ ID NO: 950), NP_001278648.1 (SEQ ID NO: 951), and NP_001278645.1 (SEQ ID NO: 952). The term “PRAME” includes recombinant PRAME or a fragment thereof. The term also encompasses PRAME or a fragment thereof coupled to, for example, histidine tag, mouse or human Fc, or a signal sequence such as ROR1. In certain embodiments, the term comprises PRAME, or a fragment thereof, in the context of HLA-A2, linked to HLA-A2 or as displayed by HLA-A2. As used herein, the numbering of certain PRAME amino acid residues within the full-length PRAME sequence is with respect to SEQ ID NO: 944.

The term “HLA” refers to the human leukocyte antigen (HLA) system or complex, which is a gene complex encoding the major histocompatibility complex (MHC) proteins in humans. These cell-surface proteins are responsible for the regulation of the immune system in humans. HLAS corresponding to MHC class I (A, B, and C) present peptides from inside the cell.

The term “HLA-A” refers to the group of human leukocyte antigens (HLA) that are coded for by the HLA-A locus. HLA-A is one of three major types of human MHC class I cell surface receptors. The receptor is a heterodimer, and is composed of a heavy a chain and smaller β chain. The α chain is encoded by a variant HLA-A gene, and the β chain (β2-microglobulin) is an invariant β2 microglobulin molecule.

The term “HLA-A2” (which may also be referred to as HLA-A2*01 or HLA-A*0201 or HLA-A*02:01) is one particular class I major histocompatibility complex (MHC) allele group at the HLA-A locus; the α chain is encoded by the HLA-A*02 gene and the β chain is encoded by the β2-microglobulin or B2M locus.

The term “specifically binds,” or “binds specifically to”, or the like, means that TCR forms a complex with an antigen that is relatively stable under physiologic conditions. Specific binding can be characterized by an equilibrium dissociation constant of at least about 1×10⁻⁶ M or less, for example, 1χ10⁻⁸ M or less (e.g., a smaller KD denotes a tighter binding). Methods for determining whether two molecules specifically bind are well known in the art and include, for example, equilibrium dialysis, surface plasmon resonance, and the like. As described herein, the TCRs of the invention bind specifically to an HLA-A2 presented cancer testis preferentially expressed antigen in melanoma (PRAME) peptide, e.g., a peptide comprising amino acid residues 312-320 or 425-433 of PRAME (e.g., of the full-length PRAME sequence of SEQ ID NO: 944).

The term “off-target peptide” refers to a peptide that differs by 1, 2, 3, 4, 5 or more amino acids from a target peptide (e.g., a PRAME 312-320 peptide or a PRAME 425-433 peptide). In certain embodiments, the term includes a peptide that differs by less than or equal to 3 amino acids than the target peptide. For example, for a 9-mer peptide, if 1, 2, or 3 amino acids are not identical to the target peptide, it is considered an “off-target” peptide. In certain embodiments, amino acid identity is expressed in terms of ‘degree of similarity’ (DoS). If 6 or more amino acids within a 9-mer peptide are identical, the DoS is 6. In certain embodiments, a peptide with DoS≤6 is considered an “off-target” peptide. The term “off-target” peptide also refers to a peptide that is similar to the target peptide based on sequence homology, is predicted to bind to HLA-A2 and is comprised in a protein that is expressed in essential, normal tissues. Accordingly, in some embodiments a TCR of the present disclosure can bind to an HLA-A2-presented PRAME peptide (e.g., a peptide comprising amino acid residues 312-320 or 425-433 of PRAME) with an affinity corresponding to a K_{D} value that is at least ten-fold lower than its affinity for binding to an off-target peptide.

The term “isolated” refers to a composition, compound, substance, or molecule altered by the hand of man from the natural state. For example, a composition or substance that occurs in nature is isolated if it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein. More particularly, an isolated TCR can refer to a TCR that has been removed from a cell, for example, a TCR that has been purified. TCRs can also be expressed by an isolated cell, e.g., a cell that has been isolated from an animal or a cell from cell culture. In this context, the isolated cell can express the TCR on its surface (i.e., the cell can “present” the TCR).

The term “recombinant”, as used herein, refers to TCRs of the invention created, expressed, isolated or obtained by technologies or methods known in the art as recombinant DNA technology which include, e.g., DNA splicing and transgenic expression. The term refers to TCRs expressed in a non-human mammal (including transgenic non-human mammals, e.g., transgenic mice), or a cell (e.g., CHO cells) expression system or isolated from a recombinant combinatorial human antibody library.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

The term “polypeptide” is meant to refer to any polymer preferably consisting essentially of any of the 20 natural amino acids regardless of its size. Although the term “protein” is often used in reference to relatively large proteins, and “peptide” is often used in reference to small polypeptides, use of these terms in the field often overlaps. The term “polypeptide” refers generally to proteins, polypeptides, and peptides unless otherwise noted. Peptides useful in accordance with the present disclosure in general will be generally between about 0.1 to 100 kDa or greater up to about 1000 kDa, preferably between about 0.1, 0.2, 0.5, 1, 2, 5, 10, 20, 30 and 50 kDa as judged by standard molecule sizing techniques such as centrifugation or SDS-polyacrylamide gel electrophoresis.

The term “vector” is a nucleic acid molecule that is able to replicate autonomously in a host cell and can accept foreign DNA. A vector carries its own origin of replication, one or more unique recognition sites for restriction endonucleases which can be used for the insertion of foreign DNA, and usually selectable markers such as genes coding for antibiotic resistance, and often recognition sequences (e.g., promoter) for the expression of the inserted DNA. Common vectors include plasmid vectors and phage vectors.

In some embodiments, TCRs of the invention may be conjugated to a moiety such as a ligand, a detectable moiety, or a therapeutic moiety (“immunoconjugate”), such as a cytotoxin, an anti-cancer drug, or any other therapeutic moiety useful for treating a disease or condition including PRAME-associated disease or disorder, such as a PRAME-associated cancer.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “KD”, also known as K_{D} or K_{d}, is intended to refer to the equilibrium dissociation constant of a particular biomolecule and its binding partner. KD measurements are particularly useful for assessing protein-protein interactions, e.g. as in an antigen-binding protein-antigen interaction. The smaller the value of the KD, the greater (or e.g. stronger) the binding interaction or affinity between the antigen-binding protein and antigen (e.g. target). The larger the value of the KD, the weaker the binding interaction or affinity between the antigen-binding protein and antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

Sequence identity can be calculated using an algorithm, for example, the Needleman Wunsch algorithm (Needleman and Wunsch 1970, J. Mol. Biol. 48:443-453) for global alignment, or the Smith Waterman algorithm (Smith and Waterman 1981, J. Mol. Biol. 147:195-197) for local alignment. Another preferred algorithm is described by Dufresne et al in Nature Biotechnology in 2002 (vol. 20, pp. 1269-71) and is used in the software GenePAST (GQ Life Sciences, Inc. Boston, MA).

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 96%, 97%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Sequences also can be compared using the Smith-Waterman homology search algorithm using an affine gap search with a gap open penalty of 12 and a gap extension penalty of 2, BLOSUM matrix of 62. Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

A “patient-derived TCR” is a TCR that is produced by isolating the alpha and beta chains of a PRAME reactive TCR isolated from the T-lymphocytes that mediated in vivo regression of a tumor in a subject having a PRAME-associated cancer.

An “affinity-matured TCR” is a TCR that is produced by mutagenesis and selection in vitro. For example, untargeted or targeted (e.g., oligonucleotide-directed) mutagenesis can be performed to introduce variation in TCR sequences, and the subsequent TCRs can then be screened for affinity against a target, e.g., by use of phage display.

The term “activates a T cell response having a signal to noise ratio stronger or equal to a patient-derived PRAME-specific TCR” or “activates a T cell response having a signal to noise ratio stronger or equal to an affinity-matured PRAME-specific TCR” is meant to refer to an increase, i.e., about 2-fold or more, an amplification, i.e., about 2-fold, an augmentation, i.e., about 2-fold, or a boost of a physiological activity, i.e., about 2-fold, i.e., T cell signaling, as measured by, for example, a luminescent bioassay. Reference to a greater T cell response, or a stronger T cell response or an activation signal, may be used interchangeably. Various measurements and assays of T cell response or T cell activation are well known to the skilled artisan.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding). The term “effective amount” is intended to encompass contexts such as a pharmaceutically effective amount or therapeutically effective amount. For example, in certain embodiments, the effective amount is capable of achieving a beneficial state, beneficial outcome, functional activity in a screening assay, or improvement of a clinical condition.

As described herein, a TCR of the invention may be “administered” to a subject. “Administering” a TCR of the invention includes, but is not limited to, administration of a cell expressing a TCR of the invention (e.g., an effector cell such as a T cell), administration of a nucleic acid expressing a TCR of the invention (e.g., a vector expressing such a TCR), and administration of a polypeptide comprising a TCR of the invention, wherein the polypeptide has been formatted for such administration (e.g., a bispecific polypeptide comprising a TCR chain and a CD3-binding antibody chain).

As used herein, the term “subject” refers to an animal, preferably a mammal, in need of amelioration, prevention and/or treatment of a PRAME-associated disease or disorder, such as a PRAME-associated cancer (e.g., a PRAME-positive cancer). The term includes human subjects who have or are at risk of having a PRAME-associated disease or disorder, such as a PRAME-associated cancer.

As used herein, “anti-cancer drug” means any agent useful to treat or ameliorate or inhibit cancer including, but not limited to, cytotoxins and agents such as antimetabolites, alkylating agents, anthracyclines, antibiotics, antimitotic agents, procarbazine, hydroxyurea, asparaginase, corticosteroids, cyclophosphamide, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “prevent”, “preventing”, “prevention”, “prophylactic treatment” and the like are meant to refer to reducing the probability of developing a disorder or condition in a subject, who does not have, but is at risk of or susceptible to developing a disorder or condition. Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

### II. PRAME T Cell (TCRs) and Compositions Comprising PRAME TCRS

T cells are a subgroup of cells which, together with other immune cell types (polymorphonuclear, eosinophils, basophils, mast cells, B-cells, NK cells), constitute the cellular component of the immune system. Under physiological conditions T cells function in immune surveillance and in the elimination of foreign antigen. However, under pathological conditions there is compelling evidence that T cells play a major role in the causation and propagation of disease. In these disorders, breakdown of T cell immunological tolerance, either central or peripheral is a fundamental process in the causation of autoimmune disease.

T cells bind epitopes on small antigenic determinants on the surface of antigen-presenting cells that are associated with a major histocompatibility complex (MHC; in mice) or human leukocyte antigen (HLA; in humans) complex. T cells bind these epitopes through a T cell receptor (TCR) complex on the surface of the T cell. T cell receptors are heterodimeric structures composed of two types of chains: an α (alpha) and β (beta) chain, or a γ (gamma) and δ (delta) chain. The α chain is encoded by the nucleic acid sequence located within the α locus (on human or mouse chromosome 14), which also encompasses the entire δ locus, and the β chain is encoded by the nucleic acid sequence located within the β locus (on mouse chromosome 6 or human chromosome 7). The majority of T cells have an αβ TCR; while a minority of T cells bears a γδ TCR.

T cell receptor α and β polypeptides (and similarly γ and δ polypeptides) are linked to each other via a disulfide bond. Each of the two polypeptides that make up the TCR contains an extracellular domain comprising constant and variable regions, a transmembrane domain, and a cytoplasmic tail (the transmembrane domain and the cytoplasmic tail also being a part of the constant region). The variable region of the TCR determines its antigen specificity, and similar to immunoglobulins, comprises three complementary determining regions (CDRs). The TCR is expressed on most T cells in the body and is known to be involved in the recognition of MHC-restricted antigens. The TCR α chain includes a covalently linked Vα and Cα region, whereas the β chain includes a Vβ region covalently linked to a CB region. The Vα and Vβ regions form a pocket or cleft that can bind an antigen in the context of a major histocompatibility complex (MHC) (or HLA in humans). TCRs are detection molecules with exquisite specificity, and exhibit, like antibodies, an enormous diversity.

The general structure of TCR molecules and methods of making and using, including binding to a peptide: Major Histocompatibility Complex have been disclosed. See, for example PCT/US98/04274; PCT/US98/20263; WO99/60120.

Non-human animals (e.g., rodents, e.g., mice or rats) can be genetically engineered to express a human or humanized T cell receptor (TCR) comprising a variable domain encoded by at least one human TCR variable region gene segment, as described in, for example, PCT Publication No. WO 2016/164492, the entire contents of which are hereby incorporated herein by reference. For example, the VelociT® mouse technology (Regeneron), a genetically modified mouse that allows for the production of fully human therapeutic TCRs against tumor and/or viral antigens, can be used to produce the TCRs of the invention. Those of skill in the art, through standard mutagenesis techniques, in conjunction with the assays described herein, can obtain altered TCR sequences and test them for particular binding affinity and/or specificity. Useful mutagenesis techniques known in the art include, without limitation, de novo gene synthesis, oligonucleotide-directed mutagenesis, region-specific mutagenesis, linker-scanning mutagenesis, and site-directed mutagenesis by PCR (see, e.g., Sambrook et al. (1989) and Ausubel et al. (1999)).

Briefly, in some embodiments, methods for generating a TCR to a PRAME 312-320 peptide or a PRAME 425-433 peptide may include immunizing a non-human animal (e.g., a rodent, e.g., a mouse or a rat), such as a genetically engineered non-human animal that comprises in its genome an un-rearranged human TCR variable gene locus, with a PRAME 312-320 peptide or a PRAME 425-433 peptide; allowing the animal to mount an immune response to the peptide; isolating from the animal a T cell reactive to the peptide; determining a nucleic acid sequence of a human TCR variable region expressed by the T cell; cloning the human TCR variable region into a nucleotide construct comprising a nucleic acid sequence of a human TCR constant region such that the human TCR variable region is operably linked to the human TCR constant region; and expressing from the construct a human T cell receptor specific for the PRAME 312-320 peptide or a PRAME 425-433 peptide, respectively. In some embodiments, the steps of isolating a T cell, determining a nucleic acid sequence of a human TCR variable region expressed by the T cell, cloning the human TCR variable region into a nucleotide construct comprising a nucleic acid sequence of a human TCR constant region, and expressing a human T cell receptor are performed using standard techniques known to those of skill the art.

In some embodiments, the nucleotide sequence encoding a T cell receptor specific for an antigen of interest is expressed in a cell. In some embodiments, the cell expressing the TCR is selected from a CHO, COS, 293, HeLa, PERC.6™ cell, etc.

In obtaining variant TCR coding sequences, those of ordinary skill in the art will recognize that TCR-derived proteins may be modified by certain amino acid substitutions, additions, deletions, and post-translational modifications, without loss or reduction of biological activity. In particular, it is well known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

In some embodiments, a TCR of the present disclosure can comprise a CDR sequence (e.g., a CDR3 sequence such as a Vα CDR3 or a Vβ CDR3) with 1 or more substitutions as compared to a CDR sequence (e.g., a CDR3 sequence such as a Vα CDR3 or a Vβ CDR3) of Table 5 or Table 8. For example, a TCR of the present disclosure can comprise a CDR sequence with 1, 2, 3, 4, 5, 6, 7, 8, 9, 10, or more substitutions as compared to a CDR sequence of Table 5 or Table 8. In general, the TCRs of the present invention function by binding to an HLA-A2 presented PRAME 312-320 peptide or a HLA-A2 presented PRAME 425-433 peptide. As used herein, an HLA presented peptide (such as an HLA-A2 presented peptide) can refer to a peptide that is bound to a human leukocyte antigen (HLA) protein, for example, an HLA protein expressed on the surface of a cell. Thus, a TCR that binds to an HLA presented peptide binds to the peptide that is bound by the HLA, and optionally also binds to the HLA itself. Interaction with the HLA can confer specificity for binding to a peptide presented by a particular HLA. In some embodiments, the TCR binds to an isolated HLA presented peptide. In some embodiments, the TCR binds to an HLA presented peptide on the surface of a cell.

In general, the TCRs of the present invention can function by binding to an HLA-A2-presented PRAME peptide (e.g., PRAME 312-320 or PRAME 425-433).

The present invention includes PRAME TCRs that bind a PRAME 312-320 peptide or a PRAME 425-433 peptide in the context of HLA-A2 with high specificity. In some embodiments, the PRAME TCRs do not bind to the PRAME 312-320 peptide or the PRAME 425-433 peptide in the absence of HLA-A2, or such binding is minimal. Further, in some embodiments, the PRAME TCRs do not bind to an off-target peptide in the context of HLA-A2, or such binding is minimal. As used herein, an off-target peptide can refer to a peptide that differs from a target peptide by 1, 2, 3, 4, 5, or more amino acids. In some embodiments, binding specificity can be determined by a) measuring on-target binding (e.g., binding to the HLA-A2 presented PRAME (312-320) peptide or the HLA-A2 presented PRAME (425-433) peptide), b) measuring off-target binding, and c) quantifying the difference between the two, e.g., by calculating a ratio. This ratio can be calculated, for example, by dividing the values obtained in a) and b). Measurement of on-target and off-target binding can be achieved, for example, by measuring % binding to a peptide/HLA tetramer reagent (e.g., a PRAME/HLA tetramer reagent), or by other techniques known in the art. In some embodiments, an on-target binding/off-target binding value (e.g., a value obtained by dividing the values obtained in a) and b) described above) of a TCR of the present disclosure can be greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 35, greater than 40, greater than 45, greater than 50, greater than 55, greater than 60, greater than 65, greater than 70, greater than 75, greater than 80, greater than 85, greater than 90, greater than 95, greater than 100, greater than 110, greater than 120, greater than 130, greater than 140, greater than 150, greater than 160, greater than 170, greater than 180, greater than 190, greater than 200, greater than 225, greater than 250, greater than 275, greater than 300, greater than 325, greater than 350, greater than 375, greater than 400, greater than 425, greater than 450, greater than 475, greater than 500, greater than 550, greater than 600, greater than 650, greater than 700, greater than 750, greater than 800, greater than 850, greater than 900, greater than 950, greater than 1000, greater than 1100, greater than 1200, greater than 1300, greater than 1400, greater than 1500, greater than 1600, greater than 1700, greater than 1800, greater than 1900, or greater than 2000. In some embodiments, an on-target binding/off-target binding value (e.g., a value obtained by dividing the values obtained in a) and b) described above) can be about 5 to about 20, about 10 to about 30, about 20 to about 80, about 30 to about 70, about 40 to about 60, about 50 to about 250, about 100 to about 200, about 100 to about 1000, about 300 to about 700, about 500 to about 1500, about 800 to about 1200, about 900 to about 1100, about 800 to about 1500, about 1000 to about 1400, or about 1100 to about 1300.

In some embodiments, the invention provides a recombinant antigen-binding protein (e.g., an isolated antigen-binding protein) that binds specifically to a conformational epitope of an HLA-A2 presented human PRAME (312-320) peptide or to a conformational epitope of an HLA-A2 presented human PRAME (425-433) peptide, wherein the antigen-binding protein has a property selected from the group consisting of: (a) binds monomeric HLA-A2: PRAME (312-320) peptide or monomeric PRAME (312-320) peptide with a binding dissociation equilibrium constant (K_{D}) of less than about 20 nM as measured in a surface plasmon resonance assay at 25° C.; (b) binds monomeric HLA-A2: PRAME (425-433) peptide or monomeric PRAME (425-433) peptide with a binding dissociation equilibrium constant (K_{D}) of less than about 25 nM as measured in a surface plasmon resonance assay at 25° C.; (c) binds to HLA-A2: PRAME (312-320) peptide-expressing cells or PRAME (425-433) peptide-expressing cells with an EC₅₀ less than about 6 nM and does not bind to cells expressing predicted off-target peptides as determined by luminescence assay; (d) binds to HLA-A2: PRAME (312-320) peptide-expressing cells or PRAME (425-433) peptide-expressing cells with an EC₅₀ less than about 1 nM and do not substantially bind to cells expressing predicted off-target peptides as determined by luminescence assay; (e) binds to HLA-A2: PRAME (312-320) peptide-expressing cells or PRAME (425-433) peptide-expressing cells with an EC₅₀ less than about 30 nM as determined by flow cytometry assay; (f) binds to HLA-A2: PRAME (312-320) peptide-expressing cells or PRAME (425-433) peptide-expressing cells with an EC₅₀ less than about 75 nM as determined by flow cytometry assay; and (g) the conformational epitope comprises one or more amino acids of SEQ ID NO: 944.

In some embodiments, the PRAME TCRs of the present disclosure have specific activity or affinity for PRAME (312-320) or for PRAME (425-433) as measured by an in vitro assay. For example, cells (such as T2 cells) expressing an HLA can be pulsed with a PRAME (312-320) or for PRAME (425-433) polypeptide, or an off-target polypeptide thereby inducing the cells to present the polypeptide bound to the HLA. Alternatively or in addition to using an off-target polypeptide as a control, an off-target HLA (an HLA other than the HLA that is recognized by the TCR of interest) can be used. For example, an off-target HLA can be used to present the PRAME peptide to test for specificity of binding to the HLA-A2-presented PRAME peptide. In addition, a control can be a cell line that expresses neither PRAME nor the target HLA (e.g., HLA-A2). Cells can be co-cultured with a T-cell population expressing the TCR of interest, and activity measured as a function of the amount of a cytokine (such as interferon gamma) produced by the cells. In certain embodiments, the assay can comprise in vitro co-cultures of a TCR-expressing T cell population with 10⁻¹⁰ M peptide-loaded T2 cells at an effector cell: target cell ratio of 1:1 (1×10⁵ effector cells/96 well), and interferon gamma measurement 24 hours after co-culture (e.g., by a Meso Scale Discovery (MSD®) Sector Imager). In certain embodiments, the assay can comprise in vitro co-cultures of a TCR-expressing T cell population and effector cell at an effector cell: target cell ratio of 5:1 (2.5×10⁵ effector cells: 5×10⁴ target cells), and interferon gamma measurement 24 hours after co-culture (e.g., by a Meso Scale Discovery (MSD®) Sector Imager).

Increasing amounts of cytokine detected can serve as an indicator of activity. The activity or specificity of a TCR of interest to its target peptide in comparison to a control (off-target) polypeptide, or the activity or specificity of a TCR of interest to its on-target HLA-bound target peptide in comparison to an off-target HLA-bound target peptide can be 2-fold or greater, 3-fold or greater, 4-fold or greater, 5-fold or greater, 6-fold or greater, 7-fold or greater, 8-fold or greater, 9-fold or greater, 10-fold or greater, 15-fold or greater, 20-fold or greater, 30-fold or greater, 40-fold or greater, 50-fold or greater, 100-fold or greater, 200-fold or greater, 300-fold or greater, 400-fold or greater, 500-fold or greater, 600-fold or greater, 700-fold or greater, 800-fold or greater, 900-fold or greater, 1,000-fold or greater, 1,500-fold or greater, 2,000-fold or greater, 2,500-fold or greater, 3,000-fold or greater, 4,000-fold or greater, 5,000-fold or greater, 10,000-fold or greater, 20,000-fold or greater, 30,000-fold or greater, 40,000-fold or greater, 50,000-fold or greater, 60,000-fold or greater, 70,000-fold or greater, 80,000-fold or greater, 90,000-fold or greater, or 100,000-fold or greater.

In certain embodiments, the PRAME TCRs of the present disclosure are useful in inhibiting the growth of a tumor or delaying the progression of cancer when administered prophylactically to a subject in need thereof and may increase survival of the subject. For example, the administration of a PRAME TCR of the present invention may lead to shrinking of a primary tumor and may prevent metastasis or development of secondary tumors. In certain embodiments, the PRAME TCRs of the present invention are useful in inhibiting the growth of a tumor when administered therapeutically to a subject in need thereof and may increase survival of the subject. For example, the administration of a therapeutically effective amount of a PRAME TCR of the invention to a subject may lead to shrinking and disappearance of an established tumor in the subject.

In some embodiments, the invention provides a TCR (e.g., an isolated TCR or a TCR expressed on an isolated cell) that specifically binds to an HLA-A2 presented PRAME 312-320 peptide, wherein the antigen-binding protein exhibits one or more of the following characteristics: (i) comprises an alpha chain variable domain comprising a complementary determining region (CDR) 3, wherein the CDR3 comprises the amino acid sequence of any one of the alpha chain variable domain CDR3 amino acid sequences set forth in Table 3; (ii) comprises a beta chain variable domain comprising a complementary determining region (CDR) 3, wherein the CDR3 comprises the amino acid sequence of any one of the beta chain variable domain CDR3 amino acid sequences set forth in Table 3; (iii) comprises a CDR1 of the alpha chain variable domain comprising any one of the CDR1 amino acid sequences set forth in Table 3, or a substantially similar sequence thereof having at least 90%, at least 95%, at least 96%, at least 97%, at least 98% or at least 99% sequence identity, and a CDR2 of the alpha chain variable domain independently comprising any one of the CDR2 amino acid sequences set forth in Table 3, or a substantially similar sequence thereof having at least 90%, at least 95%, at least 96%, at least 97%, at least 98% or at least 99% sequence identity; (iv) comprises a CDR1 of a beta chain variable domain comprising any one of the CDR1 amino acid sequences set forth in Table 3, or a substantially similar sequence thereof having at least 90%, at least 95%, at least 96%, at least 97%, at least 98% or at least 99% sequence identity, and a CDR2 of a beta chain variable domain independently comprising any one of the CDR2 amino acid sequences set forth in Table 3, or a substantially similar sequence thereof having at least 90%, at least 95%, at least 96%, at least 97%, at least 98% or at least 99% sequence identity; (v) comprises an alpha chain variable domain CDR1, CDR2 and CDR3 contained within any one of the alpha chain variable domain sequences listed in Table 5, or a substantially similar sequence thereof having at least 90%, at least 95%, at least 96%, at least 97%, at least 98% or at least 99% sequence identity; and beta chain variable domain CDR1, CDR2 and CDR3 contained within any one of the beta chain variable domain sequences listed in Table 5, or a substantially similar sequence thereof having at least 90%, at least 95%, at least 96%, at least 97%, at least 98% or at least 99% sequence identity; (vi) comprises an alpha chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the alpha chain variable domain amino acid sequences listed in Table 5; (vii) comprises a beta chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the beta chain variable domain amino acid sequences listed in Table 5; (viii) comprises (a) an alpha chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the alpha chain variable domain amino acid sequences listed in Table 5; and (b) a beta chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the beta chain variable domain amino acid sequences listed in Table 5; (ix) comprises (a) an alpha chain variable domain CDR1 domain having an amino acid sequence selected from the group consisting of SEQ ID NOs: 1, 7, 13, 19, 25, 31, 37, 43, 49, 55, 61, 67, 73, 79, 85, 91, 97, and 103, or a substantially similar sequence thereof having at least 90%, at least 95%, at least 96%, at least 97%, at least 98% or at least 99% sequence identity; (b) an alpha chain variable domain CDR2 domain having an amino acid sequence selected from the group consisting of SEQ ID NOs: 2, 8, 14, 20, 26, 32, 38, 44, 50, 56, 62, 68, 74, 80, 86, 92, 98, and 104, or a substantially similar sequence thereof having at least 90%, at least 95%, at least 96%, at least 97%, at least 98% or at least 99% sequence identity; (c) an alpha chain variable domain CDR3 domain having an amino acid sequence selected from the group consisting of SEQ ID NOs: 3, 9, 15, 21, 27, 33, 39, 45, 51, 57, 63, 69, 75, 81, 87, 93, 99, and 105, or a substantially similar sequence thereof having at least 90%, at least 95%, at least 96%, at least 97%, at least 98% or at least 99% sequence identity; (d) a beta chain variable domain CDR1 having an amino acid sequence selected from the group consisting of SEQ ID NOs: 4, 10, 16, 22, 28, 34, 40, 46, 52, 58, 64, 70, 76, 82, 88, 94, 100, and 106, or a substantially similar sequence thereof having at least 90%, at least 95%, at least 96%, at least 97%, at least 98% or at least 99% sequence identity; (e) a beta chain variable domain CDR2 having an amino acid sequence selected from the group consisting of SEQ ID NOs: 5, 11, 17, 23, 29, 35, 41, 47, 53, 59, 65, 71, 77, 83, 89, 95, 101, and 107, or a substantially similar sequence thereof having at least 90%, at least 95%, at least 96%, at least 97%, at least 98% or at least 99% sequence identity; and (f) a beta chain variable domain CDR3 having an amino acid sequence selected from the group consisting of SEQ ID NOs: 6, 12, 18, 24, 30, 36, 42, 48, 54, 60, 66, 72, 78, 84, 90, 96, 102, and 108, or a substantially similar sequence thereof having at least 90%, at least 95%, at least 96%, at least 97%, at least 98% or at least 99% sequence identity; (x) comprises an alpha chain variable domain/beta chain variable domain amino acid sequence pair selected from the group consisting of SEQ ID NOs: 217/219, 229/231, 237/239, 241/243, and 285/287, or a substantially similar sequence thereof having at least 90%, at least 95%, at least 96%, at least 97%, at least 98% or at least 99% sequence identity; (xi) comprises an alpha chain variable domain/beta chain variable domain amino acid sequence pair selected from the group consisting of SEQ ID NOs: 217/219, 221/223, 225/227, 229/231, 233/235, 237/239, 241/243, 245/247, 249/251, 253/255, 257/259, 261/263, 265/267, 269/271, 273/275, 277/279, 281/283, and 285/287, or a substantially similar sequence thereof having at least 90%, at least 95%, at least 96%, at least 97%, at least 98% or at least 99% sequence identity; (xii) does not specifically bind to cells expressing predicted off-target peptides but not an HLA-A2 presented PRAME 312-320 peptide, as determined by a luminenscence assay; and/or (xiii) activates a T cell response about two times greater than a patient-derived PRAME-specific TCR, e.g., activates a T cell response about two times greater, or about three times greater, or about four times greater than a patient-derived PRAME-specific TCR as determined by a TCR-mediated T cell signaling luminescent bioassay.

In some embodiments, the invention provides a TCR (e.g., an isolated TCR or a TCR expressed on an isolated cell) that specifically binds to an HLA-A2 presented PRAME 425-433 peptide, wherein the antigen-binding protein exhibits one or more of the following characteristics: (i) comprises an alpha chain variable domain comprising a complementary determining region (CDR) 3, wherein the CDR3 comprises the amino acid sequence of any one of the alpha chain variable domain CDR3 amino acid sequences set forth in Table 6; (ii) comprises a beta chain variable domain comprising a complementary determining region (CDR) 3, wherein the CDR3 comprises the amino acid sequence of any one of the beta chain variable domain CDR3 amino acid sequences set forth in Table 6; (iii) comprises a CDR1 of the alpha chain variable domain comprising any one of the CDR1 amino acid sequences set forth in Table 6, or a substantially similar sequence thereof having at least 90%, at least 95%, at least 96%, at least 97%, at least 98% or at least 99% sequence identity, and a CDR2 of the alpha chain variable domain independently comprising any one of the CDR2 amino acid sequences set forth in Table 6, or a substantially similar sequence thereof having at least 90%, at least 95%, at least 96%, at least 97%, at least 98% or at least 99% sequence identity; (iv) comprises a CDR1 of a beta chain variable domain comprising any one of the CDR1 amino acid sequences set forth in Table 6, or a substantially similar sequence thereof having at least 90%, at least 95%, at least 96%, at least 97%, at least 98% or at least 99% sequence identity, and a CDR2 of a beta chain variable domain independently comprising any one of the CDR2 amino acid sequences set forth in Table 6, or a substantially similar sequence thereof having at least 90%, at least 95%, at least 96%, at least 97%, at least 98% or at least 99% sequence identity; (v) comprises an alpha chain variable domain CDR1, CDR2 and CDR3 contained within any one of the alpha chain variable domain sequences listed in Table 8, or a substantially similar sequence thereof having at least 90%, at least 95%, at least 96%, at least 97%, at least 98% or at least 99% sequence identity; and beta chain variable domain CDR1, CDR2 and CDR3 contained within any one of the beta chain variable domain sequences listed in Table 8, or a substantially similar sequence thereof having at least 90%, at least 95%, at least 96%, at least 97%, at least 98% or at least 99% sequence identity; (vi) comprises an alpha chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the alpha chain variable domain amino acid sequences listed in Table 8; (vii) comprises a beta chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the beta chain variable domain amino acid sequences listed in Table 8; (viii) comprises (a) an alpha chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the alpha chain variable domain amino acid sequences listed in Table 8; and (b) a beta chain variable domain having an amino acid sequence that has at least 85%, 86%, 87%, 88%, 89%, 90%, 91%, 92%, 93%, 94%, 95%, 96%, 97%, 98%, 99% or about 100% amino acid identity to the entire amino acid sequence of any one of the amino acid sequences of the beta chain variable domain amino acid sequences listed in Table 8; (ix) comprises (a) an alpha chain variable domain CDR1 domain having an amino acid sequence selected from the group consisting of SEQ ID NOs: 289, 295, 301, 307, 313, 319, 325, 331, 337, 343, 349, 355, 361, 367, 373, 379, 385, 391, 397, 403, 409, 415, 421, 427, 433, 439, 445, 451, 457, 463, 469, 475, 481, 487, 493, 499, 505, 511, 517, and 523, or a substantially similar sequence thereof having at least 90%, at least 95%, at least 96%, at least 97%, at least 98% or at least 99% sequence identity; (b) an alpha chain variable domain CDR2 domain having an amino acid sequence selected from the group consisting of SEQ ID NOs: 290, 296, 302, 308, 314, 320, 326, 332, 338, 344, 350, 356, 362, 368, 374, 380, 386, 392, 398, 404, 410, 416, 422, 428, 434, 440, 446, 452, 458, 464, 470, 476, 482, 488, 494, 500, 506, 512, 518, and 524, or a substantially similar sequence thereof having at least 90%, at least 95%, at least 96%, at least 97%, at least 98% or at least 99% sequence identity; (c) an alpha chain variable domain CDR3 domain having an amino acid sequence selected from the group consisting of SEQ ID NOs: 291, 297, 303, 309, 315, 321, 327, 333, 339, 345, 351, 357, 363, 369, 375, 381, 387, 393, 399, 405, 411, 417, 423, 429, 435, 441, 447, 453, 459, 465, 471, 477, 483, 489, 495, 501, 507, 513, 519, and 525, or a substantially similar sequence thereof having at least 90%, at least 95%, at least 96%, at least 97%, at least 98% or at least 99% sequence identity; (d) a beta chain variable domain CDR1 having an amino acid sequence selected from the group consisting of SEQ ID NOs: 292, 298, 304, 310, 316, 322, 328, 334, 340, 346, 352, 358, 364, 370, 376, 382, 388, 394, 400, 406, 412, 418, 424, 430, 436, 442, 448, 454, 460, 466, 472, 478, 484, 490, 496, 502, 508, 514, 520, and 526, or a substantially similar sequence thereof having at least 90%, at least 95%, at least 96%, at least 97%, at least 98% or at least 99% sequence identity; (e) a beta chain variable domain CDR2 having an amino acid sequence selected from the group consisting of SEQ ID NOs: 293, 299, 305, 311, 317, 323, 329, 335, 341, 347, 353, 359, 365, 371, 377, 383, 389, 395, 401, 407, 413, 419, 425, 431, 437, 443, 449, 455, 461, 467, 473, 479, 485, 491, 497, 503, 509, 515, 521, and 527, or a substantially similar sequence thereof having at least 90%, at least 95%, at least 96%, at least 97%, at least 98% or at least 99% sequence identity; and (f) a beta chain variable domain CDR3 having an amino acid sequence selected from the group consisting of SEQ ID NOs: 294, 300, 306, 312, 318, 324, 330, 336, 342, 348, 354, 360, 366, 372, 378, 384, 390, 396, 402, 408, 414, 420, 426, 432, 438, 444, 450, 456, 462, 468, 474, 480, 486, 492, 498, 504, 510, 516, 522, and 528, or a substantially similar sequence thereof having at least 90%, at least 95%, at least 96%, at least 97%, at least 98% or at least 99% sequence identity; (x) comprises an alpha chain variable domain/beta chain variable domain amino acid sequence pair selected from the group consisting of SEQ ID NOs: 825/827, 845/847, 853/855, 857/859, 865/867, 873/875, 885/887, 893/805, 897/899, 901/903, 913/915, and 925/927, or a substantially similar sequence thereof having at least 90%, at least 95%, at least 96%, at least 97%, at least 98% or at least 99% sequence identity; (xi) comprises an alpha chain variable domain/beta chain variable domain amino acid sequence pair selected from the group consisting of SEQ ID NOs: 769/771, 773/775, 777/779, 781/783, 785/787, 789/791, 793/795, 797/799, 801/803, 805/807, 809/811, 813/815, 817/819, 821/823, 825/827, 829/831, 833/835, 837/839, 841/843, 845/847, 849/851, 853/855, 857/859, 861/863, 865/867, 869/871, 873/875, 877/879, 881/883, 885/887, 889/891, 893/805, 897/899, 901/903, 905/907, 909/911, 913/915, 917/919, 921/923, and 925/927, or a substantially similar sequence thereof having at least 90%, at least 95%, at least 96%, at least 97%, at least 98% or at least 99% sequence identity; (xii) does not specifically bind to cells expressing predicted off-target peptides but not an HLA-A2 presented PRAME 425-433 peptide, as determined by a luminenscence assay; and/or (xiii) activates a T cell response about two times greater than a patient-derived PRAME-specific TCR, e.g., activates a T cell response about two times greater, or about three times greater, or about four times greater than a patient-derived PRAME-specific TCR as determined by a TCR-mediated T cell signaling luminescent bioassay.

The TCRs of the present invention may possess one or more of the aforementioned biological characteristics, or any combinations thereof. Other biological characteristics of the antigen-binding proteins of the present invention will be evident to a person of ordinary skill in the art from a review of the present disclosure including the working Examples herein.

In certain embodiments, a polynucleotide encoding a PRAME TCR described herein is inserted into a vector. The term “vector” as used herein refers to a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF1α promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as 6x-histidine (SEQ ID NO: 954), c-Myc, and FLAG tags which are incorporated into the expressed TCRs. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the TCRs disclosed herein can be ligated into such expression vectors for the expression of the chimeric protein in mammalian cells.

In certain embodiments, the nucleic acids encoding the TCR of the present invention are provided in a viral vector. A viral vector can be those derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for a TCR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immnodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72:8463-8471 and Zufferey et al., J. Virol. 1998; 72:9873-9880).

Retroviral vectors (i.e., both lentiviral and non-lentiviral) for use in the present invention can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of a PRAME TCR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1α promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vectors provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The TCRs of the present invention are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or a may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a PRAME TCR of the invention carried by a retroviral vector can be transduced into a cell through infection and pro virus integration.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In particular, the TCRs of the present invention are introduced and expressed in immune effector cells so as to redirect their specificity to a target antigen of interest, e.g., an HLA-A2 displayed PRAME peptide, e.g., amino acid residues 312-320 or 425-433 of PRAME.

The present invention provides methods for making the immune effector cells which express the TCRs as described herein. In some embodiments, the method comprises transfecting or transducing immune effector cells, e.g., immune effector cells isolated from a subject, such as a subject having a PRAME-associated disease or disorder, such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells.

T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL separation. In some embodiments, cells from the circulating blood of an individual are obtained by apheresis. The apheresis product typically contains lymphocytes, including T cells, monocytes, granulocyte, B cells, other nucleated white blood cells, red blood cells, and platelets. In some embodiments, the cells collected by apheresis may be washed to remove the plasma fraction and to place the cells in an appropriate buffer or media for subsequent processing. In some embodiments of the invention, the cells are washed with PBS. In alternative embodiments, the washed solution lacks calcium and may lack magnesium or may lack many if not all divalent cations. As would be appreciated by those of ordinary skill in the art, a washing step may be accomplished by methods known to those in the art, such as by using a semiautomated flow-through centrifuge. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.

PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In some embodiments, the immune effector cells, such as T cells, are genetically modified with the chimeric antigen receptors described herein (e.g., transduced with a viral vector comprising a nucleic acid encoding a TCR) and then are activated and expanded in vitro. Methods for activating and expanding T cells are known in the art and are described, for example, in U.S. Pat. Nos. 6,905,874; 6,867,041; 6,797,514; WO2012079000, US 2016/0175358.

The invention provides a population of modified immune effector cells for the treatment of a PRAME-associated disease or disorder, e.g., cancer, the modified immune effector cells comprising a PRAME TCR as disclosed herein.

TCR-expressing immune effector cells prepared as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

### III. Pharmaceutical Compositions

The invention provides therapeutic compositions comprising the PRAME TCRs of the invention or immune effector cells comprising the PRAME TCRs of the invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Depending on the severity of the condition, the frequency and the duration of the treatment can be adjusted.

In certain embodiments, the initial dose may be followed by administration of a second or a plurality of subsequent doses of PRAME TCRs of the invention or immune effector cells comprising the PRAME TCRs of the invention in an amount that can be approximately the same or less than that of the initial dose,

In certain situations, the pharmaceutical composition can be delivered in a controlled release system. In some embodiments, a pump may be used.

Injectable preparations may include dosage forms for intravenous, subcutaneous, intracutaneous, intracranial, intraperitoneal and intramuscular injections, drip infusions, etc. The TCRs, pharmaceutical compositions, and cells described herein can be administered via parenteral administration. The preparations of the present disclosure may be prepared by methods publicly known. For example, the preparations may be prepared, e.g., by dissolving, suspending or emulsifying the antigen-binding protein or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

In some embodiments, TCR-expressing immune effector cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective number of cells in the composition is typically greater than 10² cells, and up to 10⁶ up to and including 10⁸ or 10⁹ cells and can be more than 10¹⁰ cells. The number of cells will depend upon the ultimate use for which the composition is intended as will the type of cells included therein.

The cells may be autologous or heterologous to the patient undergoing therapy. If desired, the treatment may also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) as described herein to enhance induction of the immune response.

The TCR expressing immune effector cell populations of the present invention may be administered either alone, or as a pharmaceutical composition in combination with diluents and/or with other components such as IL-2 or other cytokines or cell populations. Briefly, pharmaceutical compositions of the present invention may comprise a TCR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

### IV. Therapeutic Uses of PRAME TCRs or Immune Effector Cells Comprising PRAME TCRs

The anti-tumor immune response induced in a subject by administering TCR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present invention, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

Thus, the PRAME TCRs of the invention are useful, inter alia, for the treatment, prevention and/or amelioration of any disease or disorder associated with or mediated by PRAME. For example, the present invention provides methods for treating a PRAME-associated disease or disorder, such as a PRAME-associated cancer (e.g., a PRAME-positive cancer) (tumor growth inhibition) by administering a PRAME TCR (or pharmaceutical composition comprising a PRAME TCR or a plurality of cells comprising a PRAME TCR) as described herein to a patient in need of such treatment, and PRAME TCRs (or pharmaceutical composition comprising a PRAME TCR) for use in the treatment of a PRAME-associated cancer. The antigen-binding proteins of the present invention are useful for the treatment, prevention, and/or amelioration of disease or disorder or condition such as a PRAME-associated cancer and/or for ameliorating at least one symptom associated with such disease, disorder or condition. In the context of the methods of treatment described herein, the PRAME TCR (or pharmaceutical composition or plurality of cells) may be administered as a monotherapy (i.e., as the only therapeutic agent) or in combination with one or more additional therapeutic agents (examples of which are described elsewhere herein).

Accordingly, the present invention provides for methods of treating an individual diagnosed with or suspected of having, or at risk of developing, a PRAME-associated disease or disorder, e.g., a PRAME-associated cancer, comprising administering the individual a therapeutically effective amount of the TCR-expressing immune effector cells as described herein.

In some embodiments, the invention provides a method of treating a subject diagnosed with a PRAME-positive cancer comprising removing immune effector cells from a subject diagnosed with a PRAME-positive cancer, genetically modifying said immune effector cells with a vector comprising a nucleic acid encoding a TCR of the instant invention, thereby producing a population of modified immune effector cells, and administering the population of modified immune effector cells to the same subject. In some embodiments, the immune effector cells comprise T cells.

The methods for administering the cell compositions described herein includes any method which is effective to result in reintroduction of ex vivo genetically modified immune effector cells that either directly express a TCR of the invention in the subject or on reintroduction of the genetically modified progenitors of immune effector cells that on introduction into a subject differentiate into mature immune effector cells that express the TCR. One method comprises transducing peripheral blood T cells ex vivo with a nucleic acid construct in accordance with the invention and returning the transduced cells into the subject.

In some embodiments of the invention, the compositions described herein are useful for treating subjects suffering from primary or recurrent cancer, including, but not limited to, PRAME-associated cancer, e.g., PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, or a recurrent non-small cell lung cancer. In some embodiments, the PRAME-associated cancer is an ovarian cancer, a melanoma, a non-small cell lung carcinoma, a hepatocellular carcinoma, a colorectal carcinoma, an esophageal squamous cell carcinoma, an esophageal adenocarcinoma, a stomach cancer, a bladder cancer, a head and neck cancer, a gastric cancer, a synovial sarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a myxoid round cell liposarcoma.

The TCRs may be used to treat early stage or late-stage symptoms of the PRAME-associated cancer. In some embodiments, a TCR of the invention may be used to treat advanced or metastatic cancer. The TCRs are useful in reducing or inhibiting or shrinking tumor growth. In certain embodiments, treatment with a TCR of the invention leads to more than 40% regression, more than 50% regression, more than 60% regression, more than 70% regression, more than 80% regression or more than 90% regression of a tumor in a subject. In certain embodiments, the TCRs may be used to prevent relapse of a tumor. In certain embodiments, the TCRs are useful in extending progression-free survival or overall survival in a subject with PRAME-associated cancer. In some embodiments, the TCRs are useful in reducing toxicity due to chemotherapy or radiotherapy while maintaining long-term survival in a patient suffering from PRAME-associated cancer.

One or more TCRs of the present invention may be administered to relieve or prevent or decrease the severity of one or more of the symptoms or conditions of the disease or disorder.

It is also contemplated herein to use one or more TCRs of the present invention prophylactically to patients at risk for developing a disease or disorder such as PRAME-associated disease or disorder, such as a PRAME-associated cancer.

In further embodiments of the invention, the present TCRs are used for the preparation of a pharmaceutical composition for treating patients suffering from PRAME-associated disease or disorder, such as a PRAME-associated cancer. In some embodiments of the invention, the present TCRs are used as adjunct therapy with any other agent or any other therapy known to those skilled in the art useful for treating PRAME-associated cancer.

Combination therapies may include a PRAME TCR of the invention, such as immune effector cell comprising a TCR of the invention, or a pharmaceutical composition of the invention, and any additional therapeutic agent that may be advantageously combined with a TCR of the invention. The TCRs of the present invention may be combined synergistically with one or more anti-cancer drugs or therapy used to treat or inhibit a PRAME-associated disease or disorder, such as PRAME-positive cancer, e.g., a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

It is contemplated herein to use the TCRs of the invention in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more TCRs of the present invention may be used in combination with a PD-1 inhibitor (e.g., an anti-PD-1 antibody such as nivolumab, pembrolizumab, pidilizumab, BGB-A317 or REGN2810), a PD-L1 inhibitor (e.g., an anti-PD-L1 antibody such as avelumab, atezolizumab, durvalumab, MDX-1105, or REGN3504), a CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an NY-ESO-1 inhibitor (e.g., an anti-NY-ESO-1 antibody), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a costimulatory agent, a bispecific antibody (e.g., CD3xCD20 bispecific antibody, a PSMAxCD3 bispecific antibody, or a bispecific antibody that acts as a costimulatory agent, such as a bispecific antibody that binds a tumor antigen and has costimulatory activity), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, surgery, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any other therapy care to treat cancer. In certain embodiments, the TCRs of the present invention may be used in combination with cancer vaccines including dendritic cell vaccines, oncolytic viruses, tumor cell vaccines, etc. to augment the anti-tumor response.

Examples of cancer vaccines that can be used in combination with TCRs of the present invention include MAGE3 vaccine for melanoma and bladder cancer, MUC1 vaccine for breast cancer, EGFRv3 (e.g., Rindopepimut) for brain cancer (including glioblastoma multiforme), ALVAC-CEA (for CEA+ cancers), and NY-ESO-1 vaccine (e.g., for melanoma).

In certain embodiments, the PRAME TCRs of the invention may be administered in combination with radiation therapy in methods to generate long-term durable anti-tumor responses and/or enhance survival of patients with cancer. In some embodiments, the PRAME TCRs of the invention may be administered prior to, concomitantly or after administering radiation therapy to a cancer patient. For example, radiation therapy may be administered in one or more doses to tumor lesions followed by administration of one or more doses of PRAME TCRs of the invention. In some embodiments, radiation therapy may be administered locally to a tumor lesion to enhance the local immunogenicity of a patient's tumor (adjuvinating radiation) and/or to kill tumor cells (ablative radiation) followed by systemic administration of a PRAME TCRs of the invention.

The additional therapeutically active agent(s)/component(s) may be administered prior to, concurrent with, or after the administration of the PRAME TCRs of the present invention. For purposes of the present disclosure, such administration regimens are considered the administration of a PRAME TCR “in combination with” a second therapeutically active component.

The additional therapeutically active component(s) may be administered to a subject prior to administration of a PRAME TCR of the present invention. In other embodiments, the additional therapeutically active component(s) may be administered to a subject after administration of a PRAME TCR of the present invention. In yet other embodiments, the additional therapeutically active component(s) may be administered to a subject concurrent with administration of a PRAME TCR of the present invention. “Concurrent” administration, for purposes of the present invention, includes, e.g., administration of a PRAME TCR and an additional therapeutically active component to a subject in a single dosage form (e.g., co-formulated), or in separate dosage forms administered to the subject within about 30 minutes or less of each other. If administered in separate dosage forms, each dosage form may be administered via the same route; alternatively, each dosage form may be administered via a different route. In any event, administering the components in a single dosage from, in separate dosage forms by the same route, or in separate dosage forms by different routes are all considered “concurrent administration,” for purposes of the present disclosure. For purposes of the present disclosure, administration of a PRAME TCR “prior to”, “concurrent with,” or “after” (as those terms are defined herein above) administration of an additional therapeutically active component is considered administration of a PRAME TCR “in combination with” an additional therapeutically active component).

The present invention is further illustrated by the following Examples, which are not intended to be limiting in any way. The entire contents of all references, patents and published patent applications cited throughout this application are hereby incorporated herein by reference.

### EXAMPLES

### Example 1. Identification of PRAME Specific T Cell Receptors

Mice humanized for cellular immune system components, VelociT mice (see, e.g., PCT Publication No. WO 2016/164492, the entire contents of which are incorporated herein by reference), were immunized with either a PRAME (312-320) peptide (RLDQLLRHV); SEQ ID NO: 929) or a PRAME (425-433) peptide (SLLQHLIGL); SEQ ID NO:930) presented specifically by human HLA-A2, diluted in PBS and mixed with adjuvant, e.g. in equal volume with Complete Freund's Adjuvant (CFA; Chondrex, Inc.). Spleen suspensions from mice immunized with wither peptide were separately obtained and dissociated. Red blood cells were lysed in ACK lysis buffer (Life Technologies), and splenocytes were suspended in RPMI complete media. Isolated splenocytes were sorted and single T cells that bind the PRAME peptide used for immunization (either PRAME (312-320) or PRAME (425-433) in the context of MHC were isolated by fluorescent-activated cell sorting (FACS). Isolated T cells were single well plated and mixed with TCR alpha and beta variable region-specific PCR primers. cDNAs for each single T cell were synthesized via a reverse transcriptase (RT) reaction. Each resulting RT product was then split and transferred into two corresponding wells for subsequent TCR beta and alpha PCRs. One set of the resulting RT products was first amplified by PCR using a 5′ degenerate primer specific for TCR beta variable region leader sequence or a 5′ degenerate primer specific for TCR alpha chain variable region leader sequence and a 3′ primer specific for TCR constant region, to form an amplicon. The amplicons were then amplified again by PCR using a 5′ degenerate primer specific for TCR beta variable region framework 1 or a 5′ degenerate primer specific for TCR alpha chain variable region framework 1 and a 3′ primer specific for TCR constant region, to generate amplicons for cloning. The TCR beta and alpha derived PCR products were cloned into expression vectors containing beta constant region and alpha constant region, respectively. Expression vectors expressing full-length beta and alpha chain pairs were transfected into CHO cells and tested for binding to commercial PRAME/HLA tetramer reagent. CHO cells were incubated with soluble HLA-A2: PRAME (312-320) or HLA: A2: PRAME (425-433) (MBL International, Woburn, MA) tetramer and an antibody specific for mouse TCR constant region (clone H57-597) (Biolegend, San Diego, CA). Samples were then analyzed on an LSRFortessa X-20 (BD Biosciences, San Jose, CA). To calculate percentage of tetramer positive cells, antigen positive (Ag+) gates were set based on a negative control TCR that does not bind to the HLA-A2: PRAME (312-320) or HLA:A2: PRAME (425-433) (MBL International, Woburn, MA) tetramer using FlowJo (LLC, Ashland, OR). All Ag+ TCRs had a FlowJo criteria of ≥1% of cells in Ag+ gate with the mean fluorescence intensity (MFI)≥1000. Ag+ TCRs were determined by Next Generation Sequencing. The total number of TCRs that were identified with PRAME (312-320) and that express identical TCR alpha and beta nucleotide sequences are shown in Table 1 below. Cell frequency, or % tetramer positive cells in the Ag+ gate, is representative of the TCR shown in the first column of Table 1. The total number of TCRs that were identified with PRAME (425-433) and that express identical TCR alpha and beta nucleotide sequences are shown in Table 2 below. Cell frequency, or % tetramer positive cells in the Ag+ gate, is representative of the TCR shown in the first column of Table 2.

A detailed list of the beta chain variable domain CDR1, CDR2, and CDR3 amino acid sequences, and the alpha chain variable domain CDR1, CDR2, and CDR3 amino acid sequences of the TCRs that were identified with PRAME (312-320) as described above are provided in Table 3. A detailed list of the beta chain variable domain CDR1, CDR2, and CDR3 polynucleic acid sequences, and the alpha chain variable domain CDR1, CDR2, and CDR3 polynucleic acid sequences of the TCRs that were identified with PRAME (312-320) as described above are provided in Table 4. Table 5 provides the amino acid and nucleotide sequences of the beta chain variable and alpha chain variable regions of the TCRs that were identified with PRAME (312-320).

A detailed list of the beta chain variable domain CDR1, CDR2, and CDR3 amino acid sequences, and the alpha chain variable domain CDR1, CDR2, and CDR3 amino acid sequences of the TCRs that were identified with PRAME (425-433) as described above are provided in Table 6. A detailed list of the beta chain variable domain CDR1, CDR2, and CDR3 polynucleic acid sequences, and the alpha chain variable domain CDR1, CDR2, and CDR3 polynucleic acid sequences of the TCRs that were identified with PRAME (425-433) as described above are provided in Table 7. Table 8 provides the amino acid and nucleotide sequences of the beta chain variable and alpha chain variable regions of the TCRs that were identified with PRAME (425-433).

Table 9 provides the TCR gene families for the alpha and beta variable and joining regions of the isolated TCRs that were identified with PRAME (312-320) and Table 11 provides the amino acid and polynucleic acid sequence identifiers for alpha and beta variable chains and CDRs of the TCRs that were identified with PRAME (312-320).

Table 10 provides the TCR gene families for the alpha and beta variable and joining regions of the isolated TCRs that were identified with PRAME (425-433) and Table 12 provides the amino acid and polynucleic acid sequence identifiers for alpha and beta variable chains and CDRs of the TCRs that were identified with PRAME (425-433).

TABLE 1

| | | Cell |
|---|---|---|
| | Total Ag+ | Frequency ≥1000 |
| TCR ID | TCRs | MFI, ≥1% |
| PN46909 | 21 | 52.60 |
| PN46889 | 21 | 92.50 |
| PN46733 | 2 | 87.90 |
| PN46723 | 52 | 72.50 |
| PN46714 | 1 | 62.10 |
| PN46735 | 1 | 17.90 |
| PN46678 | 1 | 11.00 |
| PN46884 | 3 | 59.00 |
| PN46914 | 1 | 11.50 |
| PN46883 | 13 | 64.70 |
| PN46857 | 1 | 53.90 |
| PN46880 | 5 | 31.90 |
| PN46871 | 2 | 44.70 |
| PN46853 | 2 | 35.40 |
| PN46731 | 1 | 15.70 |
| PN46777 | 99 | 98.90 |
| PN46797 | 1 | 57.30 |
| PN46738 | 1 | 23.00 |

TABLE 2

| | | Cell |
|---|---|---|
| | Total Ag+ | Frequency ≥1000 |
| TCR ID | TCRs | MFI, ≥1% |
| PN42365 | 96 | 95.80 |
| PN42879 | 1 | 93.10 |
| PN42774 | 3 | 91.60 |
| PN42498 | 30 | 90.30 |
| PN42558 | 1 | 85.70 |
| PN42386 | 1 | 82.20 |
| PN42378 | 2 | 77.50 |
| PN42776 | 1 | 60.20 |
| PN42455 | 1 | 53.00 |
| PN42840 | 21 | 52.50 |
| PN42795 | 14 | 49.40 |
| PN42870 | 8 | 7.27 |
| PN42689 | 21 | 4.92 |
| PN42888 | 22 | 87.40 |
| PN42450 | 4 | 62.20 |
| PN42750 | 2 | 40.80 |
| PN42562 | 5 | 92.00 |
| PN42483 | 29 | 86.40 |
| PN42712 | 11 | 80.70 |
| PN42561 | 150 | 80.20 |
| PN42442 | 3 | 61.30 |
| PN42476 | 1 | 54.20 |
| PN42496 | 1 | 45.00 |
| PN42655 | 7 | 27.00 |
| PN42677 | 1 | 25.30 |
| PN42706 | 1 | 24.70 |
| PN42654 | 1 | 20.10 |
| PN42441 | 1 | 15.50 |
| PN42683 | 2 | 11.10 |
| PN42845 | 1 | 10.50 |
| PN42826 | 1 | 9.26 |
| PN42707 | 10 | 7.28 |
| PN42833 | 2 | 6.86 |
| PN42762 | 12 | 4.95 |
| PN42780 | 1 | 4.89 |
| PN42746 | 1 | 4.39 |
| PN42815 | 30 | 4.35 |
| PN42711 | 1 | 4.26 |
| PN42895 | 3 | 4.09 |
| PN42610 | 1 | 6.08 |

TABLE 3
Amino acid CDR sequences for VelociT TCRs specific for PRAME (312-320)/HLA-A2

| | | SEQ | | SEQ | | SEQ | | SEQ | | SEQ | | SEQ |
|---|---|---|---|---|---|---|---|---|---|---|---|---|
| TCR | | ID | | ID | | ID | | ID | | ID | | ID |
| ID | Vα CDR1 | NO: | Vα CDR2 | NO: | Vα CDR3 | NO: | Vβ CDR1 | NO: | Vβ CDR2 | NO: | Vβ CDR3 | NO: |
| PN46678 | TRDTTYY | 1 | RNSFDEQN | 2 | ALSEFDRGST | 3 | SGHKS | 4 | YYEKEE | 5 | ASSRDINEKLF | 6 |
| | | | | | LGRLY | | | | | | | |
| PN46714 | VSGLRG | 7 | LYSAGEE | 8 | AVQADGGSQGNLI | 9 | SGHKS | 10 | YYEKEE | 11 | ASSLDINSPLH | 12 |
| PN46723 | VSGLRG | 13 | LYSAGEE | 14 | AVQEDGGSQGNLI | 15 | SGHKS | 16 | YYEKEE | 17 | ASSRDINEKLF | 18 |
| PN46731 | DSASNY | 19 | IRSNVGE | 20 | AAWNYGQNFV | 21 | PRHDT | 22 | FYEKMQ | 23 | ASSLEGSEAF | 24 |
| PN46733 | NSASDY | 25 | IRSNMDK | 26 | AENNYGQNFV | 27 | MNHNS | 28 | SASEGT | 29 | ASSDWGQGVEAF | 30 |
| PN46735 | TISGTDY | 31 | GLTSN | 32 | ILREYMYSGGG | 33 | SGHKS | 34 | YYEKEE | 35 | ASSFQAGVN | 36 |
| | | | | | ADGLT | | | | | | YGYT | |
| PN46738 | TSENNYY | 37 | QEAYKQQN | 38 | AFGMYSGGGA | 39 | WSHSY | 40 | SAAADI | 41 | ASSDGTGYY | 42 |
| | | | | | DGLT | | | | | | GYT | |
| PN46777 | TSENNYY | 43 | QEAYKQQN | 44 | ALMEYGNKLV | 45 | WSHSY | 46 | SAAADI | 47 | ASSDGTGYYGYT | 48 |
| PN46797 | TSENNYY | 49 | QEAYKQQN | 50 | ALMEYENKLV | 51 | WSHSY | 52 | SAAADI | 53 | ASSDGTGYYGYT | 54 |
| PN46853 | SSNFYA | 55 | MTLNGDE | 56 | ACGGSGNTGKLI | 57 | PRHDT | 58 | FYEKMQ | 59 | ASSSQGQPQH | 60 |
| PN46857 | TRDTTYY | 61 | RNSFDEQN | 62 | ALSEGYGNKLV | 63 | KGHSH | 64 | LQKENI | 65 | ASSHRDDTEAF | 66 |
| PN46871 | DSAIYN | 67 | IQSSQRE | 68 | AVEGTTDSWGKFQ | 69 | PRHDT | 70 | FYEKMQ | 71 | ASSSQGQPQH | 72 |
| PN46880 | YSGSPE | 73 | HISR | 74 | ALSGASGGSYIPT | 75 | KGHSH | 76 | LQKENI | 77 | ASSHRDDTEAF | 78 |
| PN46883 | TRDTTYY | 79 | RNSFDEQN | 80 | ALSVSSYNTDKLI | 81 | KGHSH | 82 | LQKENI | 83 | ASSHRDDTEAF | 84 |
| PN46884 | TRDTTYY | 85 | RNSFDEQN | 86 | ALSEGYNTDKLI | 87 | MDHEN | 88 | SYDVKM | 89 | ASSLGGANTIY | 90 |
| PN46889 | DRGSQS | 91 | IYSNGD | 92 | AVNIPNSGYSTLT | 93 | MNHEY | 94 | SVGEGT | 95 | ASSYWEGTEAF | 96 |
| PN46909 | TSENNYY | 97 | QEAYKQQN | 98 | AFDYGQNFV | 99 | MNHNY | 100 | SVGAGI | 101 | ASSYGGGQTEAF | 102 |
| PN46914 | TRDTTYY | 103 | RNSFDEQN | 104 | ALSEGYNQGGKLI | 105 | MDHEN | 106 | SYDVKM | 107 | ASGADSNQPQH | 108 |

TABLE 4
Nucleic acid CDR sequences for VelociT TCRs specific for PRAME (312-320)/HLA-A2

| | | SEQ | | SEQ | | SEQ | | SEQ | | SEQ | | SEQ |
|---|---|---|---|---|---|---|---|---|---|---|---|---|
| TCR | | ID | | ID | | ID | | ID | | ID | | ID |
| ID | Vα CDR1 | NO: | Vα CDR2 | NO: | Vα CDR3 | NO: | Vβ CDR1 | NO: | Vβ CDR2 | NO: | Vβ CDR3 | NO: |
| PN46678 | ACCCGTG | 109 | CGGAACTCT | 110 | GCTCTGAGTGAGTTTG | 111 | TCTGGGC | 112 | TATTATG | 113 | GCCAGCAGCCGGGA | 114 |
| | ATACTAC | | TTTGATGAG | | ACAGAGGCTCAACCC | | ACAAGA | | AGAAAGA | | CATTAATGAAAAAC | |
| | TTATTAC | | CAAAAT | | TGGGGAGGCTATAC | | GT | | AGAG | | TGTTT | |
| PN46714 | GTCAGCG | 115 | CTGTATTCA | 116 | GCTGTGCAGGCCGAT | 117 | TCTGGGC | 118 | TATTATG | 119 | GCCAGCAGCTTGGA | 120 |
| | GTTTAAG | | GCTGGGGAA | | GGAGGAAGCCAAGGA | | ACAAGA | | AGAAAGA | | CATTAATTCACCCCT | |
| | AGGG | | GAA | | AATCTCATC | | GT | | AGAG | | CCAC | |
| PN46723 | GTCAGCG | 121 | CTGTATTCA | 122 | GCTGTGCAGGAGGAT | 123 | TCTGGGC | 124 | TATTATG | 125 | GCCAGCAGCCGGGA | 126 |
| | GTTTAAG | | GCTGGGGAA | | GGAGGAAGCCAAGGA | | ACAAGA | | AGAAAGA | | CATTAATGAAAAAC | |
| | AGGG | | GAA | | AATCTCATC | | GT | | AGAG | | TGTTT | |
| PN46731 | GACAGTG | 127 | ATTCGTTCA | 128 | GCAGCATGGAACTAT | 129 | CCTAGAC | 130 | TTTTATG | 131 | GCCAGCAGCTTAGA | 132 |
| | CCTCAAA | | AATGTGGGC | | GGTCAGAATTTTGTC | | ACGACA | | AAAAGAT | | GGGGTCTGAAGCTT | |
| | CTAC | | GAA | | | | CT | | GCAG | | TC | |
| PN46733 | AACAGCG | 133 | ATTCGTTCA | 134 | GCAGAGAATAACTAT | 135 | ATGAAC | 136 | TCAGCTT | 137 | GCCAGCAGTGACTG | 138 |
| | CCTCAGA | | AATATGGAC | | GGTCAGAATTTTGTC | | CATAACT | | CTGAGGG | | GGGACAGGGGGTTG | |
| | CTAC | | AAA | | | | CC | | TACC | | AAGCTTTC | |
| PN46735 | ACAATCA | 139 | GGTCTTACA | 140 | ATCCTGAGAGAATAC | 141 | TCTGGGC | 142 | TATTATG | 143 | GCCAGCAGCTTCCA | 144 |
| | GTGGAAC | | AGCAAT | | ATGTATTCAGGAGGA | | ACAAGA | | AGAAAGA | | AGCAGGGGTTAACT | |
| | TGATTAC | | | | GGTGCTGACGGACTC | | GT | | AGAG | | ATGGCTACACC | |
| | | | | | ACC | | | | | | | |
| PN46738 | ACCAGTG | 145 | CAAGAAGCT | 146 | GCTTTCGGTATGTATT | 147 | TGGAGC | 148 | TCAGCAG | 149 | GCCAGCAGTGATGG | 150 |
| | AGAATAA | | TATAAGCAA | | CAGGAGGAGGTGCTG | | CACAGCT | | CTGCTGA | | GACAGGGTACTATG | |
| | TTATTAT | | CAGAAT | | ACGGACTCACC | | AT | | TATT | | GCTACACC | |
| PN46777 | ACCAGTG | 151 | CAAGAAGCT | 152 | GCCCTTATGGAATATG | 153 | TGGAGC | 154 | TCAGCAG | 155 | GCCAGCAGTGATGG | 156 |
| | AGAATAA | | TATAAGCAA | | GAAACAAACTGGTC | | CACAGCT | | CTGCTGA | | GACAGGGTACTATG | |
| | TTATTAT | | CAGAAT | | | | AT | | TATT | | GCTACACC | |
| PN46797 | ACCAGTG | 157 | CAAGAAGCT | 158 | GCCCTTATGGAATATG | 159 | TGGAGC | 160 | TCAGCAG | 161 | GCCAGCAGTGATGG | 162 |
| | AGAATAA | | TATAAGCAA | | AAAACAAACTGGTC | | CACAGCT | | CTGCTGA | | GACAGGGTACTATG | |
| | TTATTAT | | CAGAAT | | | | AT | | TATT | | GCTACACC | |
| PN46853 | TCCAGCA | 163 | ATGACTTTA | 164 | GCCTGTGGGGGTTCTG | 165 | CCTAGAC | 166 | TTTTATG | 167 | GCCAGCAGCTCCCA | 168 |
| | ATTTTTAT | | AATGGGGAT | | GCAACACAGGCAAAC | | ACGACA | | AAAAGAT | | GGGTCAGCCCCAGC | |
| | GCC | | | | TAATC | | CT | | GCAG | | AT | |
| PN46857 | ACCCGTG | 169 | CGGAACTCT | 170 | GCTCTGAGTGAGGGA | 171 | AAAGGA | 172 | CTCCAGA | 173 | GCCAGCTCACACAG | 174 |
| | ATACTAC | | TTTGATGAG | | TATGGAAACAAACTG | | CACAGTC | | AAGAAAA | | GGACGACACTGAAG | |
| | TTATTAC | | CAAAAT | | GTC | | AT | | TATC | | CTTTC | |
| PN46871 | GATAGCG | 175 | ATTCAGTCA | 176 | GCTGTGGAGGGGACA | 177 | CCTAGAC | 178 | TTTTATG | 179 | GCCAGCAGCTCCCA | 180 |
| | CTATTTA | | AGTCAGAGA | | ACTGACAGCTGGGGG | | ACGACA | | AAAAGAT | | GGGTCAGCCCCAGC | |
| | CAAC | | GAG | | AAATTCCAG | | CT | | GCAG | | AT | |
| PN46880 | TATTCTG | 181 | CACATCTCT | 182 | GCTCTAAGTGGGGCAT | 183 | AAAGGA | 184 | CTCCAGA | 185 | GCCAGCTCACACAG | 186 |
| | GGAGTCC | | AGA | | CAGGAGGAAGCTACA | | CACAGTC | | AAGAAAA | | GGACGACACTGAAG | |
| | TGAA | | | | TACCTACA | | AT | | TATC | | CTTTC | |
| PN46883 | ACCCGTG | 187 | CGGAACTCT | 188 | GCTCTGAGTGTATCAT | 189 | AAAGGA | 190 | CTCCAGA | 191 | GCCAGCTCACACAG | 192 |
| | ATACTAC | | TTTGATGAG | | CTTATAACACCGACAA | | CACAGTC | | AAGAAAA | | GGATGACACTGAAG | |
| | TTATTAC | | CAAAAT | | GCTCATC | | AT | | TATC | | CTTTC | |
| PN46884 | ACCCGTG | 193 | CGGAACTCT | 194 | GCTCTGAGTGAGGGG | 195 | ATGGAC | 196 | TCATATG | 197 | GCCAGCAGTTTAGG | 198 |
| | ATACTAC | | TTTGATGAG | | TATAACACCGACAAG | | CATGAA | | ATGTTAA | | GGGGGCGAACACCA | |
| | TTATTAC | | CAAAAT | | CTCATC | | AAT | | AATG | | TATAT | |
| PN46889 | GACCGAG | 199 | ATATACTCC | 200 | GCCGTGAACATTCCGA | 201 | ATGAAC | 202 | TCAGTTG | 203 | GCCAGCAGTTACTG | 204 |
| | GTTCCCA | | AATGGTGAC | | ATTCAGGATACAGCA | | CATGAAT | | GTGAGGG | | GGAGGGCACTGAAG | |
| | GTCC | | | | CCCTCACC | | AC | | TACA | | CTTTC | |
| PN46909 | ACCAGTG | 205 | CAAGAAGCT | 206 | GCTTTCGACTATGGTC | 207 | ATGAAC | 208 | TCAGTTG | 209 | GCCAGCAGTTACGG | 210 |
| | AGAATAA | | TATAAGCAA | | AGAATTTTGTC | | CATAACT | | GTGCTGG | | GGGGGGGCAGACTG | |
| | TTATTAT | | CAGAAT | | | | AC | | TATC | | AAGCTTTC | |
| PN46914 | ACCCGTG | 211 | CGGAACTCT | 212 | GCTCTGAGTGAGGGTT | 213 | ATGGAC | 214 | TCATATG | 215 | GCCAGCGGGGCAGA | 216 |
| | ATACTAC | | TTTGATGAG | | ATAACCAGGGAGGAA | | CATGAA | | ATGTTAA | | TAGCAATCAGCCCC | |
| | TTATTAC | | CAAAAT | | AGCTTATC | | AAT | | AATG | | AGCAT | |

TABLE 5
Amino acid and nucleic acid sequences for VelociT TCRs
specific for PRAME (312-320)/HLA-A2

| | Domain Sequences |
|---|---|
| | Amino Acid Sequence (SEQ ID NO); CDR1, CDR2, and |
| | CDR3 sequences are underlined |
| Domain | Nucleic Acid Sequence (SEQ ID NO); CDR1, CDR2, and |
| name | CDR3 sequences are underlined |
| PN46678 | AQKVTQAQPEISVVEKEDVTLDCVYETRDTTYYLFWYKQPPSGELVFLIRRNSFD |
| Vα | EQNEISGRYSWNFQKSTSSFNFTITASQVVDSAVYFCALSEFDRGSTLGRLYFGR |
| | GTQLTVWP (SEQ ID NO: 217) |
| | GCTCAGAAGGTAACTCAAGCGCAGCCTGAAATTTCTGTGGTGGAGAAGGAG |
| | GATGTGACCTTGGACTGTGTGTATGAAACCCGTGATACTACTTATTACTTATT |
| | CTGGTACAAGCAACCACCAAGTGGAGAATTGGTTTTCCTTATTCGTCGGAAC |
| | TCTTTTGATGAGCAAAATGAAATAAGTGGTCGGTATTCTTGGAACTTCCAGA |
| | AATCCACCAGTTCCTTCAACTTCACCATCACAGCCTCACAAGTCGTGGACTCA |
| | GCAGTATACTTCTGTGCTCTGAGTGAGTTTGACAGAGGCTCAACCCTGGGGA |
| | GGCTATACTTTGGAAGAGGAACTCAGTTGACTGTCTGGCCT (SEQ ID NO: 218) |
| PN46678 | DAGVTQSPTHLIKTRGQQVTLRCSPISGHKSVSWYQQVLGQGPQFIFQYYEKEER |
| Vβ | GRGNFPDRFSARQFPNYSSELNVNALLLGDSALYLCASSRDINEKLFFGSGTQLS |
| | VL (SEQ ID NO: 219) |
| | GACGCTGGAGTCACCCAAAGTCCCACACACCTGATCAAAACGAGAGGACAG |
| | CAAGTGACTCTGAGATGCTCTCCTATCTCTGGGCACAAGAGTGTGTCCTGGTA |
| | CCAACAGGTCCTGGGTCAGGGGCCCCAGTTTATCTTTCAGTATTATGAGAAA |
| | GAAGAGAGAGGAAGAGGAAACTTCCCTGATCGATTCTCAGCTCGCCAGTTCC |
| | CTAACTATAGCTCTGAGCTGAATGTGAACGCCTTGTTGCTGGGGGACTCGGC |
| | CCTGTATCTCTGTGCCAGCAGCCGGGACATTAATGAAAAACTGTTTTTTGGCA |
| | GTGGAACCCAGCTCTCTGTCTTG (SEQ ID NO: 220) |
| PN46714 | EDQVTQSPEALRLQEGESSSLNCSYTVSGLRGLFWYRQDPGKGPEFLFTLYSAGE |
| Vα | EKEKERLKATLTKKESFLHITAPKPEDSATYLCAVQADGGSQGNLIFGKGTKLSV |
| | KP (SEQ ID NO: 221) |
| | GAAGACCAGGTGACGCAGAGTCCCGAGGCCCTGAGACTCCAGGAGGGAGAG |
| | AGTAGCAGTCTCAACTGCAGTTACACAGTCAGCGGTTTAAGAGGGCTGTTCT |
| | GGTATAGGCAAGATCCTGGGAAAGGCCCTGAATTCCTCTTCACCCTGTATTC |
| | AGCTGGGGAAGAAAAGGAGAAAGAAAGGCTAAAAGCCACATTAACAAAGA |
| | AGGAAAGCTTTCTGCACATCACAGCCCCTAAACCTGAAGACTCAGCCACTTA |
| | TCTCTGTGCTGTGCAGGCCGATGGAGGAAGCCAAGGAAATCTCATCTTTGGA |
| | AAAGGCACTAAACTCTCTGTTAAACCA (SEQ ID NO: 222) |
| PN46714 | DAGVTQSPTHLIKTRGQQVTLRCSPISGHKSVSWYQQVLGQGPQFIFQYYEKEER |
| Vβ | GRGNFPDRFSARQFPNYSSELNVNALLLGDSALYLCASSLDINSPLHFGNGTRLT |
| | VT (SEQ ID NO: 223) |
| | GACGCTGGAGTCACCCAAAGTCCCACACACCTGATCAAAACGAGAGGACAG |
| | CAAGTGACTCTGAGATGCTCTCCTATCTCTGGGCACAAGAGTGTGTCCTGGTA |
| | CCAACAGGTCCTGGGTCAGGGGCCCCAGTTTATCTTTCAGTATTATGAGAAA |
| | GAAGAGAGAGGAAGAGGAAACTTCCCTGATCGATTCTCAGCTCGCCAGTTCC |
| | CTAACTATAGCTCTGAGCTGAATGTGAACGCCTTGTTGCTGGGGGACTCGGC |
| | CCTGTATCTCTGTGCCAGCAGCTTGGACATTAATTCACCCCTCCACTTTGGGA |
| | ACGGGACCAGGCTCACTGTGACA (SEQ ID NO: 224) |
| PN46723 | EDQVTQSPEALRLQEGESSSLNCSYTVSGLRGLFWYRQDPGKGPEFLFTLYSAGE |
| Vα | EKEKERLKATLTKKESFLHITAPKPEDSATYLCAVQEDGGSQGNLIFGKGTKLSV |
| | KP (SEQ ID NO: 225) |
| | GAAGACCAGGTGACGCAGAGTCCCGAGGCCCTGAGACTCCAGGAGGGAGAG |
| | AGTAGCAGTCTCAACTGCAGTTACACAGTCAGCGGTTTAAGAGGGCTGTTCT |
| | GGTATAGGCAAGATCCTGGGAAAGGCCCTGAATTCCTCTTCACCCTGTATTC |
| | AGCTGGGGAAGAAAAGGAGAAAGAAAGGCTAAAAGCCACATTAACAAAGA |
| | AGGAAAGCTTTCTGCACATCACAGCCCCTAAACCTGAAGACTCAGCCACTTA |
| | TCTCTGTGCTGTGCAGGAGGATGGAGGAAGCCAAGGAAATCTCATCTTTGGA |
| | AAAGGCACTAAACTCTCTGTTAAACCA (SEQ ID NO: 226) |
| PN46723 | DAGVTQSPTHLIKTRGQQVTLRCSPISGHKSVSWYQQVLGQGPQFIFQYYEKEER |
| Vβ | GRGNFPDRFSARQFPNYSSELNVNALLLGDSALYLCASSRDINEKLFFGSGTQLS |
| | VL (SEQ ID NO: 227) |
| | GACGCTGGAGTCACCCAAAGTCCCACACACCTGATCAAAACGAGAGGACAG |
| | CAAGTGACTCTGAGATGCTCTCCTATCTCTGGGCACAAGAGTGTGTCCTGGTA |
| | CCAACAGGTCCTGGGTCAGGGGCCCCAGTTTATCTTTCAGTATTATGAGAAA |
| | GAAGAGAGAGGAAGAGGAAACTTCCCTGATCGATTCTCAGCTCGCCAGTTCC |
| | CTAACTATAGCTCTGAGCTGAATGTGAACGCCTTGTTGCTGGGGGACTCGGC |
| | CCTGTATCTCTGTGCCAGCAGCCGGGACATTAATGAAAAACTGTTTTTTGGCA |
| | GTGGAACCCAGCTCTCTGTCTTG (SEQ ID NO: 228) |
| PN46731 | GENVEQHPSTLSVQEGDSAVIKCTYSDSASNYFPWYKQELGKRPQLIIDIRSNVG |
| Vα | EKKDQRIAVTLNKTAKHFSLHITETQPEDSAVYFCAAWNYGQNFVFGPGTRLSV |
| | LP (SEQ ID NO: 229) |
| | GGAGAGAATGTGGAGCAGCATCCTTCAACCCTGAGTGTCCAGGAGGGAGAC |
| | AGCGCTGTTATCAAGTGTACTTATTCAGACAGTGCCTCAAACTACTTCCCTTG |
| | GTATAAGCAAGAACTTGGAAAAAGACCTCAGCTTATTATAGACATTCGTTCA |
| | AATGTGGGCGAAAAGAAAGACCAACGAATTGCTGTTACATTGAACAAGACA |
| | GCCAAACATTTCTCCCTGCACATCACAGAGACCCAACCTGAAGACTCGGCTG |
| | TCTACTTCTGTGCAGCATGGAACTATGGTCAGAATTTTGTCTTTGGTCCCGGA |
| | ACCAGATTGTCCGTGCTGCCC (SEQ ID NO: 230) |
| PN46731 | AAGVIQSPRHLIKEKRETATLKCYPIPRHDTVYWYQQGPGQDPQFLISFYEKMQS |
| Vβ | DKGSIPDRFSAQQFSDYHSELNMSSLELGDSALYFCASSLEGSEAFFGQGTRLTV |
| | V (SEQ ID NO: 231) |
| | GCTGCTGGAGTCATCCAGTCCCCAAGACATCTGATCAAAGAAAAGAGGGAA |
| | ACAGCCACTCTGAAATGCTATCCTATCCCTAGACACGACACTGTCTACTGGTA |
| | CCAGCAGGGTCCAGGTCAGGACCCCCAGTTCCTCATTTCGTTTTATGAAAAG |
| | ATGCAGAGCGATAAAGGAAGCATCCCTGATCGATTCTCAGCTCAACAGTTCA |
| | GTGACTATCATTCTGAACTGAACATGAGCTCCTTGGAGCTGGGGGACTCAGC |
| | CCTGTACTTCTGTGCCAGCAGCTTAGAGGGGTCTGAAGCTTTCTTTGGACAAG |
| | GCACCAGACTCACAGTTGTA (SEQ ID NO: 232) |
| PN46733 | GESVGLHLPTLSVQEGDNSIINCAYSNSASDYFIWYKQESGKGPQFIIDIRSNMDK |
| Vα | RQGQRVTVLLNKTVKHLSLQIAATQPGDSAVYFCAENNYGQNFVFGPGTRLSVL |
| | P (SEQ ID NO: 233) |
| | GGAGAGAGTGTGGGGCTGCATCTTCCTACCCTGAGTGTCCAGGAGGGTGACA |
| | ACTCTATTATCAACTGTGCTTATTCAAACAGCGCCTCAGACTACTTCATTTGG |
| | TACAAGCAAGAATCTGGAAAAGGTCCTCAATTCATTATAGACATTCGTTCAA |
| | ATATGGACAAAAGGCAAGGCCAAAGAGTCACCGTTTTATTGAATAAGACAGT |
| | GAAACATCTCTCTCTGCAAATTGCAGCTACTCAACCTGGAGACTCAGCTGTCT |
| | ACTTTTGTGCAGAGAATAACTATGGTCAGAATTTTGTCTTTGGTCCCGGAACC |
| | AGATTGTCCGTGCTGCCC (SEQ ID NO: 234) |
| PN46733 | NAGVTQTPKFQVLKTGQSMTLQCAQDMNHNSMYWYRQDPGMGLRLIYYSASE |
| Vβ | GTTDKGEVPNGYNVSRLNKREFSLRLESAAPSQTSVYFCASSDWGQGVEAFFGQ |
| | GTRLTVV (SEQ ID NO: 235) |
| | AATGCTGGTGTCACTCAGACCCCAAAATTCCAGGTCCTGAAGACAGGACAGA |
| | GCATGACACTGCAGTGTGCCCAGGATATGAACCATAACTCCATGTACTGGTA |
| | TCGACAAGACCCAGGCATGGGACTGAGGCTGATTTATTACTCAGCTTCTGAG |
| | GGTACCACTGACAAAGGAGAAGTCCCCAATGGCTACAATGTCTCCAGATTAA |
| | ACAAACGGGAGTTCTCGCTCAGGCTGGAGTCGGCTGCTCCCTCCCAGACATC |
| | TGTGTACTTCTGTGCCAGCAGTGACTGGGGACAGGGGGTTGAAGCTTTCTTTG |
| | GACAAGGCACCAGACTCACAGTTGTA (SEQ ID NO: 236) |
| PN46735 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILREYMYSGGGADGLTFGKGT |
| | HLIIQP (SEQ ID NO: 237) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAGAGAATACATGTATTCAGGAGGAGGTGCTGACGGACTCACCTT |
| | TGGCAAAGGGACTCATCTAATCATCCAGCCC (SEQ ID NO: 238) |
| PN46735 | DAGVTQSPTHLIKTRGQQVTLRCSPISGHKSVSWYQQVLGQGPQFIFQYYEKEER |
| Vβ | GRGNFPDRFSARQFPNYSSELNVNALLLGDSALYLCASSFQAGVNYGYTFGSGT |
| | RLTVV (SEQ ID NO: 239) |
| | GACGCTGGAGTCACCCAAAGTCCCACACACCTGATCAAAACGAGAGGACAG |
| | CAAGTGACTCTGAGATGCTCTCCTATCTCTGGGCACAAGAGTGTGTCCTGGTA |
| | CCAACAGGTCCTGGGTCAGGGGCCCCAGTTTATCTTTCAGTATTATGAGAAA |
| | GAAGAGAGAGGAAGAGGAAACTTCCCTGATCGATTCTCAGCTCGCCAGTTCC |
| | CTAACTATAGCTCTGAGCTGAATGTGAACGCCTTGTTGCTGGGGGACTCGGC |
| | CCTGTATCTCTGTGCCAGCAGCTTCCAAGCAGGGGTTAACTATGGCTACACCT |
| | TCGGTTCGGGGACCAGGTTAACCGTTGTA (SEQ ID NO: 240) |
| PN46738 | AQTVTQSQPEMSVQEAETVTLSCTYDTSENNYYLFWYKQPPSRQMILVIRQEAY |
| Vα | KQQNATENRFSVNFQKAAKSFSLKISDSQLGDTAMYFCAFGMYSGGGADGLTF |
| | GKGTHLIIQP (SEQ ID NO: 241) |
| | GCCCAGACAGTCACTCAGTCTCAACCAGAGATGTCTGTGCAGGAGGCAGAGA |
| | CTGTGACCCTGAGTTGCACATATGACACCAGTGAGAATAATTATTATTTGTTC |
| | TGGTACAAGCAGCCTCCCAGCAGGCAGATGATTCTCGTTATTCGCCAAGAAG |
| | CTTATAAGCAACAGAATGCAACGGAGAATCGTTTCTCTGTGAACTTCCAGAA |
| | AGCAGCCAAATCCTTCAGTCTCAAGATCTCAGACTCACAGCTGGGGGACACT |
| | GCGATGTATTTCTGTGCTTTCGGTATGTATTCAGGAGGAGGTGCTGACGGACT |
| | CACCTTTGGCAAAGGGACTCATCTAATCATCCAGCCC (SEQ ID NO: 242) |
| PN46738 | DAGITQSPRYKITETGRQVTLMCHQTWSHSYMFWYRQDLGHGLRLIYYSAAADI |
| Vβ | TDKGEVPDGYVVSRSKTENFPLTLESATRSQTSVYFCASSDGTGYYGYTFGSGTR |
| | LTVV (SEQ ID NO: 243) |
| | GATGCTGGAATCACCCAGAGCCCAAGATACAAGATCACAGAGACAGGAAGG |
| | CAGGTGACCTTGATGTGTCACCAGACTTGGAGCCACAGCTATATGTTCTGGT |
| | ATCGACAAGACCTGGGACATGGGCTGAGGCTGATCTATTACTCAGCAGCTGC |
| | TGATATTACAGATAAAGGAGAAGTCCCCGATGGCTATGTTGTCTCCAGATCC |
| | AAGACAGAGAATTTCCCCCTCACTCTGGAGTCAGCTACCCGCTCCCAGACAT |
| | CTGTGTATTTCTGCGCCAGCAGTGATGGGACAGGGTACTATGGCTACACCTTC |
| | GGTTCGGGGACCAGGTTAACCGTTGTA (SEQ ID NO: 244) |
| PN46777 | AQTVTQSQPEMSVQEAETVTLSCTYDTSENNYYLFWYKQPPSRQMILVIRQEAY |
| Vα | KQQNATENRFSVNFQKAAKSFSLKISDSQLGDTAMYFCALMEYGNKLVFGAGTI |
| | LRVKS (SEQ ID NO: 245) |
| | GCCCAGACAGTCACTCAGTCTCAACCAGAGATGTCTGTGCAGGAGGCAGAGA |
| | CTGTGACCCTGAGTTGCACATATGACACCAGTGAGAATAATTATTATTTGTTC |
| | TGGTACAAGCAGCCTCCCAGCAGGCAGATGATTCTCGTTATTCGCCAAGAAG |
| | CTTATAAGCAACAGAATGCAACGGAGAATCGTTTCTCTGTGAACTTCCAGAA |
| | AGCAGCCAAATCCTTCAGTCTCAAGATCTCAGACTCACAGCTGGGGGACACT |
| | GCGATGTATTTCTGTGCCCTTATGGAATATGGAAACAAACTGGTCTTTGGCGC |
| | AGGAACCATTCTGAGAGTCAAGTCC (SEQ ID NO: 246) |
| PN46777 | DAGITQSPRYKITETGRQVTLMCHQTWSHSYMFWYRQDLGHGLRLIYYSAAADI |
| Vβ | TDKGEVPDGYVVSRSKTENFPLTLESATRSQTSVYFCASSDGTGYYGYTFGSGTR |
| | LTVV (SEQ ID NO: 247) |
| | GATGCTGGAATCACCCAGAGCCCAAGATACAAGATCACAGAGACAGGAAGG |
| | CAGGTGACCTTGATGTGTCACCAGACTTGGAGCCACAGCTATATGTTCTGGT |
| | ATCGACAAGACCTGGGACATGGGCTGAGGCTGATCTATTACTCAGCAGCTGC |
| | TGATATTACAGATAAAGGAGAAGTCCCCGATGGCTATGTTGTCTCCAGATCC |
| | AAGACAGAGAATTTCCCCCTCACTCTGGAGTCAGCTACCCGCTCCCAGACAT |
| | CTGTGTATTTCTGCGCCAGCAGTGATGGGACAGGGTACTATGGCTACACCTTC |
| | GGTTCGGGGACCAGGTTAACCGTTGTA (SEQ ID NO: 248) |
| PN46797 | AQTVTQSQPEMSVQEAETVTLSCTYDTSENNYYLFWYKQPPSRQMILVIRQEAY |
| Vα | KQQNATENRFSVNFQKAAKSFSLKISDSQLGDTAMYFCALMEYENKLVFGAGTI |
| | LRVKS (SEQ ID NO: 249) |
| | GCCCAGACAGTCACTCAGTCTCAACCAGAGATGTCTGTGCAGGAGGCAGAGA |
| | CTGTGACCCTGAGTTGCACATATGACACCAGTGAGAATAATTATTATTTGTTC |
| | TGGTACAAGCAGCCTCCCAGCAGGCAGATGATTCTCGTTATTCGCCAAGAAG |
| | CTTATAAGCAACAGAATGCAACGGAGAATCGTTTCTCTGTGAACTTCCAGAA |
| | AGCAGCCAAATCCTTCAGTCTCAAGATCTCAGACTCACAGCTGGGGGACACT |
| | GCGATGTATTTCTGTGCCCTTATGGAATATGAAAACAAACTGGTCTTTGGCGC |
| | AGGAACCATTCTGAGAGTCAAGTCC (SEQ ID NO: 250) |
| PN46797 | DAGITQSPRYKITETGRQVTLMCHQTWSHSYMFWYRQDLGHGLRLIYYSAAADI |
| Vβ | TDKGEVPDGYVVSRSKTENFPLTLESATRSQTSVYFCASSDGTGYYGYTFGSGTR |
| | LTVV (SEQ ID NO: 251) |
| | GATGCTGGAATCACCCAGAGCCCAAGATACAAGATCACAGAGACAGGAAGG |
| | CAGGTGACCTTGATGTGTCACCAGACTTGGAGCCACAGCTATATGTTCTGGT |
| | ATCGACAAGACCTGGGACATGGGCTGAGGCTGATCTATTACTCAGCAGCTGC |
| | TGATATTACAGATAAAGGAGAAGTCCCCGATGGCTATGTTGTCTCCAGATCC |
| | AAGACAGAGAATTTCCCCCTCACTCTGGAGTCAGCTACCCGCTCCCAGACAT |
| | CTGTGTATTTCTGCGCCAGCAGTGATGGGACAGGGTACTATGGCTACACCTTC |
| | GGTTCGGGGACCAGGTTAACCGTTGTA (SEQ ID NO: 252) |
| PN46853 | ILNVEQSPQSLHVQEGDSTNFTCSFPSSNFYALHWYRWETAKSPEALFVMTLNG |
| Vα | DEKKKGRISATLNTKEGYSYLYIKGSQPEDSATYLCACGGSGNTGKLIFGQGTTL |
| | QVKP (SEQ ID NO: 253) |
| | ATACTGAACGTGGAACAAAGTCCTCAGTCACTGCATGTTCAGGAGGGAGACA |
| | GCACCAATTTCACCTGCAGCTTCCCTTCCAGCAATTTTTATGCCTTACACTGG |
| | TACAGATGGGAAACTGCAAAAAGCCCCGAGGCCTTGTTTGTAATGACTTTAA |
| | ATGGGGATGAAAAGAAGAAAGGACGAATAAGTGCCACTCTTAATACCAAGG |
| | AGGGTTACAGCTATTTGTACATCAAAGGATCCCAGCCTGAAGACTCAGCCAC |
| | ATACCTCTGTGCCTGTGGGGGTTCTGGCAACACAGGCAAACTAATCTTTGGG |
| | CAAGGGACAACTTTACAAGTAAAACCA (SEQ ID NO: 254) |
| PN46853 | AAGVIQSPRHLIKEKRETATLKCYPIPRHDTVYWYQQGPGQDPQFLISFYEKMQS |
| Vβ | DKGSIPDRFSAQQFSDYHSELNMSSLELGDSALYFCASSSQGQPQHFGDGTRLSIL |
| | (SEQ ID NO: 255) |
| | GCTGCTGGAGTCATCCAGTCCCCAAGACATCTGATCAAAGAAAAGAGGGAA |
| | ACAGCCACTCTGAAATGCTATCCTATCCCTAGACACGACACTGTCTACTGGTA |
| | CCAGCAGGGTCCAGGTCAGGACCCCCAGTTCCTCATTTCGTTTTATGAAAAG |
| | ATGCAGAGCGATAAAGGAAGCATCCCTGATCGATTCTCAGCTCAACAGTTCA |
| | GTGACTATCATTCTGAACTGAACATGAGCTCCTTGGAGCTGGGGGACTCAGC |
| | CCTGTACTTCTGTGCCAGCAGCTCCCAGGGTCAGCCCCAGCATTTTGGTGATG |
| | GGACTCGACTCTCCATCCTA (SEQ ID NO: 256) |
| PN46857 | AQKVTQATREISVVEKEDVTLDCVYETRDTTYYLFWYKQPPSGELVFLIRRNSFD |
| Vα | EQNEISGRYSWNFQKSTSSFNFTITASQVVDSAVYFCALSEGYGNKLVFGAGTIL |
| | RVKS (SEQ ID NO: 257) |
| | GCTCAGAAGGTAACTCAAGCGACTAGAGAAATTTCTGTGGTGGAGAAGGAG |
| | GATGTGACCTTGGACTGTGTGTATGAAACCCGTGATACTACTTATTACTTATT |
| | CTGGTACAAGCAACCACCAAGTGGAGAATTGGTTTTCCTTATTCGTCGGAAC |
| | TCTTTTGATGAGCAAAATGAAATAAGTGGTCGGTATTCTTGGAACTTCCAGA |
| | AATCCACCAGTTCCTTCAACTTCACCATCACAGCCTCACAAGTCGTGGACTCA |
| | GCAGTATACTTCTGTGCTCTGAGTGAGGGATATGGAAACAAACTGGTCTTTG |
| | GCGCAGGAACCATTCTGAGAGTCAAGTCC (SEQ ID NO: 258) |
| PN46857 | NAGVMQNPRHLVRRRGQEARLRCSPMKGHSHVYWYRQLPEEGLKFMVYLQKE |
| Vβ | NIIDESGMPKERFSAEFPKEGPSILRIQQVVRGDSAAYFCASSHRDDTEAFFGQGT |
| | RLTVV (SEQ ID NO: 259) |
| | AATGCCGGCGTCATGCAGAACCCAAGACACCTGGTCAGGAGGAGGGGACAG |
| | GAGGCAAGACTGAGATGCAGCCCAATGAAAGGACACAGTCATGTTTACTGGT |
| | ATCGGCAGCTCCCAGAGGAAGGTCTGAAATTCATGGTTTATCTCCAGAAAGA |
| | AAATATCATAGATGAGTCAGGAATGCCAAAGGAACGATTTTCTGCTGAATTT |
| | CCCAAAGAGGGCCCCAGCATCCTGAGGATCCAGCAGGTAGTGCGAGGAGAT |
| | TCGGCAGCTTATTTCTGTGCCAGCTCACACAGGGACGACACTGAAGCTTTCTT |
| | TGGACAAGGCACCAGACTCACAGTTGTA (SEQ ID NO: 260) |
| PN46871 | KQEVTQIPAALSVPEGENLVLNCSFTDSAIYNLQWFRQDPGKGLTSLLLIQSSQRE |
| Vα | QTSGRLNASLDKSSGRSTLYIAASQPGDSATYLCAVEGTTDSWGKFQFGAGTQV |
| | WTP (SEQ ID NO: 261) |
| | AAACAGGAGGTGACGCAGATTCCTGCAGCTCTGAGTGTCCCAGAAGGAGAA |
| | AACTTGGTTCTCAACTGCAGTTTCACTGATAGCGCTATTTACAACCTCCAGTG |
| | GTTTAGGCAGGACCCTGGGAAAGGTCTCACATCTCTGTTGCTTATTCAGTCAA |
| | GTCAGAGAGAGCAAACAAGTGGAAGACTTAATGCCTCGCTGGATAAATCATC |
| | AGGACGTAGTACTTTATACATTGCAGCTTCTCAGCCTGGTGACTCAGCCACCT |
| | ACCTCTGTGCTGTGGAGGGGACAACTGACAGCTGGGGGAAATTCCAGTTTGG |
| | AGCAGGGACCCAGGTTGTGGTCACCCCA (SEQ ID NO: 262) |
| PN46871 | AAGVIQSPRHLIKEKRETATLKCYPIPRHDTVYWYQQGPGQDPQFLISFYEKMQS |
| Vβ | DKGSIPDRFSAQQFSDYHSELNMSSLELGDSALYFCASSSQGQPQHFGDGTRLSIL |
| | (SEQ ID NO: 263) |
| | GCTGCTGGAGTCATCCAGTCCCCAAGACATCTGATCAAAGAAAAGAGGGAA |
| | ACAGCCACTCTGAAATGCTATCCTATCCCTAGACACGACACTGTCTACTGGTA |
| | CCAGCAGGGTCCAGGTCAGGACCCCCAGTTCCTCATTTCGTTTTATGAAAAG |
| | ATGCAGAGCGATAAAGGAAGCATCCCTGATCGATTCTCAGCTCAACAGTTCA |
| | GTGACTATCATTCTGAACTGAACATGAGCTCCTTGGAGCTGGGGGACTCAGC |
| | CCTGTACTTCTGTGCCAGCAGCTCCCAGGGTCAGCCCCAGCATTTTGGTGATG |
| | GGACTCGACTCTCCATCCTA (SEQ ID NO: 264) |
| PN46880 | AQRVTQPEKLLSVFKGAPVELKCNYSYSGSPELFWYVQYSRQRLQLLLRHISRES |
| Vα | IKGFTADLNKGETSFHLKKPFAQEEDSAMYYCALSGASGGSYIPTFGRGTSLIVH |
| | P (SEQ ID NO: 265) |
| | GCCCAGAGAGTGACTCAGCCCGAGAAGCTCCTCTCTGTCTTTAAAGGGGCCC |
| | CAGTGGAGCTGAAGTGCAACTATTCCTATTCTGGGAGTCCTGAACTCTTCTGG |
| | TATGTCCAGTACTCCAGACAACGCCTCCAGTTACTCTTGAGACACATCTCTAG |
| | AGAGAGCATCAAAGGCTTCACTGCTGACCTTAACAAAGGCGAGACATCTTTC |
| | CACCTGAAGAAACCATTTGCTCAAGAGGAAGACTCAGCCATGTATTACTGTG |
| | CTCTAAGTGGGGCATCAGGAGGAAGCTACATACCTACATTTGGAAGAGGAAC |
| | CAGCCTTATTGTTCATCCG (SEQ ID NO: 266) |
| PN46880 | NAGVMQNPRHLVRRRGQEARLRCSPMKGHSHVYWYRQLPEEGLKFMVYLQKE |
| Vβ | NIIDESGMPKERFSAEFPKEGPSILRIQQVVRGDSAAYFCASSHRDDTEAFFGQGT |
| | RLTVV (SEQ ID NO: 267) |
| | AATGCCGGCGTCATGCAGAACCCAAGACACCTGGTCAGGAGGAGGGGACAG |
| | GAGGCAAGACTGAGATGCAGCCCAATGAAAGGACACAGTCATGTTTACTGGT |
| | ATCGGCAGCTCCCAGAGGAAGGTCTGAAATTCATGGTTTATCTCCAGAAAGA |
| | AAATATCATAGATGAGTCAGGAATGCCAAAGGAACGATTTTCTGCTGAATTT |
| | CCCAAAGAGGGCCCCAGCATCCTGAGGATCCAGCAGGTAGTGCGAGGAGAT |
| | TCGGCAGCTTATTTCTGTGCCAGCTCACACAGGGACGACACTGAAGCTTTCTT |
| | TGGACAAGGCACCAGACTCACAGTTGTA (SEQ ID NO: 268) |
| PN46883 | AQKVTQAQTEISVVEKEDVTLDCVYETRDTTYYLFWYKQPPSGELVFLIRRNSF |
| Vα | DEQNEISGRYSWNFQKSTSSFNFTITASQVVDSAVYFCALSVSSYNTDKLIFGTGT |
| | RLQVFP (SEQ ID NO: 269) |
| | GCTCAGAAGGTAACTCAAGCGCAGACTGAAATTTCTGTGGTGGAGAAGGAG |
| | GATGTGACCTTGGACTGTGTGTATGAAACCCGTGATACTACTTATTACTTATT |
| | CTGGTACAAGCAACCACCAAGTGGAGAATTGGTTTTCCTTATTCGTCGGAAC |
| | TCTTTTGATGAGCAAAATGAAATAAGTGGTCGGTATTCTTGGAACTTCCAGA |
| | AATCCACCAGTTCCTTCAACTTCACCATCACAGCCTCACAAGTCGTGGACTCA |
| | GCAGTATACTTCTGTGCTCTGAGTGTATCATCTTATAACACCGACAAGCTCAT |
| | CTTTGGGACTGGGACCAGATTACAAGTCTTTCCA (SEQ ID NO: 270) |
| PN46883 | NAGVMQNPRHLVRRRGQEARLRCSPMKGHSHVYWYRQLPEEGLKFMVYLQKE |
| Vβ | NIIDESGMPKERFSAEFPKEGPSILRIQQVVRGDSAAYFCASSHRDDTEAFFGQGT |
| | RLTVV (SEQ ID NO: 271) |
| | AATGCCGGCGTCATGCAGAACCCAAGACACCTGGTCAGGAGGAGGGGACAG |
| | GAGGCAAGACTGAGATGCAGCCCAATGAAAGGACACAGTCATGTTTACTGGT |
| | ATCGGCAGCTCCCAGAGGAAGGTCTGAAATTCATGGTTTATCTCCAGAAAGA |
| | AAATATCATAGATGAGTCAGGAATGCCAAAGGAACGATTTTCTGCTGAATTT |
| | CCCAAAGAGGGCCCCAGCATCCTGAGGATCCAGCAGGTAGTGCGAGGAGAT |
| | TCGGCAGCTTATTTCTGTGCCAGCTCACACAGGGATGACACTGAAGCTTTCTT |
| | TGGACAAGGCACCAGACTCACAGTTGTA (SEQ ID NO: 272) |
| PN46884 | AQKVTQAQPEISVVEKEDVTLDCVYETRDTTYYLFWYKQPPSGELVFLIRRNSFD |
| Vα | EQNEISGRYSWNFQKSTSSFNFTITASQVVDSAVYFCALSEGYNTDKLIFGTGTRL |
| | QVFP (SEQ ID NO: 273) |
| | GCTCAGAAGGTAACTCAAGCGCAGCCTGAAATTTCTGTGGTGGAGAAGGAG |
| | GATGTGACCTTGGACTGTGTGTATGAAACCCGTGATACTACTTATTACTTATT |
| | CTGGTACAAGCAACCACCAAGTGGAGAATTGGTTTTCCTTATTCGTCGGAAC |
| | TCTTTTGATGAGCAAAATGAAATAAGTGGTCGGTATTCTTGGAACTTCCAGA |
| | AATCCACCAGTTCCTTCAACTTCACCATCACAGCCTCACAAGTCGTGGACTCA |
| | GCAGTATACTTCTGTGCTCTGAGTGAGGGGTATAACACCGACAAGCTCATCT |
| | TTGGGACTGGGACCAGATTACAAGTCTTTCCA (SEQ ID NO: 274) |
| PN46884 | DVKVTQSSRYLVKRTGEKVFLECVQDMDHENMFWYRQDPGLGLRLIYFSYDVK |
| Vβ | MKEKGDIPEGYSVSREKKERFSLILESASTNQTSMYLCASSLGGANTIYFGEGSW |
| | LTVV (SEQ ID NO: 275) |
| | GATGTGAAAGTAACCCAGAGCTCGAGATATCTAGTCAAAAGGACGGGAGAG |
| | AAAGTTTTTCTGGAATGTGTCCAGGATATGGACCATGAAAATATGTTCTGGTA |
| | TCGACAAGACCCAGGTCTGGGGCTACGGCTGATCTATTTCTCATATGATGTTA |
| | AAATGAAAGAAAAAGGAGATATTCCTGAGGGGTACAGTGTCTCTAGAGAGA |
| | AGAAGGAGCGCTTCTCCCTGATTCTGGAGTCCGCCAGCACCAACCAGACATC |
| | TATGTACCTCTGTGCCAGCAGTTTAGGGGGGGCGAACACCATATATTTTGGA |
| | GAGGGAAGTTGGCTCACTGTTGTA (SEQ ID NO: 276) |
| PN46889 | QKEVEQNSGPLSVPEGAIASLNCTYSDRGSQSFFWYRQYSGKSPELIMSIYSNGD |
| Vα | KEDGRFTAQLNKASQYVSLLIRDSQPSDSATYLCAVNIPNSGYSTLTFGKGTMLL |
| | VSP (SEQ ID NO: 277) |
| | CAGAAGGAGGTGGAGCAGAATTCTGGACCCCTCAGTGTTCCAGAGGGAGCC |
| | ATTGCCTCTCTCAACTGCACTTACAGTGACCGAGGTTCCCAGTCCTTCTTCTG |
| | GTACAGACAATATTCTGGGAAAAGCCCTGAGTTGATAATGTCCATATACTCC |
| | AATGGTGACAAAGAAGATGGAAGGTTTACAGCACAGCTCAATAAAGCCAGC |
| | CAGTATGTTTCTCTGCTCATCAGAGACTCCCAGCCCAGTGATTCAGCCACCTA |
| | CCTCTGTGCCGTGAACATTCCGAATTCAGGATACAGCACCCTCACCTTTGGGA |
| | AGGGGACTATGCTTCTAGTCTCTCCA (SEQ ID NO: 278) |
| PN46889 | NAGVTQTPKFRVLKTGQSMTLLCAQDMNHEYMYWYRQDPGMGLRLIHYSVGE |
| Vβ | GTTAKGEVPDGYNVSRLKKQNFLLGLESAAPSQTSVYFCASSYWEGTEAFFGQG |
| | TRLTVV (SEQ ID NO: 279) |
| | AATGCTGGTGTCACTCAGACCCCAAAATTCCGGGTCCTGAAGACAGGACAGA |
| | GCATGACACTGCTGTGTGCCCAGGATATGAACCATGAATACATGTACTGGTA |
| | TCGACAAGACCCAGGCATGGGGCTGAGGCTGATTCATTACTCAGTTGGTGAG |
| | GGTACAACTGCCAAAGGAGAGGTCCCTGATGGCTACAATGTCTCCAGATTAA |
| | AAAAACAGAATTTCCTGCTGGGGTTGGAGTCGGCTGCTCCCTCCCAAACATC |
| | TGTGTACTTCTGTGCCAGCAGTTACTGGGAGGGCACTGAAGCTTTCTTTGGAC |
| | AAGGCACCAGACTCACAGTTGTA (SEQ ID NO: 280) |
| PN46909 | AQTVTQSQPEMSVQEAETVTLSCTYDTSENNYYLFWYKQPPSRQMILVIRQEAY |
| Vα | KQQNATENRFSVNFQKAAKSFSLKISDSQLGDTAMYFCAFDYGQNFVFGPGTRL |
| | SVLP (SEQ ID NO: 281) |
| | GCCCAGACAGTCACTCAGTCTCAACCAGAGATGTCTGTGCAGGAGGCAGAGA |
| | CTGTGACCCTGAGTTGCACATATGACACCAGTGAGAATAATTATTATTTGTTC |
| | TGGTACAAGCAGCCTCCCAGCAGGCAGATGATTCTCGTTATTCGCCAAGAAG |
| | CTTATAAGCAACAGAATGCAACGGAGAATCGTTTCTCTGTGAACTTCCAGAA |
| | AGCAGCCAAATCCTTCAGTCTCAAGATCTCAGACTCACAGCTGGGGGACACT |
| | GCGATGTATTTCTGTGCTTTCGACTATGGTCAGAATTTTGTCTTTGGTCCCGG |
| | AACCAGATTGTCCGTGCTGCCC (SEQ ID NO: 282) |
| PN46909 | NAGVTQTPKFRILKIGQSMTLQCAQDMNHNYMYWYRQDPGMGLKLIYYSVGA |
| Vβ | GITDKGEVPNGYNVSRSTTEYFPLRLELAAPSQTSVYFCASSYGGGQTEAFFGQG |
| | TRLTVV (SEQ ID NO: 283) |
| | AATGCTGGTGTCACTCAGACCCCAAAATTCCGCATCCTGAAGATAGGACAGA |
| | GCATGACACTGCAGTGTGCCCAGGATATGAACCATAACTACATGTACTGGTA |
| | TCGACAAGACCCAGGCATGGGGCTGAAGCTGATTTATTATTCAGTTGGTGCT |
| | GGTATCACTGATAAAGGAGAAGTCCCGAATGGCTACAACGTCTCCAGATCAA |
| | CCACAGAGTATTTCCCGCTCAGGCTGGAGTTGGCTGCTCCCTCCCAGACATCT |
| | GTGTACTTCTGTGCCAGCAGTTACGGGGGGGGGCAGACTGAAGCTTTCTTTG |
| | GACAAGGCACCAGACTCACAGTTGTA (SEQ ID NO: 284) |
| PN46914 | AQKVTQAQPEISVVEKEDVTLDCVYETRDTTYYLFWYKQPPSGELVFLIRRNSFD |
| Vα | EQNEISGRYSWNFQKSTSSFNFTITASQVVDSAVYFCALSEGYNQGGKLIFGQGT |
| | ELSVKP (SEQ ID NO: 285) |
| | GCTCAGAAGGTAACTCAAGCGCAGCCTGAAATTTCTGTGGTGGAGAAGGAG |
| | GATGTGACCTTGGACTGTGTGTATGAAACCCGTGATACTACTTATTACTTATT |
| | CTGGTACAAGCAACCACCAAGTGGAGAATTGGTTTTCCTTATTCGTCGGAAC |
| | TCTTTTGATGAGCAAAATGAAATAAGTGGTCGGTATTCTTGGAACTTCCAGA |
| | AATCCACCAGTTCCTTCAACTTCACCATCACAGCCTCACAAGTCGTGGACTCA |
| | GCAGTATACTTCTGTGCTCTGAGTGAGGGTTATAACCAGGGAGGAAAGCTTA |
| | TCTTCGGACAGGGAACGGAGTTATCTGTGAAACCC (SEQ ID NO: 286) |
| PN46914 | DVKVTQSSRYLVKRTGEKVFLECVQDMDHENMFWYRQDPGLGLRLIYFSYDVK |
| Vβ | MKEKGDIPEGYSVSREKKERFSLILESASTNQTSMYLCASGADSNQPQHFGDGTR |
| | LSIL (SEQ ID NO: 287) |
| | GATGTGAAAGTAACCCAGAGCTCGAGATATCTAGTCAAAAGGACGGGAGAG |
| | AAAGTTTTTCTGGAATGTGTCCAGGATATGGACCATGAAAATATGTTCTGGTA |
| | TCGACAAGACCCAGGTCTGGGGCTACGGCTGATCTATTTCTCATATGATGTTA |
| | AAATGAAAGAAAAAGGAGATATTCCTGAGGGGTACAGTGTCTCTAGAGAGA |
| | AGAAGGAGCGCTTCTCCCTGATTCTGGAGTCCGCCAGCACCAACCAGACATC |
| | TATGTACCTCTGTGCCAGCGGGGCAGATAGCAATCAGCCCCAGCATTTTGGT |
| | GATGGGACTCGACTCTCCATCCTA (SEQ ID NO: 288) |

TABLE 6
Amino acid CDR sequences for VelociT TCRs specific for PRAME (425-433)/HLA-A2

| | | SEQ | | SEQ | | SEQ | | SEQ | | SEQ | | SEQ |
|---|---|---|---|---|---|---|---|---|---|---|---|---|
| TCR | | ID | | ID | | ID | | ID | | ID | | ID |
| ID | Vα CDR1 | NO: | Vα CDR2 | NO: | Vα CDR3 | NO: | Vβ CDR1 | NO: | Vβ CDR2 | NO: | Vβ CDR3 | NO: |
| PN42365 | SVFSS | 289 | VVTGGEV | 290 | AANGGSQGNLI | 291 | SGHDT | 292 | YYEEEE | 293 | ASSLQDYGYT | 294 |
| PN42378 | TISGTDY | 295 | GLTSN | 296 | ILRPDSWGKFQ | 297 | SGHDT | 298 | YYEEEE | 299 | ASSLQDYGYT | 300 |
| PN42386 | SVFSS | 301 | VVTGGEV | 302 | ATNGGSQGNLI | 303 | SGHDT | 304 | YYEEEE | 305 | ASSLQDYGYT | 306 |
| PN42441 | TISGTDY | 307 | GLTSN | 308 | IRRPGNQFY | 309 | MNHEY | 310 | SMNVEV | 311 | ASSLWTGSEAF | 312 |
| PN42442 | SVFSS | 313 | VVTGGEV | 314 | AGGTSGTYKYI | 315 | MNHEY | 316 | SMNVEV | 317 | ASSPGTANYGYT | 318 |
| PN42450 | YSGSPE | 319 | HISR | 320 | ALGNTDKLI | 321 | MDHEN | 322 | SYDVKM | 323 | ASSSPRTGWYGYT | 324 |
| PN42455 | TISGTDY | 325 | GLTSN | 326 | ILRPDSWGKFQ | 327 | SGHDT | 328 | YYEEEE | 329 | ASSLVDYGYT | 330 |
| PN42476 | TISGTDY | 331 | GLTSN | 332 | ILRPDSWGKFQ | 333 | MDHEY | 334 | SMNVEV | 335 | ASSLGGVDERLS | 336 |
| PN42483 | SVFSS | 337 | VVTGGEV | 338 | AGDGGSQGNLI | 339 | MNHEY | 340 | SMNVEV | 341 | ASSLGGADEKLF | 342 |
| PN42496 | TISGTDY | 343 | GLTSN | 344 | ILGQGAQKLV | 345 | MNHEY | 346 | SMNVEV | 347 | ASSLWTGGGYT | 348 |
| PN42498 | SVFSS | 349 | VVTGGEV | 350 | AGDGGSQGNLI | 351 | SGHDT | 352 | YYEEEE | 353 | ASSLVDYGYT | 354 |
| PN42558 | DSVNN | 355 | IPSGT | 356 | VLGGGSQGNLI | 357 | SGHDT | 358 | YYEEEE | 359 | ASSFTDYGYT | 360 |
| PN42561 | TISGTDY | 361 | GLTSN | 362 | ILRDGTGNQFY | 363 | MNHEY | 364 | SMNVEV | 365 | ASSLWTGGGYT | 366 |
| PN42562 | SVFSS | 367 | VVTGGEV | 368 | AGGPSGTYKYI | 369 | MNHEY | 370 | SMNVEV | 371 | ASSPGTPNYGYT | 372 |
| PN42610 | DRGSQS | 373 | IYSNGD | 374 | AGNYGQNFV | 375 | PRHDT | 376 | FYEKMQ | 377 | ASSIGLNQPQH | 378 |
| PN42654 | TISGTDY | 379 | GLTSN | 380 | ILRDGIGNQFY | 381 | MNHEY | 382 | SMNVEV | 383 | ASSLWTGGGYT | 384 |
| PN42655 | TISGTDY | 385 | GLTSN | 386 | ILRPDSWGKFQ | 387 | MNHEY | 388 | SMNVEV | 389 | ASSLWTGGGYT | 390 |
| PN42677 | TISGTDY | 391 | GLTSN | 392 | ILRDGTGNQFY | 393 | MNHEY | 394 | SMNVEV | 395 | ASSSTGYYGYT | 396 |
| PN42683 | TISGTDY | 397 | GLTSN | 398 | ILRDREYGNKLV | 399 | MNHEY | 400 | SMNVEV | 401 | ASSLWTGGGYT | 402 |
| PN42689 | SVFSS | 403 | VVTGGEV | 404 | AEDGGSQGNLI | 405 | SGHDT | 406 | YYEEEE | 407 | ASSLSDYGYT | 408 |
| PN42706 | DSVNN | 409 | IPSGT | 410 | AVEASGTYKYI | 411 | MNHEY | 412 | SMNVEV | 413 | ASSWGTGGYGYT | 414 |
| PN42707 | NSASDY | 415 | IRSNMDK | 416 | AENKRDNYGQNFV | 417 | MNHEY | 418 | SMNVEV | 419 | ASSFWVNTEAF | 420 |
| PN42711 | DSVNN | 421 | IPSGT | 422 | AVGSSNGYKLS | 423 | MNHEY | 424 | SMNVEV | 425 | ASSPGTGGFSPLH | 426 |
| PN42712 | DSVNN | 427 | IPSGT | 428 | AVGSSNDYKLS | 429 | MNHEY | 430 | SMNVEV | 431 | ASSPGTGGFSPLH | 432 |
| PN42746 | NSASDY | 433 | IRSNMDK | 434 | AENRQDNYGQNFV | 435 | MNHEY | 436 | SMNVEV | 437 | ASSLWVNTEAF | 438 |
| PN42750 | TISGTDY | 439 | GLTSN | 440 | ILRPDSWGKFQ | 441 | MDHEN | 442 | SYDVKM | 443 | ASSTVRQGNYGYT | 444 |
| PN42762 | TISGTDY | 445 | GLTSN | 446 | ILNTGTASKLT | 447 | MNHEY | 448 | SMNVEV | 449 | ASSLSSNTEAF | 450 |
| PN42774 | DRGSQS | 451 | IYSNGD | 452 | AVNRGTDKLI | 453 | SGHDT | 454 | YYEEEE | 455 | ASSWTDYGYT | 456 |
| PN42776 | TISGTDY | 457 | GLTSN | 458 | ILRPDSWGKFQ | 459 | SGHDT | 460 | YYEEEE | 461 | ASSWTDYGYT | 462 |
| PN42780 | SVFSS | 463 | VVTGGEV | 464 | AGGTSGTYKYI | 465 | MNHEY | 466 | SMNVEV | 467 | ASSPGTPNYGYT | 468 |
| PN42795 | NSASQS | 469 | VYSSGN | 470 | VVNGGSQGNLI | 471 | SGHDT | 472 | YYEEEE | 473 | ASSVGDYGYT | 474 |
| PN42815 | NSASDY | 475 | IRSNMDK | 476 | AENNYGQNFV | 477 | MNHEY | 478 | SMNVEV | 479 | ASSLWDSSPLH | 480 |
| PN42826 | NSMFDY | 481 | ISSIKDK | 482 | AASAGSARQLT | 483 | MNHEY | 484 | SMNVEV | 485 | ASSLYTHTEAF | 486 |
| PN42833 | TISGNEY | 487 | GLKNN | 488 | IVRDTTSGTYKYI | 489 | MNHEY | 490 | SMNVEV | 491 | ASSLSSTGFSPLH | 492 |
| PN42840 | SVFSS | 493 | VVTGGEV | 494 | AEGGGSQGNLI | 495 | SGHDT | 496 | YYEEEE | 497 | ASSWTDYGYT | 498 |
| PN42845 | SVFSS | 499 | VVTGGEV | 500 | AGIRSNDYKLS | 501 | MNHEY | 502 | SMNVEV | 503 | ASSSWTAHTEAF | 504 |
| PN42870 | SVFSS | 505 | VVTGGEV | 506 | AENSGGGADGLT | 507 | SGHDT | 508 | YYEEEE | 509 | ASSFTDYGYT | 510 |
| PN42879 | SVFSS | 511 | VVTGGEV | 512 | AGEDFGNEKLT | 513 | SGHDT | 514 | YYEEEE | 515 | ASSWADYGYT | 516 |
| PN42888 | SSNFYA | 517 | MTLNGDE | 518 | AFLTGNQFY | 519 | MDHEN | 520 | SYDVKM | 521 | ASSTVRQGNYGYT | 522 |
| PN42895 | NSMFDY | 523 | ISSIKDK | 524 | AASAGSARQLT | 525 | MNHEY | 526 | SMNVEV | 527 | ASSLWSNTEAF | 528 |

TABLE 7
Nucleic acid CDR sequences for VelociT TCRs specific for PRAME (425-433)/HLA-A2

| | | SEQ | | SEQ | | SEQ | | SEQ | | SEQ | | SEQ |
|---|---|---|---|---|---|---|---|---|---|---|---|---|
| TCR | | ID | | ID | | ID | | ID | | ID | | ID |
| ID | Vα CDR1 | NO: | Vα CDR2 | NO: | Vα CDR3 | NO: | Vβ CDR1 | NO: | Vβ CDR2 | NO: | Vβ CDR3 | NO: |
| PN42365 | AGTGTTT | 529 | GTAGTTACG | 530 | GCCGCTAATGGAGGA | 531 | TCTGGGC | 532 | TATTATG | 533 | GCCAGCAGCTTACA | 534 |
| | TTTCCAG | | GGTGGAGAA | | AGCCAAGGAAATCTC | | ATGACA | | AGGAGGA | | GGACTATGGCTACA | |
| | C | | GTG | | ATC | | CT | | AGAG | | CC | |
| PN42378 | ACAATCA | 535 | GGTCTTACA | 536 | ATCCTGCGGCCTGACA | 537 | TCTGGGC | 538 | TATTATG | 539 | GCCAGCAGCTTACA | 540 |
| | GTGGAAC | | AGCAAT | | GCTGGGGGAAATTCC | | ATGACA | | AGGAGGA | | GGACTATGGCTACA | |
| | TGATTAC | | | | AG | | CT | | AGAG | | CC | |
| PN42386 | AGTGTTT | 541 | GTAGTTACG | 542 | GCCACTAATGGAGGA | 543 | TCTGGGC | 544 | TATTATG | 545 | GCCAGCAGCTTACA | 546 |
| | TTTCCAG | | GGTGGAGAA | | AGCCAAGGAAATCTC | | ATGACA | | AGGAGGA | | GGACTATGGCTACA | |
| | C | | GTG | | ATC | | CT | | AGAG | | CC | |
| PN42441 | ACAATCA | 547 | GGTCTTACA | 548 | ATCAGGCGGCCCGGT | 549 | ATGAAC | 550 | TCAATGA | 551 | GCCAGCAGTTTATG | 552 |
| | GTGGAAC | | AGCAAT | | AACCAGTTCTAT | | CATGAGT | | ATGTTGA | | GACAGGGTCTGAAG | |
| | TGATTAC | | | | | | AT | | GGTG | | CTTTC | |
| PN42442 | AGTGTTT | 553 | GTAGTTACT | 554 | GCAGGAGGGACCTCA | 555 | ATGAAC | 556 | TCAATGA | 557 | GCCAGCAGTCCAGG | 558 |
| | TTTCCAG | | GGTGGAGAA | | GGAACCTACAAATAC | | CATGAGT | | ATGTTGA | | GACAGCTAACTATG | |
| | C | | GTG | | ATC | | AT | | GGTG | | GCTACACC | |
| PN42450 | TATTCTG | 559 | CACATCTCT | 560 | GCTCTAGGGAACACC | 561 | ATGGAC | 562 | TCATATG | 563 | GCCAGCAGTTCGCC | 564 |
| | GGAGTCC | | AGA | | GACAAGCTCATC | | CATGAA | | ATGTTAA | | CAGGACAGGGTGGT | |
| | TGAA | | | | | | AAT | | AATG | | ATGGCTACACC | |
| PN42455 | ACAATCA | 565 | GGTCTTACA | 566 | ATCCTGCGGCCTGACA | 567 | TCTGGGC | 568 | TATTATG | 569 | GCCAGCAGCTTGGT | 570 |
| | GTGGAAC | | AGCAAT | | GCTGGGGGAAATTCC | | ATGACA | | AGGAGGA | | GGATTATGGCTACA | |
| | TGATTAC | | | | AG | | CT | | AGAG | | CC | |
| PN42476 | ACAATCA | 571 | GGTCTTACA | 572 | ATCCTGCGGCCTGACA | 573 | ATGGAC | 574 | TCAATGA | 575 | GCCAGCAGTTTAGG | 576 |
| | GTGGAAC | | AGCAAT | | GCTGGGGGAAATTCC | | CATGAGT | | ATGTTGA | | GGGCGTGGATGAAA | |
| | TGATTAC | | | | AG | | AT | | GGTG | | GACTGTCT | |
| PN42483 | AGTGTTT | 577 | GTAGTTACG | 578 | GCAGGGGATGGAGGA | 579 | ATGAAC | 580 | TCAATGA | 581 | GCCAGCAGTTTAGG | 582 |
| | TTTCCAG | | GGTGGAGAA | | AGCCAAGGAAATCTC | | CATGAGT | | ATGTTGA | | GGGCGCGGATGAAA | |
| | C | | GTG | | ATC | | AT | | GGTG | | AACTGTTT | |
| PN42496 | ACAATCA | 583 | GGTCTTACA | 584 | ATCCTGGGTCAGGGA | 585 | ATGAAC | 586 | TCAATGA | 587 | GCCAGCAGTTTATG | 588 |
| | GTGGAAC | | AGCAAT | | GCCCAGAAGCTGGTA | | CATGAGT | | ATGTTGA | | GACAGGGGGCGGCT | |
| | TGATTAC | | | | | | AT | | GGTG | | ACACC | |
| PN42498 | AGTGTTT | 589 | GTAGTTACG | 590 | GCAGGGGATGGAGGA | 591 | TCTGGGC | 592 | TATTATG | 593 | GCCAGCAGCTTGGT | 594 |
| | TTTCCAG | | GGTGGAGAA | | AGCCAAGGAAATCTC | | ATGACA | | AGGAGGA | | GGATTATGGCTACA | |
| | C | | GTG | | ATC | | CT | | AGAG | | CC | |
| PN42558 | GACTCTG | 595 | ATTCCCTCA | 596 | GTCCTAGGGGGAGGA | 597 | TCTGGGC | 598 | TATTATG | 599 | GCCAGCAGCTTTAC | 600 |
| | TGAACAA | | GGGACA | | AGCCAAGGAAATCTC | | ATGACA | | AGGAGGA | | AGACTATGGCTACA | |
| | T | | | | ATC | | CT | | AGAG | | CC | |
| PN42561 | ACAATCA | 601 | GGTCTTACA | 602 | ATCCTGAGAGACGGG | 603 | ATGAAC | 604 | TCAATGA | 605 | GCCAGCAGTTTATG | 606 |
| | GTGGAAC | | AGCAAT | | ACCGGTAACCAGTTCT | | CATGAGT | | ATGTTGA | | GACAGGGGGTGGCT | |
| | TGATTAC | | | | AT | | AT | | GGTG | | ACACC | |
| PN42562 | AGTGTTT | 607 | GTAGTTACG | 608 | GCAGGAGGACCCTCA | 609 | ATGAAC | 610 | TCAATGA | 611 | GCCAGCAGTCCCGG | 612 |
| | TTTCCAG | | GGTGGAGAA | | GGAACCTACAAATAC | | CATGAGT | | ATGTTGA | | GACACCTAACTATG | |
| | C | | GTG | | ATC | | AT | | GGTG | | GCTACACC | |
| PN42610 | GACCGAG | 613 | ATATACTCC | 614 | GCCGGGAACTATGGT | 615 | CCTAGAC | 616 | TTTTATG | 617 | GCCAGCAGCATCGG | 618 |
| | GTTCCCA | | AATGGTGAC | | CAGAATTTTGTC | | ACGACA | | AAAAGAT | | ACTTAATCAGCCCC | |
| | GTCC | | | | | | CT | | GCAG | | AGCAT | |
| PN42654 | ACAATCA | 619 | GGTCTTACA | 620 | ATCCTGAGAGACGGC | 621 | ATGAAC | 622 | TCAATGA | 623 | GCCAGCAGTTTATG | 624 |
| | GTGGAAC | | AGCAAT | | ATCGGTAACCAGTTCT | | CATGAGT | | ATGTTGA | | GACAGGGGGAGGCT | |
| | TGATTA | | | | AT | | AT | | GGTG | | ACACC | |
| PN42655 | ACAATCA | 625 | GGTCTTACA | 626 | ATCCTGCGGCCTGACA | 627 | ATGAAC | 628 | TCAATGA | 629 | GCCAGCAGTTTATG | 630 |
| | GTGGAAC | | AGCAAT | | GCTGGGGGAAATTCC | | CATGAGT | | ATGTTGA | | GACAGGGGGAGGCT | |
| | TGATTAC | | | | AG | | AT | | GGTG | | ACACC | |
| PN42677 | ACAATCA | 631 | GGTCTTACA | 632 | ATCCTGAGAGACGGC | 633 | ATGAAC | 634 | TCAATGA | 635 | GCCAGCAGTTCGAC | 636 |
| | GTGGAAC | | AGCAAT | | ACCGGTAACCAGTTCT | | CATGAGT | | ATGTTGA | | AGGGTACTATGGCT | |
| | TGATTAC | | | | AT | | AT | | GGTG | | ACACC | |
| PN42683 | ACAATCA | 637 | GGTCTTACA | 638 | ATCCTGAGAGACCGG | 639 | ATGAAC | 640 | TCAATGA | 641 | GCCAGCAGTTTATG | 642 |
| | GTGGAAC | | AGCAAT | | GAATATGGAAACAAA | | CATGAGT | | ATGTTGA | | GACAGGGGGCGGCT | |
| | TGATTAC | | | | CTGGTC | | AT | | GGTG | | ACACC | |
| PN42689 | AGTGTTT | 643 | GTAGTTACG | 644 | GCAGAAGATGGAGGA | 645 | TCTGGGC | 646 | TATTATG | 647 | GCCAGCAGCTTATC | 648 |
| | TTTCCAG | | GGTGGAGAA | | AGCCAAGGAAATCTC | | ATGACA | | AGGAGGA | | AGACTATGGCTACA | |
| | C | | GTG | | ATC | | CT | | AGAG | | CC | |
| PN42706 | GACTCTG | 649 | ATTCCCTCA | 650 | GCTGTGGAGGCCTCA | 651 | ATGAAC | 652 | TCAATGA | 653 | GCCAGCAGTTGGGG | 654 |
| | TGAACAA | | GGGACA | | GGAACCTACAAATAC | | CATGAGT | | ATGTTGA | | GACAGGGGGCTATG | |
| | T | | | | ATC | | AT | | GGTG | | GCTACACC | |
| PN42707 | AACAGCG | 655 | ATTCGTTCA | 656 | GCAGAGAATAAGCGG | 657 | ATGAAC | 658 | TCAATGA | 659 | GCCAGCAGTTTCTG | 660 |
| | CCTCAGA | | AATATGGAC | | GATAACTATGGTCAG | | CATGAGT | | ATGTTGA | | GGTGAACACTGAAG | |
| | CTAC | | AAA | | AATTTTGTC | | AT | | GGTG | | CTTTC | |
| PN42711 | GACTCTG | 661 | ATTCCCTCA | 662 | GCTGTGGGGAGTTCTA | 663 | ATGAAC | 664 | TCAATGA | 665 | GCCAGCAGTCCCGG | 666 |
| | TGAACAA | | GGGACA | | ACGGCTACAAGCTCA | | CATGAGT | | ATGTTGA | | GACAGGGGGATTTT | |
| | T | | | | GC | | AT | | GGTG | | CACCCCTCCAC | |
| PN42712 | GACTCTG | 667 | ATTCCCTCA | 668 | GCTGTGGGGAGTTCTA | 669 | ATGAAC | 670 | TCAATGA | 671 | GCCAGCAGTCCCGG | 672 |
| | TGAACAA | | GGGACA | | ACGACTACAAGCTCA | | CATGAGT | | ATGTTGA | | GACAGGGGGATTTT | |
| | T | | | | GC | | AT | | GGTG | | CACCCCTCCAC | |
| PN42746 | AACAGCG | 673 | ATTCGTTCA | 674 | GCAGAGAATAGGCAG | 675 | ATGAAC | 676 | TCAATGA | 677 | GCCAGCAGTTTATG | 678 |
| | CCTCAGA | | AATATGGAC | | GATAACTATGGTCAG | | CATGAGT | | ATGTTGA | | GGTTAACACTGAAG | |
| | CTAC | | AAA | | AATTTTGTC | | AT | | GGTG | | CTTTC | |
| PN42750 | ACAATCA | 679 | GGTCTTACA | 680 | ATCCTGCGGCCTGACA | 681 | ATGGAC | 682 | TCATATG | 683 | GCCAGCAGTACCGT | 684 |
| | GTGGAAC | | AGCAAT | | GCTGGGGGAAGTTCC | | CATGAA | | ATGTTAA | | GAGGCAGGGGAACT | |
| | TGATTAC | | | | AG | | AAT | | AATG | | ATGGCTACACC | |
| PN42762 | ACAATCA | 685 | GGTCTTACA | 686 | ATCCTGAATACCGGCA | 687 | ATGAAC | 688 | TCAATGA | 689 | GCCAGCAGTTTATC | 690 |
| | GTGGAAC | | AGCAAT | | CTGCCAGTAAACTCAC | | CATGAGT | | ATGTTGA | | GTCGAACACTGAAG | |
| | TGATTAC | | | | c | | AT | | GGTG | | CTTTC | |
| PN42774 | GACCGAG | 691 | ATATACTCC | 692 | GCCGTGAACAGAGGC | 693 | TCTGGGC | 694 | TATTATG | 695 | GCCAGCAGCTGGAC | 696 |
| | GTTCCCA | | AATGGTGAC | | ACCGACAAGCTCATC | | ATGACA | | AGGAGGA | | AGACTATGGCTACA | |
| | GTCC | | | | | | CT | | AGAG | | CC | |
| PN42776 | ACAATCA | 697 | GGTCTTACA | 698 | ATCCTGCGGCCTGACA | 699 | TCTGGGC | 700 | TATTATG | 701 | GCCAGCAGCTGGAC | 702 |
| | GTGGAAC | | AGCAAT | | GCTGGGGGAAATTCC | | ATGACA | | AGGAGGA | | AGACTATGGCTACA | |
| | TGATTAC | | | | AG | | CT | | AGAG | | CC | |
| PN42780 | AGTGTTT | 703 | GTAGTTACG | 704 | GCAGGAGGAACCTCA | 705 | ATGAAC | 706 | TCAATGA | 707 | GCCAGCAGTCCCGG | 708 |
| | TTTCCAG | | GGTGGAGAA | | GGAACCTACAAATAC | | CATGAGT | | ATGTTGA | | GACACCCAACTATG | |
| | C | | GTG | | ATC | | AT | | GGTG | | GCTACACC | |
| PN42795 | AACAGTG | 709 | GTATACTCC | 710 | GTGGTGAATGGAGGA | 711 | TCTGGGC | 712 | TATTATG | 713 | GCCAGCAGCGTAGG | 714 |
| | CTTCTCA | | AGTGGTAAT | | AGCCAAGGAAATCTC | | ATGACA | | AGGAGGA | | GGACTATGGCTACA | |
| | GTCT | | | | ATC | | CT | | AGAG | | CC | |
| PN42815 | AACAGCG | 715 | ATTCGTTCA | 716 | GCAGAGAACAACTAT | 717 | ATGAAC | 718 | TCAATGA | 719 | GCCAGCAGTTTATG | 720 |
| | CCTCAGA | | AATATGGAC | | GGTCAGAATTTTGTC | | CATGAGT | | ATGTTGA | | GGACAGTTCACCCC | |
| | CTAC | | AAA | | | | AT | | GGTG | | TCCAC | |
| PN42826 | AACAGCA | 721 | ATAAGTTCC | 722 | GCAGCAAGCGCCGGT | 723 | ATGAAC | 724 | TCAATGA | 725 | GCCAGCAGTTTATA | 726 |
| | TGTTTGA | | ATTAAGGAT | | TCTGCAAGGCAACTG | | CATGAGT | | ATGTTGA | | CACCCACACTGAAG | |
| | TTAT | | AAA | | ACC | | AT | | GGTG | | CTTTC | |
| PN42833 | ACCATCA | 727 | GGTCTAAAA | 728 | ATCGTCAGAGACACT | 729 | ATGAAC | 730 | TCAATGA | 731 | GCCAGCAGTTTATC | 732 |
| | GTGGAAA | | AACAAT | | ACCTCAGGAACCTAC | | CATGAGT | | ATGTTGA | | CTCGACAGGTTTTT | |
| | TGAGTAT | | | | AAATACATC | | AT | | GGTG | | CACCCCTCCAC | |
| PN42840 | AGTGTTT | 733 | GTAGTTACG | 734 | GCAGAGGGGGGAGGA | 735 | TCTGGGC | 736 | TATTATG | 737 | GCCAGCAGCTGGAC | 738 |
| | TTTCCAG | | GGTGGAGAA | | AGCCAAGGAAATCTC | | ATGACA | | AGGAGGA | | AGACTATGGCTACA | |
| | C | | GTG | | ATC | | CT | | AGAG | | CC | |
| PN42845 | AGTGTTT | 739 | GTAGTTACG | 740 | GCAGGGATACGTTCTA | 741 | ATGAAC | 742 | TCAATGA | 743 | GCCAGCAGTTCCTG | 744 |
| | TTTCCAG | | GGTGGAGAA | | ACGACTACAAGCTCA | | CATGAGT | | ATGTTGA | | GACAGCCCACACTG | |
| | C | | GTG | | GC | | AT | | GGTG | | AAGCTTTC | |
| PN42870 | AGTGTTT | 745 | GTAGTTACG | 746 | GCAGAAAATTCAGGA | 747 | TCTGGGC | 748 | TATTATG | 749 | GCCAGCAGCTTCAC | 750 |
| | TTTCCAG | | GGTGGAGAA | | GGAGGTGCTGACGGA | | ATGACA | | AGGAGGA | | AGACTATGGCTACA | |
| | C | | GTG | | CTCACC | | CT | | AGAG | | CC | |
| PN42879 | AGTGTTT | 751 | GTAGTTACG | 752 | GCAGGAGAGGACTTT | 753 | TCTGGGC | 754 | TATTATG | 755 | GCCAGCAGCTGGGC | 756 |
| | TTTCCAG | | GGTGGAGAA | | GGAAATGAGAAATTA | | ATGACA | | AGGAGGA | | GGATTATGGCTACA | |
| | C | | GTG | | ACC | | CT | | AGAG | | CC | |
| PN42888 | TCCAGCA | 757 | ATGACTTTA | 758 | GCCTTTCTCACCGGTA | 759 | ATGGAC | 760 | TCATATG | 761 | GCCAGCAGTACCGT | 762 |
| | ATTTTTAT | | AATGGGGAT | | ACCAGTTCTAT | | CATGAA | | ATGTTAA | | GAGGCAGGGGAACT | |
| | GCC | | GAA | | | | AAT | | AATG | | ATGGCTACACC | |
| PN42895 | AACAGCA | 763 | ATAAGTTCC | 764 | GCAGCAAGCGCCGGT | 765 | ATGAAC | 766 | TCAATGA | 767 | GCCAGCAGTTTATG | 768 |
| | TGTTTGA | | ATTAAGGAT | | TCTGCAAGGCAACTG | | CATGAGT | | ATGTTGA | | GTCGAACACTGAAG | |
| | TTAT | | AAA | | ACC | | AT | | GGTG | | CTTTC | |

TABLE 8
Amino acid and nucleic acid sequences for VelociT TCRs
specific for PRAME (425-433)/HLA-A2

| | Domain Sequences |
|---|---|
| | Amino Acid Sequence (SEQ ID NO); CDR1, CDR2, and CDR3 |
| | sequences are underlined |
| Domain | Nucleic Acid Sequence (SEQ ID NO); CDR1, CDR2, and CDR3 |
| name | sequences are underlined |
| PN42365 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAANGGSQGNLIFGKGTKLSV |
| | KP (SEQ ID NO: 769) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCCGCTAATGGAGGAAGCCAAGGAAATCTCATCTTTGGAAAAGGC |
| | ACTAAACTCTCTGTTAAACCA (SEQ ID NO: 770) |
| PN42365 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLQDYGYTFGSGTRL |
| | TVV (SEQ ID NO: 771) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTACAGGACTATGGCTACACCTTCGGTTCGG |
| | GGACCAGGTTAACCGTTGTA (SEQ ID NO: 772) |
| PN42378 | DAKTTQPNSMESNEEEPVHLPCNHSTISGTDYIHWYRQLPSQGPEYVIHGLTSNV |
| Vα | NNRMASLAIAEDRKSSTLILHRSTLRDAAVYYCILRPDSWGKFQFGAGTQVVVT |
| | P (SEQ ID NO: 773) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAATTCCAGTTTGGAGCAGGGACCCA |
| | GGTTGTGGTCACCCCA (SEQ ID NO: 774) |
| PN42378 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLQDYGYTFGSGTRL |
| | TVV (SEQ ID NO: 775) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTACAGGACTATGGCTACACCTTCGGTTCGG |
| | GGACCAGGTTAACCGTTGTA (SEQ ID NO: 776) |
| PN42386 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCATNGGSQGNLIFGKGTKLSV |
| | KP (SEQ ID NO: 777) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCCACTAATGGAGGAAGCCAAGGAAATCTCATCTTTGGAAAAGGC |
| | ACTAAACTCTCTGTTAAACCA (SEQ ID NO: 778) |
| PN42386 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLQDYGYTFGSGTRL |
| | TVV (SEQ ID NO: 779) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTACAGGACTATGGCTACACCTTCGGTTCGG |
| | GGACCAGGTTAACCGTTGTA (SEQ ID NO: 780) |
| PN42441 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCIRRPGNQFYFGTGTSLTVIP |
| | (SEQ ID NO: 781) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCAGGCGGCCCGGTAACCAGTTCTATTTTGGGACAGGGACAAGTTTGAC |
| | GGTCATTCCA (SEQ ID NO: 782) |
| PN42441 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGSEAFFGQGTR |
| | LTVV (SEQ ID NO: 783) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGTCTGAAGCTTTCTTTGGA |
| | CAAGGCACCAGACTCACAGTTGTA (SEQ ID NO: 784) |
| PN42442 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGGTSGTYKYIFGTGTRLKV |
| | LA (SEQ ID NO: 785) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACTGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGAGGGACCTCAGGAACCTACAAATACATCTTTGGAACAGGC |
| | ACCAGGCTGAAGGTTTTAGCA (SEQ ID NO: 786) |
| PN42442 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSPGTANYGYTFGSGT |
| | RLTVV (SEQ ID NO: 787) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTCCAGGGACAGCTAACTATGGCTACACCTTC |
| | GGTTCGGGGACCAGGTTAACCGTTGTA (SEQ ID NO: 788) |
| PN42450 | AQRVTQPEKLLSVFKGAPVELKCNYSYSGSPELFWYVQYSRQRLQLLLRHISRES |
| Vα | IKGFTADLNKGETSFHLKKPFAQEEDSAMYYCALGNTDKLIFGTGTRLQVFP |
| | (SEQ ID NO: 789) |
| | GCCCAGAGAGTGACTCAGCCCGAGAAGCTCCTCTCTGTCTTTAAAGGGGCCC |
| | CAGTGGAGCTGAAGTGCAACTATTCCTATTCTGGGAGTCCTGAACTCTTCTGG |
| | TATGTCCAGTACTCCAGACAACGCCTCCAGTTACTCTTGAGACACATCTCTAG |
| | AGAGAGCATCAAAGGCTTCACTGCTGACCTTAACAAAGGCGAGACATCTTTC |
| | CACCTGAAGAAACCATTTGCTCAAGAGGAAGACTCAGCCATGTATTACTGTG |
| | CTCTAGGGAACACCGACAAGCTCATCTTTGGGACTGGGACCAGATTACAAGT |
| | CTTTCCA (SEQ ID NO: 790) |
| PN42450 | DVKVTQSSRYLVKRTGEKVFLECVQDMDHENMFWYRQDPGLGLRLIYFSYDVK |
| Vβ | MKEKGDIPEGYSVSREKKERFSLILESASTNQTSMYLCASSSPRTGWYGYTFGSG |
| | TRLTVV (SEQ ID NO: 791) |
| | GATGTGAAAGTAACCCAGAGCTCGAGATATCTAGTCAAAAGGACGGGAGAG |
| | AAAGTTTTTCTGGAATGTGTCCAGGATATGGACCATGAAAATATGTTCTGGTA |
| | TCGACAAGACCCAGGTCTGGGGCTACGGCTGATCTATTTCTCATATGATGTTA |
| | AAATGAAAGAAAAAGGAGATATTCCTGAGGGGTACAGTGTCTCTAGAGAGA |
| | AGAAGGAGCGCTTCTCCCTGATTCTGGAGTCCGCCAGCACCAACCAGACATC |
| | TATGTACCTCTGTGCCAGCAGTTCGCCCAGGACAGGGTGGTATGGCTACACC |
| | TTCGGTTCGGGGACCAGGTTAACCGTTGTA (SEQ ID NO: 792) |
| PN42455 | DAKTTQPNSMESNEEEPVHLPCNHSTISGTDYIHWYRQLPSQGPEYVIHGLTSNV |
| Vα | NNRMASLAIAEDRKSSTLILHRSTLRDAAVYYCILRPDSWGKFQFGAGTQVVVT |
| | P (SEQ ID NO: 793) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAATTCCAGTTTGGAGCAGGGACCCA |
| | GGTTGTGGTCACCCCA (SEQ ID NO: 794) |
| PN42455 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLVDYGYTFGSGTRL |
| | TVV (SEQ ID NO: 795) |
| | GACGCTGGAGTCACCCAAAGCCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTGGTGGATTATGGCTACACCTTCGGTTCGG |
| | GGACCAGGTTAACCGTTGTA (SEQ ID NO: 796) |
| PN42476 | DAKTTQPNSMESNEEEPVHLPCNHSTISGTDYIHWYRQLPSQGPEYVIHGLTSNV |
| Vα | NNRMASLAIAEDRKSSTLILHRSTLRDAAVYYCILRPDSWGKFQFGAGTQVVVT |
| | P (SEQ ID NO: 797) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAATTCCAGTTTGGAGCAGGGACCCA |
| | GGTTGTGGTCACCCCA (SEQ ID NO: 798) |
| PN42476 | EAQVTQSPRYLITVTGKKLTVTCSQNMDHEYMSWYRQDPGLGLRQIYYSMNVE |
| Vβ | VTDKGDVPEGYKVPRKEKRSFPLILESPCCSQTPLYLCASSLGGVDERLSFGSGT |
| | QLSVL (SEQ ID NO: 799) |
| | GAAGCCCAAGTGACCCAGAGCCCAAGATACCTCATCACAGTGACTGGAAAG |
| | AAGTTAACAGTGACTTGTTCTCAGAATATGGACCATGAGTATATGTCCTGGTA |
| | TCGACAGGACCCGGGGCTGGGCCTAAGGCAGATCTACTATTCAATGAATGTT |
| | GAGGTGACAGATAAGGGAGATGTTCCTGAAGGGTACAAAGTCCCTCGAAAA |
| | GAGAAGAGGAGTTTCCCCCTGATCCTGGAGTCGCCCTGCTGCAGCCAGACCC |
| | CTCTGTACCTCTGTGCCAGCAGTTTAGGGGGCGTGGATGAAAGACTGTCTTTT |
| | GGCAGTGGAACCCAGCTCTCCGTCTTG (SEQ ID NO: 800) |
| PN42483 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGDGGSQGNLIFGKGTKLSV |
| | KP (SEQ ID NO: 801) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGGGATGGAGGAAGCCAAGGAAATCTCATCTTTGGAAAAGGC |
| | ACTAAACTCTCTGTTAAACCA (SEQ ID NO: 802) |
| PN42483 | EAQVTQSPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLGGADEKLFFGSGT |
| | QLSVL (SEQ ID NO: 803) |
| | GAAGCCCAAGTGACCCAGAGCCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTAGGGGGCGCGGATGAAAAACTGTTTTTT |
| | GGCAGTGGAACCCAGCTCTCTGTCTTG (SEQ ID NO: 804) |
| PN42496 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILGQGAQKLVFGQGTRLTINP |
| | (SEQ ID NO: 805) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGGGTCAGGGAGCCCAGAAGCTGGTATTTGGCCAAGGAACCAGGCT |
| | GACTATCAACCCA (SEQ ID NO: 806) |
| PN42496 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGGGYTFGSGTR |
| | LTVV (SEQ ID NO: 807) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGGGCGGCTACACCTTCGGT |
| | TCGGGGACCAGGTTAACCGTTGTA (SEQ ID NO: 808) |
| PN42498 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGDGGSQGNLIFGKGTKLSV |
| | KP (SEQ ID NO: 809) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGGGATGGAGGAAGCCAAGGAAATCTCATCTTTGGAAAAGGC |
| | ACTAAACTCTCTGTTAAACCA (SEQ ID NO: 810) |
| PN42498 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLVDYGYTFGSGTRL |
| | TVV (SEQ ID NO: 811) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTGGTGGATTATGGCTACACCTTCGGTTCGG |
| | GGACCAGGTTAACCGTTGTA (SEQ ID NO: 812) |
| PN42558 | GIQVEQSPPDLILQEGANSTLRCNFSDSVNNLQWFHQNPWGQLINLFYIPSGTKQ |
| Vα | NGRLSATTVATERYSLLYISSSQTTDSGVYFCVLGGGSQGNLIFGKGTKLSVKP |
| | (SEQ ID NO: 813) |
| | GGAATACAAGTGGAGCAGAGTCCTCCAGACCTGATTCTCCAGGAGGGAGCC |
| | AATTCCACGCTGCGGTGCAATTTTTCTGACTCTGTGAACAATTTGCAGTGGTT |
| | TCATCAAAACCCTTGGGGACAGCTCATCAACCTGTTTTACATTCCCTCAGGGA |
| | CAAAACAGAATGGAAGATTAAGCGCCACGACTGTCGCTACGGAACGCTACA |
| | GCTTATTGTACATTTCCTCTTCCCAGACCACAGACTCAGGCGTTTATTTCTGT |
| | GTCCTAGGGGGAGGAAGCCAAGGAAATCTCATCTTTGGAAAAGGCACTAAA |
| | CTCTCTGTTAAACCA (SEQ ID NO: 814) |
| PN42558 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSFTDYGYTFGSGTRLT |
| | W (SEQ ID NO: 815) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTTACAGACTATGGCTACACCTTCGGTTCGG |
| | GGACCAGGTTAACCGTTGTA (SEQ ID NO: 816) |
| PN42561 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILRDGTGNQFYFGTGTSLTVIP |
| | (SEQ ID NO: 817) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAGAGACGGGACCGGTAACCAGTTCTATTTTGGGACAGGGACAAG |
| | TTTGACGGTCATTCCA (SEQ ID NO: 818) |
| PN42561 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGGGYTFGSGTR |
| | LTVV(SEQ ID NO: 819) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGGGTGGCTACACCTTCGGT |
| | TCGGGGACCAGGTTAACCGTTGTA (SEQ ID NO: 820) |
| PN42562 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGGPSGTYKYIFGTGTRLKV |
| | LA (SEQ ID NO: 821) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGAGGACCCTCAGGAACCTACAAATACATCTTTGGAACAGGC |
| | ACCAGGCTGAAGGTTTTAGCA (SEQ ID NO: 822) |
| PN42562 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSPGTPNYGYTFGSGT |
| | RLTVV (SEQ ID NO: 823) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTCCCGGGACACCTAACTATGGCTACACCTTC |
| | GGTTCGGGGACCAGGTTAACCGTTGTA (SEQ ID NO: 824) |
| PN42610 | QKEVEQNSGPLSVPEGAIASLNCTYSDRGSQSFFWYRQYSGKSPELIMSIYSNGD |
| Vα | KEDGRFTAQLNKASQYVSLLIRDSQPSDSATYLCAGNYGQNFVFGPGTRLSVLP |
| | (SEQ ID NO: 825) |
| | CAGAAGGAGGTGGAGCAGAATTCTGGACCCCTCAGTGTTCCAGAGGGAGCC |
| | ATTGCCTCTCTCAACTGCACTTACAGTGACCGAGGTTCCCAGTCCTTCTTCTG |
| | GTACAGACAATATTCTGGGAAAAGCCCTGAGTTGATAATGTCCATATACTCC |
| | AATGGTGACAAAGAAGATGGAAGGTTTACAGCACAGCTCAATAAAGCCAGC |
| | CAGTATGTTTCTCTGCTCATCAGAGACTCCCAGCCCAGTGATTCAGCCACCTA |
| | CCTCTGTGCCGGGAACTATGGTCAGAATTTTGTCTTTGGTCCCGGAACCAGAT |
| | TGTCCGTGCTGCCC (SEQ ID NO: 826) |
| PN42610 | AAGVIQSPRHLIKEKRETATLKCYPIPRHDTVYWYQQGPGQDPQFLISFYEKMQS |
| Vβ | DKGSIPDRFSAQQFSDYHSELNMSSLELGDSALYFCASSIGLNQPQHFGDGTRLSI |
| | L (SEQ ID NO: 827) |
| | GCTGCTGGAGTCATCCAGTCCCCAAGACATCTGATCAAAGAAAAGAGGGAA |
| | ACAGCCACTCTGAAATGCTATCCTATCCCTAGACACGACACTGTCTACTGGTA |
| | CCAGCAGGGTCCAGGTCAGGACCCCCAGTTCCTCATTTCGTTTTATGAAAAG |
| | ATGCAGAGCGATAAAGGAAGCATCCCTGATCGATTCTCAGCTCAACAGTTCA |
| | GTGACTATCATTCTGAACTGAACATGAGCTCCTTGGAGCTGGGGGACTCAGC |
| | CCTGTACTTCTGTGCCAGCAGCATCGGACTTAATCAGCCCCAGCATTTTGGTG |
| | ATGGGACTCGACTCTCCATCCTA (SEQ ID NO: 828) |
| PN42654 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILRDGIGNQFYFGTGTSLTVIP |
| | (SEQ ID NO: 829) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAGAGACGGCATCGGTAACCAGTTCTATTTTGGGACAGGGACAAG |
| | TTTGACGGTCATTCCA (SEQ ID NO: 830) |
| PN42654 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGGGYTFGSGTR |
| | LTVV(SEQ ID NO: 831) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGGGAGGCTACACCTTCGGT |
| | TCGGGGACCAGGTTAACCGTTGTA (SEQ ID NO: 832) |
| PN42655 | DAKTTQPNSMESNEEEPVHLPCNHSTISGTDYIHWYRQLPSQGPEYVIHGLTSNV |
| Vα | NNRMASLAIAEDRKSSTLILHRSTLRDAAVYYCILRPDSWGKFQFGAGTQVVVT |
| | P (SEQ ID NO: 833) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAATTCCAGTTTGGAGCAGGGACCCA |
| | GGTTGTGGTCACCCCA (SEQ ID NO: 834) |
| PN42655 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGGGYTFGSGTR |
| | LTVV (SEQ ID NO: 835) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGGGAGGCTACACCTTCGGT |
| | TCGGGGACCAGGTTAACCGTTGTA (SEQ ID NO: 836) |
| PN42677 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILRDGTGNQFYFGTGTSLTVIP |
| | (SEQ ID NO: 837) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAGAGACGGCACCGGTAACCAGTTCTATTTTGGGACAGGGACAAG |
| | TTTGACGGTCATTCCA (SEQ ID NO: 838) |
| PN42677 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSSTGYYGYTFGSGTR |
| | LTVV (SEQ ID NO: 839) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTCGACAGGGTACTATGGCTACACCTTCGGT |
| | TCGGGGACCAGGTTAACCGTTGTA (SEQ ID NO: 840) |
| PN42683 | DAKTTQPNSMESNEEEPVHLPCNHSTISGTDYIHWYRQLPSQGPEYVIHGLTSNV |
| Vα | NNRMASLAIAEDRKSSTLILHRATLRDAAVYYCILRDREYGNKLVFGAGTILRVK |
| | S (SEQ ID NO: 841) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAGAGACCGGGAATATGGAAACAAACTGGTCTTTGGCGCAGGAA |
| | CCATTCTGAGAGTCAAGTCC (SEQ ID NO: 842) |
| PN42683 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWTGGGYTFGSGTR |
| | LTVV (SEQ ID NO: 843) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGACAGGGGGCGGCTACACCTTCGGT |
| | TCGGGGACCAGGTTAACCGTTGTA (SEQ ID NO: 844) |
| PN42689 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAEDGGSQGNLIFGKGTKLSV |
| | KP (SEQ ID NO: 845) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGAAGATGGAGGAAGCCAAGGAAATCTCATCTTTGGAAAAGGC |
| | ACTAAACTCTCTGTTAAACCA (SEQ ID NO: 846) |
| PN42689 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSLSDYGYTFGSGTRLT |
| | VV (SEQ ID NO: 847) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTATCAGACTATGGCTACACCTTCGGTTCGG |
| | GGACCAGGTTAACCGTTGTA (SEQ ID NO: 848) |
| PN42706 | GIQVEQSPPDLILQEGANSTLRCNFSDSVNNLQWFHQNPWGQLINLFYIPSGTKQ |
| Vα | NGRLSATTVATERYSLLYISSSQTTDSGVYFCAVEASGTYKYIFGTGTRLKVLA |
| | (SEQ ID NO: 849) |
| | GGAATACAAGTGGAGCAGAGTCCTCCAGACCTGATTCTCCAGGAGGGAGCC |
| | AATTCCACGCTGCGGTGCAATTTTTCTGACTCTGTGAACAATTTGCAGTGGTT |
| | TCATCAAAACCCTTGGGGACAGCTCATCAACCTGTTTTACATTCCCTCAGGGA |
| | CAAAACAGAATGGAAGATTAAGCGCCACGACTGTCGCTACGGAACGCTACA |
| | GCTTATTGTACATTTCCTCTTCCCAGACCACAGACTCAGGCGTTTATTTCTGT |
| | GCTGTGGAGGCCTCAGGAACCTACAAATACATCTTTGGAACAGGCACCAGGC |
| | TGAAGGTTTTAGCA (SEQ ID NO: 850) |
| PN42706 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSWGTGGYGYTFGSGT |
| | RLTVV (SEQ ID NO: 851) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTGGGGGACAGGGGGCTATGGCTACACCTT |
| | CGGTTCGGGGACCAGGTTAACCGTTGTA (SEQ ID NO: 852) |
| PN42707 | GESVGLHLPTLSVQEGDNSIINCAYSNSASDYFIWYKQESGKGPQFIIDIRSNMDK |
| Vα | RQGQRVTVLLNKTVKHLSLQIAATQPGDSAVYFCAENKRDNYGQNFVFGPGTR |
| | LSVLP (SEQ ID NO: 853) |
| | GGAGAGAGTGTGGGGCTGCATCTTCCTACCCTGAGTGTCCAGGAGGGTGACA |
| | ACTCTATTATCAACTGTGCTTATTCAAACAGCGCCTCAGACTACTTCATTTGG |
| | TACAAGCAAGAATCTGGAAAAGGTCCTCAATTCATTATAGACATTCGTTCAA |
| | ATATGGACAAAAGGCAAGGCCAAAGAGTCACCGTTTTATTGAATAAGACAGT |
| | GAAACATCTCTCTCTGCAAATTGCAGCTACTCAACCTGGAGACTCAGCTGTCT |
| | ACTTTTGTGCAGAGAATAAGCGGGATAACTATGGTCAGAATTTTGTCTTTGGT |
| | CCCGGAACCAGATTGTCCGTGCTGCCC (SEQ ID NO: 854) |
| PN42707 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSFWVNTEAFFGQGTR |
| | LTVV (SEQ ID NO: 855) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTCTGGGTGAACACTGAAGCTTTCTTTGGA |
| | CAAGGCACCAGACTCACAGTTGTA (SEQ ID NO: 856) |
| PN42711 | GIQVEQSPPDLILQEGVNSTLRCNFSDSVNNLQWFHQNPWGQLINLFYIPSGTKQ |
| Vα | NGRLSATTVATERYSLLYISSSRTTDSGVYFCAVGSSNGYKLSFGAGTTVTVRA |
| | (SEQ ID NO: 857) |
| | GGAATACAAGTGGAGCAGAGTCCTCCAGACCTGATTCTCCAGGAGGGAGTCA |
| | ATTCCACGCTGCGGTGCAATTTTTCTGACTCTGTGAACAATTTGCAGTGGTTT |
| | CATCAAAACCCTTGGGGACAGCTCATCAACCTGTTTTACATTCCCTCAGGGAC |
| | AAAACAGAATGGAAGATTAAGCGCCACGACTGTGGCTACGGAACGCTACAG |
| | CTTATTGTACATTTCCTCTTCCCGGACCACAGACTCAGGCGTTTATTTCTGTGC |
| | TGTGGGGAGTTCTAACGGCTACAAGCTCAGCTTTGGAGCTGGAACCACAGTA |
| | ACTGTAAGAGCA (SEQ ID NO: 858) |
| PN42711 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSPGTGGFSPLHFGNGT |
| | RLTVT (SEQ ID NO: 859) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTCCCGGGACAGGGGGATTTTCACCCCTCCAC |
| | TTTGGGAACGGGACCAGGCTCACTGTGACA (SEQ ID NO: 860) |
| PN42712 | GIQVEQSPPDLILQEGANSTLRCNFSDSVNNLQWFHQNPWGQLINLFYIPSGTKQ |
| Vα | NGRLSATTVATERYSLLYISSSQTTDSGVYFCAVGSSNDYKLSFGAGTTVTVRA |
| | (SEQ ID NO: 861) |
| | GGAATACAAGTGGAGCAGAGTCCTCCAGACCTGATTCTCCAGGAGGGAGCC |
| | AATTCCACGCTGCGGTGCAATTTTTCTGACTCTGTGAACAATTTGCAGTGGTT |
| | TCATCAAAACCCTTGGGGACAGCTCATCAACCTGTTTTACATTCCCTCAGGGA |
| | CAAAACAGAATGGAAGATTAAGCGCCACGACTGTCGCTACGGAACGCTACA |
| | GCTTATTGTACATTTCCTCTTCCCAGACCACAGACTCAGGCGTTTATTTCTGT |
| | GCTGTGGGGAGTTCTAACGACTACAAGCTCAGCTTTGGAGCCGGAACCACAG |
| | TAACTGTAAGAGCA (SEQ ID NO: 862) |
| PN42712 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSPGTGGFSPLHFGNGT |
| | RLTVT (SEQ ID NO: 863) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTCCCGGGACAGGGGGATTTTCACCCCTCCAC |
| | TTTGGGAACGGGACCAGGCTCACTGTGACA (SEQ ID NO: 864) |
| PN42746 | GESVGLHLPTLSVQEGDNSIINCAYSNSASDYFIWYKQESGKGPQFIIDIRSNMDK |
| Vα | RQGQRVTVLLNKTVKHLSLQIAATQPGDSAVYFCAENRQDNYGQNFVFGPGTR |
| | LSVLP (SEQ ID NO: 865) |
| | GGAGAGAGTGTGGGGCTGCATCTTCCTACCCTGAGTGTCCAGGAGGGTGACA |
| | ACTCTATTATCAACTGTGCTTATTCAAACAGCGCCTCAGACTACTTCATTTGG |
| | TACAAGCAAGAATCTGGAAAAGGTCCTCAATTCATTATAGACATTCGTTCAA |
| | ATATGGACAAAAGGCAAGGCCAAAGAGTCACCGTTTTATTGAATAAGACAGT |
| | GAAACATCTCTCTCTGCAAATTGCAGCTACTCAACCTGGAGACTCAGCTGTCT |
| | ACTTTTGTGCAGAGAATAGGCAGGATAACTATGGTCAGAATTTTGTCTTTGGT |
| | CCCGGAACCAGATTGTCCGTGCTGCCC (SEQ ID NO: 866) |
| PN42746 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWVNTEAFFGQGTR |
| | LTVV (SEQ ID NO: 867) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGGTTAACACTGAAGCTTTCTTTGGA |
| | CAAGGCACCAGACTCACAGTTGTA (SEQ ID NO: 868) |
| PN42750 | DAKTTQPNSMESNEEEPVHLPCNHSTISGTDYIHWYRQLPSQGPEYVIHGLTSN |
| Vα | NNRMASLAIAEDRKSSTLILHRSTLRDAAVYYCILRPDSWGKFQFGAGTQVVVT |
| | P (SEQ ID NO: 869) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAGTTCCAGTTTGGAGCAGGGACCCA |
| | GGTTGTGGTCACCCCA (SEQ ID NO: 870) |
| PN42750 | DVKVTQSSRYLVKRTGEKVFLECVQDMDHENMFWYRQDPGLGLRLIYFSYDVK |
| Vβ | MKEKGDIPEGYSVSREKKERFSLILESASTNQTSMYLCASSTVRQGNYGYTFGSG |
| | TRLTVV (SEQ ID NO: 871) |
| | GATGTGAAAGTAACCCAGAGCTCGAGATATCTAGTCAAAAGGACGGGAGAG |
| | AAAGTTTTTCTGGAATGTGTCCAGGATATGGACCATGAAAATATGTTCTGGTA |
| | TCGACAAGACCCAGGTCTGGGGCTACGGCTGATCTATTTCTCATATGATGTTA |
| | AAATGAAAGAAAAAGGAGATATTCCTGAGGGGTACAGTGTCTCTAGAGAGA |
| | AGAAGGAGCGCTTCTCCCTGATTCTGGAGTCCGCCAGCACCAACCAGACATC |
| | TATGTACCTCTGTGCCAGCAGTACCGTGAGGCAGGGGAACTATGGCTACACC |
| | TTCGGTTCGGGGACCAGGTTAACCGTTGTA (SEQ ID NO: 872) |
| PN42762 | DAKTTQPNSMESNEEEPVHLPCNHSTISGTDYIHWYRQLPSQGPEYVIHGLTSN |
| Vα | NNRMASLAIAEDRKSSTLILHRATLRDAAVYYCILNTGTASKLTFGTGTRLQVTL |
| | (SEQ ID NO: 873) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTGCTACCTTGAGAGATGCTGCTGTGTACTACT |
| | GCATCCTGAATACCGGCACTGCCAGTAAACTCACCTTTGGGACTGGAACAAG |
| | ACTTCAGGTCACGCTC (SEQ ID NO: 874) |
| PN42762 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLSSNTEAFFGQGTRL |
| | TVV (SEQ ID NO: 875) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATCGTCGAACACTGAAGCTTTCTTTGGA |
| | CAAGGCACCAGACTCACAGTTGTA (SEQ ID NO: 876) |
| PN42774 | QKEVEQNSGPLSVPEGAIASLNCTYSDRGSQSFFWYRQYSGKSPELIMSIYSNGD |
| Vα | KEDGRFTAQLNKASQYVSLLIRDSQPSDSATYLCAVNRGTDKLIFGTGTRLQVFP |
| | (SEQ ID NO: 877) |
| | CAGAAGGAGGTGGAGCAGAATTCTGGACCCCTCAGTGTTCCAGAGGGAGCC |
| | ATTGCCTCTCTCAACTGCACTTACAGTGACCGAGGTTCCCAGTCCTTCTTCTG |
| | GTACAGACAATATTCTGGGAAAAGCCCTGAGTTGATAATGTCCATATACTCC |
| | AATGGTGACAAAGAAGATGGAAGGTTTACAGCACAGCTCAATAAAGCCAGC |
| | CAGTATGTTTCTCTGCTCATCAGAGACTCCCAGCCCAGTGATTCAGCCACCTA |
| | CCTCTGTGCCGTGAACAGAGGCACCGACAAGCTCATCTTTGGGACTGGGACC |
| | AGATTACAAGTCTTTCCA (SEQ ID NO: 878) |
| PN42774 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSWTDYGYTFGSGTRL |
| | TVV (SEQ ID NO: 879) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTGGACAGACTATGGCTACACCTTCGGTTCGG |
| | GGACCAGGTTAACCGTTGTA (SEQ ID NO: 880) |
| PN42776 | DAKTTQPNSMESNEEEPVHLPCNHSTISGTDYIHWYRQLPSQGPEYVIHGLTSNV |
| Vα | NNRMASLAIAEDRKSSTLILHRSTLRDAAVYYCILRPDSWGKFQFGAGTQVVVT |
| | P (SEQ ID NO: 881) |
| | GATGCTAAGACCACACAGCCAAATTCAATGGAGAGTAACGAAGAAGAGCCT |
| | GTTCACTTGCCTTGTAACCACTCCACAATCAGTGGAACTGATTACATACATTG |
| | GTATCGACAGCTTCCCTCCCAGGGTCCAGAGTACGTGATTCATGGTCTTACAA |
| | GCAATGTGAACAACAGAATGGCCTCTCTGGCAATCGCTGAAGACAGAAAGTC |
| | CAGTACCTTGATCCTGCACCGTTCTACCTTGAGAGATGCTGCTGTGTACTACT |
| | GCATCCTGCGGCCTGACAGCTGGGGGAAATTCCAGTTTGGAGCAGGGACCCA |
| | GGTTGTGGTCACCCCA (SEQ ID NO: 882) |
| PN42776 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSWTDYGYTFGSGTRL |
| | TVV (SEQ ID NO: 883) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTGGACAGACTATGGCTACACCTTCGGTTCGG |
| | GGACCAGGTTAACCGTTGTA (SEQ ID NO: 884) |
| PN42780 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGGTSGTYKYIFGTGTRLKV |
| | LA (SEQ ID NO: 885) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGAGGAACCTCAGGAACCTACAAATACATCTTTGGAACAGGC |
| | ACCAGGCTGAAGGTTTTAGCA (SEQ ID NO: 886) |
| PN42780 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSPGTPNYGYTFGSGT |
| | RLTVV (SEQ ID NO: 887) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTCCCGGGACACCCAACTATGGCTACACCTTC |
| | GGTTCGGGGACCAGGTTAACCGTTGTA (SEQ ID NO: 888) |
| PN42795 | RKEVEQDPGPFNVPEGATVAFNCTYSNSASQSFFWYRQDCRKEPKLLMSVYSSG |
| Vα | NEDGRFTAQLNRASQYISLLIRDSKLSDSATYLCVVNGGSQGNLIFGKGTKLSVK |
| | P (SEQ ID NO: 889) |
| | CGGAAGGAGGTGGAGCAGGATCCTGGACCCTTCAATGTTCCAGAGGGAGCC |
| | ACTGTCGCTTTCAACTGTACTTACAGCAACAGTGCTTCTCAGTCTTTCTTCTG |
| | GTACAGACAGGATTGCAGGAAAGAACCTAAGTTGCTGATGTCCGTATACTCC |
| | AGTGGTAATGAAGATGGAAGGTTTACAGCACAGCTCAATAGAGCCAGCCAG |
| | TATATTTCCCTGCTCATCAGAGACTCCAAGCTCAGTGATTCAGCCACCTACCT |
| | CTGTGTGGTGAATGGAGGAAGCCAAGGAAATCTCATCTTTGGAAAAGGCACT |
| | AAACTCTCTGTTAAACCA (SEQ ID NO: 890) |
| PN42795 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSVGDYGYTFGSGTRL |
| | TVV (SEQ ID NO: 891) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCGTAGGGGACTATGGCTACACCTTCGGTTCGG |
| | GGACCAGGTTAACCGTTGTA (SEQ ID NO: 892) |
| PN42815 | GESVGLHLPTLSVQEGDNSIINCAYSNSASDYFIWYKQESGKGPQFIIDIRSNMDK |
| Vα | RQGQRVTVLLNKTVKHLSLQIAATQPGDSAVYFCAENNYGQNFVFGPGTRLSVL |
| | P (SEQ ID NO: 893) |
| | GGAGAGAGTGTGGGGCTGCATCTTCCTACCCTGAGTGTCCAGGAGGGTGACA |
| | ACTCTATTATCAACTGTGCTTATTCAAACAGCGCCTCAGACTACTTCATTTGG |
| | TACAAGCAAGAATCTGGAAAAGGTCCTCAATTCATTATAGACATTCGTTCAA |
| | ATATGGACAAAAGGCAAGGCCAAAGAGTCACCGTTTTATTGAATAAGACAGT |
| | GAAACATCTCTCTCTGCAAATTGCAGCTACTCAACCTGGAGACTCAGCTGTCT |
| | ACTTTTGTGCAGAGAACAACTATGGTCAGAATTTTGTCTTTGGTCCCGGAACC |
| | AGATTGTCCGTGCTGCCC (SEQ ID NO: 894) |
| PN42815 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWDSSPLHFGNGTR |
| | LTVT (SEQ ID NO: 895) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGGACAGTTCACCCCTCCACTTTGGG |
| | AACGGGACCAGGCTCACTGTGACA (SEQ ID NO: 896) |
| PN42826 | DQQVKQNSPSLSVQEGRISILNCDYTNSMFDYFLWYKKYPAEGPTFLISISSIKDK |
| Vα | NEDGRFTVFLNKSAKHLSLHIVPSQPGDSAVYFCAASAGSARQLTFGSGTQLTVL |
| | P (SEQ ID NO: 897) |
| | GACCAGCAAGTTAAGCAAAATTCACCATCCCTGAGCGTCCAGGAAGGAAGA |
| | ATTTCTATTCTGAACTGTGACTATACTAACAGCATGTTTGATTATTTCCTATGG |
| | TACAAAAAATACCCTGCTGAAGGTCCTACATTCCTGATATCTATAAGTTCCAT |
| | TAAGGATAAAAATGAAGATGGAAGATTCACTGTTTTCTTAAACAAAAGTGCC |
| | AAGCACCTCTCTCTGCACATTGTGCCCTCCCAGCCTGGAGACTCTGCAGTGTA |
| | CTTCTGTGCAGCAAGCGCCGGTTCTGCAAGGCAACTGACCTTTGGATCTGGG |
| | ACACAATTGACTGTTTTACCT (SEQ ID NO: 898) |
| PN42826 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLYTHTEAFFGQGTR |
| | LTVV (SEQ ID NO: 899) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATACACCCACACTGAAGCTTTCTTTGGA |
| | CAAGGCACCAGACTCACAGTTGTA (SEQ ID NO: 900) |
| PN42833 | DAKTTQPPSMDCAEGRAANLPCNHSTISGNEYVYWYRQIHSQGPQYIIHGLKNN |
| Vα | ETNEMASLIITEDRKSSTLILPHATLRDTAVYYCIVRDTTSGTYKYIFGTGTRLKV |
| | LA (SEQ ID NO: 901) |
| | GATGCTAAGACCACCCAGCCCCCCTCCATGGATTGCGCTGAAGGAAGAGCTG |
| | CAAACCTGCCTTGTAATCACTCTACCATCAGTGGAAATGAGTATGTGTATTGG |
| | TATCGACAGATTCACTCCCAGGGGCCACAGTATATCATTCATGGTCTAAAAA |
| | ACAATGAAACCAATGAAATGGCCTCTCTGATCATCACAGAAGACAGAAAGTC |
| | CAGCACCTTGATCCTGCCCCACGCTACGCTGAGAGACACTGCTGTGTACTATT |
| | GCATCGTCAGAGACACTACCTCAGGAACCTACAAATACATCTTTGGAACAGG |
| | CACCAGGCTGAAGGTTTTAGCA (SEQ ID NO: 902) |
| PN42833 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLSSTGFSPLHFGNGT |
| | RLTVT (SEQ ID NO: 903) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATCCTCGACAGGTTTTTCACCCCTCCACT |
| | TTGGGAACGGGACCAGGCTCACTGTGACA (SEQ ID NO: 904) |
| PN42840 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAEGGGSQGNLIFGKGTKLSV |
| | KP (SEQ ID NO: 905) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGAGGGGGGAGGAAGCCAAGGAAATCTCATCTTTGGAAAAGG |
| | CACTAAACTCTCTGTTAAACCA (SEQ ID NO: 906) |
| PN42840 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSWTDYGYTFGSGTRL |
| | TVV (SEQ ID NO: 907) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTGGACAGACTATGGCTACACCTTCGGTTCGG |
| | GGACCAGGTTAACCGTTGTA (SEQ ID NO: 908) |
| PN42845 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGIRSNDYKLSFGAGTTVTV |
| | RA (SEQ ID NO: 909) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGGATACGTTCTAACGACTACAAGCTCAGCTTTGGAGCCGGA |
| | ACCACAGTAACTGTAAGAGCA (SEQ ID NO: 910) |
| PN42845 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSSWTAHTEAFFGQGT |
| | RLTVV (SEQ ID NO: 911) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTCCTGGACAGCCCACACTGAAGCTTTCTTT |
| | GGACAAGGCACCAGACTCACAGTTGTA (SEQ ID NO: 912) |
| PN42870 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAENSGGGADGLTFGKGTHLII |
| | QP (SEQ ID NO: 913) |
| | ACCCAGCTGCTGGAGCAGAGCCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGAAAATTCAGGAGGAGGTGCTGACGGACTCACCTTTGGCAAA |
| | GGGACTCATCTAATCATCCAGCCC (SEQ ID NO: 914) |
| PN42870 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSFTDYGYTFGSGTRLT |
| | W (SEQ ID NO: 915) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTTCACAGACTATGGCTACACCTTCGGTTCGG |
| | GGACCAGGTTAACCGTTGTA (SEQ ID NO: 916) |
| PN42879 | TQLLEQSPQFLSIQEGENLTVYCNSSSVFSSLQWYRQEPGEGPVLLVTVVTGGEV |
| Vα | KKLKRLTFQFGDARKDSSLHITAAQPGDTGLYLCAGEDFGNEKLTFGTGTRLTII |
| | P (SEQ ID NO: 917) |
| | ACCCAGCTGCTGGAGCAGAGTCCTCAGTTTCTAAGCATCCAAGAGGGAGAAA |
| | ATCTCACTGTGTACTGCAACTCCTCAAGTGTTTTTTCCAGCTTACAATGGTAC |
| | AGACAGGAGCCTGGGGAAGGTCCTGTCCTCCTGGTGACAGTAGTTACGGGTG |
| | GAGAAGTGAAGAAGCTGAAGAGACTAACCTTTCAGTTTGGTGATGCAAGAA |
| | AGGACAGTTCTCTCCACATCACTGCGGCCCAGCCTGGTGATACAGGCCTCTA |
| | CCTCTGTGCAGGAGAGGACTTTGGAAATGAGAAATTAACCTTTGGGACTGGA |
| | ACAAGACTCACCATCATACCC (SEQ ID NO: 918) |
| PN42879 | DAGVTQSPTHLIKTRGQQVTLRCSPKSGHDTVSWYQQALGQGPQFIFQYYEEEE |
| Vβ | RQRGNFPDRFSGHQFPNYSSELNVNALLLGDSALYLCASSWADYGYTFGSGTRL |
| | TVV(SEQ ID NO: 919) |
| | GACGCTGGAGTCACCCAAAGTCCCACACACCTGATCAAAACGAGAGGACAG |
| | CAAGTGACTCTGAGATGCTCTCCTAAGTCTGGGCATGACACTGTGTCCTGGTA |
| | CCAACAGGCCCTGGGTCAGGGGCCCCAGTTTATCTTTCAGTATTATGAGGAG |
| | GAAGAGAGACAGAGAGGCAACTTCCCTGATCGATTCTCAGGTCACCAGTTCC |
| | CTAACTATAGCTCTGAGCTGAATGTGAACGCCTTGTTGCTGGGGGACTCGGC |
| | CCTCTATCTCTGTGCCAGCAGCTGGGCGGATTATGGCTACACCTTCGGTTCGG |
| | GGACCAGGTTAACCGTTGTA (SEQ ID NO: 920) |
| PN42888 | ILNVEQSPQSLHVQEGDSTNFTCSFPSSNFYALHWYRWETAKSPEALFVMTLNG |
| Vα | DEKKKGRISATLNTKEGYSYLYIKGSQPEDSATYLCAFLTGNQFYFGTGTSLTVIP |
| | (SEQ ID NO: 921) |
| | ATACTGAACGTGGAACAAAGTCCTCAGTCACTGCATGTTCAGGAGGGAGACA |
| | GCACCAATTTCACCTGCAGCTTCCCTTCCAGCAATTTTTATGCCTTACACTGG |
| | TACAGATGGGAAACTGCAAAAAGCCCCGAGGCCTTGTTTGTAATGACTTTAA |
| | ATGGGGATGAAAAGAAGAAAGGACGAATAAGTGCCACTCTTAATACCAAGG |
| | AGGGTTACAGCTATTTGTACATCAAAGGATCCCAGCCTGAAGACTCAGCCAC |
| | ATACCTCTGTGCCTTTCTCACCGGTAACCAGTTCTATTTTGGGACAGGGACAA |
| | GTTTGACGGTCATTCCA (SEQ ID NO: 922) |
| PN42888 | DVKVTQSSRYLVKRTGEKVFLECVQDMDHENMFWYRQDPGLGLRLIYFSYDVK |
| Vβ | MKEKGDIPEGYSVSREKKERFSLILESASTNQTSMYLCASSTVRQGNYGYTFGSG |
| | TRLTVV (SEQ ID NO: 923) |
| | GATGTGAAAGTAACCCAGAGCTCGAGATATCTAGTCAAAAGGACGGGAGAG |
| | AAAGTTTTTCTGGAATGTGTCCAGGATATGGACCATGAAAATATGTTCTGGTA |
| | TCGACAAGACCCAGGTCTGGGGCTACGGCTGATCTATTTCTCATATGATGTTA |
| | AAATGAAAGAAAAAGGAGATATTCCTGAGGGGTACAGTGTCTCTAGAGAGA |
| | AGAAGGAGCGCTTCTCCCTGATTCTGGAGTCCGCCAGCACCAACCAGACATC |
| | TATGTACCTCTGTGCCAGCAGTACCGTGAGGCAGGGGAACTATGGCTACACC |
| | TTCGGTTCGGGGACCAGGTTAACCGTTGTA (SEQ ID NO: 924) |
| PN42895 | DQQVKQSSPSLSVQEGRISILNCDYTNSMFDYFLWYKKYPAEGPTFLISISSIKDK |
| Vα | NEDGRFTVFLNKSAKHLSLHIVPSQPGDSAVYFCAASAGSARQLTFGSGTQLTVL |
| | P (SEQ ID NO: 925) |
| | GACCAGCAAGTTAAGCAAAGTTCACCATCCCTGAGCGTCCAGGAAGGAAGA |
| | ATTTCTATTCTGAACTGTGACTATACTAACAGCATGTTTGATTATTTCCTATGG |
| | TACAAAAAATACCCTGCTGAAGGTCCTACATTCCTGATATCTATAAGTTCCAT |
| | TAAGGATAAAAATGAAGATGGAAGATTCACTGTTTTCTTAAACAAAAGTGCC |
| | AAGCACCTCTCTCTGCACATTGTGCCCTCCCAGCCTGGAGACTCTGCAGTGTA |
| | CTTCTGTGCAGCAAGCGCCGGTTCTGCAAGGCAACTGACCTTTGGATCTGGG |
| | ACACAATTGACTGTTTTACCT (SEQ ID NO: 926) |
| PN42895 | EAQVTQNPRYLITVTGKKLTVTCSQNMNHEYMSWYRQDPGLGLRQIYYSMNVE |
| Vβ | VTDKGDVPEGYKVSRKEKRNFPLILESPSPNQTSLYFCASSLWSNTEAFFGQGTR |
| | LTVV (SEQ ID NO: 927) |
| | GAAGCCCAAGTGACCCAGAACCCAAGATACCTCATCACAGTGACTGGAAAG |
| | AAGTTAACAGTGACTTGTTCTCAGAATATGAACCATGAGTATATGTCCTGGTA |
| | TCGACAAGACCCAGGGCTGGGCTTAAGGCAGATCTACTATTCAATGAATGTT |
| | GAGGTGACTGATAAGGGAGATGTTCCTGAAGGGTACAAAGTCTCTCGAAAA |
| | GAGAAGAGGAATTTCCCCCTGATCCTGGAGTCGCCCAGCCCCAACCAGACCT |
| | CTCTGTACTTCTGTGCCAGCAGTTTATGGTCGAACACTGAAGCTTTCTTTGGA |
| | CAAGGCACCAGACTCACAGTTGTA (SEQ ID NO: 928) |

TABLE 9
Variable (V) and joining (J) region gene families for the α and β
chains of VelociT TCRs specific for PRAME (312-320)/HLA-A2

| TCR ID | Vβ | Jβ | Vα | Jα |
|---|---|---|---|---|
| PN46909 | 6-6 | 1-1 | 38-1 | 26 |
| PN46889 | 6-2 | 1-1 | 12-2 | 11 |
| PN46733 | 6-1 | 1-1 | 13-2 | 26 |
| PN46723 | 5-5 | 1-4 | 20-1 | 42 |
| PN46714 | 5-5 | 1-6 | 20-1 | 42 |
| PN46735 | 5-5 | 1-2 | 26-2 | 45 |
| PN46678 | 5-5 | 1-4 | 19-1 | 18 |
| PN46884 | 28-1 | 1-3 | 19-1 | 34 |
| PN46914 | 28-1 | 1-5 | 19-1 | 23 |
| PN46883 | 18-1 | 1-1 | 19-1 | 34 |
| PN46857 | 18-1 | 1-1 | 19-1 | 47 |
| PN46880 | 18-1 | 1-1 | 16-1 | 6 |
| PN46871 | 13-1 | 1-5 | 21-1 | 24 |
| PN46853 | 13-1 | 1-5 | 24-1 | 37 |
| PN46731 | 13-1 | 1-1 | 13-1 | 26 |
| PN46777 | 10-2 | 1-2 | 38-1 | 47 |
| PN46797 | 10-2 | 1-2 | 38-1 | 47 |
| PN46738 | 10-2 | 1-2 | 38-1 | 45 |

TABLE 10
Variable (V) and joining (J) region gene families for the α and β
chains of VelociT TCRs specific for PRAME (425-433)/HLA-A2

| TCR ID | Vb | Jb | Va | Ja |
|---|---|---|---|---|
| PN42365 | 5-6 | 1-2 | 27-1 | 42-1 |
| PN42879 | 5-6 | 1-2 | 27-1 | 48-1 |
| PN42774 | 5-6 | 1-2 | 12-2 | 34-1 |
| PN42498 | 5-6 | 1-2 | 27-1 | 42-1 |
| PN42558 | 5-6 | 1-2 | 22-1 | 42-1 |
| PN42386 | 5-6 | 1-2 | 27-1 | 42-1 |
| PN42378 | 5-6 | 1-2 | 26-2 | 24-1 |
| PN42776 | 5-6 | 1-2 | 26-2 | 24-1 |
| PN42455 | 5-6 | 1-2 | 26-2 | 24-1 |
| PN42840 | 5-6 | 1-2 | 27-1 | 42-1 |
| PN42795 | 5-6 | 1-2 | 12-1 | 42-1 |
| PN42870 | 5-6 | 1-2 | 27-1 | 45-1 |
| PN42689 | 5-6 | 1-2 | 27-1 | 42-1 |
| PN42888 | 28-1 | 1-2 | 24-1 | 49-1 |
| PN42450 | 28-1 | 1-2 | 16-1 | 34-1 |
| PN42750 | 28-1 | 1-2 | 26-2 | 24-1 |
| PN42562 | 27-1 | 1-2 | 27-1 | 40-1 |
| PN42483 | 27-1 | 1-4 | 27-1 | 42-1 |
| PN42712 | 27-1 | 1-6 | 22-1 | 20-1 |
| PN42561 | 27-1 | 1-2 | 26-2 | 49-1 |
| PN42442 | 27-1 | 1-2 | 27-1 | 40-1 |
| PN42476 | 27-1 | 1-4 | 26-2 | 24-1 |
| PN42496 | 27-1 | 1-2 | 26-2 | 54-1 |
| PN42655 | 27-1 | 1-2 | 26-2 | 24-1 |
| PN42677 | 27-1 | 1-2 | 26-2 | 49-1 |
| PN42706 | 27-1 | 1-2 | 22-1 | 40-1 |
| PN42654 | 27-1 | 1-2 | 26-2 | 49-1 |
| PN42441 | 27-1 | 1-1 | 26-2 | 49-1 |
| PN42683 | 27-1 | 1-2 | 26-2 | 47-1 |
| PN42845 | 27-1 | 1-1 | 27-1 | 20-1 |
| PN42826 | 27-1 | 1-1 | 29/DV5 | 22-1 |
| PN42707 | 27-1 | 1-1 | 13-2 | 26-1 |
| PN42833 | 27-1 | 1-6 | 26-1 | 40-1 |
| PN42762 | 27-1 | 1-1 | 26-2 | 44-1 |
| PN42780 | 27-1 | 1-2 | 27-1 | 40-1 |
| PN42746 | 27-1 | 1-1 | 13-2 | 26-1 |
| PN42815 | 27-1 | 1-6 | 13-2 | 26-1 |
| PN42711 | 27-1 | 1-6 | 22-1 | 20-1 |
| PN42895 | 27-1 | 1-1 | 29/DV5 | 22-1 |
| PN42610 | 13-1 | 1-5 | 12-2 | 26-1 |

TABLE 11
Amino acid and polynucleic acid sequence identifiers for TCR alpha and beta
variable chains and CDRs that were identified with PRAME (312-320).

| | Amino Acid Sequences | | | | | | | |
|---|---|---|---|---|---|---|---|---|
| | | α CDRs | | | | β CDRs | | |
| | Vα | CDR1 | CDR2 | CDR3 | Vβ | CDR1 | CDR2 | CDR3 |
| TCR ID | SEQ ID NO: | | | | | | | |
| PN46678 | 217 | 1 | 2 | 3 | 219 | 4 | 5 | 6 |
| PN46714 | 221 | 7 | 8 | 9 | 223 | 10 | 11 | 12 |
| PN46723 | 225 | 13 | 14 | 15 | 227 | 16 | 17 | 18 |
| PN46731 | 229 | 19 | 20 | 21 | 231 | 22 | 23 | 24 |
| PN46733 | 233 | 25 | 26 | 27 | 235 | 28 | 29 | 30 |
| PN46735 | 237 | 31 | 32 | 33 | 239 | 34 | 35 | 36 |
| PN46738 | 241 | 37 | 38 | 39 | 243 | 40 | 41 | 42 |
| PN46777 | 245 | 43 | 44 | 45 | 247 | 46 | 47 | 48 |
| PN46797 | 249 | 49 | 50 | 51 | 251 | 52 | 53 | 54 |
| PN46853 | 253 | 55 | 56 | 57 | 255 | 58 | 59 | 60 |
| PN46857 | 257 | 61 | 62 | 63 | 259 | 64 | 65 | 66 |
| PN46871 | 261 | 67 | 68 | 69 | 263 | 70 | 71 | 72 |
| PN46880 | 265 | 73 | 74 | 75 | 267 | 76 | 77 | 78 |
| PN46883 | 269 | 79 | 80 | 81 | 271 | 82 | 83 | 84 |
| PN46884 | 273 | 85 | 86 | 87 | 275 | 88 | 89 | 90 |
| PN46889 | 277 | 91 | 92 | 93 | 279 | 94 | 95 | 96 |
| PN46909 | 281 | 97 | 98 | 99 | 283 | 100 | 101 | 102 |
| PN46914 | 285 | 103 | 104 | 105 | 287 | 106 | 107 | 108 |

| Polynucleic Acid Sequences | | | | | | | |
|---|---|---|---|---|---|---|---|
| | α CDRs | | | | β CDRs | | |
| Vα | CDR1 | CDR2 | CDR3 | Vβ | CDR1 | CDR2 | CDR3 |

| | |
|---|---|
| TCR ID | SEQ ID NO: |

| | | | | | | | | |
|---|---|---|---|---|---|---|---|---|
| PN46678 | 218 | 109 | 110 | 111 | 220 | 112 | 113 | 114 |
| PN46714 | 222 | 115 | 116 | 117 | 224 | 118 | 119 | 120 |
| PN46723 | 226 | 121 | 122 | 123 | 228 | 124 | 125 | 126 |
| PN46731 | 230 | 127 | 128 | 129 | 232 | 130 | 131 | 132 |
| PN46733 | 234 | 133 | 134 | 135 | 236 | 136 | 137 | 138 |
| PN46735 | 238 | 139 | 140 | 141 | 240 | 142 | 143 | 144 |
| PN46738 | 242 | 145 | 146 | 147 | 244 | 148 | 149 | 150 |
| PN46777 | 246 | 151 | 152 | 153 | 248 | 154 | 155 | 156 |
| PN46797 | 250 | 157 | 158 | 159 | 252 | 160 | 161 | 162 |
| PN46853 | 254 | 163 | 164 | 165 | 256 | 166 | 167 | 168 |
| PN46857 | 258 | 169 | 170 | 171 | 260 | 172 | 173 | 174 |
| PN46871 | 262 | 175 | 176 | 177 | 264 | 178 | 179 | 180 |
| PN46880 | 266 | 181 | 182 | 183 | 268 | 184 | 185 | 186 |
| PN46883 | 270 | 187 | 188 | 189 | 272 | 190 | 191 | 192 |
| PN46884 | 274 | 193 | 194 | 195 | 276 | 196 | 197 | 198 |
| PN46889 | 278 | 199 | 200 | 201 | 280 | 202 | 203 | 204 |
| PN46909 | 282 | 205 | 206 | 207 | 284 | 208 | 209 | 210 |
| PN46914 | 286 | 211 | 212 | 213 | 288 | 214 | 215 | 216 |

TABLE 12
Amino acid and polynucleic acid sequence identifiers for TCR alpha and beta
variable chains and CDRs that were identified with PRAME (425-433).

| | Amino Acid Sequences | | | | | | | |
|---|---|---|---|---|---|---|---|---|
| | | α CDRs | | | | β CDRs | | |
| | Vα | CDR1 | CDR2 | CDR3 | Vβ | CDR1 | CDR2 | CDR3 |
| TCR ID | SEQ ID NO: | | | | | | | |
| PN42365 | 769 | 289 | 290 | 291 | 771 | 292 | 293 | 294 |
| PN42378 | 773 | 295 | 296 | 297 | 775 | 298 | 299 | 300 |
| PN42386 | 777 | 301 | 302 | 303 | 779 | 304 | 305 | 306 |
| PN42441 | 781 | 307 | 308 | 309 | 783 | 310 | 311 | 312 |
| PN42442 | 785 | 313 | 314 | 315 | 787 | 316 | 317 | 318 |
| PN42450 | 789 | 319 | 320 | 321 | 791 | 322 | 323 | 324 |
| PN42455 | 793 | 325 | 326 | 327 | 795 | 328 | 329 | 330 |
| PN42476 | 797 | 331 | 332 | 333 | 799 | 334 | 335 | 336 |
| PN42483 | 801 | 337 | 338 | 339 | 803 | 340 | 341 | 342 |
| PN42496 | 805 | 343 | 344 | 345 | 807 | 346 | 347 | 348 |
| PN42498 | 809 | 349 | 350 | 351 | 811 | 352 | 353 | 354 |
| PN42558 | 813 | 355 | 356 | 357 | 815 | 358 | 359 | 360 |
| PN42561 | 817 | 361 | 362 | 363 | 819 | 364 | 365 | 366 |
| PN42562 | 821 | 367 | 368 | 369 | 823 | 370 | 371 | 372 |
| PN42610 | 825 | 373 | 374 | 375 | 827 | 376 | 377 | 378 |
| PN42654 | 829 | 379 | 380 | 381 | 831 | 382 | 383 | 384 |
| PN42655 | 833 | 385 | 386 | 387 | 835 | 388 | 389 | 390 |
| PN42677 | 837 | 391 | 392 | 393 | 839 | 394 | 395 | 396 |
| PN42683 | 841 | 397 | 398 | 399 | 843 | 400 | 401 | 402 |
| PN42689 | 845 | 403 | 404 | 405 | 847 | 406 | 407 | 408 |
| PN42706 | 849 | 409 | 410 | 411 | 851 | 412 | 413 | 414 |
| PN42707 | 853 | 415 | 416 | 417 | 855 | 418 | 419 | 420 |
| PN42711 | 857 | 421 | 422 | 423 | 859 | 424 | 425 | 426 |
| PN42712 | 861 | 427 | 428 | 429 | 863 | 430 | 431 | 432 |
| PN42746 | 865 | 433 | 434 | 435 | 867 | 436 | 437 | 438 |
| PN42750 | 869 | 439 | 440 | 441 | 871 | 442 | 443 | 444 |
| PN42762 | 873 | 445 | 446 | 447 | 875 | 448 | 449 | 450 |
| PN42774 | 877 | 451 | 452 | 453 | 879 | 454 | 455 | 456 |
| PN42776 | 881 | 457 | 458 | 459 | 883 | 460 | 461 | 462 |
| PN42780 | 885 | 463 | 464 | 465 | 887 | 466 | 467 | 468 |
| PN42795 | 889 | 469 | 470 | 471 | 891 | 472 | 473 | 474 |
| PN42815 | 893 | 475 | 476 | 477 | 895 | 478 | 479 | 480 |
| PN42826 | 897 | 481 | 482 | 483 | 899 | 484 | 485 | 486 |
| PN42833 | 901 | 487 | 488 | 489 | 903 | 490 | 491 | 492 |
| PN42840 | 905 | 493 | 494 | 495 | 907 | 496 | 497 | 498 |
| PN42845 | 909 | 499 | 500 | 501 | 911 | 502 | 503 | 504 |
| PN42870 | 913 | 505 | 506 | 507 | 915 | 508 | 509 | 510 |
| PN42879 | 917 | 511 | 512 | 513 | 919 | 514 | 515 | 516 |
| PN42888 | 921 | 517 | 518 | 519 | 923 | 520 | 521 | 522 |
| PN42895 | 925 | 523 | 524 | 525 | 927 | 526 | 527 | 528 |

| Polynucleic Acid Sequences | | | | | | | |
|---|---|---|---|---|---|---|---|
| | α CDRs | | | | β CDRs | | |
| Vα | CDR1 | CDR2 | CDR3 | Vβ | CDR1 | CDR2 | CDR3 |

| | |
|---|---|
| TCR ID | SEQ ID NO: |

| | | | | | | | | |
|---|---|---|---|---|---|---|---|---|
| PN42365 | 770 | 529 | 530 | 531 | 772 | 532 | 533 | 534 |
| PN42378 | 774 | 535 | 536 | 537 | 776 | 538 | 539 | 540 |
| PN42386 | 778 | 541 | 542 | 543 | 780 | 544 | 545 | 546 |
| PN42441 | 782 | 547 | 548 | 549 | 784 | 550 | 551 | 552 |
| PN42442 | 786 | 553 | 554 | 555 | 788 | 556 | 557 | 558 |
| PN42450 | 790 | 559 | 560 | 561 | 792 | 562 | 563 | 564 |
| PN42455 | 794 | 565 | 566 | 567 | 796 | 568 | 569 | 570 |
| PN42476 | 798 | 571 | 572 | 573 | 800 | 574 | 575 | 576 |
| PN42483 | 802 | 577 | 578 | 579 | 804 | 580 | 581 | 582 |
| PN42496 | 806 | 583 | 584 | 585 | 808 | 586 | 587 | 588 |
| PN42498 | 810 | 589 | 590 | 591 | 812 | 592 | 593 | 594 |
| PN42558 | 814 | 595 | 596 | 597 | 816 | 598 | 599 | 600 |
| PN42561 | 818 | 601 | 602 | 603 | 820 | 604 | 605 | 606 |
| PN42562 | 822 | 607 | 608 | 609 | 824 | 610 | 611 | 612 |
| PN42610 | 826 | 613 | 614 | 615 | 828 | 616 | 617 | 618 |
| PN42654 | 830 | 619 | 620 | 621 | 832 | 622 | 623 | 624 |
| PN42655 | 834 | 625 | 626 | 627 | 836 | 628 | 629 | 630 |
| PN42677 | 838 | 631 | 632 | 633 | 840 | 634 | 635 | 636 |
| PN42683 | 842 | 637 | 638 | 639 | 844 | 640 | 641 | 642 |
| PN42689 | 846 | 643 | 644 | 645 | 848 | 646 | 647 | 648 |
| PN42706 | 850 | 649 | 650 | 651 | 852 | 652 | 653 | 654 |
| PN42707 | 854 | 655 | 656 | 657 | 856 | 658 | 659 | 660 |
| PN42711 | 858 | 661 | 662 | 663 | 860 | 664 | 665 | 666 |
| PN42712 | 862 | 667 | 668 | 669 | 864 | 670 | 671 | 672 |
| PN42746 | 866 | 673 | 674 | 675 | 868 | 676 | 677 | 678 |
| PN42750 | 870 | 679 | 680 | 681 | 872 | 682 | 683 | 684 |
| PN42762 | 874 | 685 | 686 | 687 | 876 | 688 | 689 | 690 |
| PN42774 | 878 | 691 | 692 | 693 | 880 | 694 | 695 | 696 |
| PN42776 | 882 | 697 | 698 | 699 | 884 | 700 | 701 | 702 |
| PN42780 | 886 | 703 | 704 | 705 | 888 | 706 | 707 | 708 |
| PN42795 | 890 | 709 | 710 | 711 | 892 | 712 | 713 | 714 |
| PN42815 | 894 | 715 | 716 | 717 | 896 | 718 | 719 | 720 |
| PN42826 | 898 | 721 | 722 | 723 | 900 | 724 | 725 | 726 |
| PN42833 | 902 | 727 | 728 | 729 | 904 | 730 | 731 | 732 |
| PN42840 | 906 | 733 | 734 | 735 | 908 | 736 | 737 | 738 |
| PN42845 | 910 | 739 | 740 | 741 | 912 | 742 | 743 | 744 |
| PN42870 | 914 | 745 | 746 | 747 | 916 | 748 | 749 | 750 |
| PN42879 | 918 | 751 | 752 | 753 | 920 | 754 | 755 | 756 |
| PN42888 | 922 | 757 | 758 | 759 | 924 | 760 | 761 | 762 |
| PN42895 | 926 | 763 | 764 | 765 | 928 | 766 | 767 | 768 |

### Example 2. Dose-Dependent T Cell Receptor Activation

A Jurkat cell line lacking endogenous TCRα and TCRβ expression was generated by gene disruption. These cells were then engineered with a genomic landing pad site that allowed single copy Cre recombinase-mediated insertion of transgenic TCR constructs. An Activator Protein 1 (AP1) response element-driven luciferase reporter was subsequently incorporated into this parental bioassay cell line. Specific TCR bioassay lines were generated by Cre-mediated insertion of constructs expressing the VelociT®-derived TCRα and TCRβ sequences.

Jurkat bioassay lines expressing TCR constructs were sorted to homogeneity using Flourescence-Activated Cell Sorting (FACS), then tested in peptide-MHC stimulation assays. HEK293T cells (HLA-A2*01) were plated in assay wells with varying dilutions of antigenic PRAME peptide (PRAME 425-433; SEQ ID NO: 930) or an irrelevant HLA-A2 restricted peptide (SLLMWITQC; SEQ ID NO: 953). These dilutions were made as indicated in Table 13.

TABLE 13
Peptide dilutions for testing dose response of anti-PRAME TCRs

| | | Irrelevant | PRAME (425- |
|---|---|---|---|
| | Concentration | peptide | 433) peptide |
| Dilution | (μM) | (μg/ml) | (μg/ml) |
| 1 | 100 | 109 | 98.9 |
| 2 | 20 | 21.8 | 19.8 |
| 3 | 4 | 4.4 | 4.0 |
| 4 | 0.8 | 0.87 | 0.79 |
| 5 | 0.16 | 0.17 | 0.16 |
| 6 | 0.032 | 0.035 | 0.032 |
| 7 | 0.0064 | 0.0070 | 0.0063 |
| 8 | 0.00128 | 0.0014 | 0.0013 |
| 9 | 0.000256 | 0.00028 | 0.00025 |
| 10 | 0.0000512 | 5.6E−5 | 5.1E−5 |
| 11 | 0.00001024 | 1.1E−5 | 1.0E−5 |
| 12 | 0 (no peptide) | 0 | 0 |

After a 2 hour incubation, engineered Jurkat cells were added to wells at a 3:1 Jurkat: 293T cell ratio and incubated a further 5 hours. Luciferase reporter activity was determined by measuring endpoint luminescence output in assay wells. PRAME-specific TCRs mediated dose-dependent AP1 reporter activation in response to HLA-A2*01 HEK293T cells pulsed with cognate peptide, but not those cells pulsed with the irrelevant peptide. EC50 data is shown in Table 14, below.

TABLE 14
Antigen-specific response of HLA-A2/PRAME (425-433)-specific TCRs
in a Jurkat cell bioassay, normalized to negative control

| TCR ID | EC50 (μM) |
|---|---|
| PN42365 | 0.01594 |
| PN42386 | 0.02711 |
| PN42441 | 0.8205 |
| PN42442 | 0.2324 |
| PN42450 | 0.03355 |
| PN42483 | * |
| PN42496 | 0.02937 |
| PN42498 | 0.002418 |
| PN42558 | 0.2614 |
| PN42561 | 0.0137 |
| PN42562 | 0.02807 |
| PN42610 | * |
| PN42654 | * |
| PN42655 | |
| PN42677 | * |
| PN42683 | * |
| PN42689 | 0.0095 |
| PN42706 | 0.7274 |
| PN42707 | 0.01182 |
| PN42711 | 0.05688 |
| PN42712 | 0.01329 |
| PN42746 | 0.04598 |
| PN42762 | 114.8 |
| PN42774 | 0.8757 |
| PN42780 | 1.255 |
| PN42795 | 0.2454 |
| PN42815 | 0.1261 |
| PN42826 | 4.915 |
| PN42833 | 0.04786 |
| PN42840 | 0.03092 |
| PN42845 | 0.911 |
| PN42870 | 0.1302 |
| PN42879 | * |
| PN42888 | 0.0119 |
| PN42895 | 0.94 |

* Not above negative control/did not respond in assay

### EQUIVALENTS

Those skilled in the art will recognize or be able to ascertain, using no more than routine experimentation, many equivalents to the specific embodiments of the invention described herein, such equivalents are intended to be encompassed by the following claims. The contents of all references, patents and published patent applications cited throughout this application are incorporated herein by reference.

We claim:

1. A T cell receptor (TCR) that binds specifically to an HLA-A2 presented preferentially expressed antigen in melanoma (PRAME) peptide,
wherein the TCR comprises a TCR alpha chain and a TCR beta chain, and
wherein the TCR alpha chain comprises an alpha chain variable domain comprising a complementarity determining region (CDR) 1, a CDR2, and a CDR3 comprising the amino acid sequences of SEQ ID NOs: 319, 320, and 321, respectively, and the TCR beta chain comprises a beta chain variable domain comprising a CDR1, a CDR2, and a CDR3 comprising the amino acid sequences of SEQ ID NOs: 322, 323, and 324, respectively.

2. The TCR of claim 1, wherein the alpha chain variable domain comprises the amino acid sequence of SEQ ID NO: 789 and the beta chain variable domain comprises the amino acid sequence of SEQ ID NO: 791.

3. The TCR of claim 1, further comprising a detectable moiety.

4. A pharmaceutical composition comprising the TCR of claim 1 and a pharmaceutically acceptable carrier or diluent.

5. A cell presenting the TCR of claim 1.

6. A polynucleotide molecule comprising
a) a polynucleotide sequence that encodes the alpha chain variable domain of the TCR as set forth in claim 1; and/or
b) a polynucleotide sequence that encodes the beta chain variable domain of the TCR as set forth in claim 1.

7. A vector comprising the polynucleotide molecule of claim 6.

8. An cell expressing the vector of claim 7.

9. A pharmaceutical composition comprising the isolated cell of claim 8 and a pharmaceutically acceptable carrier or diluent.

10. The polynucleotide molecule of claim 6, wherein the polynucleotide sequence that encodes the alpha chain variable domain comprises the nucleotide sequence of SEQ ID NO: 790.

11. The polynucleotide molecule of claim 6, wherein the polynucleotide sequence that encodes the beta chain variable domain comprises the nucleotide sequence of SEQ ID NO: 792.

12. A cell expressing the polynucleotide molecule of claim 6.

13. A cell comprising
a first polynucleotide molecule, wherein the first polynucleotide molecule comprises a polynucleotide sequence that encodes the alpha chain variable domain of the TCR as set forth in claim 1; and
a second polynucleotide molecule, wherein the second polynucleotide molecule comprises a polynucleotide sequence that encodes the beta chain variable domain of the TCR as set forth in claim 1.

14. The cell of claim 13, wherein the polynucleotide sequence that encodes the alpha chain variable domain of the TCR comprises the nucleotide sequence of SEQ ID NO: 906, and wherein the polynucleotide sequence that encodes the beta chain variable domain of the TCR comprises the nucleotide sequence of SEQ ID NO: 908.

15. The cell of claim 13, wherein the first polynucleotide molecule is contained within a vector.

16. The cell of claim 13, wherein the second polynucleotide molecule is contained within a vector.

17. The cell of claim 13, wherein the first polynucleotide molecule and the second polynucleotide molecule are contained within a vector.

15. A pharmaceutical composition comprising the cell of claim 5.

16. A pharmaceutical composition comprising the cell of claim 12.

17. A pharmaceutical composition comprising the cell of any one of claims 13-17.

18. A method of treating a subject having a PRAME-associated disease or disorder, comprising administering to the subject a therapeutically effective amount of the TCR as set forth in claim 1, thereby treating the subject.

19. The method of claim 18, wherein the PRAME-associated disease or disorder is PRAME-associated cancer.

20. The method of claim 19, wherein the PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

21. The method of claim 18, wherein the TCR is administered to the subject in combination with a second therapeutic agent.

22. The method of claim 18, wherein the TCR administered subcutaneously, intravenously, intradermally, intraperitoneally, orally, intramuscularly or intracranially to the subject.

23. A method of treating a subject having an PRAME-associated disease or disorder, comprising administering to the subject a plurality of the cells of any one of claims 8, 12, and 13-17, thereby treating the subject.

24. The method of claim 23, wherein the PRAME-associated disease or disorder is PRAME-associated cancer.

25. The method of claim 24, wherein the PRAME-associated cancer is a liposarcoma, a neuroblastoma, a myeloma, a melanoma, a metastatic melanoma, a synovial sarcoma, a bladder cancer, an esophageal cancer, an esophageal squamous cell carcinoma, a hepatocellular cancer, a head and neck cancer, a non-small cell lung cancer, an ovarian cancer, an ovarian epithelial cancer, a prostate cancer, a breast cancer, an astrocytic tumor, a glioblastoma multiforme, an anaplastic astrocytoma, a brain tumor, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumors, a colorectal carcinoma, a stomach cancer, a gastric cancer, a rhabdomyosarcoma, a myxoid round cell liposarcoma, uterine corpus endometrial carcinoma, uterine carcinosarcoma, testicular germ cell tumor, uveal melanoma, kidney renal papillary cell carcinoma, kidney renal clear cell carcinoma, thymoma, colon adenocarcinoma, cervical squamous cell carcinoma, cervical tumor, pancreatic adenocarcinoma, liver cancer, hepatocellular carcinoma, mesothelioma, or a recurrent non-small cell lung cancer.

26. The method of claim 23, wherein the plurality of cells is administered to the subject in combination with a second therapeutic agent.

27. The method of claim 23, wherein the plurality of cells is administered subcutaneously, intravenously, intradermally, intraperitoneally, orally, intramuscularly or intracranially to the subject.


SEQUENCE LISTING (954 sequences)

SEQ ID NO: 1 - protein, 7 aa, synthetic construct
  REGION 1..7: note = source = /note="Description of Artificial Sequence: Synthetic peptide"
TRDTTYY

SEQ ID NO: 2 - protein, 8 aa, synthetic construct
  REGION 1..8: note = source = /note="Description of Artificial Sequence: Synthetic peptide"
RNSFDEQN

SEQ ID NO: 3 - protein, 15 aa, synthetic construct
  REGION 1..15: note = source = /note="Description of Artificial Sequence: Synthetic peptide"
ALSEFDRGSTLGRLY

SEQ ID NO: 4 - protein, 5 aa, synthetic construct
  REGION 1..5: note = source = /note="Description of Artificial Sequence: Synthetic peptide"
SGHKS

SEQ ID NO: 5 - protein, 6 aa, synthetic construct
  REGION 1..6: note = source = /note="Description of Artificial Sequence: Synthetic peptide"
YYEKEE

SEQ ID NO: 6 - protein, 11 aa, synthetic construct
  REGION 1..11: note = source = /note="Description of Artificial Sequence: Synthetic peptide"
ASSRDINEKLF

SEQ ID NO: 7 - protein, 6 aa, synthetic construct
  REGION 1..6: note = source = /note="Description of Artificial Sequence: Synthetic peptide"
VSGLRG

SEQ ID NO: 8 - protein, 7 aa, synthetic construct
  REGION 1..7: note = source = /note="Description of Artificial Sequence: Synthetic peptide"
LYSAGEE

SEQ ID NO: 9 - protein, 13 aa, synthetic construct
  REGION 1..13: note = source = /note="Description of Artificial Sequence: Synthetic peptide"
AVQADGGSQGNLI

SEQ ID NO: 10 - protein, 5 aa, synthetic construct
  REGION 1..5: note = source = /note="Description of Artificial Sequence: Synthetic peptide"
SGHKS

SEQ ID NO: 11 - protein, 6 aa, synthetic construct
  REGION 1..6: note = source = /note="Description of Artificial Sequence: Synthetic peptide"
YYEKEE

SEQ ID NO: 12 - protein, 11 aa, synthetic construct
  REGION 1..11: note = source = /note="Description of Artificial Sequence: Synthetic peptide"
ASSLDINSPLH

SEQ ID NO: 13 - protein, 6 aa, synthetic construct
  REGION 1..6: note = source = /note="Description of Artificial Sequence: Synthetic peptide"
VSGLRG

SEQ ID NO: 14 - protein, 7 aa, synthetic construct
  REGION 1..7: note = source = /note="Description of Artificial Sequence: Synthetic peptide"
LYSAGEE

SEQ ID NO: 15 - protein, 13 aa, synthetic construct
  REGION 1..13: note = source = /note="Description of Artificial Sequence: Synthetic peptide"
AVQEDGGSQGNLI

SEQ ID NO: 16 - protein, 5 aa, synthetic construct
  REGION 1..5: note = source = /note="Description of Artificial Sequence: Synthetic peptide"
SGHKS

SEQ ID NO: 17 - protein, 6 aa, synthetic construct
  REGION 1..6: note = source = /note="Description of Artificial Sequence: Synthetic peptide"
YYEKEE

SEQ ID NO: 18 - protein, 11 aa, synthetic construct
  REGION 1..11: note = source = /note="Description of Artificial Sequence: Synthetic peptide"
ASSRDINEKLF

SEQ ID NO: 19 - protein, 6 aa, synthetic construct
  REGION 1..6: note = source = /note="Description of Artificial Sequence: Synthetic peptide"
DSASNY

SEQ ID NO: 20 - protein, 7 aa, synthetic construct
  REGION 1..7: note = source = /note="Description of Artificial Sequence: Synthetic peptide"
IRSNVGE

SEQ ID NO: 21 - protein, 10 aa, synthetic construct
  REGION 1..10: note = source = /note="Description of Artificial Sequence: Synthetic peptide"
AAWNYGQNFV

SEQ ID NO: 22 - protein, 5 aa, synthetic construct
  REGION 1..5: note = source = /note="Description of Artificial Sequence: Synthetic peptide"
PRHDT

SEQ ID NO: 23 - protein, 6 aa, synthetic construct
  REGION 1..6: note = source = /note="Description of Artificial Sequence: Synthetic peptide"
FYEKMQ

SEQ ID NO: 24 - protein, 10 aa, synthetic construct
  REGION 1..10: note = source = /note="Description of Artificial Sequence: Synthetic peptide"
ASSLEGSEAF

SEQ ID NO: 25 - protein, 6 aa, synthetic construct
  REGION 1..6: note = source = /note="Description of Artificial Sequence: Synthetic peptide"
NSASDY

SEQ ID NO: 26 - protein, 7 aa, synthetic construct
  REGION 1..7: note = source = /note="Description of Artificial Sequence: Synthetic peptide"
IRSNMDK

SEQ ID NO: 27 - protein, 10 aa, synthetic construct
  REGION 1..10: note = source = /note="Description of Artificial Sequence: Synthetic peptide"
AENNYGQNFV

SEQ ID NO: 28 - protein, 5 aa, synthetic construct
  REGION 1..5: note = source = /note="Description of Artificial Sequence: Synthetic peptide"
MNHNS

SEQ ID NO: 29 - protein, 6 aa, synthetic construct
  REGION 1..6: note = source = /note="Description of Artificial Sequence: Synthetic peptide"
SASEGT

SEQ ID NO: 30 - protein, 12 aa, synthetic construct
  REGION 1..12: note = source = /note="Description of Artificial Sequence: Synthetic peptide"
ASSDWGQGVEAF

SEQ ID NO: 31 - protein, 7 aa, synthetic construct
  REGION 1..7: note = source = /note="Description of Artificial Sequence: Synthetic peptide"
TISGTDY

SEQ ID NO: 32 - protein, 5 aa, synthetic construct
  REGION 1..5: note = source = /note="Description of Artificial Sequence: Synthetic peptide"
GLTSN

SEQ ID NO: 33 - protein, 16 aa, synthetic construct
  REGION 1..16: note = source = /note="Description of Artificial Sequence: Synthetic peptide"
ILREYMYSGGGADGLT

SEQ ID NO: 34 - protein, 5 aa, synthetic construct
  REGION 1..5: note = source = /note="Description of Artificial Sequence: Synthetic peptide"
SGHKS

SEQ ID NO: 35 - protein, 6 aa, synthetic construct
  REGION 1..6: note = source = /note="Description of Artificial Sequence: Synthetic peptide"
YYEKEE

SEQ ID NO: 36 - protein, 13 aa, synthetic construct
  REGION 1..13: note = source = /note="Description of Artificial Sequence: Synthetic peptide"
ASSFQAGVNYGYT

SEQ ID NO: 37 - protein, 7 aa, synthetic construct
  REGION 1..7: note = source = /note="Description of Artificial Sequence: Synthetic peptide"
TSENNYY

SEQ ID NO: 38 - protein, 8 aa, synthetic construct
  REGION 1..8: note = source = /note="Description of Artificial Sequence: Synthetic peptide"
QEAYKQQN

SEQ ID NO: 39 - protein, 14 aa, synthetic construct
  REGION 1..14: note = source = /note="Description of Artificial Sequence: Synthetic peptide"
AFGMYSGGGADGLT

SEQ ID NO: 40 - protein, 5 aa, synthetic construct
  REGION 1..5: note = source = /note="Description of Artificial Sequence: Synthetic peptide"
WSHSY

SEQ ID NO: 41 - protein, 6 aa, synthetic construct
  REGION 1..6: note = source = /note="Description of Artificial Sequence: Synthetic peptide"
SAAADI

SEQ ID NO: 42 - protein, 12 aa, synthetic construct
  REGION 1..12: note = source = /note="Description of Artificial Sequence: Synthetic peptide"
ASSDGTGYYGYT

SEQ ID NO: 43 - protein, 7 aa, synthetic construct
  REGION 1..7: note = source = /note="Description of Artificial Sequence: Synthetic peptide"
TSENNYY

SEQ ID NO: 44 - protein, 8 aa, synthetic construct
  REGION 1..8: note = source = /note="Description of Artificial Sequence: Synthetic peptide"
QEAYKQQN

SEQ ID NO: 45 - protein, 10 aa, synthetic construct
  REGION 1..10: note = source = /note="Description of Artificial Sequence: Synthetic peptide"
ALMEYGNKLV

SEQ ID NO: 46 - protein, 5 aa, synthetic construct
  REGION 1..5: note = source = /note="Description of Artificial Sequence: Synthetic peptide"
WSHSY

SEQ ID NO: 47 - protein, 6 aa, synthetic construct
  REGION 1..6: note = source = /note="Description of Artificial Sequence: Synthetic peptide"
SAAADI

SEQ ID NO: 48 - protein, 12 aa, synthetic construct
  REGION 1..12: note = source = /note="Description of Artificial Sequence: Synthetic peptide"
ASSDGTGYYGYT

SEQ ID NO: 49 - protein, 7 aa, synthetic construct
  REGION 1..7: note = source = /note="Description of Artificial Sequence: Synthetic peptide"
TSENNYY

SEQ ID NO: 50 - protein, 8 aa, synthetic construct
  REGION 1..8: note = source = /note="Description of Artificial Sequence: Synthetic peptide"
QEAYKQQN

SEQ ID NO: 51 - protein, 10 aa, synthetic construct
  REGION 1..10: note = source = /note="Description of Artificial Sequence: Synthetic peptide"
ALMEYENKLV

SEQ ID NO: 52 - protein, 5 aa, synthetic construct
  REGION 1..5: note = source = /note="Description of Artificial Sequence: Synthetic peptide"
WSHSY

SEQ ID NO: 53 - protein, 6 aa, synthetic construct
  REGION 1..6: note = source = /note="Description of Artificial Sequence: Synthetic peptide"
SAAADI

SEQ ID NO: 54 - protein, 12 aa, synthetic construct
  REGION 1..12: note = source = /note="Description of Artificial Sequence: Synthetic peptide"
ASSDGTGYYGYT

SEQ ID NO: 55 - protein, 6 aa, synthetic construct
  REGION 1..6: note = source = /note="Description of Artificial Sequence: Synthetic peptide"
SSNFYA

SEQ ID NO: 56 - protein, 7 aa, synthetic construct
  REGION 1..7: note = source = /note="Description of Artificial Sequence: Synthetic peptide"
MTLNGDE

SEQ ID NO: 57 - protein, 12 aa, synthetic construct
  REGION 1..12: note = source = /note="Description of Artificial Sequence: Synthetic peptide"
ACGGSGNTGKLI

SEQ ID NO: 58 - protein, 5 aa, synthetic construct
  REGION 1..5: note = source = /note="Description of Artificial Sequence: Synthetic peptide"
PRHDT

SEQ ID NO: 59 - protein, 6 aa, synthetic construct
  REGION 1..6: note = source = /note="Description of Artificial Sequence: Synthetic peptide"
FYEKMQ

SEQ ID NO: 60 - protein, 10 aa, synthetic construct
  REGION 1..10: note = source = /note="Description of Artificial Sequence: Synthetic peptide"
ASSSQGQPQH

SEQ ID NO: 61 - protein, 7 aa, synthetic construct
  REGION 1..7: note = source = /note="Description of Artificial Sequence: Synthetic peptide"
TRDTTYY

SEQ ID NO: 62 - protein, 8 aa, synthetic construct
  REGION 1..8: note = source = /note="Description of Artificial Sequence: Synthetic peptide"
RNSFDEQN

SEQ ID NO: 63 - protein, 11 aa, synthetic construct
  REGION 1..11: note = source = /note="Description of Artificial Sequence: Synthetic peptide"
ALSEGYGNKLV

SEQ ID NO: 64 - protein, 5 aa, synthetic construct
  REGION 1..5: note = source = /note="Description of Artificial Sequence: Synthetic peptide"
KGHSH

SEQ ID NO: 65 - protein, 6 aa, synthetic construct
  REGION 1..6: note = source = /note="Description of Artificial Sequence: Synthetic peptide"
LQKENI

SEQ ID NO: 66 - protein, 11 aa, synthetic construct
  REGION 1..11: note = source = /note="Description of Artificial Sequence: Synthetic peptide"
ASSHRDDTEAF

SEQ ID NO: 67 - protein, 6 aa, synthetic construct
  REGION 1..6: note = source = /note="Description of Artificial Sequence: Synthetic peptide"
DSAIYN

SEQ ID NO: 68 - protein, 7 aa, synthetic construct
  REGION 1..7: note = source = /note="Description of Artificial Sequence: Synthetic peptide"
IQSSQRE

SEQ ID NO: 69 - protein, 13 aa, synthetic construct
  REGION 1..13: note = source = /note="Description of Artificial Sequence: Synthetic peptide"
AVEGTTDSWGKFQ

SEQ ID NO: 70 - protein, 5 aa, synthetic construct
  REGION 1..5: note = source = /note="Description of Artificial Sequence: Synthetic peptide"
PRHDT

SEQ ID NO: 71 - protein, 6 aa, synthetic construct
  REGION 1..6: note = source = /note="Description of Artificial Sequence: Synthetic peptide"
FYEKMQ

SEQ ID NO: 72 - protein, 10 aa, synthetic construct
  REGION 1..10: note = source = /note="Description of Artificial Sequence: Synthetic peptide"
ASSSQGQPQH

SEQ ID NO: 73 - protein, 6 aa, synthetic construct
  REGION 1..6: note = source = /note="Description of Artificial Sequence: Synthetic peptide"
YSGSPE

SEQ ID NO: 74 - protein, 4 aa, synthetic construct
  REGION 1..4: note = source = /note="Description of Artificial Sequence: Synthetic peptide"
HISR

SEQ ID NO: 75 - protein, 13 aa, synthetic construct
  REGION 1..13: note = source = /note="Description of Artificial Sequence: Synthetic peptide"
ALSGASGGSYIPT

SEQ ID NO: 76 - protein, 5 aa, synthetic construct
  REGION 1..5: note = source = /note="Description of Artificial Sequence: Synthetic peptide"
KGHSH

SEQ ID NO: 77 - protein, 6 aa, synthetic construct
  REGION 1..6: note = source = /note="Description of Artificial Sequence: Synthetic peptide"
LQKENI

SEQ ID NO: 78 - protein, 11 aa, synthetic construct
  REGION 1..11: note = source = /note="Description of Artificial Sequence: Synthetic peptide"
ASSHRDDTEAF

SEQ ID NO: 79 - protein, 7 aa, synthetic construct
  REGION 1..7: note = source = /note="Description of Artificial Sequence: Synthetic peptide"
TRDTTYY

SEQ ID NO: 80 - protein, 8 aa, synthetic construct
  REGION 1..8: note = source = /note="Description of Artificial Sequence: Synthetic peptide"
RNSFDEQN

SEQ ID NO: 81 - protein, 13 aa, synthetic construct
  REGION 1..13: note = source = /note="Description of Artificial Sequence: Synthetic peptide"
ALSVSSYNTDKLI

SEQ ID NO: 82 - protein, 5 aa, synthetic construct
  REGION 1..5: note = source = /note="Description of Artificial Sequence: Synthetic peptide"
KGHSH

SEQ ID NO: 83 - protein, 6 aa, synthetic construct
  REGION 1..6: note = source = /note="Description of Artificial Sequence: Synthetic peptide"
LQKENI

SEQ ID NO: 84 - protein, 11 aa, synthetic construct
  REGION 1..11: note = source = /note="Description of Artificial Sequence: Synthetic peptide"
ASSHRDDTEAF

SEQ ID NO: 85 - protein, 7 aa, synthetic construct
  REGION 1..7: note = source = /note="Description of Artificial Sequence: Synthetic peptide"
TRDTTYY

SEQ ID NO: 86 - protein, 8 aa, synthetic construct
  REGION 1..8: note = source = /note="Description of Artificial Sequence: Synthetic peptide"
RNSFDEQN

SEQ ID NO: 87 - protein, 12 aa, synthetic construct
  REGION 1..12: note = source = /note="Description of Artificial Sequence: Synthetic peptide"
ALSEGYNTDKLI

SEQ ID NO: 88 - protein, 5 aa, synthetic construct
  REGION 1..5: note = source = /note="Description of Artificial Sequence: Synthetic peptide"
MDHEN

SEQ ID NO: 89 - protein, 6 aa, synthetic construct
  REGION 1..6: note = source = /note="Description of Artificial Sequence: Synthetic peptide"
SYDVKM

SEQ ID NO: 90 - protein, 11 aa, synthetic construct
  REGION 1..11: note = source = /note="Description of Artificial Sequence: Synthetic peptide"
ASSLGGANTIY

SEQ ID NO: 91 - protein, 6 aa, synthetic construct
  REGION 1..6: note = source = /note="Description of Artificial Sequence: Synthetic peptide"
DRGSQS

SEQ ID NO: 92 - protein, 6 aa, synthetic construct
  REGION 1..6: note = source = /note="Description of Artificial Sequence: Synthetic peptide"
IYSNGD

SEQ ID NO: 93 - protein, 13 aa, synthetic construct
  REGION 1..13: note = source = /note="Description of Artificial Sequence: Synthetic peptide"
AVNIPNSGYSTLT

SEQ ID NO: 94 - protein, 5 aa, synthetic construct
  REGION 1..5: note = source = /note="Description of Artificial Sequence: Synthetic peptide"
MNHEY

SEQ ID NO: 95 - protein, 6 aa, synthetic construct
  REGION 1..6: note = source = /note="Description of Artificial Sequence: Synthetic peptide"
SVGEGT

SEQ ID NO: 96 - protein, 11 aa, synthetic construct
  REGION 1..11: note = source = /note="Description of Artificial Sequence: Synthetic peptide"
ASSYWEGTEAF

SEQ ID NO: 97 - protein, 7 aa, synthetic construct
  REGION 1..7: note = source = /note="Description of Artificial Sequence: Synthetic peptide"
TSENNYY

SEQ ID NO: 98 - protein, 8 aa, synthetic construct
  REGION 1..8: note = source = /note="Description of Artificial Sequence: Synthetic peptide"
QEAYKQQN

SEQ ID NO: 99 - protein, 9 aa, synthetic construct
  REGION 1..9: note = source = /note="Description of Artificial Sequence: Synthetic peptide"
AFDYGQNFV

SEQ ID NO: 100 - protein, 5 aa, synthetic construct
  REGION 1..5: note = source = /note="Description of Artificial Sequence: Synthetic peptide"
MNHNY

SEQ ID NO: 101 - protein, 6 aa, synthetic construct
  REGION 1..6: note = source = /note="Description of Artificial Sequence: Synthetic peptide"
SVGAGI

SEQ ID NO: 102 - protein, 12 aa, synthetic construct
  REGION 1..12: note = source = /note="Description of Artificial Sequence: Synthetic peptide"
ASSYGGGQTEAF

SEQ ID NO: 103 - protein, 7 aa, synthetic construct
  REGION 1..7: note = source = /note="Description of Artificial Sequence: Synthetic peptide"
TRDTTYY

SEQ ID NO: 104 - protein, 8 aa, synthetic construct
  REGION 1..8: note = source = /note="Description of Artificial Sequence: Synthetic peptide"
RNSFDEQN

SEQ ID NO: 105 - protein, 13 aa, synthetic construct
  REGION 1..13: note = source = /note="Description of Artificial Sequence: Synthetic peptide"
ALSEGYNQGGKLI

SEQ ID NO: 106 - protein, 5 aa, synthetic construct
  REGION 1..5: note = source = /note="Description of Artificial Sequence: Synthetic peptide"
MDHEN

SEQ ID NO: 107 - protein, 6 aa, synthetic construct
  REGION 1..6: note = source = /note="Description of Artificial Sequence: Synthetic peptide"
SYDVKM

SEQ ID NO: 108 - protein, 11 aa, synthetic construct
  REGION 1..11: note = source = /note="Description of Artificial Sequence: Synthetic peptide"
ASGADSNQPQH

SEQ ID NO: 109 - DNA, 21 bp, synthetic construct
  misc_feature 1..21: note = source = /note="Description of Artificial Sequence: Synthetic oligonucleotide"
  source 1..21: mol_type = other DNA
acccgtgatactacttattac

SEQ ID NO: 110 - DNA, 24 bp, synthetic construct
  misc_feature 1..24: note = source = /note="Description of Artificial Sequence: Synthetic oligonucleotide"
  source 1..24: mol_type = other DNA
cggaactcttttgatgagcaaaat

SEQ ID NO: 111 - DNA, 45 bp, synthetic construct
  misc_feature 1..45: note = source = /note="Description of Artificial Sequence: Synthetic oligonucleotide"
  source 1..45: mol_type = other DNA
gctctgagtgagtttgacagaggctcaaccctggggaggctatac

SEQ ID NO: 112 - DNA, 15 bp, synthetic construct
  misc_feature 1..15: note = source = /note="Description of Artificial Sequence: Synthetic oligonucleotide"
  source 1..15: mol_type = other DNA
tctgggcacaagagt

SEQ ID NO: 113 - DNA, 18 bp, synthetic construct
  misc_feature 1..18: note = source = /note="Description of Artificial Sequence: Synthetic oligonucleotide"
  source 1..18: mol_type = other DNA
tattatgagaaagaagag

SEQ ID NO: 114 - DNA, 33 bp, synthetic construct
  misc_feature 1..33: note = source = /note="Description of Artificial Sequence: Synthetic oligonucleotide"
  source 1..33: mol_type = other DNA
gccagcagccgggacattaatgaaaaactgttt

SEQ ID NO: 115 - DNA, 18 bp, synthetic construct
  misc_feature 1..18: note = source = /note="Description of Artificial Sequence: Synthetic oligonucleotide"
  source 1..18: mol_type = other DNA
gtcagcggtttaagaggg

SEQ ID NO: 116 - DNA, 21 bp, synthetic construct
  misc_feature 1..21: note = source = /note="Description of Artificial Sequence: Synthetic oligonucleotide"
  source 1..21: mol_type = other DNA
ctgtattcagctggggaagaa

SEQ ID NO: 117 - DNA, 39 bp, synthetic construct
  misc_feature 1..39: note = source = /note="Description of Artificial Sequence: Synthetic oligonucleotide"
  source 1..39: mol_type = other DNA
gctgtgcaggccgatggaggaagccaaggaaatctcatc

SEQ ID NO: 118 - DNA, 15 bp, synthetic construct
  misc_feature 1..15: note = source = /note="Description of Artificial Sequence: Synthetic oligonucleotide"
  source 1..15: mol_type = other DNA
tctgggcacaagagt

SEQ ID NO: 119 - DNA, 18 bp, synthetic construct
  misc_feature 1..18: note = source = /note="Description of Artificial Sequence: Synthetic oligonucleotide"
  source 1..18: mol_type = other DNA
tattatgagaaagaagag

SEQ ID NO: 120 - DNA, 33 bp, synthetic construct
  misc_feature 1..33: note = source = /note="Description of Artificial Sequence: Synthetic oligonucleotide"
  source 1..33: mol_type = other DNA
gccagcagcttggacattaattcacccctccac

SEQ ID NO: 121 - DNA, 18 bp, synthetic construct
  misc_feature 1..18: note = source = /note="Description of Artificial Sequence: Synthetic oligonucleotide"
  source 1..18: mol_type = other DNA
gtcagcggtttaagaggg

SEQ ID NO: 122 - DNA, 21 bp, synthetic construct
  misc_feature 1..21: note = source = /note="Description of Artificial Sequence: Synthetic oligonucleotide"
  source 1..21: mol_type = other DNA
ctgtattcagctggggaagaa

SEQ ID NO: 123 - DNA, 39 bp, synthetic construct
  misc_feature 1..39: note = source = /note="Description of Artificial Sequence: Synthetic oligonucleotide"
  source 1..39: mol_type = other DNA
gctgtgcaggaggatggaggaagccaaggaaatctcatc

SEQ ID NO: 124 - DNA, 15 bp, synthetic construct
  misc_feature 1..15: note = source = /note="Description of Artificial Sequence: Synthetic oligonucleotide"
  source 1..15: mol_type = other DNA
tctgggcacaagagt

SEQ ID NO: 125 - DNA, 18 bp, synthetic construct
  misc_feature 1..18: note = source = /note="Description of Artificial Sequence: Synthetic oligonucleotide"
  source 1..18: mol_type = other DNA
tattatgagaaagaagag

SEQ ID NO: 126 - DNA, 33 bp, synthetic construct; identical to SEQ ID NO: 114
  misc_feature 1..33: note = source = /note="Description of Artificial Sequence: Synthetic oligonucleotide"
  source 1..33: mol_type = other DNA

SEQ ID NO: 127 - DNA, 18 bp, synthetic construct
  misc_feature 1..18: note = source = /note="Description of Artificial Sequence: Synthetic oligonucleotide"
  source 1..18: mol_type = other DNA
gacagtgcctcaaactac

SEQ ID NO: 128 - DNA, 21 bp, synthetic construct
  misc_feature 1..21: note = source = /note="Description of Artificial Sequence: Synthetic oligonucleotide"
  source 1..21: mol_type = other DNA
attcgttcaaatgtgggcgaa

SEQ ID NO: 129 - DNA, 30 bp, synthetic construct
  misc_feature 1..30: note = source = /note="Description of Artificial Sequence: Synthetic oligonucleotide"
  source 1..30: mol_type = other DNA
gcagcatggaactatggtcagaattttgtc

SEQ ID NO: 130 - DNA, 15 bp, synthetic construct
  misc_feature 1..15: note = source = /note="Description of Artificial Sequence: Synthetic oligonucleotide"
  source 1..15: mol_type = other DNA
cctagacacgacact

SEQ ID NO: 131 - DNA, 18 bp, synthetic construct
  misc_feature 1..18: note = source = /note="Description of Artificial Sequence: Synthetic oligonucleotide"
  source 1..18: mol_type = other DNA
ttttatgaaaagatgcag

SEQ ID NO: 132 - DNA, 30 bp, synthetic construct
  misc_feature 1..30: note = source = /note="Description of Artificial Sequence: Synthetic oligonucleotide"
  source 1..30: mol_type = other DNA
gccagcagcttagaggggtctgaagctttc

SEQ ID NO: 133 - DNA, 18 bp, synthetic construct
  misc_feature 1..18: note = source = /note="Description of Artificial Sequence: Synthetic oligonucleotide"
  source 1..18: mol_type = other DNA
aacagcgcctcagactac

SEQ ID NO: 134 - DNA, 21 bp, synthetic construct
  misc_feature 1..21: note = source = /note="Description of Artificial Sequence: Synthetic oligonucleotide"
  source 1..21: mol_type = other DNA
attcgttcaaatatggacaaa

SEQ ID NO: 135 - DNA, 30 bp, synthetic construct
  misc_feature 1..30: note = source = /note="Description of Artificial Sequence: Synthetic oligonucleotide"
  source 1..30: mol_type = other DNA
gcagagaataactatggtcagaattttgtc

SEQ ID NO: 136 - DNA, 15 bp, synthetic construct
  misc_feature 1..15: note = source = /note="Description of Artificial Sequence: Synthetic oligonucleotide"
  source 1..15: mol_type = other DNA
atgaaccataactcc

SEQ ID NO: 137 - DNA, 18 bp, synthetic construct
  misc_feature 1..18: note = source = /note="Description of Artificial Sequence: Synthetic oligonucleotide"
  source 1..18: mol_type = other DNA
tcagcttctgagggtacc

SEQ ID NO: 138 - DNA, 36 bp, synthetic construct
  misc_feature 1..36: note = source = /note="Description of Artificial Sequence: Synthetic oligonucleotide"
  source 1..36: mol_type = other DNA
gccagcagtgactggggacagggggttgaagctttc

SEQ ID NO: 139 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 140 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 141 - DNA, 48 bp, synthetic construct
  misc_feature 1..48: note = source = /note="Description of Artificial Sequence: Synthetic oligonucleotide"
  source 1..48: mol_type = other DNA
atcctgagagaatacatgtattcaggaggaggtgctgacggactcacc

SEQ ID NO: 142 - DNA, 15 bp, synthetic construct
  misc_feature 1..15: note = source = /note="Description of Artificial Sequence: Synthetic oligonucleotide"
  source 1..15: mol_type = other DNA
tctgggcacaagagt

SEQ ID NO: 143 - DNA, 18 bp, synthetic construct
  misc_feature 1..18: note = source = /note="Description of Artificial Sequence: Synthetic oligonucleotide"
  source 1..18: mol_type = other DNA
tattatgagaaagaagag

SEQ ID NO: 144 - DNA, 39 bp, synthetic construct
  misc_feature 1..39: note = source = /note="Description of Artificial Sequence: Synthetic oligonucleotide"
  source 1..39: mol_type = other DNA
gccagcagcttccaagcaggggttaactatggctacacc

SEQ ID NO: 145 - DNA, 21 bp, synthetic construct
  misc_feature 1..21: note = source = /note="Description of Artificial Sequence: Synthetic oligonucleotide"
  source 1..21: mol_type = other DNA
accagtgagaataattattat

SEQ ID NO: 146 - DNA, 24 bp, synthetic construct
  misc_feature 1..24: note = source = /note="Description of Artificial Sequence: Synthetic oligonucleotide"
  source 1..24: mol_type = other DNA
caagaagcttataagcaacagaat

SEQ ID NO: 147 - DNA, 42 bp, synthetic construct
  misc_feature 1..42: note = source = /note="Description of Artificial Sequence: Synthetic oligonucleotide"
  source 1..42: mol_type = other DNA
gctttcggtatgtattcaggaggaggtgctgacggactcacc

SEQ ID NO: 148 - DNA, 15 bp, synthetic construct
  misc_feature 1..15: note = source = /note="Description of Artificial Sequence: Synthetic oligonucleotide"
  source 1..15: mol_type = other DNA
tggagccacagctat

SEQ ID NO: 149 - DNA, 18 bp, synthetic construct
  misc_feature 1..18: note = source = /note="Description of Artificial Sequence: Synthetic oligonucleotide"
  source 1..18: mol_type = other DNA
tcagcagctgctgatatt

SEQ ID NO: 150 - DNA, 36 bp, synthetic construct
  misc_feature 1..36: note = source = /note="Description of Artificial Sequence: Synthetic oligonucleotide"
  source 1..36: mol_type = other DNA
gccagcagtgatgggacagggtactatggctacacc

SEQ ID NO: 151 - DNA, 21 bp, synthetic construct
  misc_feature 1..21: note = source = /note="Description of Artificial Sequence: Synthetic oligonucleotide"
  source 1..21: mol_type = other DNA
accagtgagaataattattat

SEQ ID NO: 152 - DNA, 24 bp, synthetic construct
  misc_feature 1..24: note = source = /note="Description of Artificial Sequence: Synthetic oligonucleotide"
  source 1..24: mol_type = other DNA
caagaagcttataagcaacagaat

SEQ ID NO: 153 - DNA, 30 bp, synthetic construct
  misc_feature 1..30: note = source = /note="Description of Artificial Sequence: Synthetic oligonucleotide"
  source 1..30: mol_type = other DNA
gcccttatggaatatggaaacaaactggtc

SEQ ID NO: 154 - DNA, 15 bp, synthetic construct
  misc_feature 1..15: note = source = /note="Description of Artificial Sequence: Synthetic oligonucleotide"
  source 1..15: mol_type = other DNA
tggagccacagctat

SEQ ID NO: 155 - DNA, 18 bp, synthetic construct
  misc_feature 1..18: note = source = /note="Description of Artificial Sequence: Synthetic oligonucleotide"
  source 1..18: mol_type = other DNA
tcagcagctgctgatatt

SEQ ID NO: 156 - DNA, 36 bp, synthetic construct; identical to SEQ ID NO: 150
  misc_feature 1..36: note = source = /note="Description of Artificial Sequence: Synthetic oligonucleotide"
  source 1..36: mol_type = other DNA

SEQ ID NO: 157 - DNA, 21 bp, synthetic construct
  misc_feature 1..21: note = source = /note="Description of Artificial Sequence: Synthetic oligonucleotide"
  source 1..21: mol_type = other DNA
accagtgagaataattattat

SEQ ID NO: 158 - DNA, 24 bp, synthetic construct
  misc_feature 1..24: note = source = /note="Description of Artificial Sequence: Synthetic oligonucleotide"
  source 1..24: mol_type = other DNA
caagaagcttataagcaacagaat

SEQ ID NO: 159 - DNA, 30 bp, synthetic construct
  misc_feature 1..30: note = source = /note="Description of Artificial Sequence: Synthetic oligonucleotide"
  source 1..30: mol_type = other DNA
gcccttatggaatatgaaaacaaactggtc

SEQ ID NO: 160 - DNA, 15 bp, synthetic construct
  misc_feature 1..15: note = source = /note="Description of Artificial Sequence: Synthetic oligonucleotide"
  source 1..15: mol_type = other DNA
tggagccacagctat

SEQ ID NO: 161 - DNA, 18 bp, synthetic construct
  misc_feature 1..18: note = source = /note="Description of Artificial Sequence: Synthetic oligonucleotide"
  source 1..18: mol_type = other DNA
tcagcagctgctgatatt

SEQ ID NO: 162 - DNA, 36 bp, synthetic construct; identical to SEQ ID NO: 150
  misc_feature 1..36: note = source = /note="Description of Artificial Sequence: Synthetic oligonucleotide"
  source 1..36: mol_type = other DNA

SEQ ID NO: 163 - DNA, 18 bp, synthetic construct
  misc_feature 1..18: note = source = /note="Description of Artificial Sequence: Synthetic oligonucleotide"
  source 1..18: mol_type = other DNA
tccagcaatttttatgcc

SEQ ID NO: 164 - DNA, 18 bp, synthetic construct
  misc_feature 1..18: note = source = /note="Description of Artificial Sequence: Synthetic oligonucleotide"
  source 1..18: mol_type = other DNA
atgactttaaatggggat

SEQ ID NO: 165 - DNA, 36 bp, synthetic construct
  misc_feature 1..36: note = source = /note="Description of Artificial Sequence: Synthetic oligonucleotide"
  source 1..36: mol_type = other DNA
gcctgtgggggttctggcaacacaggcaaactaatc

SEQ ID NO: 166 - DNA, 15 bp, synthetic construct
  misc_feature 1..15: note = source = /note="Description of Artificial Sequence: Synthetic oligonucleotide"
  source 1..15: mol_type = other DNA
cctagacacgacact

SEQ ID NO: 167 - DNA, 18 bp, synthetic construct
  misc_feature 1..18: note = source = /note="Description of Artificial Sequence: Synthetic oligonucleotide"
  source 1..18: mol_type = other DNA
ttttatgaaaagatgcag

SEQ ID NO: 168 - DNA, 30 bp, synthetic construct
  misc_feature 1..30: note = source = /note="Description of Artificial Sequence: Synthetic oligonucleotide"
  source 1..30: mol_type = other DNA
gccagcagctcccagggtcagccccagcat

SEQ ID NO: 169 - DNA, 21 bp, synthetic construct
  misc_feature 1..21: note = source = /note="Description of Artificial Sequence: Synthetic oligonucleotide"
  source 1..21: mol_type = other DNA
acccgtgatactacttattac

SEQ ID NO: 170 - DNA, 24 bp, synthetic construct
  misc_feature 1..24: note = source = /note="Description of Artificial Sequence: Synthetic oligonucleotide"
  source 1..24: mol_type = other DNA
cggaactcttttgatgagcaaaat

SEQ ID NO: 171 - DNA, 33 bp, synthetic construct
  misc_feature 1..33: note = source = /note="Description of Artificial Sequence: Synthetic oligonucleotide"
  source 1..33: mol_type = other DNA
gctctgagtgagggatatggaaacaaactggtc

SEQ ID NO: 172 - DNA, 15 bp, synthetic construct
  misc_feature 1..15: note = source = /note="Description of Artificial Sequence: Synthetic oligonucleotide"
  source 1..15: mol_type = other DNA
aaaggacacagtcat

SEQ ID NO: 173 - DNA, 18 bp, synthetic construct
  misc_feature 1..18: note = source = /note="Description of Artificial Sequence: Synthetic oligonucleotide"
  source 1..18: mol_type = other DNA
ctccagaaagaaaatatc

SEQ ID NO: 174 - DNA, 33 bp, synthetic construct
  misc_feature 1..33: note = source = /note="Description of Artificial Sequence: Synthetic oligonucleotide"
  source 1..33: mol_type = other DNA
gccagctcacacagggacgacactgaagctttc

SEQ ID NO: 175 - DNA, 18 bp, synthetic construct
  misc_feature 1..18: note = source = /note="Description of Artificial Sequence: Synthetic oligonucleotide"
  source 1..18: mol_type = other DNA
gatagcgctatttacaac

SEQ ID NO: 176 - DNA, 21 bp, synthetic construct
  misc_feature 1..21: note = source = /note="Description of Artificial Sequence: Synthetic oligonucleotide"
  source 1..21: mol_type = other DNA
attcagtcaagtcagagagag

SEQ ID NO: 177 - DNA, 39 bp, synthetic construct
  misc_feature 1..39: note = source = /note="Description of Artificial Sequence: Synthetic oligonucleotide"
  source 1..39: mol_type = other DNA
gctgtggaggggacaactgacagctgggggaaattccag

SEQ ID NO: 178 - DNA, 15 bp, synthetic construct
  misc_feature 1..15: note = source = /note="Description of Artificial Sequence: Synthetic oligonucleotide"
  source 1..15: mol_type = other DNA
cctagacacgacact

SEQ ID NO: 179 - DNA, 18 bp, synthetic construct
  misc_feature 1..18: note = source = /note="Description of Artificial Sequence: Synthetic oligonucleotide"
  source 1..18: mol_type = other DNA
ttttatgaaaagatgcag

SEQ ID NO: 180 - DNA, 30 bp, synthetic construct; identical to SEQ ID NO: 168
  misc_feature 1..30: note = source = /note="Description of Artificial Sequence: Synthetic oligonucleotide"
  source 1..30: mol_type = other DNA

SEQ ID NO: 181 - DNA, 18 bp, synthetic construct
  misc_feature 1..18: note = source = /note="Description of Artificial Sequence: Synthetic oligonucleotide"
  source 1..18: mol_type = other DNA
tattctgggagtcctgaa

SEQ ID NO: 182 - DNA, 12 bp, synthetic construct
  misc_feature 1..12: note = source = /note="Description of Artificial Sequence: Synthetic oligonucleotide"
  source 1..12: mol_type = other DNA
cacatctctaga

SEQ ID NO: 183 - DNA, 39 bp, synthetic construct
  misc_feature 1..39: note = source = /note="Description of Artificial Sequence: Synthetic oligonucleotide"
  source 1..39: mol_type = other DNA
gctctaagtggggcatcaggaggaagctacatacctaca

SEQ ID NO: 184 - DNA, 15 bp, synthetic construct
  misc_feature 1..15: note = source = /note="Description of Artificial Sequence: Synthetic oligonucleotide"
  source 1..15: mol_type = other DNA
aaaggacacagtcat

SEQ ID NO: 185 - DNA, 18 bp, synthetic construct
  misc_feature 1..18: note = source = /note="Description of Artificial Sequence: Synthetic oligonucleotide"
  source 1..18: mol_type = other DNA
ctccagaaagaaaatatc

SEQ ID NO: 186 - DNA, 33 bp, synthetic construct; identical to SEQ ID NO: 174
  misc_feature 1..33: note = source = /note="Description of Artificial Sequence: Synthetic oligonucleotide"
  source 1..33: mol_type = other DNA

SEQ ID NO: 187 - DNA, 21 bp, synthetic construct
  misc_feature 1..21: note = source = /note="Description of Artificial Sequence: Synthetic oligonucleotide"
  source 1..21: mol_type = other DNA
acccgtgatactacttattac

SEQ ID NO: 188 - DNA, 24 bp, synthetic construct
  misc_feature 1..24: note = source = /note="Description of Artificial Sequence: Synthetic oligonucleotide"
  source 1..24: mol_type = other DNA
cggaactcttttgatgagcaaaat

SEQ ID NO: 189 - DNA, 39 bp, synthetic construct
  misc_feature 1..39: note = source = /note="Description of Artificial Sequence: Synthetic oligonucleotide"
  source 1..39: mol_type = other DNA
gctctgagtgtatcatcttataacaccgacaagctcatc

SEQ ID NO: 190 - DNA, 15 bp, synthetic construct
  misc_feature 1..15: note = source = /note="Description of Artificial Sequence: Synthetic oligonucleotide"
  source 1..15: mol_type = other DNA
aaaggacacagtcat

SEQ ID NO: 191 - DNA, 18 bp, synthetic construct
  misc_feature 1..18: note = source = /note="Description of Artificial Sequence: Synthetic oligonucleotide"
  source 1..18: mol_type = other DNA
ctccagaaagaaaatatc

SEQ ID NO: 192 - DNA, 33 bp, synthetic construct
  misc_feature 1..33: note = source = /note="Description of Artificial Sequence: Synthetic oligonucleotide"
  source 1..33: mol_type = other DNA
gccagctcacacagggatgacactgaagctttc

SEQ ID NO: 193 - DNA, 21 bp, synthetic construct
  misc_feature 1..21: note = source = /note="Description of Artificial Sequence: Synthetic oligonucleotide"
  source 1..21: mol_type = other DNA
acccgtgatactacttattac

SEQ ID NO: 194 - DNA, 24 bp, synthetic construct
  misc_feature 1..24: note = source = /note="Description of Artificial Sequence: Synthetic oligonucleotide"
  source 1..24: mol_type = other DNA
cggaactcttttgatgagcaaaat

SEQ ID NO: 195 - DNA, 36 bp, synthetic construct
  misc_feature 1..36: note = source = /note="Description of Artificial Sequence: Synthetic oligonucleotide"
  source 1..36: mol_type = other DNA
gctctgagtgaggggtataacaccgacaagctcatc

SEQ ID NO: 196 - DNA, 15 bp, synthetic construct
  misc_feature 1..15: note = source = /note="Description of Artificial Sequence: Synthetic oligonucleotide"
  source 1..15: mol_type = other DNA
atggaccatgaaaat

SEQ ID NO: 197 - DNA, 18 bp, synthetic construct
  misc_feature 1..18: note = source = /note="Description of Artificial Sequence: Synthetic oligonucleotide"
  source 1..18: mol_type = other DNA
tcatatgatgttaaaatg

SEQ ID NO: 198 - DNA, 33 bp, synthetic construct
  misc_feature 1..33: note = source = /note="Description of Artificial Sequence: Synthetic oligonucleotide"
  source 1..33: mol_type = other DNA
gccagcagtttagggggggcgaacaccatatat

SEQ ID NO: 199 - DNA, 18 bp, synthetic construct
  misc_feature 1..18: note = source = /note="Description of Artificial Sequence: Synthetic oligonucleotide"
  source 1..18: mol_type = other DNA
gaccgaggttcccagtcc

SEQ ID NO: 200 - DNA, 18 bp, synthetic construct
  misc_feature 1..18: note = source = /note="Description of Artificial Sequence: Synthetic oligonucleotide"
  source 1..18: mol_type = other DNA
atatactccaatggtgac

SEQ ID NO: 201 - DNA, 39 bp, synthetic construct
  misc_feature 1..39: note = source = /note="Description of Artificial Sequence: Synthetic oligonucleotide"
  source 1..39: mol_type = other DNA
gccgtgaacattccgaattcaggatacagcaccctcacc

SEQ ID NO: 202 - DNA, 15 bp, synthetic construct
  misc_feature 1..15: note = source = /note="Description of Artificial Sequence: Synthetic oligonucleotide"
  source 1..15: mol_type = other DNA
atgaaccatgaatac

SEQ ID NO: 203 - DNA, 18 bp, synthetic construct
  misc_feature 1..18: note = source = /note="Description of Artificial Sequence: Synthetic oligonucleotide"
  source 1..18: mol_type = other DNA
tcagttggtgagggtaca

SEQ ID NO: 204 - DNA, 33 bp, synthetic construct
  misc_feature 1..33: note = source = /note="Description of Artificial Sequence: Synthetic oligonucleotide"
  source 1..33: mol_type = other DNA
gccagcagttactgggagggcactgaagctttc

SEQ ID NO: 205 - DNA, 21 bp, synthetic construct
  misc_feature 1..21: note = source = /note="Description of Artificial Sequence: Synthetic oligonucleotide"
  source 1..21: mol_type = other DNA
accagtgagaataattattat

SEQ ID NO: 206 - DNA, 24 bp, synthetic construct
  misc_feature 1..24: note = source = /note="Description of Artificial Sequence: Synthetic oligonucleotide"
  source 1..24: mol_type = other DNA
caagaagcttataagcaacagaat

SEQ ID NO: 207 - DNA, 27 bp, synthetic construct
  misc_feature 1..27: note = source = /note="Description of Artificial Sequence: Synthetic oligonucleotide"
  source 1..27: mol_type = other DNA
gctttcgactatggtcagaattttgtc

SEQ ID NO: 208 - DNA, 15 bp, synthetic construct
  misc_feature 1..15: note = source = /note="Description of Artificial Sequence: Synthetic oligonucleotide"
  source 1..15: mol_type = other DNA
atgaaccataactac

SEQ ID NO: 209 - DNA, 18 bp, synthetic construct
  misc_feature 1..18: note = source = /note="Description of Artificial Sequence: Synthetic oligonucleotide"
  source 1..18: mol_type = other DNA
tcagttggtgctggtatc

SEQ ID NO: 210 - DNA, 36 bp, synthetic construct
  misc_feature 1..36: note = source = /note="Description of Artificial Sequence: Synthetic oligonucleotide"
  source 1..36: mol_type = other DNA
gccagcagttacgggggggggcagactgaagctttc

SEQ ID NO: 211 - DNA, 21 bp, synthetic construct
  misc_feature 1..21: note = source = /note="Description of Artificial Sequence: Synthetic oligonucleotide"
  source 1..21: mol_type = other DNA
acccgtgatactacttattac

SEQ ID NO: 212 - DNA, 24 bp, synthetic construct
  misc_feature 1..24: note = source = /note="Description of Artificial Sequence: Synthetic oligonucleotide"
  source 1..24: mol_type = other DNA
cggaactcttttgatgagcaaaat

SEQ ID NO: 213 - DNA, 39 bp, synthetic construct
  misc_feature 1..39: note = source = /note="Description of Artificial Sequence: Synthetic oligonucleotide"
  source 1..39: mol_type = other DNA
gctctgagtgagggttataaccagggaggaaagcttatc

SEQ ID NO: 214 - DNA, 15 bp, synthetic construct
  misc_feature 1..15: note = source = /note="Description of Artificial Sequence: Synthetic oligonucleotide"
  source 1..15: mol_type = other DNA
atggaccatgaaaat

SEQ ID NO: 215 - DNA, 18 bp, synthetic construct
  misc_feature 1..18: note = source = /note="Description of Artificial Sequence: Synthetic oligonucleotide"
  source 1..18: mol_type = other DNA
tcatatgatgttaaaatg

SEQ ID NO: 216 - DNA, 33 bp, synthetic construct
  misc_feature 1..33: note = source = /note="Description of Artificial Sequence: Synthetic oligonucleotide"
  source 1..33: mol_type = other DNA
gccagcggggcagatagcaatcagccccagcat

SEQ ID NO: 217 - protein, 118 aa, synthetic construct
  REGION 1..118: note = source = /note="Description of Artificial Sequence: Synthetic polypeptide"
AQKVTQAQPEISVVEKEDVTLDCVYETRDTTYYLFWYKQPPSGELVFLIRRNSFDEQNEISGRYSWNFQKSTSSFNFTITASQVVDSAVYFCALSEFDRGSTLGRLYFGRGTQLTVWP

SEQ ID NO: 218 - DNA, 354 bp, synthetic construct
  misc_feature 1..354: note = source = /note="Description of Artificial Sequence: Synthetic polynucleotide"
  source 1..354: mol_type = other DNA
gctcagaaggtaactcaagcgcagcctgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagtttgacagaggctcaaccctggggaggctatactttggaagaggaactcagttgactgtctggcct

SEQ ID NO: 219 - protein, 112 aa, synthetic construct
  REGION 1..112: note = source = /note="Description of Artificial Sequence: Synthetic polypeptide"
DAGVTQSPTHLIKTRGQQVTLRCSPISGHKSVSWYQQVLGQGPQFIFQYYEKEERGRGNFPDRFSARQFPNYSSELNVNALLLGDSALYLCASSRDINEKLFFGSGTQLSVL

SEQ ID NO: 220 - DNA, 336 bp, synthetic construct
  misc_feature 1..336: note = source = /note="Description of Artificial Sequence: Synthetic polynucleotide"
  source 1..336: mol_type = other DNA
gacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagccgggacattaatgaaaaactgttttttggcagtggaacccagctctctgtcttg

SEQ ID NO: 221 - protein, 112 aa, synthetic construct
  REGION 1..112: note = source = /note="Description of Artificial Sequence: Synthetic polypeptide"
EDQVTQSPEALRLQEGESSSLNCSYTVSGLRGLFWYRQDPGKGPEFLFTLYSAGEEKEKERLKATLTKKESFLHITAPKPEDSATYLCAVQADGGSQGNLIFGKGTKLSVKP

SEQ ID NO: 222 - DNA, 336 bp, synthetic construct
  misc_feature 1..336: note = source = /note="Description of Artificial Sequence: Synthetic polynucleotide"
  source 1..336: mol_type = other DNA
gaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcaggccgatggaggaagccaaggaaatctcatctttggaaaaggcactaaactctctgttaaacca

SEQ ID NO: 223 - protein, 112 aa, synthetic construct
  REGION 1..112: note = source = /note="Description of Artificial Sequence: Synthetic polypeptide"
DAGVTQSPTHLIKTRGQQVTLRCSPISGHKSVSWYQQVLGQGPQFIFQYYEKEERGRGNFPDRFSARQFPNYSSELNVNALLLGDSALYLCASSLDINSPLHFGNGTRLTVT

SEQ ID NO: 224 - DNA, 336 bp, synthetic construct
  misc_feature 1..336: note = source = /note="Description of Artificial Sequence: Synthetic polynucleotide"
  source 1..336: mol_type = other DNA
gacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagcttggacattaattcacccctccactttgggaacgggaccaggctcactgtgaca

SEQ ID NO: 225 - protein, 112 aa, synthetic construct
  REGION 1..112: note = source = /note="Description of Artificial Sequence: Synthetic polypeptide"
EDQVTQSPEALRLQEGESSSLNCSYTVSGLRGLFWYRQDPGKGPEFLFTLYSAGEEKEKERLKATLTKKESFLHITAPKPEDSATYLCAVQEDGGSQGNLIFGKGTKLSVKP

SEQ ID NO: 226 - DNA, 336 bp, synthetic construct
  misc_feature 1..336: note = source = /note="Description of Artificial Sequence: Synthetic polynucleotide"
  source 1..336: mol_type = other DNA
gaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcaggaggatggaggaagccaaggaaatctcatctttggaaaaggcactaaactctctgttaaacca

SEQ ID NO: 227 - protein, 112 aa, synthetic construct; identical to SEQ ID NO: 219
  REGION 1..112: note = source = /note="Description of Artificial Sequence: Synthetic polypeptide"

SEQ ID NO: 228 - DNA, 336 bp, synthetic construct; identical to SEQ ID NO: 220
  misc_feature 1..336: note = source = /note="Description of Artificial Sequence: Synthetic polynucleotide"
  source 1..336: mol_type = other DNA

SEQ ID NO: 229 - protein, 111 aa, synthetic construct
  REGION 1..111: note = source = /note="Description of Artificial Sequence: Synthetic polypeptide"
GENVEQHPSTLSVQEGDSAVIKCTYSDSASNYFPWYKQELGKRPQLIIDIRSNVGEKKDQRIAVTLNKTAKHFSLHITETQPEDSAVYFCAAWNYGQNFVFGPGTRLSVLP

SEQ ID NO: 230 - DNA, 333 bp, synthetic construct
  misc_feature 1..333: note = source = /note="Description of Artificial Sequence: Synthetic polynucleotide"
  source 1..333: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcatggaactatggtcagaattttgtctttggtcccggaaccagattgtccgtgctgccc

SEQ ID NO: 231 - protein, 111 aa, synthetic construct
  REGION 1..111: note = source = /note="Description of Artificial Sequence: Synthetic polypeptide"
AAGVIQSPRHLIKEKRETATLKCYPIPRHDTVYWYQQGPGQDPQFLISFYEKMQSDKGSIPDRFSAQQFSDYHSELNMSSLELGDSALYFCASSLEGSEAFFGQGTRLTVV

SEQ ID NO: 232 - DNA, 333 bp, synthetic construct
  misc_feature 1..333: note = source = /note="Description of Artificial Sequence: Synthetic polynucleotide"
  source 1..333: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttagaggggtctgaagctttctttggacaaggcaccagactcacagttgta

SEQ ID NO: 233 - protein, 111 aa, synthetic construct
  REGION 1..111: note = source = /note="Description of Artificial Sequence: Synthetic polypeptide"
GESVGLHLPTLSVQEGDNSIINCAYSNSASDYFIWYKQESGKGPQFIIDIRSNMDKRQGQRVTVLLNKTVKHLSLQIAATQPGDSAVYFCAENNYGQNFVFGPGTRLSVLP

SEQ ID NO: 234 - DNA, 333 bp, synthetic construct
  misc_feature 1..333: note = source = /note="Description of Artificial Sequence: Synthetic polynucleotide"
  source 1..333: mol_type = other DNA
ggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaataactatggtcagaattttgtctttggtcccggaaccagattgtccgtgctgccc

SEQ ID NO: 235 - protein, 113 aa, synthetic construct
  REGION 1..113: note = source = /note="Description of Artificial Sequence: Synthetic polypeptide"
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSDWGQGVEAFFGQGTRLTVV

SEQ ID NO: 236 - DNA, 339 bp, synthetic construct
  misc_feature 1..339: note = source = /note="Description of Artificial Sequence: Synthetic polynucleotide"
  source 1..339: mol_type = other DNA
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgactggggacagggggttgaagctttctttggacaaggcaccagactcacagttgta

SEQ ID NO: 237 - protein, 115 aa, synthetic construct
  REGION 1..115: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILREYMYSGGGADGLTFGKGTHLIIQP

SEQ ID NO: 238 - DNA, 345 bp, synthetic construct
  misc_feature 1..345: note = source = /note="Description of Artificial Sequence: Synthetic polynucleotide"
  source 1..345: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagaatacatgtattcaggaggaggtgctgacggactcacctttggcaaagggactcatctaatcatccagccc

SEQ ID NO: 239 - protein, 114 aa, synthetic construct
  REGION 1..114: note = source = /note="Description of Artificial Sequence: Synthetic polypeptide"
DAGVTQSPTHLIKTRGQQVTLRCSPISGHKSVSWYQQVLGQGPQFIFQYYEKEERGRGNFPDRFSARQFPNYSSELNVNALLLGDSALYLCASSFQAGVNYGYTFGSGTRLTVV

SEQ ID NO: 240 - DNA, 342 bp, synthetic construct
  misc_feature 1..342: note = source = /note="Description of Artificial Sequence: Synthetic polynucleotide"
  source 1..342: mol_type = other DNA
gacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagcttccaagcaggggttaactatggctacaccttcggttcggggaccaggttaaccgttgta

SEQ ID NO: 241 - protein, 117 aa, synthetic construct
  REGION 1..117: note = source = /note="Description of Artificial Sequence: Synthetic polypeptide"
AQTVTQSQPEMSVQEAETVTLSCTYDTSENNYYLFWYKQPPSRQMILVIRQEAYKQQNATENRFSVNFQKAAKSFSLKISDSQLGDTAMYFCAFGMYSGGGADGLTFGKGTHLIIQP

SEQ ID NO: 242 - DNA, 351 bp, synthetic construct
  misc_feature 1..351: note = source = /note="Description of Artificial Sequence: Synthetic polynucleotide"
  source 1..351: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcggtatgtattcaggaggaggtgctgacggactcacctttggcaaagggactcatctaatcatccagccc

SEQ ID NO: 243 - protein, 113 aa, synthetic construct
  REGION 1..113: note = source = /note="Description of Artificial Sequence: Synthetic polypeptide"
DAGITQSPRYKITETGRQVTLMCHQTWSHSYMFWYRQDLGHGLRLIYYSAAADITDKGEVPDGYVVSRSKTENFPLTLESATRSQTSVYFCASSDGTGYYGYTFGSGTRLTVV

SEQ ID NO: 244 - DNA, 339 bp, synthetic construct
  misc_feature 1..339: note = source = /note="Description of Artificial Sequence: Synthetic polynucleotide"
  source 1..339: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgatgggacagggtactatggctacaccttcggttcggggaccaggttaaccgttgta

SEQ ID NO: 245 - protein, 113 aa, synthetic construct
  REGION 1..113: note = source = /note="Description of Artificial Sequence: Synthetic polypeptide"
AQTVTQSQPEMSVQEAETVTLSCTYDTSENNYYLFWYKQPPSRQMILVIRQEAYKQQNATENRFSVNFQKAAKSFSLKISDSQLGDTAMYFCALMEYGNKLVFGAGTILRVKS

SEQ ID NO: 246 - DNA, 339 bp, synthetic construct
  misc_feature 1..339: note = source = /note="Description of Artificial Sequence: Synthetic polynucleotide"
  source 1..339: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcccttatggaatatggaaacaaactggtctttggcgcaggaaccattctgagagtcaagtcc

SEQ ID NO: 247 - protein, 113 aa, synthetic construct; identical to SEQ ID NO: 243
  REGION 1..113: note = source = /note="Description of Artificial Sequence: Synthetic polypeptide"

SEQ ID NO: 248 - DNA, 339 bp, synthetic construct; identical to SEQ ID NO: 244
  misc_feature 1..339: note = source = /note="Description of Artificial Sequence: Synthetic polynucleotide"
  source 1..339: mol_type = other DNA

SEQ ID NO: 249 - protein, 113 aa, synthetic construct
  REGION 1..113: note = source = /note="Description of Artificial Sequence: Synthetic polypeptide"
AQTVTQSQPEMSVQEAETVTLSCTYDTSENNYYLFWYKQPPSRQMILVIRQEAYKQQNATENRFSVNFQKAAKSFSLKISDSQLGDTAMYFCALMEYENKLVFGAGTILRVKS

SEQ ID NO: 250 - DNA, 339 bp, synthetic construct
  misc_feature 1..339: note = source = /note="Description of Artificial Sequence: Synthetic polynucleotide"
  source 1..339: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgcccttatggaatatgaaaacaaactggtctttggcgcaggaaccattctgagagtcaagtcc

SEQ ID NO: 251 - protein, 113 aa, synthetic construct; identical to SEQ ID NO: 243
  REGION 1..113: note = source = /note="Description of Artificial Sequence: Synthetic polypeptide"

SEQ ID NO: 252 - DNA, 339 bp, synthetic construct; identical to SEQ ID NO: 244
  misc_feature 1..339: note = source = /note="Description of Artificial Sequence: Synthetic polynucleotide"
  source 1..339: mol_type = other DNA

SEQ ID NO: 253 - protein, 113 aa, synthetic construct
  REGION 1..113: note = source = /note="Description of Artificial Sequence: Synthetic polypeptide"
ILNVEQSPQSLHVQEGDSTNFTCSFPSSNFYALHWYRWETAKSPEALFVMTLNGDEKKKGRISATLNTKEGYSYLYIKGSQPEDSATYLCACGGSGNTGKLIFGQGTTLQVKP

SEQ ID NO: 254 - DNA, 339 bp, synthetic construct
  misc_feature 1..339: note = source = /note="Description of Artificial Sequence: Synthetic polynucleotide"
  source 1..339: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctgtgggggttctggcaacacaggcaaactaatctttgggcaagggacaactttacaagtaaaacca

SEQ ID NO: 255 - protein, 111 aa, synthetic construct
  REGION 1..111: note = source = /note="Description of Artificial Sequence: Synthetic polypeptide"
AAGVIQSPRHLIKEKRETATLKCYPIPRHDTVYWYQQGPGQDPQFLISFYEKMQSDKGSIPDRFSAQQFSDYHSELNMSSLELGDSALYFCASSSQGQPQHFGDGTRLSIL

SEQ ID NO: 256 - DNA, 333 bp, synthetic construct
  misc_feature 1..333: note = source = /note="Description of Artificial Sequence: Synthetic polynucleotide"
  source 1..333: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagctcccagggtcagccccagcattttggtgatgggactcgactctccatccta

SEQ ID NO: 257 - protein, 114 aa, synthetic construct
  REGION 1..114: note = source = /note="Description of Artificial Sequence: Synthetic polypeptide"
AQKVTQATREISVVEKEDVTLDCVYETRDTTYYLFWYKQPPSGELVFLIRRNSFDEQNEISGRYSWNFQKSTSSFNFTITASQVVDSAVYFCALSEGYGNKLVFGAGTILRVKS

SEQ ID NO: 258 - DNA, 342 bp, synthetic construct
  misc_feature 1..342: note = source = /note="Description of Artificial Sequence: Synthetic polynucleotide"
  source 1..342: mol_type = other DNA
gctcagaaggtaactcaagcgactagagaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagggatatggaaacaaactggtctttggcgcaggaaccattctgagagtcaagtcc

SEQ ID NO: 259 - protein, 113 aa, synthetic construct
  REGION 1..113: note = source = /note="Description of Artificial Sequence: Synthetic polypeptide"
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSHRDDTEAFFGQGTRLTVV

SEQ ID NO: 260 - DNA, 339 bp, synthetic construct
  misc_feature 1..339: note = source = /note="Description of Artificial Sequence: Synthetic polynucleotide"
  source 1..339: mol_type = other DNA
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcacacagggacgacactgaagctttctttggacaaggcaccagactcacagttgta

SEQ ID NO: 261 - protein, 114 aa, synthetic construct
  REGION 1..114: note = source = /note="Description of Artificial Sequence: Synthetic polypeptide"
KQEVTQIPAALSVPEGENLVLNCSFTDSAIYNLQWFRQDPGKGLTSLLLIQSSQREQTSGRLNASLDKSSGRSTLYIAASQPGDSATYLCAVEGTTDSWGKFQFGAGTQVVVTP

SEQ ID NO: 262 - DNA, 342 bp, synthetic construct
  misc_feature 1..342: note = source = /note="Description of Artificial Sequence: Synthetic polynucleotide"
  source 1..342: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtggaggggacaactgacagctgggggaaattccagtttggagcagggacccaggttgtggtcacccca

SEQ ID NO: 263 - protein, 111 aa, synthetic construct; identical to SEQ ID NO: 255
  REGION 1..111: note = source = /note="Description of Artificial Sequence: Synthetic polypeptide"

SEQ ID NO: 264 - DNA, 333 bp, synthetic construct; identical to SEQ ID NO: 256
  misc_feature 1..333: note = source = /note="Description of Artificial Sequence: Synthetic polynucleotide"
  source 1..333: mol_type = other DNA

SEQ ID NO: 265 - protein, 111 aa, synthetic construct
  REGION 1..111: note = source = /note="Description of Artificial Sequence: Synthetic polypeptide"
AQRVTQPEKLLSVFKGAPVELKCNYSYSGSPELFWYVQYSRQRLQLLLRHISRESIKGFTADLNKGETSFHLKKPFAQEEDSAMYYCALSGASGGSYIPTFGRGTSLIVHP

SEQ ID NO: 266 - DNA, 333 bp, synthetic construct
  misc_feature 1..333: note = source = /note="Description of Artificial Sequence: Synthetic polynucleotide"
  source 1..333: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaagtggggcatcaggaggaagctacatacctacatttggaagaggaaccagccttattgttcatccg

SEQ ID NO: 267 - protein, 113 aa, synthetic construct; identical to SEQ ID NO: 259
  REGION 1..113: note = source = /note="Description of Artificial Sequence: Synthetic polypeptide"

SEQ ID NO: 268 - DNA, 339 bp, synthetic construct; identical to SEQ ID NO: 260
  misc_feature 1..339: note = source = /note="Description of Artificial Sequence: Synthetic polynucleotide"
  source 1..339: mol_type = other DNA

SEQ ID NO: 269 - protein, 116 aa, synthetic construct
  REGION 1..116: note = source = /note="Description of Artificial Sequence: Synthetic polypeptide"
AQKVTQAQTEISVVEKEDVTLDCVYETRDTTYYLFWYKQPPSGELVFLIRRNSFDEQNEISGRYSWNFQKSTSSFNFTITASQVVDSAVYFCALSVSSYNTDKLIFGTGTRLQVFP

SEQ ID NO: 270 - DNA, 348 bp, synthetic construct
  misc_feature 1..348: note = source = /note="Description of Artificial Sequence: Synthetic polynucleotide"
  source 1..348: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgtatcatcttataacaccgacaagctcatctttgggactgggaccagattacaagtctttcca

SEQ ID NO: 271 - protein, 113 aa, synthetic construct; identical to SEQ ID NO: 259
  REGION 1..113: note = source = /note="Description of Artificial Sequence: Synthetic polypeptide"

SEQ ID NO: 272 - DNA, 339 bp, synthetic construct
  misc_feature 1..339: note = source = /note="Description of Artificial Sequence: Synthetic polynucleotide"
  source 1..339: mol_type = other DNA
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcacacagggatgacactgaagctttctttggacaaggcaccagactcacagttgta

SEQ ID NO: 273 - protein, 115 aa, synthetic construct
  REGION 1..115: note = source = /note="Description of Artificial Sequence: Synthetic polypeptide"
AQKVTQAQPEISVVEKEDVTLDCVYETRDTTYYLFWYKQPPSGELVFLIRRNSFDEQNEISGRYSWNFQKSTSSFNFTITASQVVDSAVYFCALSEGYNTDKLIFGTGTRLQVFP

SEQ ID NO: 274 - DNA, 345 bp, synthetic construct
  misc_feature 1..345: note = source = /note="Description of Artificial Sequence: Synthetic polynucleotide"
  source 1..345: mol_type = other DNA
gctcagaaggtaactcaagcgcagcctgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggggtataacaccgacaagctcatctttgggactgggaccagattacaagtctttcca

SEQ ID NO: 275 - protein, 112 aa, synthetic construct
  REGION 1..112: note = source = /note="Description of Artificial Sequence: Synthetic polypeptide"
DVKVTQSSRYLVKRTGEKVFLECVQDMDHENMFWYRQDPGLGLRLIYFSYDVKMKEKGDIPEGYSVSREKKERFSLILESASTNQTSMYLCASSLGGANTIYFGEGSWLTVV

SEQ ID NO: 276 - DNA, 336 bp, synthetic construct
  misc_feature 1..336: note = source = /note="Description of Artificial Sequence: Synthetic polynucleotide"
  source 1..336: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttagggggggcgaacaccatatattttggagagggaagttggctcactgttgta

SEQ ID NO: 277 - protein, 113 aa, synthetic construct
  REGION 1..113: note = source = /note="Description of Artificial Sequence: Synthetic polypeptide"
QKEVEQNSGPLSVPEGAIASLNCTYSDRGSQSFFWYRQYSGKSPELIMSIYSNGDKEDGRFTAQLNKASQYVSLLIRDSQPSDSATYLCAVNIPNSGYSTLTFGKGTMLLVSP

SEQ ID NO: 278 - DNA, 339 bp, synthetic construct
  misc_feature 1..339: note = source = /note="Description of Artificial Sequence: Synthetic polynucleotide"
  source 1..339: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacattccgaattcaggatacagcaccctcacctttgggaaggggactatgcttctagtctctcca

SEQ ID NO: 279 - protein, 112 aa, synthetic construct
  REGION 1..112: note = source = /note="Description of Artificial Sequence: Synthetic polypeptide"
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYWEGTEAFFGQGTRLTVV

SEQ ID NO: 280 - DNA, 336 bp, synthetic construct
  misc_feature 1..336: note = source = /note="Description of Artificial Sequence: Synthetic polynucleotide"
  source 1..336: mol_type = other DNA
aatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttactgggagggcactgaagctttctttggacaaggcaccagactcacagttgta

SEQ ID NO: 281 - protein, 112 aa, synthetic construct
  REGION 1..112: note = source = /note="Description of Artificial Sequence: Synthetic polypeptide"
AQTVTQSQPEMSVQEAETVTLSCTYDTSENNYYLFWYKQPPSRQMILVIRQEAYKQQNATENRFSVNFQKAAKSFSLKISDSQLGDTAMYFCAFDYGQNFVFGPGTRLSVLP

SEQ ID NO: 282 - DNA, 336 bp, synthetic construct
  misc_feature 1..336: note = source = /note="Description of Artificial Sequence: Synthetic polynucleotide"
  source 1..336: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcgactatggtcagaattttgtctttggtcccggaaccagattgtccgtgctgccc

SEQ ID NO: 283 - protein, 113 aa, synthetic construct
  REGION 1..113: note = source = /note="Description of Artificial Sequence: Synthetic polypeptide"
NAGVTQTPKFRILKIGQSMTLQCAQDMNHNYMYWYRQDPGMGLKLIYYSVGAGITDKGEVPNGYNVSRSTTEYFPLRLELAAPSQTSVYFCASSYGGGQTEAFFGQGTRLTVV

SEQ ID NO: 284 - DNA, 339 bp, synthetic construct
  misc_feature 1..339: note = source = /note="Description of Artificial Sequence: Synthetic polynucleotide"
  source 1..339: mol_type = other DNA
aatgctggtgtcactcagaccccaaaattccgcatcctgaagataggacagagcatgacactgcagtgtgcccaggatatgaaccataactacatgtactggtatcgacaagacccaggcatggggctgaagctgatttattattcagttggtgctggtatcactgataaaggagaagtcccgaatggctacaacgtctccagatcaaccacagagtatttcccgctcaggctggagttggctgctccctcccagacatctgtgtacttctgtgccagcagttacgggggggggcagactgaagctttctttggacaaggcaccagactcacagttgta

SEQ ID NO: 285 - protein, 116 aa, synthetic construct
  REGION 1..116: note = source = /note="Description of Artificial Sequence: Synthetic polypeptide"
AQKVTQAQPEISVVEKEDVTLDCVYETRDTTYYLFWYKQPPSGELVFLIRRNSFDEQNEISGRYSWNFQKSTSSFNFTITASQVVDSAVYFCALSEGYNQGGKLIFGQGTELSVKP

SEQ ID NO: 286 - DNA, 348 bp, synthetic construct
  misc_feature 1..348: note = source = /note="Description of Artificial Sequence: Synthetic polynucleotide"
  source 1..348: mol_type = other DNA
gctcagaaggtaactcaagcgcagcctgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagggttataaccagggaggaaagcttatcttcggacagggaacggagttatctgtgaaaccc

SEQ ID NO: 287 - protein, 112 aa, synthetic construct
  REGION 1..112: note = source = /note="Description of Artificial Sequence: Synthetic polypeptide"
DVKVTQSSRYLVKRTGEKVFLECVQDMDHENMFWYRQDPGLGLRLIYFSYDVKMKEKGDIPEGYSVSREKKERFSLILESASTNQTSMYLCASGADSNQPQHFGDGTRLSIL

SEQ ID NO: 288 - DNA, 336 bp, synthetic construct
  misc_feature 1..336: note = source = /note="Description of Artificial Sequence: Synthetic polynucleotide"
  source 1..336: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcggggcagatagcaatcagccccagcattttggtgatgggactcgactctccatccta

SEQ ID NO: 289 - protein, 5 aa, synthetic construct
  REGION 1..5: note = source = /note="Description of Artificial Sequence: Synthetic peptide"
SVFSS

SEQ ID NO: 290 - protein, 7 aa, synthetic construct
  REGION 1..7: note = source = /note="Description of Artificial Sequence: Synthetic peptide"
VVTGGEV

SEQ ID NO: 291 - protein, 11 aa, synthetic construct
  REGION 1..11: note = source = /note="Description of Artificial Sequence: Synthetic peptide"
AANGGSQGNLI

SEQ ID NO: 292 - protein, 5 aa, synthetic construct
  REGION 1..5: note = source = /note="Description of Artificial Sequence: Synthetic peptide"
SGHDT

SEQ ID NO: 293 - protein, 6 aa, synthetic construct
  REGION 1..6: note = source = /note="Description of Artificial Sequence: Synthetic peptide"
YYEEEE

SEQ ID NO: 294 - protein, 10 aa, synthetic construct
  REGION 1..10: note = source = /note="Description of Artificial Sequence: Synthetic peptide"
ASSLQDYGYT

SEQ ID NO: 295 - protein, 7 aa, synthetic construct
  REGION 1..7: note = source = /note="Description of Artificial Sequence: Synthetic peptide"
TISGTDY

SEQ ID NO: 296 - protein, 5 aa, synthetic construct
  REGION 1..5: note = source = /note="Description of Artificial Sequence: Synthetic peptide"
GLTSN

SEQ ID NO: 297 - protein, 11 aa, synthetic construct
  REGION 1..11: note = source = /note="Description of Artificial Sequence: Synthetic peptide"
ILRPDSWGKFQ

SEQ ID NO: 298 - protein, 5 aa, synthetic construct
  REGION 1..5: note = source = /note="Description of Artificial Sequence: Synthetic peptide"
SGHDT

SEQ ID NO: 299 - protein, 6 aa, synthetic construct
  REGION 1..6: note = source = /note="Description of Artificial Sequence: Synthetic peptide"
YYEEEE

SEQ ID NO: 300 - protein, 10 aa, synthetic construct
  REGION 1..10: note = source = /note="Description of Artificial Sequence: Synthetic peptide"
ASSLQDYGYT

SEQ ID NO: 301 - protein, 5 aa, synthetic construct
  REGION 1..5: note = source = /note="Description of Artificial Sequence: Synthetic peptide"
SVFSS

SEQ ID NO: 302 - protein, 7 aa, synthetic construct
  REGION 1..7: note = source = /note="Description of Artificial Sequence: Synthetic peptide"
VVTGGEV

SEQ ID NO: 303 - protein, 11 aa, synthetic construct
  REGION 1..11: note = source = /note="Description of Artificial Sequence: Synthetic peptide"
ATNGGSQGNLI

SEQ ID NO: 304 - protein, 5 aa, synthetic construct
  REGION 1..5: note = source = /note="Description of Artificial Sequence: Synthetic peptide"
SGHDT

SEQ ID NO: 305 - protein, 6 aa, synthetic construct
  REGION 1..6: note = source = /note="Description of Artificial Sequence: Synthetic peptide"
YYEEEE

SEQ ID NO: 306 - protein, 10 aa, synthetic construct
  REGION 1..10: note = source = /note="Description of Artificial Sequence: Synthetic peptide"
ASSLQDYGYT

SEQ ID NO: 307 - protein, 7 aa, synthetic construct
  REGION 1..7: note = source = /note="Description of Artificial Sequence: Synthetic peptide"
TISGTDY

SEQ ID NO: 308 - protein, 5 aa, synthetic construct
  REGION 1..5: note = source = /note="Description of Artificial Sequence: Synthetic peptide"
GLTSN

SEQ ID NO: 309 - protein, 9 aa, synthetic construct
  REGION 1..9: note = source = /note="Description of Artificial Sequence: Synthetic peptide"
IRRPGNQFY

SEQ ID NO: 310 - protein, 5 aa, synthetic construct
  REGION 1..5: note = source = /note="Description of Artificial Sequence: Synthetic peptide"
MNHEY

SEQ ID NO: 311 - protein, 6 aa, synthetic construct
  REGION 1..6: note = source = /note="Description of Artificial Sequence: Synthetic peptide"
SMNVEV

SEQ ID NO: 312 - protein, 11 aa, synthetic construct
  REGION 1..11: note = source = /note="Description of Artificial Sequence: Synthetic peptide"
ASSLWTGSEAF

SEQ ID NO: 313 - protein, 5 aa, synthetic construct
  REGION 1..5: note = source = /note="Description of Artificial Sequence: Synthetic peptide"
SVFSS

SEQ ID NO: 314 - protein, 7 aa, synthetic construct
  REGION 1..7: note = source = /note="Description of Artificial Sequence: Synthetic peptide"
VVTGGEV

SEQ ID NO: 315 - protein, 11 aa, synthetic construct
  REGION 1..11: note = source = /note="Description of Artificial Sequence: Synthetic peptide"
AGGTSGTYKYI

SEQ ID NO: 316 - protein, 5 aa, synthetic construct
  REGION 1..5: note = source = /note="Description of Artificial Sequence: Synthetic peptide"
MNHEY

SEQ ID NO: 317 - protein, 6 aa, synthetic construct
  REGION 1..6: note = source = /note="Description of Artificial Sequence: Synthetic peptide"
SMNVEV

SEQ ID NO: 318 - protein, 12 aa, synthetic construct
  REGION 1..12: note = source = /note="Description of Artificial Sequence: Synthetic peptide"
ASSPGTANYGYT

SEQ ID NO: 319 - protein, 6 aa, synthetic construct
  REGION 1..6: note = source = /note="Description of Artificial Sequence: Synthetic peptide"
YSGSPE

SEQ ID NO: 320 - protein, 4 aa, synthetic construct
  REGION 1..4: note = source = /note="Description of Artificial Sequence: Synthetic peptide"
HISR

SEQ ID NO: 321 - protein, 9 aa, synthetic construct
  REGION 1..9: note = source = /note="Description of Artificial Sequence: Synthetic peptide"
ALGNTDKLI

SEQ ID NO: 322 - protein, 5 aa, synthetic construct
  REGION 1..5: note = source = /note="Description of Artificial Sequence: Synthetic peptide"
MDHEN

SEQ ID NO: 323 - protein, 6 aa, synthetic construct
  REGION 1..6: note = source = /note="Description of Artificial Sequence: Synthetic peptide"
SYDVKM

SEQ ID NO: 324 - protein, 13 aa, synthetic construct
  REGION 1..13: note = source = /note="Description of Artificial Sequence: Synthetic peptide"
ASSSPRTGWYGYT

SEQ ID NO: 325 - protein, 7 aa, synthetic construct
  REGION 1..7: note = source = /note="Description of Artificial Sequence: Synthetic peptide"
TISGTDY

SEQ ID NO: 326 - protein, 5 aa, synthetic construct
  REGION 1..5: note = source = /note="Description of Artificial Sequence: Synthetic peptide"
GLTSN

SEQ ID NO: 327 - protein, 11 aa, synthetic construct
  REGION 1..11: note = source = /note="Description of Artificial Sequence: Synthetic peptide"
ILRPDSWGKFQ

SEQ ID NO: 328 - protein, 5 aa, synthetic construct
  REGION 1..5: note = source = /note="Description of Artificial Sequence: Synthetic peptide"
SGHDT

SEQ ID NO: 329 - protein, 6 aa, synthetic construct
  REGION 1..6: note = source = /note="Description of Artificial Sequence: Synthetic peptide"
YYEEEE

SEQ ID NO: 330 - protein, 10 aa, synthetic construct
  REGION 1..10: note = source = /note="Description of Artificial Sequence: Synthetic peptide"
ASSLVDYGYT

SEQ ID NO: 331 - protein, 7 aa, synthetic construct
  REGION 1..7: note = source = /note="Description of Artificial Sequence: Synthetic peptide"
TISGTDY

SEQ ID NO: 332 - protein, 5 aa, synthetic construct
  REGION 1..5: note = source = /note="Description of Artificial Sequence: Synthetic peptide"
GLTSN

SEQ ID NO: 333 - protein, 11 aa, synthetic construct
  REGION 1..11: note = source = /note="Description of Artificial Sequence: Synthetic peptide"
ILRPDSWGKFQ

SEQ ID NO: 334 - protein, 5 aa, synthetic construct
  REGION 1..5: note = source = /note="Description of Artificial Sequence: Synthetic peptide"
MDHEY

SEQ ID NO: 335 - protein, 6 aa, synthetic construct
  REGION 1..6: note = source = /note="Description of Artificial Sequence: Synthetic peptide"
SMNVEV

SEQ ID NO: 336 - protein, 12 aa, synthetic construct
  REGION 1..12: note = source = /note="Description of Artificial Sequence: Synthetic peptide"
ASSLGGVDERLS

SEQ ID NO: 337 - protein, 5 aa, synthetic construct
  REGION 1..5: note = source = /note="Description of Artificial Sequence: Synthetic peptide"
SVFSS

SEQ ID NO: 338 - protein, 7 aa, synthetic construct
  REGION 1..7: note = source = /note="Description of Artificial Sequence: Synthetic peptide"
VVTGGEV

SEQ ID NO: 339 - protein, 11 aa, synthetic construct
  REGION 1..11: note = source = /note="Description of Artificial Sequence: Synthetic peptide"
AGDGGSQGNLI

SEQ ID NO: 340 - protein, 5 aa, synthetic construct
  REGION 1..5: note = source = /note="Description of Artificial Sequence: Synthetic peptide"
MNHEY

SEQ ID NO: 341 - protein, 6 aa, synthetic construct
  REGION 1..6: note = source = /note="Description of Artificial Sequence: Synthetic peptide"
SMNVEV

SEQ ID NO: 342 - protein, 12 aa, synthetic construct
  REGION 1..12: note = source = /note="Description of Artificial Sequence: Synthetic peptide"
ASSLGGADEKLF

SEQ ID NO: 343 - protein, 7 aa, synthetic construct
  REGION 1..7: note = source = /note="Description of Artificial Sequence: Synthetic peptide"
TISGTDY

SEQ ID NO: 344 - protein, 5 aa, synthetic construct
  REGION 1..5: note = source = /note="Description of Artificial Sequence: Synthetic peptide"
GLTSN

SEQ ID NO: 345 - protein, 10 aa, synthetic construct
  REGION 1..10: note = source = /note="Description of Artificial Sequence: Synthetic peptide"
ILGQGAQKLV

SEQ ID NO: 346 - protein, 5 aa, synthetic construct
  REGION 1..5: note = source = /note="Description of Artificial Sequence: Synthetic peptide"
MNHEY

SEQ ID NO: 347 - protein, 6 aa, synthetic construct
  REGION 1..6: note = source = /note="Description of Artificial Sequence: Synthetic peptide"
SMNVEV

SEQ ID NO: 348 - protein, 11 aa, synthetic construct
  REGION 1..11: note = source = /note="Description of Artificial Sequence: Synthetic peptide"
ASSLWTGGGYT

SEQ ID NO: 349 - protein, 5 aa, synthetic construct
  REGION 1..5: note = source = /note="Description of Artificial Sequence: Synthetic peptide"
SVFSS

SEQ ID NO: 350 - protein, 7 aa, synthetic construct
  REGION 1..7: note = source = /note="Description of Artificial Sequence: Synthetic peptide"
VVTGGEV

SEQ ID NO: 351 - protein, 11 aa, synthetic construct
  REGION 1..11: note = source = /note="Description of Artificial Sequence: Synthetic peptide"
AGDGGSQGNLI

SEQ ID NO: 352 - protein, 5 aa, synthetic construct
  REGION 1..5: note = source = /note="Description of Artificial Sequence: Synthetic peptide"
SGHDT

SEQ ID NO: 353 - protein, 6 aa, synthetic construct
  REGION 1..6: note = source = /note="Description of Artificial Sequence: Synthetic peptide"
YYEEEE

SEQ ID NO: 354 - protein, 10 aa, synthetic construct
  REGION 1..10: note = source = /note="Description of Artificial Sequence: Synthetic peptide"
ASSLVDYGYT

SEQ ID NO: 355 - protein, 5 aa, synthetic construct
  REGION 1..5: note = source = /note="Description of Artificial Sequence: Synthetic peptide"
DSVNN

SEQ ID NO: 356 - protein, 5 aa, synthetic construct
  REGION 1..5: note = source = /note="Description of Artificial Sequence: Synthetic peptide"
IPSGT

SEQ ID NO: 357 - protein, 11 aa, synthetic construct
  REGION 1..11: note = source = /note="Description of Artificial Sequence: Synthetic peptide"
VLGGGSQGNLI

SEQ ID NO: 358 - protein, 5 aa, synthetic construct
  REGION 1..5: note = source = /note="Description of Artificial Sequence: Synthetic peptide"
SGHDT

SEQ ID NO: 359 - protein, 6 aa, synthetic construct
  REGION 1..6: note = source = /note="Description of Artificial Sequence: Synthetic peptide"
YYEEEE

SEQ ID NO: 360 - protein, 10 aa, synthetic construct
  REGION 1..10: note = source = /note="Description of Artificial Sequence: Synthetic peptide"
ASSFTDYGYT

SEQ ID NO: 361 - protein, 7 aa, synthetic construct
  REGION 1..7: note = source = /note="Description of Artificial Sequence: Synthetic peptide"
TISGTDY

SEQ ID NO: 362 - protein, 5 aa, synthetic construct
  REGION 1..5: note = source = /note="Description of Artificial Sequence: Synthetic peptide"
GLTSN

SEQ ID NO: 363 - protein, 11 aa, synthetic construct
  REGION 1..11: note = source = /note="Description of Artificial Sequence: Synthetic peptide"
ILRDGTGNQFY

SEQ ID NO: 364 - protein, 5 aa, synthetic construct
  REGION 1..5: note = source = /note="Description of Artificial Sequence: Synthetic peptide"
MNHEY

SEQ ID NO: 365 - protein, 6 aa, synthetic construct
  REGION 1..6: note = source = /note="Description of Artificial Sequence: Synthetic peptide"
SMNVEV

SEQ ID NO: 366 - protein, 11 aa, synthetic construct
  REGION 1..11: note = source = /note="Description of Artificial Sequence: Synthetic peptide"
ASSLWTGGGYT

SEQ ID NO: 367 - protein, 5 aa, synthetic construct
  REGION 1..5: note = source = /note="Description of Artificial Sequence: Synthetic peptide"
SVFSS

SEQ ID NO: 368 - protein, 7 aa, synthetic construct
  REGION 1..7: note = source = /note="Description of Artificial Sequence: Synthetic peptide"
VVTGGEV

SEQ ID NO: 369 - protein, 11 aa, synthetic construct
  REGION 1..11: note = source = /note="Description of Artificial Sequence: Synthetic peptide"
AGGPSGTYKYI

SEQ ID NO: 370 - protein, 5 aa, synthetic construct
  REGION 1..5: note = source = /note="Description of Artificial Sequence: Synthetic peptide"
MNHEY

SEQ ID NO: 371 - protein, 6 aa, synthetic construct
  REGION 1..6: note = source = /note="Description of Artificial Sequence: Synthetic peptide"
SMNVEV

SEQ ID NO: 372 - protein, 12 aa, synthetic construct
  REGION 1..12: note = source = /note="Description of Artificial Sequence: Synthetic peptide"
ASSPGTPNYGYT

SEQ ID NO: 373 - protein, 6 aa, synthetic construct
  REGION 1..6: note = source = /note="Description of Artificial Sequence: Synthetic peptide"
DRGSQS

SEQ ID NO: 374 - protein, 6 aa, synthetic construct
  REGION 1..6: note = source = /note="Description of Artificial Sequence: Synthetic peptide"
IYSNGD

SEQ ID NO: 375 - protein, 9 aa, synthetic construct
  REGION 1..9: note = source = /note="Description of Artificial Sequence: Synthetic peptide"
AGNYGQNFV

SEQ ID NO: 376 - protein, 5 aa, synthetic construct
  REGION 1..5: note = source = /note="Description of Artificial Sequence: Synthetic peptide"
PRHDT

SEQ ID NO: 377 - protein, 6 aa, synthetic construct
  REGION 1..6: note = source = /note="Description of Artificial Sequence: Synthetic peptide"
FYEKMQ

SEQ ID NO: 378 - protein, 11 aa, synthetic construct
  REGION 1..11: note = source = /note="Description of Artificial Sequence: Synthetic peptide"
ASSIGLNQPQH

SEQ ID NO: 379 - protein, 7 aa, synthetic construct
  REGION 1..7: note = source = /note="Description of Artificial Sequence: Synthetic peptide"
TISGTDY

SEQ ID NO: 380 - protein, 5 aa, synthetic construct
  REGION 1..5: note = source = /note="Description of Artificial Sequence: Synthetic peptide"
GLTSN

SEQ ID NO: 381 - protein, 11 aa, synthetic construct
  REGION 1..11: note = source = /note="Description of Artificial Sequence: Synthetic peptide"
ILRDGIGNQFY

SEQ ID NO: 382 - protein, 5 aa, synthetic construct
  REGION 1..5: note = source = /note="Description of Artificial Sequence: Synthetic peptide"
MNHEY

SEQ ID NO: 383 - protein, 6 aa, synthetic construct
  REGION 1..6: note = source = /note="Description of Artificial Sequence: Synthetic peptide"
SMNVEV

SEQ ID NO: 384 - protein, 11 aa, synthetic construct
  REGION 1..11: note = source = /note="Description of Artificial Sequence: Synthetic peptide"
ASSLWTGGGYT

SEQ ID NO: 385 - protein, 7 aa, synthetic construct
  REGION 1..7: note = source = /note="Description of Artificial Sequence: Synthetic peptide"
TISGTDY

SEQ ID NO: 386 - protein, 5 aa, synthetic construct
  REGION 1..5: note = source = /note="Description of Artificial Sequence: Synthetic peptide"
GLTSN

SEQ ID NO: 387 - protein, 11 aa, synthetic construct
  REGION 1..11: note = source = /note="Description of Artificial Sequence: Synthetic peptide"
ILRPDSWGKFQ

SEQ ID NO: 388 - protein, 5 aa, synthetic construct
  REGION 1..5: note = source = /note="Description of Artificial Sequence: Synthetic peptide"
MNHEY

SEQ ID NO: 389 - protein, 6 aa, synthetic construct
  REGION 1..6: note = source = /note="Description of Artificial Sequence: Synthetic peptide"
SMNVEV

SEQ ID NO: 390 - protein, 11 aa, synthetic construct
  REGION 1..11: note = source = /note="Description of Artificial Sequence: Synthetic peptide"
ASSLWTGGGYT

SEQ ID NO: 391 - protein, 7 aa, synthetic construct
  REGION 1..7: note = source = /note="Description of Artificial Sequence: Synthetic peptide"
TISGTDY

SEQ ID NO: 392 - protein, 5 aa, synthetic construct
  REGION 1..5: note = source = /note="Description of Artificial Sequence: Synthetic peptide"
GLTSN

SEQ ID NO: 393 - protein, 11 aa, synthetic construct
  REGION 1..11: note = source = /note="Description of Artificial Sequence: Synthetic peptide"
ILRDGTGNQFY

SEQ ID NO: 394 - protein, 5 aa, synthetic construct
  REGION 1..5: note = source = /note="Description of Artificial Sequence: Synthetic peptide"
MNHEY

SEQ ID NO: 395 - protein, 6 aa, synthetic construct
  REGION 1..6: note = source = /note="Description of Artificial Sequence: Synthetic peptide"
SMNVEV

SEQ ID NO: 396 - protein, 11 aa, synthetic construct
  REGION 1..11: note = source = /note="Description of Artificial Sequence: Synthetic peptide"
ASSSTGYYGYT

SEQ ID NO: 397 - protein, 7 aa, synthetic construct
  REGION 1..7: note = source = /note="Description of Artificial Sequence: Synthetic peptide"
TISGTDY

SEQ ID NO: 398 - protein, 5 aa, synthetic construct
  REGION 1..5: note = source = /note="Description of Artificial Sequence: Synthetic peptide"
GLTSN

SEQ ID NO: 399 - protein, 12 aa, synthetic construct
  REGION 1..12: note = source = /note="Description of Artificial Sequence: Synthetic peptide"
ILRDREYGNKLV

SEQ ID NO: 400 - protein, 5 aa, synthetic construct
  REGION 1..5: note = source = /note="Description of Artificial Sequence: Synthetic peptide"
MNHEY

SEQ ID NO: 401 - protein, 6 aa, synthetic construct
  REGION 1..6: note = source = /note="Description of Artificial Sequence: Synthetic peptide"
SMNVEV

SEQ ID NO: 402 - protein, 11 aa, synthetic construct
  REGION 1..11: note = source = /note="Description of Artificial Sequence: Synthetic peptide"
ASSLWTGGGYT

SEQ ID NO: 403 - protein, 5 aa, synthetic construct
  REGION 1..5: note = source = /note="Description of Artificial Sequence: Synthetic peptide"
SVFSS

SEQ ID NO: 404 - protein, 7 aa, synthetic construct
  REGION 1..7: note = source = /note="Description of Artificial Sequence: Synthetic peptide"
VVTGGEV

SEQ ID NO: 405 - protein, 11 aa, synthetic construct
  REGION 1..11: note = source = /note="Description of Artificial Sequence: Synthetic peptide"
AEDGGSQGNLI

SEQ ID NO: 406 - protein, 5 aa, synthetic construct
  REGION 1..5: note = source = /note="Description of Artificial Sequence: Synthetic peptide"
SGHDT

SEQ ID NO: 407 - protein, 6 aa, synthetic construct
  REGION 1..6: note = source = /note="Description of Artificial Sequence: Synthetic peptide"
YYEEEE

SEQ ID NO: 408 - protein, 10 aa, synthetic construct
  REGION 1..10: note = source = /note="Description of Artificial Sequence: Synthetic peptide"
ASSLSDYGYT

SEQ ID NO: 409 - protein, 5 aa, synthetic construct
  REGION 1..5: note = source = /note="Description of Artificial Sequence: Synthetic peptide"
DSVNN

SEQ ID NO: 410 - protein, 5 aa, synthetic construct
  REGION 1..5: note = source = /note="Description of Artificial Sequence: Synthetic peptide"
IPSGT

SEQ ID NO: 411 - protein, 11 aa, synthetic construct
  REGION 1..11: note = source = /note="Description of Artificial Sequence: Synthetic peptide"
AVEASGTYKYI

SEQ ID NO: 412 - protein, 5 aa, synthetic construct
  REGION 1..5: note = source = /note="Description of Artificial Sequence: Synthetic peptide"
MNHEY

SEQ ID NO: 413 - protein, 6 aa, synthetic construct
  REGION 1..6: note = source = /note="Description of Artificial Sequence: Synthetic peptide"
SMNVEV

SEQ ID NO: 414 - protein, 12 aa, synthetic construct
  REGION 1..12: note = source = /note="Description of Artificial Sequence: Synthetic peptide"
ASSWGTGGYGYT

SEQ ID NO: 415 - protein, 6 aa, synthetic construct
  REGION 1..6: note = source = /note="Description of Artificial Sequence: Synthetic peptide"
NSASDY

SEQ ID NO: 416 - protein, 7 aa, synthetic construct
  REGION 1..7: note = source = /note="Description of Artificial Sequence: Synthetic peptide"
IRSNMDK

SEQ ID NO: 417 - protein, 13 aa, synthetic construct
  REGION 1..13: note = source = /note="Description of Artificial Sequence: Synthetic peptide"
AENKRDNYGQNFV

SEQ ID NO: 418 - protein, 5 aa, synthetic construct
  REGION 1..5: note = source = /note="Description of Artificial Sequence: Synthetic peptide"
MNHEY

SEQ ID NO: 419 - protein, 6 aa, synthetic construct
  REGION 1..6: note = source = /note="Description of Artificial Sequence: Synthetic peptide"
SMNVEV

SEQ ID NO: 420 - protein, 11 aa, synthetic construct
  REGION 1..11: note = source = /note="Description of Artificial Sequence: Synthetic peptide"
ASSFWVNTEAF

SEQ ID NO: 421 - protein, 5 aa, synthetic construct
  REGION 1..5: note = source = /note="Description of Artificial Sequence: Synthetic peptide"
DSVNN

SEQ ID NO: 422 - protein, 5 aa, synthetic construct
  REGION 1..5: note = source = /note="Description of Artificial Sequence: Synthetic peptide"
IPSGT

SEQ ID NO: 423 - protein, 11 aa, synthetic construct
  REGION 1..11: note = source = /note="Description of Artificial Sequence: Synthetic peptide"
AVGSSNGYKLS

SEQ ID NO: 424 - protein, 5 aa, synthetic construct
  REGION 1..5: note = source = /note="Description of Artificial Sequence: Synthetic peptide"
MNHEY

SEQ ID NO: 425 - protein, 6 aa, synthetic construct
  REGION 1..6: note = source = /note="Description of Artificial Sequence: Synthetic peptide"
SMNVEV

SEQ ID NO: 426 - protein, 13 aa, synthetic construct
  REGION 1..13: note = source = /note="Description of Artificial Sequence: Synthetic peptide"
ASSPGTGGFSPLH

SEQ ID NO: 427 - protein, 5 aa, synthetic construct
  REGION 1..5: note = source = /note="Description of Artificial Sequence: Synthetic peptide"
DSVNN

SEQ ID NO: 428 - protein, 5 aa, synthetic construct
  REGION 1..5: note = source = /note="Description of Artificial Sequence: Synthetic peptide"
IPSGT

SEQ ID NO: 429 - protein, 11 aa, synthetic construct
  REGION 1..11: note = source = /note="Description of Artificial Sequence: Synthetic peptide"
AVGSSNDYKLS

SEQ ID NO: 430 - protein, 5 aa, synthetic construct
  REGION 1..5: note = source = /note="Description of Artificial Sequence: Synthetic peptide"
MNHEY

SEQ ID NO: 431 - protein, 6 aa, synthetic construct
  REGION 1..6: note = source = /note="Description of Artificial Sequence: Synthetic peptide"
SMNVEV

SEQ ID NO: 432 - protein, 13 aa, synthetic construct
  REGION 1..13: note = source = /note="Description of Artificial Sequence: Synthetic peptide"
ASSPGTGGFSPLH

SEQ ID NO: 433 - protein, 6 aa, synthetic construct
  REGION 1..6: note = source = /note="Description of Artificial Sequence: Synthetic peptide"
NSASDY

SEQ ID NO: 434 - protein, 7 aa, synthetic construct
  REGION 1..7: note = source = /note="Description of Artificial Sequence: Synthetic peptide"
IRSNMDK

SEQ ID NO: 435 - protein, 13 aa, synthetic construct
  REGION 1..13: note = source = /note="Description of Artificial Sequence: Synthetic peptide"
AENRQDNYGQNFV

SEQ ID NO: 436 - protein, 5 aa, synthetic construct
  REGION 1..5: note = source = /note="Description of Artificial Sequence: Synthetic peptide"
MNHEY

SEQ ID NO: 437 - protein, 6 aa, synthetic construct
  REGION 1..6: note = source = /note="Description of Artificial Sequence: Synthetic peptide"
SMNVEV

SEQ ID NO: 438 - protein, 11 aa, synthetic construct
  REGION 1..11: note = source = /note="Description of Artificial Sequence: Synthetic peptide"
ASSLWVNTEAF

SEQ ID NO: 439 - protein, 7 aa, synthetic construct
  REGION 1..7: note = source = /note="Description of Artificial Sequence: Synthetic peptide"
TISGTDY

SEQ ID NO: 440 - protein, 5 aa, synthetic construct
  REGION 1..5: note = source = /note="Description of Artificial Sequence: Synthetic peptide"
GLTSN

SEQ ID NO: 441 - protein, 11 aa, synthetic construct
  REGION 1..11: note = source = /note="Description of Artificial Sequence: Synthetic peptide"
ILRPDSWGKFQ

SEQ ID NO: 442 - protein, 5 aa, synthetic construct
  REGION 1..5: note = source = /note="Description of Artificial Sequence: Synthetic peptide"
MDHEN

SEQ ID NO: 443 - protein, 6 aa, synthetic construct
  REGION 1..6: note = source = /note="Description of Artificial Sequence: Synthetic peptide"
SYDVKM

SEQ ID NO: 444 - protein, 13 aa, synthetic construct
  REGION 1..13: note = source = /note="Description of Artificial Sequence: Synthetic peptide"
ASSTVRQGNYGYT

SEQ ID NO: 445 - protein, 7 aa, synthetic construct
  REGION 1..7: note = source = /note="Description of Artificial Sequence: Synthetic peptide"
TISGTDY

SEQ ID NO: 446 - protein, 5 aa, synthetic construct
  REGION 1..5: note = source = /note="Description of Artificial Sequence: Synthetic peptide"
GLTSN

SEQ ID NO: 447 - protein, 11 aa, synthetic construct
  REGION 1..11: note = source = /note="Description of Artificial Sequence: Synthetic peptide"
ILNTGTASKLT

SEQ ID NO: 448 - protein, 5 aa, synthetic construct
  REGION 1..5: note = source = /note="Description of Artificial Sequence: Synthetic peptide"
MNHEY

SEQ ID NO: 449 - protein, 6 aa, synthetic construct
  REGION 1..6: note = source = /note="Description of Artificial Sequence: Synthetic peptide"
SMNVEV

SEQ ID NO: 450 - protein, 11 aa, synthetic construct
  REGION 1..11: note = source = /note="Description of Artificial Sequence: Synthetic peptide"
ASSLSSNTEAF

SEQ ID NO: 451 - protein, 6 aa, synthetic construct
  REGION 1..6: note = source = /note="Description of Artificial Sequence: Synthetic peptide"
DRGSQS

SEQ ID NO: 452 - protein, 6 aa, synthetic construct
  REGION 1..6: note = source = /note="Description of Artificial Sequence: Synthetic peptide"
IYSNGD

SEQ ID NO: 453 - protein, 10 aa, synthetic construct
  REGION 1..10: note = source = /note="Description of Artificial Sequence: Synthetic peptide"
AVNRGTDKLI

SEQ ID NO: 454 - protein, 5 aa, synthetic construct
  REGION 1..5: note = source = /note="Description of Artificial Sequence: Synthetic peptide"
SGHDT

SEQ ID NO: 455 - protein, 6 aa, synthetic construct
  REGION 1..6: note = source = /note="Description of Artificial Sequence: Synthetic peptide"
YYEEEE

SEQ ID NO: 456 - protein, 10 aa, synthetic construct
  REGION 1..10: note = source = /note="Description of Artificial Sequence: Synthetic peptide"
ASSWTDYGYT

SEQ ID NO: 457 - protein, 7 aa, synthetic construct
  REGION 1..7: note = source = /note="Description of Artificial Sequence: Synthetic peptide"
TISGTDY

SEQ ID NO: 458 - protein, 5 aa, synthetic construct
  REGION 1..5: note = source = /note="Description of Artificial Sequence: Synthetic peptide"
GLTSN

SEQ ID NO: 459 - protein, 11 aa, synthetic construct
  REGION 1..11: note = source = /note="Description of Artificial Sequence: Synthetic peptide"
ILRPDSWGKFQ

SEQ ID NO: 460 - protein, 5 aa, synthetic construct
  REGION 1..5: note = source = /note="Description of Artificial Sequence: Synthetic peptide"
SGHDT

SEQ ID NO: 461 - protein, 6 aa, synthetic construct
  REGION 1..6: note = source = /note="Description of Artificial Sequence: Synthetic peptide"
YYEEEE

SEQ ID NO: 462 - protein, 10 aa, synthetic construct
  REGION 1..10: note = source = /note="Description of Artificial Sequence: Synthetic peptide"
ASSWTDYGYT

SEQ ID NO: 463 - protein, 5 aa, synthetic construct
  REGION 1..5: note = source = /note="Description of Artificial Sequence: Synthetic peptide"
SVFSS

SEQ ID NO: 464 - protein, 7 aa, synthetic construct
  REGION 1..7: note = source = /note="Description of Artificial Sequence: Synthetic peptide"
VVTGGEV

SEQ ID NO: 465 - protein, 11 aa, synthetic construct
  REGION 1..11: note = source = /note="Description of Artificial Sequence: Synthetic peptide"
AGGTSGTYKYI

SEQ ID NO: 466 - protein, 5 aa, synthetic construct
  REGION 1..5: note = source = /note="Description of Artificial Sequence: Synthetic peptide"
MNHEY

SEQ ID NO: 467 - protein, 6 aa, synthetic construct
  REGION 1..6: note = source = /note="Description of Artificial Sequence: Synthetic peptide"
SMNVEV

SEQ ID NO: 468 - protein, 12 aa, synthetic construct
  REGION 1..12: note = source = /note="Description of Artificial Sequence: Synthetic peptide"
ASSPGTPNYGYT

SEQ ID NO: 469 - protein, 6 aa, synthetic construct
  REGION 1..6: note = source = /note="Description of Artificial Sequence: Synthetic peptide"
NSASQS

SEQ ID NO: 470 - protein, 6 aa, synthetic construct
  REGION 1..6: note = source = /note="Description of Artificial Sequence: Synthetic peptide"
VYSSGN

SEQ ID NO: 471 - protein, 11 aa, synthetic construct
  REGION 1..11: note = source = /note="Description of Artificial Sequence: Synthetic peptide"
VVNGGSQGNLI

SEQ ID NO: 472 - protein, 5 aa, synthetic construct
  REGION 1..5: note = source = /note="Description of Artificial Sequence: Synthetic peptide"
SGHDT

SEQ ID NO: 473 - protein, 6 aa, synthetic construct
  REGION 1..6: note = source = /note="Description of Artificial Sequence: Synthetic peptide"
YYEEEE

SEQ ID NO: 474 - protein, 10 aa, synthetic construct
  REGION 1..10: note = source = /note="Description of Artificial Sequence: Synthetic peptide"
ASSVGDYGYT

SEQ ID NO: 475 - protein, 6 aa, synthetic construct
  REGION 1..6: note = source = /note="Description of Artificial Sequence: Synthetic peptide"
NSASDY

SEQ ID NO: 476 - protein, 7 aa, synthetic construct
  REGION 1..7: note = source = /note="Description of Artificial Sequence: Synthetic peptide"
IRSNMDK

SEQ ID NO: 477 - protein, 10 aa, synthetic construct
  REGION 1..10: note = source = /note="Description of Artificial Sequence: Synthetic peptide"
AENNYGQNFV

SEQ ID NO: 478 - protein, 5 aa, synthetic construct
  REGION 1..5: note = source = /note="Description of Artificial Sequence: Synthetic peptide"
MNHEY

SEQ ID NO: 479 - protein, 6 aa, synthetic construct
  REGION 1..6: note = source = /note="Description of Artificial Sequence: Synthetic peptide"
SMNVEV

SEQ ID NO: 480 - protein, 11 aa, synthetic construct
  REGION 1..11: note = source = /note="Description of Artificial Sequence: Synthetic peptide"
ASSLWDSSPLH

SEQ ID NO: 481 - protein, 6 aa, synthetic construct
  REGION 1..6: note = source = /note="Description of Artificial Sequence: Synthetic peptide"
NSMFDY

SEQ ID NO: 482 - protein, 7 aa, synthetic construct
  REGION 1..7: note = source = /note="Description of Artificial Sequence: Synthetic peptide"
ISSIKDK

SEQ ID NO: 483 - protein, 11 aa, synthetic construct
  REGION 1..11: note = source = /note="Description of Artificial Sequence: Synthetic peptide"
AASAGSARQLT

SEQ ID NO: 484 - protein, 5 aa, synthetic construct
  REGION 1..5: note = source = /note="Description of Artificial Sequence: Synthetic peptide"
MNHEY

SEQ ID NO: 485 - protein, 6 aa, synthetic construct
  REGION 1..6: note = source = /note="Description of Artificial Sequence: Synthetic peptide"
SMNVEV

SEQ ID NO: 486 - protein, 11 aa, synthetic construct
  REGION 1..11: note = source = /note="Description of Artificial Sequence: Synthetic peptide"
ASSLYTHTEAF

SEQ ID NO: 487 - protein, 7 aa, synthetic construct
  REGION 1..7: note = source = /note="Description of Artificial Sequence: Synthetic peptide"
TISGNEY

SEQ ID NO: 488 - protein, 5 aa, synthetic construct
  REGION 1..5: note = source = /note="Description of Artificial Sequence: Synthetic peptide"
GLKNN

SEQ ID NO: 489 - protein, 13 aa, synthetic construct
  REGION 1..13: note = source = /note="Description of Artificial Sequence: Synthetic peptide"
IVRDTTSGTYKYI

SEQ ID NO: 490 - protein, 5 aa, synthetic construct
  REGION 1..5: note = source = /note="Description of Artificial Sequence: Synthetic peptide"
MNHEY

SEQ ID NO: 491 - protein, 6 aa, synthetic construct
  REGION 1..6: note = source = /note="Description of Artificial Sequence: Synthetic peptide"
SMNVEV

SEQ ID NO: 492 - protein, 13 aa, synthetic construct
  REGION 1..13: note = source = /note="Description of Artificial Sequence: Synthetic peptide"
ASSLSSTGFSPLH

SEQ ID NO: 493 - protein, 5 aa, synthetic construct
  REGION 1..5: note = source = /note="Description of Artificial Sequence: Synthetic peptide"
SVFSS

SEQ ID NO: 494 - protein, 7 aa, synthetic construct
  REGION 1..7: note = source = /note="Description of Artificial Sequence: Synthetic peptide"
VVTGGEV

SEQ ID NO: 495 - protein, 11 aa, synthetic construct
  REGION 1..11: note = source = /note="Description of Artificial Sequence: Synthetic peptide"
AEGGGSQGNLI

SEQ ID NO: 496 - protein, 5 aa, synthetic construct
  REGION 1..5: note = source = /note="Description of Artificial Sequence: Synthetic peptide"
SGHDT

SEQ ID NO: 497 - protein, 6 aa, synthetic construct
  REGION 1..6: note = source = /note="Description of Artificial Sequence: Synthetic peptide"
YYEEEE

SEQ ID NO: 498 - protein, 10 aa, synthetic construct
  REGION 1..10: note = source = /note="Description of Artificial Sequence: Synthetic peptide"
ASSWTDYGYT

SEQ ID NO: 499 - protein, 5 aa, synthetic construct
  REGION 1..5: note = source = /note="Description of Artificial Sequence: Synthetic peptide"
SVFSS

SEQ ID NO: 500 - protein, 7 aa, synthetic construct
  REGION 1..7: note = source = /note="Description of Artificial Sequence: Synthetic peptide"
VVTGGEV

SEQ ID NO: 501 - protein, 11 aa, synthetic construct
  REGION 1..11: note = source = /note="Description of Artificial Sequence: Synthetic peptide"
AGIRSNDYKLS

SEQ ID NO: 502 - protein, 5 aa, synthetic construct
  REGION 1..5: note = source = /note="Description of Artificial Sequence: Synthetic peptide"
MNHEY

SEQ ID NO: 503 - protein, 6 aa, synthetic construct
  REGION 1..6: note = source = /note="Description of Artificial Sequence: Synthetic peptide"
SMNVEV

SEQ ID NO: 504 - protein, 12 aa, synthetic construct
  REGION 1..12: note = source = /note="Description of Artificial Sequence: Synthetic peptide"
ASSSWTAHTEAF

SEQ ID NO: 505 - protein, 5 aa, synthetic construct
  REGION 1..5: note = source = /note="Description of Artificial Sequence: Synthetic peptide"
SVFSS

SEQ ID NO: 506 - protein, 7 aa, synthetic construct
  REGION 1..7: note = source = /note="Description of Artificial Sequence: Synthetic peptide"
VVTGGEV

SEQ ID NO: 507 - protein, 12 aa, synthetic construct
  REGION 1..12: note = source = /note="Description of Artificial Sequence: Synthetic peptide"
AENSGGGADGLT

SEQ ID NO: 508 - protein, 5 aa, synthetic construct
  REGION 1..5: note = source = /note="Description of Artificial Sequence: Synthetic peptide"
SGHDT

SEQ ID NO: 509 - protein, 6 aa, synthetic construct
  REGION 1..6: note = source = /note="Description of Artificial Sequence: Synthetic peptide"
YYEEEE

SEQ ID NO: 510 - protein, 10 aa, synthetic construct
  REGION 1..10: note = source = /note="Description of Artificial Sequence: Synthetic peptide"
ASSFTDYGYT

SEQ ID NO: 511 - protein, 5 aa, synthetic construct
  REGION 1..5: note = source = /note="Description of Artificial Sequence: Synthetic peptide"
SVFSS

SEQ ID NO: 512 - protein, 7 aa, synthetic construct
  REGION 1..7: note = source = /note="Description of Artificial Sequence: Synthetic peptide"
VVTGGEV

SEQ ID NO: 513 - protein, 11 aa, synthetic construct
  REGION 1..11: note = source = /note="Description of Artificial Sequence: Synthetic peptide"
AGEDFGNEKLT

SEQ ID NO: 514 - protein, 5 aa, synthetic construct
  REGION 1..5: note = source = /note="Description of Artificial Sequence: Synthetic peptide"
SGHDT

SEQ ID NO: 515 - protein, 6 aa, synthetic construct
  REGION 1..6: note = source = /note="Description of Artificial Sequence: Synthetic peptide"
YYEEEE

SEQ ID NO: 516 - protein, 10 aa, synthetic construct
  REGION 1..10: note = source = /note="Description of Artificial Sequence: Synthetic peptide"
ASSWADYGYT

SEQ ID NO: 517 - protein, 6 aa, synthetic construct
  REGION 1..6: note = source = /note="Description of Artificial Sequence: Synthetic peptide"
SSNFYA

SEQ ID NO: 518 - protein, 7 aa, synthetic construct
  REGION 1..7: note = source = /note="Description of Artificial Sequence: Synthetic peptide"
MTLNGDE

SEQ ID NO: 519 - protein, 9 aa, synthetic construct
  REGION 1..9: note = source = /note="Description of Artificial Sequence: Synthetic peptide"
AFLTGNQFY

SEQ ID NO: 520 - protein, 5 aa, synthetic construct
  REGION 1..5: note = source = /note="Description of Artificial Sequence: Synthetic peptide"
MDHEN

SEQ ID NO: 521 - protein, 6 aa, synthetic construct
  REGION 1..6: note = source = /note="Description of Artificial Sequence: Synthetic peptide"
SYDVKM

SEQ ID NO: 522 - protein, 13 aa, synthetic construct
  REGION 1..13: note = source = /note="Description of Artificial Sequence: Synthetic peptide"
ASSTVRQGNYGYT

SEQ ID NO: 523 - protein, 6 aa, synthetic construct
  REGION 1..6: note = source = /note="Description of Artificial Sequence: Synthetic peptide"
NSMFDY

SEQ ID NO: 524 - protein, 7 aa, synthetic construct
  REGION 1..7: note = source = /note="Description of Artificial Sequence: Synthetic peptide"
ISSIKDK

SEQ ID NO: 525 - protein, 11 aa, synthetic construct
  REGION 1..11: note = source = /note="Description of Artificial Sequence: Synthetic peptide"
AASAGSARQLT

SEQ ID NO: 526 - protein, 5 aa, synthetic construct
  REGION 1..5: note = source = /note="Description of Artificial Sequence: Synthetic peptide"
MNHEY

SEQ ID NO: 527 - protein, 6 aa, synthetic construct
  REGION 1..6: note = source = /note="Description of Artificial Sequence: Synthetic peptide"
SMNVEV

SEQ ID NO: 528 - protein, 11 aa, synthetic construct
  REGION 1..11: note = source = /note="Description of Artificial Sequence: Synthetic peptide"
ASSLWSNTEAF

SEQ ID NO: 529 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 530 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 531 - DNA, 33 bp, synthetic construct
  misc_feature 1..33: note = source = /note="Description of Artificial Sequence: Synthetic oligonucleotide"
  source 1..33: mol_type = other DNA
gccgctaatggaggaagccaaggaaatctcatc

SEQ ID NO: 532 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 533 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 534 - DNA, 30 bp, synthetic construct
  misc_feature 1..30: note = source = /note="Description of Artificial Sequence: Synthetic oligonucleotide"
  source 1..30: mol_type = other DNA
gccagcagcttacaggactatggctacacc

SEQ ID NO: 535 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 536 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 537 - DNA, 33 bp, synthetic construct
  misc_feature 1..33: note = source = /note="Description of Artificial Sequence: Synthetic oligonucleotide"
  source 1..33: mol_type = other DNA
atcctgcggcctgacagctgggggaaattccag

SEQ ID NO: 538 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 539 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 540 - DNA, 30 bp, synthetic construct; identical to SEQ ID NO: 534
  misc_feature 1..30: note = source = /note="Description of Artificial Sequence: Synthetic oligonucleotide"
  source 1..30: mol_type = other DNA

SEQ ID NO: 541 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 542 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 543 - DNA, 33 bp, synthetic construct
  misc_feature 1..33: note = source = /note="Description of Artificial Sequence: Synthetic oligonucleotide"
  source 1..33: mol_type = other DNA
gccactaatggaggaagccaaggaaatctcatc

SEQ ID NO: 544 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 545 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 546 - DNA, 30 bp, synthetic construct; identical to SEQ ID NO: 534
  misc_feature 1..30: note = source = /note="Description of Artificial Sequence: Synthetic oligonucleotide"
  source 1..30: mol_type = other DNA

SEQ ID NO: 547 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 548 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 549 - DNA, 27 bp, synthetic construct
  misc_feature 1..27: note = source = /note="Description of Artificial Sequence: Synthetic oligonucleotide"
  source 1..27: mol_type = other DNA
atcaggcggcccggtaaccagttctat

SEQ ID NO: 550 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 551 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 552 - DNA, 33 bp, synthetic construct
  misc_feature 1..33: note = source = /note="Description of Artificial Sequence: Synthetic oligonucleotide"
  source 1..33: mol_type = other DNA
gccagcagtttatggacagggtctgaagctttc

SEQ ID NO: 553 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 554 - DNA, 21 bp, synthetic construct
  misc_feature 1..21: note = source = /note="Description of Artificial Sequence: Synthetic oligonucleotide"
  source 1..21: mol_type = other DNA
gtagttactggtggagaagtg

SEQ ID NO: 555 - DNA, 33 bp, synthetic construct
  misc_feature 1..33: note = source = /note="Description of Artificial Sequence: Synthetic oligonucleotide"
  source 1..33: mol_type = other DNA
gcaggagggacctcaggaacctacaaatacatc

SEQ ID NO: 556 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 557 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 558 - DNA, 36 bp, synthetic construct
  misc_feature 1..36: note = source = /note="Description of Artificial Sequence: Synthetic oligonucleotide"
  source 1..36: mol_type = other DNA
gccagcagtccagggacagctaactatggctacacc

SEQ ID NO: 559 - DNA, 18 bp, synthetic construct
  misc_feature 1..18: note = source = /note="Description of Artificial Sequence: Synthetic oligonucleotide"
  source 1..18: mol_type = other DNA
tattctgggagtcctgaa

SEQ ID NO: 560 - DNA, 12 bp, synthetic construct
  misc_feature 1..12: note = source = /note="Description of Artificial Sequence: Synthetic oligonucleotide"
  source 1..12: mol_type = other DNA
cacatctctaga

SEQ ID NO: 561 - DNA, 27 bp, synthetic construct
  misc_feature 1..27: note = source = /note="Description of Artificial Sequence: Synthetic oligonucleotide"
  source 1..27: mol_type = other DNA
gctctagggaacaccgacaagctcatc

SEQ ID NO: 562 - DNA, 15 bp, synthetic construct
  misc_feature 1..15: note = source = /note="Description of Artificial Sequence: Synthetic oligonucleotide"
  source 1..15: mol_type = other DNA
atggaccatgaaaat

SEQ ID NO: 563 - DNA, 18 bp, synthetic construct
  misc_feature 1..18: note = source = /note="Description of Artificial Sequence: Synthetic oligonucleotide"
  source 1..18: mol_type = other DNA
tcatatgatgttaaaatg

SEQ ID NO: 564 - DNA, 39 bp, synthetic construct
  misc_feature 1..39: note = source = /note="Description of Artificial Sequence: Synthetic oligonucleotide"
  source 1..39: mol_type = other DNA
gccagcagttcgcccaggacagggtggtatggctacacc

SEQ ID NO: 565 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 566 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 567 - DNA, 33 bp, synthetic construct; identical to SEQ ID NO: 537
  misc_feature 1..33: note = source = /note="Description of Artificial Sequence: Synthetic oligonucleotide"
  source 1..33: mol_type = other DNA

SEQ ID NO: 568 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 569 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 570 - DNA, 30 bp, synthetic construct
  misc_feature 1..30: note = source = /note="Description of Artificial Sequence: Synthetic oligonucleotide"
  source 1..30: mol_type = other DNA
gccagcagcttggtggattatggctacacc

SEQ ID NO: 571 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 572 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 573 - DNA, 33 bp, synthetic construct; identical to SEQ ID NO: 537
  misc_feature 1..33: note = source = /note="Description of Artificial Sequence: Synthetic oligonucleotide"
  source 1..33: mol_type = other DNA

SEQ ID NO: 574 - DNA, 15 bp, synthetic construct
  misc_feature 1..15: note = source = /note="Description of Artificial Sequence: Synthetic oligonucleotide"
  source 1..15: mol_type = other DNA
atggaccatgagtat

SEQ ID NO: 575 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 576 - DNA, 36 bp, synthetic construct
  misc_feature 1..36: note = source = /note="Description of Artificial Sequence: Synthetic oligonucleotide"
  source 1..36: mol_type = other DNA
gccagcagtttagggggcgtggatgaaagactgtct

SEQ ID NO: 577 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 578 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 579 - DNA, 33 bp, synthetic construct
  misc_feature 1..33: note = source = /note="Description of Artificial Sequence: Synthetic oligonucleotide"
  source 1..33: mol_type = other DNA
gcaggggatggaggaagccaaggaaatctcatc

SEQ ID NO: 580 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 581 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 582 - DNA, 36 bp, synthetic construct
  misc_feature 1..36: note = source = /note="Description of Artificial Sequence: Synthetic oligonucleotide"
  source 1..36: mol_type = other DNA
gccagcagtttagggggcgcggatgaaaaactgttt

SEQ ID NO: 583 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 584 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 585 - DNA, 30 bp, synthetic construct
  misc_feature 1..30: note = source = /note="Description of Artificial Sequence: Synthetic oligonucleotide"
  source 1..30: mol_type = other DNA
atcctgggtcagggagcccagaagctggta

SEQ ID NO: 586 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 587 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 588 - DNA, 33 bp, synthetic construct
  misc_feature 1..33: note = source = /note="Description of Artificial Sequence: Synthetic oligonucleotide"
  source 1..33: mol_type = other DNA
gccagcagtttatggacagggggcggctacacc

SEQ ID NO: 589 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 590 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 591 - DNA, 33 bp, synthetic construct; identical to SEQ ID NO: 579
  misc_feature 1..33: note = source = /note="Description of Artificial Sequence: Synthetic oligonucleotide"
  source 1..33: mol_type = other DNA

SEQ ID NO: 592 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 593 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 594 - DNA, 30 bp, synthetic construct; identical to SEQ ID NO: 570
  misc_feature 1..30: note = source = /note="Description of Artificial Sequence: Synthetic oligonucleotide"
  source 1..30: mol_type = other DNA

SEQ ID NO: 595 - DNA, 15 bp, synthetic construct
  misc_feature 1..15: note = source = /note="Description of Artificial Sequence: Synthetic oligonucleotide"
  source 1..15: mol_type = other DNA
gactctgtgaacaat

SEQ ID NO: 596 - DNA, 15 bp, synthetic construct
  misc_feature 1..15: note = source = /note="Description of Artificial Sequence: Synthetic oligonucleotide"
  source 1..15: mol_type = other DNA
attccctcagggaca

SEQ ID NO: 597 - DNA, 33 bp, synthetic construct
  misc_feature 1..33: note = source = /note="Description of Artificial Sequence: Synthetic oligonucleotide"
  source 1..33: mol_type = other DNA
gtcctagggggaggaagccaaggaaatctcatc

SEQ ID NO: 598 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 599 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 600 - DNA, 30 bp, synthetic construct
  misc_feature 1..30: note = source = /note="Description of Artificial Sequence: Synthetic oligonucleotide"
  source 1..30: mol_type = other DNA
gccagcagctttacagactatggctacacc

SEQ ID NO: 601 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 602 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 603 - DNA, 33 bp, synthetic construct
  misc_feature 1..33: note = source = /note="Description of Artificial Sequence: Synthetic oligonucleotide"
  source 1..33: mol_type = other DNA
atcctgagagacgggaccggtaaccagttctat

SEQ ID NO: 604 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 605 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 606 - DNA, 33 bp, synthetic construct
  misc_feature 1..33: note = source = /note="Description of Artificial Sequence: Synthetic oligonucleotide"
  source 1..33: mol_type = other DNA
gccagcagtttatggacagggggtggctacacc

SEQ ID NO: 607 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 608 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 609 - DNA, 33 bp, synthetic construct
  misc_feature 1..33: note = source = /note="Description of Artificial Sequence: Synthetic oligonucleotide"
  source 1..33: mol_type = other DNA
gcaggaggaccctcaggaacctacaaatacatc

SEQ ID NO: 610 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 611 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 612 - DNA, 36 bp, synthetic construct
  misc_feature 1..36: note = source = /note="Description of Artificial Sequence: Synthetic oligonucleotide"
  source 1..36: mol_type = other DNA
gccagcagtcccgggacacctaactatggctacacc

SEQ ID NO: 613 - DNA, 18 bp, synthetic construct
  misc_feature 1..18: note = source = /note="Description of Artificial Sequence: Synthetic oligonucleotide"
  source 1..18: mol_type = other DNA
gaccgaggttcccagtcc

SEQ ID NO: 614 - DNA, 18 bp, synthetic construct
  misc_feature 1..18: note = source = /note="Description of Artificial Sequence: Synthetic oligonucleotide"
  source 1..18: mol_type = other DNA
atatactccaatggtgac

SEQ ID NO: 615 - DNA, 27 bp, synthetic construct
  misc_feature 1..27: note = source = /note="Description of Artificial Sequence: Synthetic oligonucleotide"
  source 1..27: mol_type = other DNA
gccgggaactatggtcagaattttgtc

SEQ ID NO: 616 - DNA, 15 bp, synthetic construct
  misc_feature 1..15: note = source = /note="Description of Artificial Sequence: Synthetic oligonucleotide"
  source 1..15: mol_type = other DNA
cctagacacgacact

SEQ ID NO: 617 - DNA, 18 bp, synthetic construct
  misc_feature 1..18: note = source = /note="Description of Artificial Sequence: Synthetic oligonucleotide"
  source 1..18: mol_type = other DNA
ttttatgaaaagatgcag

SEQ ID NO: 618 - DNA, 33 bp, synthetic construct
  misc_feature 1..33: note = source = /note="Description of Artificial Sequence: Synthetic oligonucleotide"
  source 1..33: mol_type = other DNA
gccagcagcatcggacttaatcagccccagcat

SEQ ID NO: 619 - DNA, 20 bp, synthetic construct
  misc_feature 1..20: note = source = /note="Description of Artificial Sequence: Synthetic oligonucleotide"
  source 1..20: mol_type = other DNA
acaatcagtggaactgatta

SEQ ID NO: 620 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 621 - DNA, 33 bp, synthetic construct
  misc_feature 1..33: note = source = /note="Description of Artificial Sequence: Synthetic oligonucleotide"
  source 1..33: mol_type = other DNA
atcctgagagacggcatcggtaaccagttctat

SEQ ID NO: 622 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 623 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 624 - DNA, 33 bp, synthetic construct
  misc_feature 1..33: note = source = /note="Description of Artificial Sequence: Synthetic oligonucleotide"
  source 1..33: mol_type = other DNA
gccagcagtttatggacagggggaggctacacc

SEQ ID NO: 625 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 626 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 627 - DNA, 33 bp, synthetic construct; identical to SEQ ID NO: 537
  misc_feature 1..33: note = source = /note="Description of Artificial Sequence: Synthetic oligonucleotide"
  source 1..33: mol_type = other DNA

SEQ ID NO: 628 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 629 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 630 - DNA, 33 bp, synthetic construct; identical to SEQ ID NO: 624
  misc_feature 1..33: note = source = /note="Description of Artificial Sequence: Synthetic oligonucleotide"
  source 1..33: mol_type = other DNA

SEQ ID NO: 631 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 632 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 633 - DNA, 33 bp, synthetic construct
  misc_feature 1..33: note = source = /note="Description of Artificial Sequence: Synthetic oligonucleotide"
  source 1..33: mol_type = other DNA
atcctgagagacggcaccggtaaccagttctat

SEQ ID NO: 634 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 635 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 636 - DNA, 33 bp, synthetic construct
  misc_feature 1..33: note = source = /note="Description of Artificial Sequence: Synthetic oligonucleotide"
  source 1..33: mol_type = other DNA
gccagcagttcgacagggtactatggctacacc

SEQ ID NO: 637 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 638 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 639 - DNA, 36 bp, synthetic construct
  misc_feature 1..36: note = source = /note="Description of Artificial Sequence: Synthetic oligonucleotide"
  source 1..36: mol_type = other DNA
atcctgagagaccgggaatatggaaacaaactggtc

SEQ ID NO: 640 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 641 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 642 - DNA, 33 bp, synthetic construct; identical to SEQ ID NO: 588
  misc_feature 1..33: note = source = /note="Description of Artificial Sequence: Synthetic oligonucleotide"
  source 1..33: mol_type = other DNA

SEQ ID NO: 643 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 644 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 645 - DNA, 33 bp, synthetic construct
  misc_feature 1..33: note = source = /note="Description of Artificial Sequence: Synthetic oligonucleotide"
  source 1..33: mol_type = other DNA
gcagaagatggaggaagccaaggaaatctcatc

SEQ ID NO: 646 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 647 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 648 - DNA, 30 bp, synthetic construct
  misc_feature 1..30: note = source = /note="Description of Artificial Sequence: Synthetic oligonucleotide"
  source 1..30: mol_type = other DNA
gccagcagcttatcagactatggctacacc

SEQ ID NO: 649 - DNA, 15 bp, synthetic construct
  misc_feature 1..15: note = source = /note="Description of Artificial Sequence: Synthetic oligonucleotide"
  source 1..15: mol_type = other DNA
gactctgtgaacaat

SEQ ID NO: 650 - DNA, 15 bp, synthetic construct
  misc_feature 1..15: note = source = /note="Description of Artificial Sequence: Synthetic oligonucleotide"
  source 1..15: mol_type = other DNA
attccctcagggaca

SEQ ID NO: 651 - DNA, 33 bp, synthetic construct
  misc_feature 1..33: note = source = /note="Description of Artificial Sequence: Synthetic oligonucleotide"
  source 1..33: mol_type = other DNA
gctgtggaggcctcaggaacctacaaatacatc

SEQ ID NO: 652 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 653 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 654 - DNA, 36 bp, synthetic construct
  misc_feature 1..36: note = source = /note="Description of Artificial Sequence: Synthetic oligonucleotide"
  source 1..36: mol_type = other DNA
gccagcagttgggggacagggggctatggctacacc

SEQ ID NO: 655 - DNA, 18 bp, synthetic construct
  misc_feature 1..18: note = source = /note="Description of Artificial Sequence: Synthetic oligonucleotide"
  source 1..18: mol_type = other DNA
aacagcgcctcagactac

SEQ ID NO: 656 - DNA, 21 bp, synthetic construct
  misc_feature 1..21: note = source = /note="Description of Artificial Sequence: Synthetic oligonucleotide"
  source 1..21: mol_type = other DNA
attcgttcaaatatggacaaa

SEQ ID NO: 657 - DNA, 39 bp, synthetic construct
  misc_feature 1..39: note = source = /note="Description of Artificial Sequence: Synthetic oligonucleotide"
  source 1..39: mol_type = other DNA
gcagagaataagcgggataactatggtcagaattttgtc

SEQ ID NO: 658 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 659 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 660 - DNA, 33 bp, synthetic construct
  misc_feature 1..33: note = source = /note="Description of Artificial Sequence: Synthetic oligonucleotide"
  source 1..33: mol_type = other DNA
gccagcagtttctgggtgaacactgaagctttc

SEQ ID NO: 661 - DNA, 15 bp, synthetic construct
  misc_feature 1..15: note = source = /note="Description of Artificial Sequence: Synthetic oligonucleotide"
  source 1..15: mol_type = other DNA
gactctgtgaacaat

SEQ ID NO: 662 - DNA, 15 bp, synthetic construct
  misc_feature 1..15: note = source = /note="Description of Artificial Sequence: Synthetic oligonucleotide"
  source 1..15: mol_type = other DNA
attccctcagggaca

SEQ ID NO: 663 - DNA, 33 bp, synthetic construct
  misc_feature 1..33: note = source = /note="Description of Artificial Sequence: Synthetic oligonucleotide"
  source 1..33: mol_type = other DNA
gctgtggggagttctaacggctacaagctcagc

SEQ ID NO: 664 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 665 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 666 - DNA, 39 bp, synthetic construct
  misc_feature 1..39: note = source = /note="Description of Artificial Sequence: Synthetic oligonucleotide"
  source 1..39: mol_type = other DNA
gccagcagtcccgggacagggggattttcacccctccac

SEQ ID NO: 667 - DNA, 15 bp, synthetic construct
  misc_feature 1..15: note = source = /note="Description of Artificial Sequence: Synthetic oligonucleotide"
  source 1..15: mol_type = other DNA
gactctgtgaacaat

SEQ ID NO: 668 - DNA, 15 bp, synthetic construct
  misc_feature 1..15: note = source = /note="Description of Artificial Sequence: Synthetic oligonucleotide"
  source 1..15: mol_type = other DNA
attccctcagggaca

SEQ ID NO: 669 - DNA, 33 bp, synthetic construct
  misc_feature 1..33: note = source = /note="Description of Artificial Sequence: Synthetic oligonucleotide"
  source 1..33: mol_type = other DNA
gctgtggggagttctaacgactacaagctcagc

SEQ ID NO: 670 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 671 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 672 - DNA, 39 bp, synthetic construct; identical to SEQ ID NO: 666
  misc_feature 1..39: note = source = /note="Description of Artificial Sequence: Synthetic oligonucleotide"
  source 1..39: mol_type = other DNA

SEQ ID NO: 673 - DNA, 18 bp, synthetic construct
  misc_feature 1..18: note = source = /note="Description of Artificial Sequence: Synthetic oligonucleotide"
  source 1..18: mol_type = other DNA
aacagcgcctcagactac

SEQ ID NO: 674 - DNA, 21 bp, synthetic construct
  misc_feature 1..21: note = source = /note="Description of Artificial Sequence: Synthetic oligonucleotide"
  source 1..21: mol_type = other DNA
attcgttcaaatatggacaaa

SEQ ID NO: 675 - DNA, 39 bp, synthetic construct
  misc_feature 1..39: note = source = /note="Description of Artificial Sequence: Synthetic oligonucleotide"
  source 1..39: mol_type = other DNA
gcagagaataggcaggataactatggtcagaattttgtc

SEQ ID NO: 676 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 677 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 678 - DNA, 33 bp, synthetic construct
  misc_feature 1..33: note = source = /note="Description of Artificial Sequence: Synthetic oligonucleotide"
  source 1..33: mol_type = other DNA
gccagcagtttatgggttaacactgaagctttc

SEQ ID NO: 679 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 680 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 681 - DNA, 33 bp, synthetic construct
  misc_feature 1..33: note = source = /note="Description of Artificial Sequence: Synthetic oligonucleotide"
  source 1..33: mol_type = other DNA
atcctgcggcctgacagctgggggaagttccag

SEQ ID NO: 682 - DNA, 15 bp, synthetic construct
  misc_feature 1..15: note = source = /note="Description of Artificial Sequence: Synthetic oligonucleotide"
  source 1..15: mol_type = other DNA
atggaccatgaaaat

SEQ ID NO: 683 - DNA, 18 bp, synthetic construct
  misc_feature 1..18: note = source = /note="Description of Artificial Sequence: Synthetic oligonucleotide"
  source 1..18: mol_type = other DNA
tcatatgatgttaaaatg

SEQ ID NO: 684 - DNA, 39 bp, synthetic construct
  misc_feature 1..39: note = source = /note="Description of Artificial Sequence: Synthetic oligonucleotide"
  source 1..39: mol_type = other DNA
gccagcagtaccgtgaggcaggggaactatggctacacc

SEQ ID NO: 685 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 686 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 687 - DNA, 33 bp, synthetic construct
  misc_feature 1..33: note = source = /note="Description of Artificial Sequence: Synthetic oligonucleotide"
  source 1..33: mol_type = other DNA
atcctgaataccggcactgccagtaaactcacc

SEQ ID NO: 688 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 689 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 690 - DNA, 33 bp, synthetic construct
  misc_feature 1..33: note = source = /note="Description of Artificial Sequence: Synthetic oligonucleotide"
  source 1..33: mol_type = other DNA
gccagcagtttatcgtcgaacactgaagctttc

SEQ ID NO: 691 - DNA, 18 bp, synthetic construct
  misc_feature 1..18: note = source = /note="Description of Artificial Sequence: Synthetic oligonucleotide"
  source 1..18: mol_type = other DNA
gaccgaggttcccagtcc

SEQ ID NO: 692 - DNA, 18 bp, synthetic construct
  misc_feature 1..18: note = source = /note="Description of Artificial Sequence: Synthetic oligonucleotide"
  source 1..18: mol_type = other DNA
atatactccaatggtgac

SEQ ID NO: 693 - DNA, 30 bp, synthetic construct
  misc_feature 1..30: note = source = /note="Description of Artificial Sequence: Synthetic oligonucleotide"
  source 1..30: mol_type = other DNA
gccgtgaacagaggcaccgacaagctcatc

SEQ ID NO: 694 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 695 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 696 - DNA, 30 bp, synthetic construct
  misc_feature 1..30: note = source = /note="Description of Artificial Sequence: Synthetic oligonucleotide"
  source 1..30: mol_type = other DNA
gccagcagctggacagactatggctacacc

SEQ ID NO: 697 - DNA, 21 bp, synthetic construct
  misc_feature 1..21: note = source = /note="Description of Artificial Sequence: Synthetic oligonucleotide"
  source 1..21: mol_type = other DNA
acaatcagtggaactgattac

SEQ ID NO: 698 - DNA, 15 bp, synthetic construct
  misc_feature 1..15: note = source = /note="Description of Artificial Sequence: Synthetic oligonucleotide"
  source 1..15: mol_type = other DNA
ggtcttacaagcaat

SEQ ID NO: 699 - DNA, 33 bp, synthetic construct; identical to SEQ ID NO: 537
  misc_feature 1..33: note = source = /note="Description of Artificial Sequence: Synthetic oligonucleotide"
  source 1..33: mol_type = other DNA

SEQ ID NO: 700 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 701 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 702 - DNA, 30 bp, synthetic construct; identical to SEQ ID NO: 696
  misc_feature 1..30: note = source = /note="Description of Artificial Sequence: Synthetic oligonucleotide"
  source 1..30: mol_type = other DNA

SEQ ID NO: 703 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 704 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 705 - DNA, 33 bp, synthetic construct
  misc_feature 1..33: note = source = /note="Description of Artificial Sequence: Synthetic oligonucleotide"
  source 1..33: mol_type = other DNA
gcaggaggaacctcaggaacctacaaatacatc

SEQ ID NO: 706 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 707 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 708 - DNA, 36 bp, synthetic construct
  misc_feature 1..36: note = source = /note="Description of Artificial Sequence: Synthetic oligonucleotide"
  source 1..36: mol_type = other DNA
gccagcagtcccgggacacccaactatggctacacc

SEQ ID NO: 709 - DNA, 18 bp, synthetic construct
  misc_feature 1..18: note = source = /note="Description of Artificial Sequence: Synthetic oligonucleotide"
  source 1..18: mol_type = other DNA
aacagtgcttctcagtct

SEQ ID NO: 710 - DNA, 18 bp, synthetic construct
  misc_feature 1..18: note = source = /note="Description of Artificial Sequence: Synthetic oligonucleotide"
  source 1..18: mol_type = other DNA
gtatactccagtggtaat

SEQ ID NO: 711 - DNA, 33 bp, synthetic construct
  misc_feature 1..33: note = source = /note="Description of Artificial Sequence: Synthetic oligonucleotide"
  source 1..33: mol_type = other DNA
gtggtgaatggaggaagccaaggaaatctcatc

SEQ ID NO: 712 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 713 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 714 - DNA, 30 bp, synthetic construct
  misc_feature 1..30: note = source = /note="Description of Artificial Sequence: Synthetic oligonucleotide"
  source 1..30: mol_type = other DNA
gccagcagcgtaggggactatggctacacc

SEQ ID NO: 715 - DNA, 18 bp, synthetic construct
  misc_feature 1..18: note = source = /note="Description of Artificial Sequence: Synthetic oligonucleotide"
  source 1..18: mol_type = other DNA
aacagcgcctcagactac

SEQ ID NO: 716 - DNA, 21 bp, synthetic construct
  misc_feature 1..21: note = source = /note="Description of Artificial Sequence: Synthetic oligonucleotide"
  source 1..21: mol_type = other DNA
attcgttcaaatatggacaaa

SEQ ID NO: 717 - DNA, 30 bp, synthetic construct
  misc_feature 1..30: note = source = /note="Description of Artificial Sequence: Synthetic oligonucleotide"
  source 1..30: mol_type = other DNA
gcagagaacaactatggtcagaattttgtc

SEQ ID NO: 718 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 719 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 720 - DNA, 33 bp, synthetic construct
  misc_feature 1..33: note = source = /note="Description of Artificial Sequence: Synthetic oligonucleotide"
  source 1..33: mol_type = other DNA
gccagcagtttatgggacagttcacccctccac

SEQ ID NO: 721 - DNA, 18 bp, synthetic construct
  misc_feature 1..18: note = source = /note="Description of Artificial Sequence: Synthetic oligonucleotide"
  source 1..18: mol_type = other DNA
aacagcatgtttgattat

SEQ ID NO: 722 - DNA, 21 bp, synthetic construct
  misc_feature 1..21: note = source = /note="Description of Artificial Sequence: Synthetic oligonucleotide"
  source 1..21: mol_type = other DNA
ataagttccattaaggataaa

SEQ ID NO: 723 - DNA, 33 bp, synthetic construct
  misc_feature 1..33: note = source = /note="Description of Artificial Sequence: Synthetic oligonucleotide"
  source 1..33: mol_type = other DNA
gcagcaagcgccggttctgcaaggcaactgacc

SEQ ID NO: 724 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 725 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 726 - DNA, 33 bp, synthetic construct
  misc_feature 1..33: note = source = /note="Description of Artificial Sequence: Synthetic oligonucleotide"
  source 1..33: mol_type = other DNA
gccagcagtttatacacccacactgaagctttc

SEQ ID NO: 727 - DNA, 21 bp, synthetic construct
  misc_feature 1..21: note = source = /note="Description of Artificial Sequence: Synthetic oligonucleotide"
  source 1..21: mol_type = other DNA
accatcagtggaaatgagtat

SEQ ID NO: 728 - DNA, 15 bp, synthetic construct
  misc_feature 1..15: note = source = /note="Description of Artificial Sequence: Synthetic oligonucleotide"
  source 1..15: mol_type = other DNA
ggtctaaaaaacaat

SEQ ID NO: 729 - DNA, 39 bp, synthetic construct
  misc_feature 1..39: note = source = /note="Description of Artificial Sequence: Synthetic oligonucleotide"
  source 1..39: mol_type = other DNA
atcgtcagagacactacctcaggaacctacaaatacatc

SEQ ID NO: 730 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 731 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 732 - DNA, 39 bp, synthetic construct
  misc_feature 1..39: note = source = /note="Description of Artificial Sequence: Synthetic oligonucleotide"
  source 1..39: mol_type = other DNA
gccagcagtttatcctcgacaggtttttcacccctccac

SEQ ID NO: 733 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 734 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 735 - DNA, 33 bp, synthetic construct
  misc_feature 1..33: note = source = /note="Description of Artificial Sequence: Synthetic oligonucleotide"
  source 1..33: mol_type = other DNA
gcagaggggggaggaagccaaggaaatctcatc

SEQ ID NO: 736 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 737 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 738 - DNA, 30 bp, synthetic construct; identical to SEQ ID NO: 696
  misc_feature 1..30: note = source = /note="Description of Artificial Sequence: Synthetic oligonucleotide"
  source 1..30: mol_type = other DNA

SEQ ID NO: 739 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 740 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 741 - DNA, 33 bp, synthetic construct
  misc_feature 1..33: note = source = /note="Description of Artificial Sequence: Synthetic oligonucleotide"
  source 1..33: mol_type = other DNA
gcagggatacgttctaacgactacaagctcagc

SEQ ID NO: 742 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 743 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 744 - DNA, 36 bp, synthetic construct
  misc_feature 1..36: note = source = /note="Description of Artificial Sequence: Synthetic oligonucleotide"
  source 1..36: mol_type = other DNA
gccagcagttcctggacagcccacactgaagctttc

SEQ ID NO: 745 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 746 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 747 - DNA, 36 bp, synthetic construct
  misc_feature 1..36: note = source = /note="Description of Artificial Sequence: Synthetic oligonucleotide"
  source 1..36: mol_type = other DNA
gcagaaaattcaggaggaggtgctgacggactcacc

SEQ ID NO: 748 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 749 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 750 - DNA, 30 bp, synthetic construct
  misc_feature 1..30: note = source = /note="Description of Artificial Sequence: Synthetic oligonucleotide"
  source 1..30: mol_type = other DNA
gccagcagcttcacagactatggctacacc

SEQ ID NO: 751 - DNA, 15 bp, synthetic construct
  misc_feature 1..15: note = source = /note="Description of Artificial Sequence: Synthetic oligonucleotide"
  source 1..15: mol_type = other DNA
agtgttttttccagc

SEQ ID NO: 752 - DNA, 21 bp, synthetic construct
  misc_feature 1..21: note = source = /note="Description of Artificial Sequence: Synthetic oligonucleotide"
  source 1..21: mol_type = other DNA
gtagttacgggtggagaagtg

SEQ ID NO: 753 - DNA, 33 bp, synthetic construct
  misc_feature 1..33: note = source = /note="Description of Artificial Sequence: Synthetic oligonucleotide"
  source 1..33: mol_type = other DNA
gcaggagaggactttggaaatgagaaattaacc

SEQ ID NO: 754 - DNA, 15 bp, synthetic construct
  misc_feature 1..15: note = source = /note="Description of Artificial Sequence: Synthetic oligonucleotide"
  source 1..15: mol_type = other DNA
tctgggcatgacact

SEQ ID NO: 755 - DNA, 18 bp, synthetic construct
  misc_feature 1..18: note = source = /note="Description of Artificial Sequence: Synthetic oligonucleotide"
  source 1..18: mol_type = other DNA
tattatgaggaggaagag

SEQ ID NO: 756 - DNA, 30 bp, synthetic construct
  misc_feature 1..30: note = source = /note="Description of Artificial Sequence: Synthetic oligonucleotide"
  source 1..30: mol_type = other DNA
gccagcagctgggcggattatggctacacc

SEQ ID NO: 757 - DNA, 18 bp, synthetic construct
  misc_feature 1..18: note = source = /note="Description of Artificial Sequence: Synthetic oligonucleotide"
  source 1..18: mol_type = other DNA
tccagcaatttttatgcc

SEQ ID NO: 758 - DNA, 21 bp, synthetic construct
  misc_feature 1..21: note = source = /note="Description of Artificial Sequence: Synthetic oligonucleotide"
  source 1..21: mol_type = other DNA
atgactttaaatggggatgaa

SEQ ID NO: 759 - DNA, 27 bp, synthetic construct
  misc_feature 1..27: note = source = /note="Description of Artificial Sequence: Synthetic oligonucleotide"
  source 1..27: mol_type = other DNA
gcctttctcaccggtaaccagttctat

SEQ ID NO: 760 - DNA, 15 bp, synthetic construct
  misc_feature 1..15: note = source = /note="Description of Artificial Sequence: Synthetic oligonucleotide"
  source 1..15: mol_type = other DNA
atggaccatgaaaat

SEQ ID NO: 761 - DNA, 18 bp, synthetic construct
  misc_feature 1..18: note = source = /note="Description of Artificial Sequence: Synthetic oligonucleotide"
  source 1..18: mol_type = other DNA
tcatatgatgttaaaatg

SEQ ID NO: 762 - DNA, 39 bp, synthetic construct; identical to SEQ ID NO: 684
  misc_feature 1..39: note = source = /note="Description of Artificial Sequence: Synthetic oligonucleotide"
  source 1..39: mol_type = other DNA

SEQ ID NO: 763 - DNA, 18 bp, synthetic construct
  misc_feature 1..18: note = source = /note="Description of Artificial Sequence: Synthetic oligonucleotide"
  source 1..18: mol_type = other DNA
aacagcatgtttgattat

SEQ ID NO: 764 - DNA, 21 bp, synthetic construct
  misc_feature 1..21: note = source = /note="Description of Artificial Sequence: Synthetic oligonucleotide"
  source 1..21: mol_type = other DNA
ataagttccattaaggataaa

SEQ ID NO: 765 - DNA, 33 bp, synthetic construct; identical to SEQ ID NO: 723
  misc_feature 1..33: note = source = /note="Description of Artificial Sequence: Synthetic oligonucleotide"
  source 1..33: mol_type = other DNA

SEQ ID NO: 766 - DNA, 15 bp, synthetic construct
  misc_feature 1..15: note = source = /note="Description of Artificial Sequence: Synthetic oligonucleotide"
  source 1..15: mol_type = other DNA
atgaaccatgagtat

SEQ ID NO: 767 - DNA, 18 bp, synthetic construct
  misc_feature 1..18: note = source = /note="Description of Artificial Sequence: Synthetic oligonucleotide"
  source 1..18: mol_type = other DNA
tcaatgaatgttgaggtg

SEQ ID NO: 768 - DNA, 33 bp, synthetic construct
  misc_feature 1..33: note = source = /note="Description of Artificial Sequence: Synthetic oligonucleotide"
  source 1..33: mol_type = other DNA
gccagcagtttatggtcgaacactgaagctttc

SEQ ID NO: 769 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAANGGSQGNLIFGKGTKLSVKP

SEQ ID NO: 770 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgccgctaatggaggaagccaaggaaatctcatctttggaaaaggcactaaactctctgttaaacca

SEQ ID NO: 771 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLQDYGYTFGSGTRLTVV

SEQ ID NO: 772 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttacaggactatggctacaccttcggttcggggaccaggttaaccgttgta

SEQ ID NO: 773 - protein, 110 aa, synthetic construct
  REGION 1..110: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRSTLRDAAVYYCILRPDSWGKFQFGAGTQVVVTP

SEQ ID NO: 774 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgttctaccttgagagatgctgctgtgtactactgcatcctgcggcctgacagctgggggaaattccagtttggagcagggacccaggttgtggtcacccca

SEQ ID NO: 775 - protein, 111 aa, synthetic construct; identical to SEQ ID NO: 771
  REGION 1..111: note = source = /note="Description of Artificial Sequence: Synthetic polypeptide"

SEQ ID NO: 776 - DNA, 333 bp, synthetic construct; identical to SEQ ID NO: 772
  misc_feature 1..333: note = source = /note="Description of Artificial Sequence: Synthetic polynucleotide"
  source 1..333: mol_type = other DNA

SEQ ID NO: 777 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCATNGGSQGNLIFGKGTKLSVKP

SEQ ID NO: 778 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgccactaatggaggaagccaaggaaatctcatctttggaaaaggcactaaactctctgttaaacca

SEQ ID NO: 779 - protein, 111 aa, synthetic construct; identical to SEQ ID NO: 771
  REGION 1..111: note = source = /note="Description of Artificial Sequence: Synthetic polypeptide"

SEQ ID NO: 780 - DNA, 333 bp, synthetic construct; identical to SEQ ID NO: 772
  misc_feature 1..333: note = source = /note="Description of Artificial Sequence: Synthetic polynucleotide"
  source 1..333: mol_type = other DNA

SEQ ID NO: 781 - protein, 108 aa, synthetic construct
  REGION 1..108: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCIRRPGNQFYFGTGTSLTVIP

SEQ ID NO: 782 - DNA, 324 bp, synthetic construct
  misc_feature 1..324: note = source = /note="Description of Artificial Sequence: Synthetic polynucleotide"
  source 1..324: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcaggcggcccggtaaccagttctattttgggacagggacaagtttgacggtcattcca

SEQ ID NO: 783 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWTGSEAFFGQGTRLTVV

SEQ ID NO: 784 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatggacagggtctgaagctttctttggacaaggcaccagactcacagttgta

SEQ ID NO: 785 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAGGTSGTYKYIFGTGTRLKVLA

SEQ ID NO: 786 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttactggtggagaagtgaagaagctgaagagactaacctttcagtttggtgatgcaagaaaggacagttctctccacatcactgcggcccagcctggtgatacaggcctctacctctgtgcaggagggacctcaggaacctacaaatacatctttggaacaggcaccaggctgaaggttttagca

SEQ ID NO: 787 - protein, 113 aa, synthetic construct
  REGION 1..113: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGTANYGYTFGSGTRLTVV

SEQ ID NO: 788 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtccagggacagctaactatggctacaccttcggttcggggaccaggttaaccgttgta

SEQ ID NO: 789 - protein, 107 aa, synthetic construct
  REGION 1..107: note = source = /note="Description of Artificial Sequence: Synthetic polypeptide"
AQRVTQPEKLLSVFKGAPVELKCNYSYSGSPELFWYVQYSRQRLQLLLRHISRESIKGFTADLNKGETSFHLKKPFAQEEDSAMYYCALGNTDKLIFGTGTRLQVFP

SEQ ID NO: 790 - DNA, 321 bp, synthetic construct
  misc_feature 1..321: note = source = /note="Description of Artificial Sequence: Synthetic polynucleotide"
  source 1..321: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctagggaacaccgacaagctcatctttgggactgggaccagattacaagtctttcca

SEQ ID NO: 791 - protein, 114 aa, synthetic construct
  REGION 1..114: note = source = /note="Description of Artificial Sequence: Synthetic polypeptide"
DVKVTQSSRYLVKRTGEKVFLECVQDMDHENMFWYRQDPGLGLRLIYFSYDVKMKEKGDIPEGYSVSREKKERFSLILESASTNQTSMYLCASSSPRTGWYGYTFGSGTRLTVV

SEQ ID NO: 792 - DNA, 342 bp, synthetic construct
  misc_feature 1..342: note = source = /note="Description of Artificial Sequence: Synthetic polynucleotide"
  source 1..342: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagttcgcccaggacagggtggtatggctacaccttcggttcggggaccaggttaaccgttgta

SEQ ID NO: 793 - protein, 110 aa, synthetic construct; identical to SEQ ID NO: 773
  REGION 1..110: note = source = /note="Description of Artificial Sequence: Synthetic polypeptide"

SEQ ID NO: 794 - DNA, 330 bp, synthetic construct; identical to SEQ ID NO: 774
  misc_feature 1..330: note = source = /note="Description of Artificial Sequence: Synthetic polynucleotide"
  source 1..330: mol_type = other DNA

SEQ ID NO: 795 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLVDYGYTFGSGTRLTVV

SEQ ID NO: 796 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagccccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttggtggattatggctacaccttcggttcggggaccaggttaaccgttgta

SEQ ID NO: 797 - protein, 110 aa, synthetic construct; identical to SEQ ID NO: 773
  REGION 1..110: note = source = /note="Description of Artificial Sequence: Synthetic polypeptide"

SEQ ID NO: 798 - DNA, 330 bp, synthetic construct; identical to SEQ ID NO: 774
  misc_feature 1..330: note = source = /note="Description of Artificial Sequence: Synthetic polynucleotide"
  source 1..330: mol_type = other DNA

SEQ ID NO: 799 - protein, 113 aa, synthetic construct
  REGION 1..113: note = source = /note="Description of Artificial Sequence: Synthetic polypeptide"
EAQVTQSPRYLITVTGKKLTVTCSQNMDHEYMSWYRQDPGLGLRQIYYSMNVEVTDKGDVPEGYKVPRKEKRSFPLILESPCCSQTPLYLCASSLGGVDERLSFGSGTQLSVL

SEQ ID NO: 800 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagagcccaagatacctcatcacagtgactggaaagaagttaacagtgacttgttctcagaatatggaccatgagtatatgtcctggtatcgacaggacccggggctgggcctaaggcagatctactattcaatgaatgttgaggtgacagataagggagatgttcctgaagggtacaaagtccctcgaaaagagaagaggagtttccccctgatcctggagtcgccctgctgcagccagacccctctgtacctctgtgccagcagtttagggggcgtggatgaaagactgtcttttggcagtggaacccagctctccgtcttg

SEQ ID NO: 801 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAGDGGSQGNLIFGKGTKLSVKP

SEQ ID NO: 802 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggggatggaggaagccaaggaaatctcatctttggaaaaggcactaaactctctgttaaacca

SEQ ID NO: 803 - protein, 113 aa, synthetic construct
  REGION 1..113: note = source = /note="Description of Artificial Sequence: Synthetic polypeptide"
EAQVTQSPRYLITVTGKKLTVTCSQNMNHEYMSWYRQDPGLGLRQIYYSMNVEVTDKGDVPEGYKVSRKEKRNFPLILESPSPNQTSLYFCASSLGGADEKLFFGSGTQLSVL

SEQ ID NO: 804 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagagcccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttagggggcgcggatgaaaaactgttttttggcagtggaacccagctctctgtcttg

SEQ ID NO: 805 - protein, 109 aa, synthetic construct
  REGION 1..109: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILGQGAQKLVFGQGTRLTINP

SEQ ID NO: 806 - DNA, 327 bp, synthetic construct
  misc_feature 1..327: note = source = /note="Description of Artificial Sequence: Synthetic polynucleotide"
  source 1..327: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgggtcagggagcccagaagctggtatttggccaaggaaccaggctgactatcaaccca

SEQ ID NO: 807 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWTGGGYTFGSGTRLTVV

SEQ ID NO: 808 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatggacagggggcggctacaccttcggttcggggaccaggttaaccgttgta

SEQ ID NO: 809 - protein, 111 aa, synthetic construct; identical to SEQ ID NO: 801
  REGION 1..111: note = source = /note="Description of Artificial Sequence: Synthetic polypeptide"

SEQ ID NO: 810 - DNA, 333 bp, synthetic construct; identical to SEQ ID NO: 802
  misc_feature 1..333: note = source = /note="Description of Artificial Sequence: Synthetic polynucleotide"
  source 1..333: mol_type = other DNA

SEQ ID NO: 811 - protein, 111 aa, synthetic construct; identical to SEQ ID NO: 795
  REGION 1..111: note = source = /note="Description of Artificial Sequence: Synthetic polypeptide"

SEQ ID NO: 812 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttggtggattatggctacaccttcggttcggggaccaggttaaccgttgta

SEQ ID NO: 813 - protein, 109 aa, synthetic construct
  REGION 1..109: note = source = /note="Description of Artificial Sequence: Synthetic polypeptide"
GIQVEQSPPDLILQEGANSTLRCNFSDSVNNLQWFHQNPWGQLINLFYIPSGTKQNGRLSATTVATERYSLLYISSSQTTDSGVYFCVLGGGSQGNLIFGKGTKLSVKP

SEQ ID NO: 814 - DNA, 327 bp, synthetic construct
  misc_feature 1..327: note = source = /note="Description of Artificial Sequence: Synthetic polynucleotide"
  source 1..327: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgtcctagggggaggaagccaaggaaatctcatctttggaaaaggcactaaactctctgttaaacca

SEQ ID NO: 815 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFTDYGYTFGSGTRLTVV

SEQ ID NO: 816 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctttacagactatggctacaccttcggttcggggaccaggttaaccgttgta

SEQ ID NO: 817 - protein, 110 aa, synthetic construct
  REGION 1..110: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILRDGTGNQFYFGTGTSLTVIP

SEQ ID NO: 818 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagacgggaccggtaaccagttctattttgggacagggacaagtttgacggtcattcca

SEQ ID NO: 819 - protein, 112 aa, synthetic construct; identical to SEQ ID NO: 807
  REGION 1..112: note = source = /note="Description of Artificial Sequence: Synthetic polypeptide"

SEQ ID NO: 820 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatggacagggggtggctacaccttcggttcggggaccaggttaaccgttgta

SEQ ID NO: 821 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAGGPSGTYKYIFGTGTRLKVLA

SEQ ID NO: 822 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggaggaccctcaggaacctacaaatacatctttggaacaggcaccaggctgaaggttttagca

SEQ ID NO: 823 - protein, 113 aa, synthetic construct
  REGION 1..113: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGTPNYGYTFGSGTRLTVV

SEQ ID NO: 824 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtcccgggacacctaactatggctacaccttcggttcggggaccaggttaaccgttgta

SEQ ID NO: 825 - protein, 109 aa, synthetic construct
  REGION 1..109: note = source = /note="Description of Artificial Sequence: Synthetic polypeptide"
QKEVEQNSGPLSVPEGAIASLNCTYSDRGSQSFFWYRQYSGKSPELIMSIYSNGDKEDGRFTAQLNKASQYVSLLIRDSQPSDSATYLCAGNYGQNFVFGPGTRLSVLP

SEQ ID NO: 826 - DNA, 327 bp, synthetic construct
  misc_feature 1..327: note = source = /note="Description of Artificial Sequence: Synthetic polynucleotide"
  source 1..327: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgggaactatggtcagaattttgtctttggtcccggaaccagattgtccgtgctgccc

SEQ ID NO: 827 - protein, 112 aa, synthetic construct
  REGION 1..112: note = source = /note="Description of Artificial Sequence: Synthetic polypeptide"
AAGVIQSPRHLIKEKRETATLKCYPIPRHDTVYWYQQGPGQDPQFLISFYEKMQSDKGSIPDRFSAQQFSDYHSELNMSSLELGDSALYFCASSIGLNQPQHFGDGTRLSIL

SEQ ID NO: 828 - DNA, 336 bp, synthetic construct
  misc_feature 1..336: note = source = /note="Description of Artificial Sequence: Synthetic polynucleotide"
  source 1..336: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcatcggacttaatcagccccagcattttggtgatgggactcgactctccatccta

SEQ ID NO: 829 - protein, 110 aa, synthetic construct
  REGION 1..110: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILRDGIGNQFYFGTGTSLTVIP

SEQ ID NO: 830 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagacggcatcggtaaccagttctattttgggacagggacaagtttgacggtcattcca

SEQ ID NO: 831 - protein, 112 aa, synthetic construct; identical to SEQ ID NO: 807
  REGION 1..112: note = source = /note="Description of Artificial Sequence: Synthetic polypeptide"

SEQ ID NO: 832 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatggacagggggaggctacaccttcggttcggggaccaggttaaccgttgta

SEQ ID NO: 833 - protein, 110 aa, synthetic construct; identical to SEQ ID NO: 773
  REGION 1..110: note = source = /note="Description of Artificial Sequence: Synthetic polypeptide"

SEQ ID NO: 834 - DNA, 330 bp, synthetic construct; identical to SEQ ID NO: 774
  misc_feature 1..330: note = source = /note="Description of Artificial Sequence: Synthetic polynucleotide"
  source 1..330: mol_type = other DNA

SEQ ID NO: 835 - protein, 112 aa, synthetic construct; identical to SEQ ID NO: 807
  REGION 1..112: note = source = /note="Description of Artificial Sequence: Synthetic polypeptide"

SEQ ID NO: 836 - DNA, 336 bp, synthetic construct; identical to SEQ ID NO: 832
  misc_feature 1..336: note = source = /note="Description of Artificial Sequence: Synthetic polynucleotide"
  source 1..336: mol_type = other DNA

SEQ ID NO: 837 - protein, 110 aa, synthetic construct; identical to SEQ ID NO: 817
  REGION 1..110: note = source = /note="Description of Artificial Sequence: Synthetic polypeptide"

SEQ ID NO: 838 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagacggcaccggtaaccagttctattttgggacagggacaagtttgacggtcattcca

SEQ ID NO: 839 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTGYYGYTFGSGTRLTVV

SEQ ID NO: 840 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagttcgacagggtactatggctacaccttcggttcggggaccaggttaaccgttgta

SEQ ID NO: 841 - protein, 111 aa, synthetic construct
  REGION 1..111: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILRDREYGNKLVFGAGTILRVKS

SEQ ID NO: 842 - DNA, 333 bp, synthetic construct
  misc_feature 1..333: note = source = /note="Description of Artificial Sequence: Synthetic polynucleotide"
  source 1..333: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagaccgggaatatggaaacaaactggtctttggcgcaggaaccattctgagagtcaagtcc

SEQ ID NO: 843 - protein, 112 aa, synthetic construct; identical to SEQ ID NO: 807
  REGION 1..112: note = source = /note="Description of Artificial Sequence: Synthetic polypeptide"

SEQ ID NO: 844 - DNA, 336 bp, synthetic construct; identical to SEQ ID NO: 808
  misc_feature 1..336: note = source = /note="Description of Artificial Sequence: Synthetic polynucleotide"
  source 1..336: mol_type = other DNA

SEQ ID NO: 845 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAEDGGSQGNLIFGKGTKLSVKP

SEQ ID NO: 846 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcagaagatggaggaagccaaggaaatctcatctttggaaaaggcactaaactctctgttaaacca

SEQ ID NO: 847 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLSDYGYTFGSGTRLTVV

SEQ ID NO: 848 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttatcagactatggctacaccttcggttcggggaccaggttaaccgttgta

SEQ ID NO: 849 - protein, 109 aa, synthetic construct
  REGION 1..109: note = source = /note="Description of Artificial Sequence: Synthetic polypeptide"
GIQVEQSPPDLILQEGANSTLRCNFSDSVNNLQWFHQNPWGQLINLFYIPSGTKQNGRLSATTVATERYSLLYISSSQTTDSGVYFCAVEASGTYKYIFGTGTRLKVLA

SEQ ID NO: 850 - DNA, 327 bp, synthetic construct
  misc_feature 1..327: note = source = /note="Description of Artificial Sequence: Synthetic polynucleotide"
  source 1..327: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggaggcctcaggaacctacaaatacatctttggaacaggcaccaggctgaaggttttagca

SEQ ID NO: 851 - protein, 113 aa, synthetic construct
  REGION 1..113: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSWGTGGYGYTFGSGTRLTVV

SEQ ID NO: 852 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagttgggggacagggggctatggctacaccttcggttcggggaccaggttaaccgttgta

SEQ ID NO: 853 - protein, 114 aa, synthetic construct
  REGION 1..114: note = source = /note="Description of Artificial Sequence: Synthetic polypeptide"
GESVGLHLPTLSVQEGDNSIINCAYSNSASDYFIWYKQESGKGPQFIIDIRSNMDKRQGQRVTVLLNKTVKHLSLQIAATQPGDSAVYFCAENKRDNYGQNFVFGPGTRLSVLP

SEQ ID NO: 854 - DNA, 342 bp, synthetic construct
  misc_feature 1..342: note = source = /note="Description of Artificial Sequence: Synthetic polynucleotide"
  source 1..342: mol_type = other DNA
ggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaataagcgggataactatggtcagaattttgtctttggtcccggaaccagattgtccgtgctgccc

SEQ ID NO: 855 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFWVNTEAFFGQGTRLTVV

SEQ ID NO: 856 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttctgggtgaacactgaagctttctttggacaaggcaccagactcacagttgta

SEQ ID NO: 857 - protein, 109 aa, synthetic construct
  REGION 1..109: note = source = /note="Description of Artificial Sequence: Synthetic polypeptide"
GIQVEQSPPDLILQEGVNSTLRCNFSDSVNNLQWFHQNPWGQLINLFYIPSGTKQNGRLSATTVATERYSLLYISSSRTTDSGVYFCAVGSSNGYKLSFGAGTTVTVRA

SEQ ID NO: 858 - DNA, 327 bp, synthetic construct
  misc_feature 1..327: note = source = /note="Description of Artificial Sequence: Synthetic polynucleotide"
  source 1..327: mol_type = other DNA
ggaatacaagtggagcagagtcctccagacctgattctccaggagggagtcaattccacgctgcggtgcaatttttctgactctgtgaacaatttgcagtggtttcatcaaaacccttggggacagctcatcaacctgttttacattccctcagggacaaaacagaatggaagattaagcgccacgactgtggctacggaacgctacagcttattgtacatttcctcttcccggaccacagactcaggcgtttatttctgtgctgtggggagttctaacggctacaagctcagctttggagctggaaccacagtaactgtaagagca

SEQ ID NO: 859 - protein, 114 aa, synthetic construct
  REGION 1..114: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGTGGFSPLHFGNGTRLTVT

SEQ ID NO: 860 - DNA, 342 bp, synthetic construct
  misc_feature 1..342: note = source = /note="Description of Artificial Sequence: Synthetic polynucleotide"
  source 1..342: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtcccgggacagggggattttcacccctccactttgggaacgggaccaggctcactgtgaca

SEQ ID NO: 861 - protein, 109 aa, synthetic construct
  REGION 1..109: note = source = /note="Description of Artificial Sequence: Synthetic polypeptide"
GIQVEQSPPDLILQEGANSTLRCNFSDSVNNLQWFHQNPWGQLINLFYIPSGTKQNGRLSATTVATERYSLLYISSSQTTDSGVYFCAVGSSNDYKLSFGAGTTVTVRA

SEQ ID NO: 862 - DNA, 327 bp, synthetic construct
  misc_feature 1..327: note = source = /note="Description of Artificial Sequence: Synthetic polynucleotide"
  source 1..327: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggggagttctaacgactacaagctcagctttggagccggaaccacagtaactgtaagagca

SEQ ID NO: 863 - protein, 114 aa, synthetic construct; identical to SEQ ID NO: 859
  REGION 1..114: note = source = /note="Description of Artificial Sequence: Synthetic polypeptide"

SEQ ID NO: 864 - DNA, 342 bp, synthetic construct; identical to SEQ ID NO: 860
  misc_feature 1..342: note = source = /note="Description of Artificial Sequence: Synthetic polynucleotide"
  source 1..342: mol_type = other DNA

SEQ ID NO: 865 - protein, 114 aa, synthetic construct
  REGION 1..114: note = source = /note="Description of Artificial Sequence: Synthetic polypeptide"
GESVGLHLPTLSVQEGDNSIINCAYSNSASDYFIWYKQESGKGPQFIIDIRSNMDKRQGQRVTVLLNKTVKHLSLQIAATQPGDSAVYFCAENRQDNYGQNFVFGPGTRLSVLP

SEQ ID NO: 866 - DNA, 342 bp, synthetic construct
  misc_feature 1..342: note = source = /note="Description of Artificial Sequence: Synthetic polynucleotide"
  source 1..342: mol_type = other DNA
ggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaataggcaggataactatggtcagaattttgtctttggtcccggaaccagattgtccgtgctgccc

SEQ ID NO: 867 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWVNTEAFFGQGTRLTVV

SEQ ID NO: 868 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgggttaacactgaagctttctttggacaaggcaccagactcacagttgta

SEQ ID NO: 869 - protein, 110 aa, synthetic construct; identical to SEQ ID NO: 773
  REGION 1..110: note = source = /note="Description of Artificial Sequence: Synthetic polypeptide"

SEQ ID NO: 870 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgttctaccttgagagatgctgctgtgtactactgcatcctgcggcctgacagctgggggaagttccagtttggagcagggacccaggttgtggtcacccca

SEQ ID NO: 871 - protein, 114 aa, synthetic construct
  REGION 1..114: note = source = /note="Description of Artificial Sequence: Synthetic polypeptide"
DVKVTQSSRYLVKRTGEKVFLECVQDMDHENMFWYRQDPGLGLRLIYFSYDVKMKEKGDIPEGYSVSREKKERFSLILESASTNQTSMYLCASSTVRQGNYGYTFGSGTRLTVV

SEQ ID NO: 872 - DNA, 342 bp, synthetic construct
  misc_feature 1..342: note = source = /note="Description of Artificial Sequence: Synthetic polynucleotide"
  source 1..342: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtaccgtgaggcaggggaactatggctacaccttcggttcggggaccaggttaaccgttgta

SEQ ID NO: 873 - protein, 110 aa, synthetic construct
  REGION 1..110: note = source = /note="Description of Artificial Sequence: Synthetic polypeptide"
DAKTTQPNSMESNEEEPVHLPCNHSTISGTDYIHWYRQLPSQGPEYVIHGLTSNVNNRMASLAIAEDRKSSTLILHRATLRDAAVYYCILNTGTASKLTFGTGTRLQVTL

SEQ ID NO: 874 - DNA, 330 bp, synthetic construct
  misc_feature 1..330: note = source = /note="Description of Artificial Sequence: Synthetic polynucleotide"
  source 1..330: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgaataccggcactgccagtaaactcacctttgggactggaacaagacttcaggtcacgctc

SEQ ID NO: 875 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSSNTEAFFGQGTRLTVV

SEQ ID NO: 876 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcgtcgaacactgaagctttctttggacaaggcaccagactcacagttgta

SEQ ID NO: 877 - protein, 110 aa, synthetic construct
  REGION 1..110: note = source = /note="Description of Artificial Sequence: Synthetic polypeptide"
QKEVEQNSGPLSVPEGAIASLNCTYSDRGSQSFFWYRQYSGKSPELIMSIYSNGDKEDGRFTAQLNKASQYVSLLIRDSQPSDSATYLCAVNRGTDKLIFGTGTRLQVFP

SEQ ID NO: 878 - DNA, 330 bp, synthetic construct
  misc_feature 1..330: note = source = /note="Description of Artificial Sequence: Synthetic polynucleotide"
  source 1..330: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacagaggcaccgacaagctcatctttgggactgggaccagattacaagtctttcca

SEQ ID NO: 879 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSWTDYGYTFGSGTRLTVV

SEQ ID NO: 880 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctggacagactatggctacaccttcggttcggggaccaggttaaccgttgta

SEQ ID NO: 881 - protein, 110 aa, synthetic construct; identical to SEQ ID NO: 773
  REGION 1..110: note = source = /note="Description of Artificial Sequence: Synthetic polypeptide"

SEQ ID NO: 882 - DNA, 330 bp, synthetic construct; identical to SEQ ID NO: 774
  misc_feature 1..330: note = source = /note="Description of Artificial Sequence: Synthetic polynucleotide"
  source 1..330: mol_type = other DNA

SEQ ID NO: 883 - protein, 111 aa, synthetic construct; identical to SEQ ID NO: 879
  REGION 1..111: note = source = /note="Description of Artificial Sequence: Synthetic polypeptide"

SEQ ID NO: 884 - DNA, 333 bp, synthetic construct; identical to SEQ ID NO: 880
  misc_feature 1..333: note = source = /note="Description of Artificial Sequence: Synthetic polynucleotide"
  source 1..333: mol_type = other DNA

SEQ ID NO: 885 - protein, 111 aa, synthetic construct; identical to SEQ ID NO: 785
  REGION 1..111: note = source = /note="Description of Artificial Sequence: Synthetic polypeptide"

SEQ ID NO: 886 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggaggaacctcaggaacctacaaatacatctttggaacaggcaccaggctgaaggttttagca

SEQ ID NO: 887 - protein, 113 aa, synthetic construct; identical to SEQ ID NO: 823
  REGION 1..113: note = source = /note="Description of Artificial Sequence: Synthetic polypeptide"

SEQ ID NO: 888 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtcccgggacacccaactatggctacaccttcggttcggggaccaggttaaccgttgta

SEQ ID NO: 889 - protein, 110 aa, synthetic construct
  REGION 1..110: note = source = /note="Description of Artificial Sequence: Synthetic polypeptide"
RKEVEQDPGPFNVPEGATVAFNCTYSNSASQSFFWYRQDCRKEPKLLMSVYSSGNEDGRFTAQLNRASQYISLLIRDSKLSDSATYLCVVNGGSQGNLIFGKGTKLSVKP

SEQ ID NO: 890 - DNA, 330 bp, synthetic construct
  misc_feature 1..330: note = source = /note="Description of Artificial Sequence: Synthetic polynucleotide"
  source 1..330: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaatggaggaagccaaggaaatctcatctttggaaaaggcactaaactctctgttaaacca

SEQ ID NO: 891 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSVGDYGYTFGSGTRLTVV

SEQ ID NO: 892 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcgtaggggactatggctacaccttcggttcggggaccaggttaaccgttgta

SEQ ID NO: 893 - protein, 111 aa, synthetic construct; identical to SEQ ID NO: 233
  REGION 1..111: note = source = /note="Description of Artificial Sequence: Synthetic polypeptide"

SEQ ID NO: 894 - DNA, 333 bp, synthetic construct
  misc_feature 1..333: note = source = /note="Description of Artificial Sequence: Synthetic polynucleotide"
  source 1..333: mol_type = other DNA
ggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaacaactatggtcagaattttgtctttggtcccggaaccagattgtccgtgctgccc

SEQ ID NO: 895 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWDSSPLHFGNGTRLTVT

SEQ ID NO: 896 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgggacagttcacccctccactttgggaacgggaccaggctcactgtgaca

SEQ ID NO: 897 - protein, 112 aa, synthetic construct
  REGION 1..112: note = source = /note="Description of Artificial Sequence: Synthetic polypeptide"
DQQVKQNSPSLSVQEGRISILNCDYTNSMFDYFLWYKKYPAEGPTFLISISSIKDKNEDGRFTVFLNKSAKHLSLHIVPSQPGDSAVYFCAASAGSARQLTFGSGTQLTVLP

SEQ ID NO: 898 - DNA, 336 bp, synthetic construct
  misc_feature 1..336: note = source = /note="Description of Artificial Sequence: Synthetic polynucleotide"
  source 1..336: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgccggttctgcaaggcaactgacctttggatctgggacacaattgactgttttacct

SEQ ID NO: 899 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLYTHTEAFFGQGTRLTVV

SEQ ID NO: 900 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatacacccacactgaagctttctttggacaaggcaccagactcacagttgta

SEQ ID NO: 901 - protein, 112 aa, synthetic construct
  REGION 1..112: note = source = /note="Description of Artificial Sequence: Synthetic polypeptide"
DAKTTQPPSMDCAEGRAANLPCNHSTISGNEYVYWYRQIHSQGPQYIIHGLKNNETNEMASLIITEDRKSSTLILPHATLRDTAVYYCIVRDTTSGTYKYIFGTGTRLKVLA

SEQ ID NO: 902 - DNA, 336 bp, synthetic construct
  misc_feature 1..336: note = source = /note="Description of Artificial Sequence: Synthetic polynucleotide"
  source 1..336: mol_type = other DNA
gatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagacactacctcaggaacctacaaatacatctttggaacaggcaccaggctgaaggttttagca

SEQ ID NO: 903 - protein, 114 aa, synthetic construct
  REGION 1..114: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSSTGFSPLHFGNGTRLTVT

SEQ ID NO: 904 - DNA, 342 bp, synthetic construct
  misc_feature 1..342: note = source = /note="Description of Artificial Sequence: Synthetic polynucleotide"
  source 1..342: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcctcgacaggtttttcacccctccactttgggaacgggaccaggctcactgtgaca

SEQ ID NO: 905 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAEGGGSQGNLIFGKGTKLSVKP

SEQ ID NO: 906 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcagaggggggaggaagccaaggaaatctcatctttggaaaaggcactaaactctctgttaaacca

SEQ ID NO: 907 - protein, 111 aa, synthetic construct; identical to SEQ ID NO: 879
  REGION 1..111: note = source = /note="Description of Artificial Sequence: Synthetic polypeptide"

SEQ ID NO: 908 - DNA, 333 bp, synthetic construct; identical to SEQ ID NO: 880
  misc_feature 1..333: note = source = /note="Description of Artificial Sequence: Synthetic polynucleotide"
  source 1..333: mol_type = other DNA

SEQ ID NO: 909 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAGIRSNDYKLSFGAGTTVTVRA

SEQ ID NO: 910 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcagggatacgttctaacgactacaagctcagctttggagccggaaccacagtaactgtaagagca

SEQ ID NO: 911 - protein, 113 aa, synthetic construct
  REGION 1..113: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSWTAHTEAFFGQGTRLTVV

SEQ ID NO: 912 - DNA, 339 bp, synthetic construct
  misc_feature 1..339: note = source = /note="Description of Artificial Sequence: Synthetic polynucleotide"
  source 1..339: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagttcctggacagcccacactgaagctttctttggacaaggcaccagactcacagttgta

SEQ ID NO: 913 - protein, 112 aa, synthetic construct
  REGION 1..112: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAENSGGGADGLTFGKGTHLIIQP

SEQ ID NO: 914 - DNA, 336 bp, synthetic construct
  misc_feature 1..336: note = source = /note="Description of Artificial Sequence: Synthetic polynucleotide"
  source 1..336: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcagaaaattcaggaggaggtgctgacggactcacctttggcaaagggactcatctaatcatccagccc

SEQ ID NO: 915 - protein, 111 aa, synthetic construct; identical to SEQ ID NO: 815
  REGION 1..111: note = source = /note="Description of Artificial Sequence: Synthetic polypeptide"

SEQ ID NO: 916 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttcacagactatggctacaccttcggttcggggaccaggttaaccgttgta

SEQ ID NO: 917 - protein, 111 aa, synthetic construct
  REGION 1..111: note = source = /note="Description of Artificial Sequence: Synthetic polypeptide"
TQLLEQSPQFLSIQEGENLTVYCNSSSVFSSLQWYRQEPGEGPVLLVTVVTGGEVKKLKRLTFQFGDARKDSSLHITAAQPGDTGLYLCAGEDFGNEKLTFGTGTRLTIIP

SEQ ID NO: 918 - DNA, 333 bp, synthetic construct
  misc_feature 1..333: note = source = /note="Description of Artificial Sequence: Synthetic polynucleotide"
  source 1..333: mol_type = other DNA
acccagctgctggagcagagtcctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagaggactttggaaatgagaaattaacctttgggactggaacaagactcaccatcataccc

SEQ ID NO: 919 - protein, 111 aa, synthetic construct
  REGION 1..111: note = source = /note="Description of Artificial Sequence: Synthetic polypeptide"
DAGVTQSPTHLIKTRGQQVTLRCSPKSGHDTVSWYQQALGQGPQFIFQYYEEEERQRGNFPDRFSGHQFPNYSSELNVNALLLGDSALYLCASSWADYGYTFGSGTRLTVV

SEQ ID NO: 920 - DNA, 333 bp, synthetic construct
  misc_feature 1..333: note = source = /note="Description of Artificial Sequence: Synthetic polynucleotide"
  source 1..333: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctgggcggattatggctacaccttcggttcggggaccaggttaaccgttgta

SEQ ID NO: 921 - protein, 110 aa, synthetic construct
  REGION 1..110: note = source = /note="Description of Artificial Sequence: Synthetic polypeptide"
ILNVEQSPQSLHVQEGDSTNFTCSFPSSNFYALHWYRWETAKSPEALFVMTLNGDEKKKGRISATLNTKEGYSYLYIKGSQPEDSATYLCAFLTGNQFYFGTGTSLTVIP

SEQ ID NO: 922 - DNA, 330 bp, synthetic construct
  misc_feature 1..330: note = source = /note="Description of Artificial Sequence: Synthetic polynucleotide"
  source 1..330: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcctttctcaccggtaaccagttctattttgggacagggacaagtttgacggtcattcca

SEQ ID NO: 923 - protein, 114 aa, synthetic construct; identical to SEQ ID NO: 871
  REGION 1..114: note = source = /note="Description of Artificial Sequence: Synthetic polypeptide"

SEQ ID NO: 924 - DNA, 342 bp, synthetic construct; identical to SEQ ID NO: 872
  misc_feature 1..342: note = source = /note="Description of Artificial Sequence: Synthetic polynucleotide"
  source 1..342: mol_type = other DNA

SEQ ID NO: 925 - protein, 112 aa, synthetic construct
  REGION 1..112: note = source = /note="Description of Artificial Sequence: Synthetic polypeptide"
DQQVKQSSPSLSVQEGRISILNCDYTNSMFDYFLWYKKYPAEGPTFLISISSIKDKNEDGRFTVFLNKSAKHLSLHIVPSQPGDSAVYFCAASAGSARQLTFGSGTQLTVLP

SEQ ID NO: 926 - DNA, 336 bp, synthetic construct
  misc_feature 1..336: note = source = /note="Description of Artificial Sequence: Synthetic polynucleotide"
  source 1..336: mol_type = other DNA
gaccagcaagttaagcaaagttcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgccggttctgcaaggcaactgacctttggatctgggacacaattgactgttttacct

SEQ ID NO: 927 - protein, 112 aa, synthetic construct
  REGION 1..112: note = source = /note="Description of Artificial Sequence: Synthetic polypeptide"
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLWSNTEAFFGQGTRLTVV

SEQ ID NO: 928 - DNA, 336 bp, synthetic construct
  misc_feature 1..336: note = source = /note="Description of Artificial Sequence: Synthetic polynucleotide"
  source 1..336: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatggtcgaacactgaagctttctttggacaaggcaccagactcacagttgta

SEQ ID NO: 929 - protein, 9 aa, Homo sapiens
RLDQLLRHV

SEQ ID NO: 930 - protein, 9 aa, Homo sapiens
SLLQHLIGL

SEQ ID NO: 931 - DNA, 2082 bp, Homo sapiens
  source 1..2082: mol_type = genomic DNA
acgcagcggctagggcgcttagctgagccattgtctcgttctttccttccagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 932 - DNA, 2061 bp, Homo sapiens
  source 1..2061: mol_type = genomic DNA
acgcagcggctagggcgcttagctgagccattgtctcgttctttccttccagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 933 - DNA, 2206 bp, Homo sapiens
  source 1..2206: mol_type = genomic DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 934 - DNA, 1963 bp, Homo sapiens
  source 1..1963: mol_type = genomic DNA
acgcagcggctagggcgcttagctgagccattgtctcgttctttccttccagcaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 935 - DNA, 1980 bp, Homo sapiens
  source 1..1980: mol_type = genomic DNA
agacgcaaaagcccacacttccagtggtgtcagagagtatgagctccaggaggctttgatctcactgccccatgggcttgggccattagggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 936 - DNA, 2242 bp, Homo sapiens
  source 1..2242: mol_type = genomic DNA
aaaagcaccagtgggtgatcaggcccaggataggaatcatccctaagcagatccttaaatgccaaatataacttcaattttttgtgacggaaaatgtatttgtgcactgtgcctagcatggaaatagagaaaaactgagtctaattgataggcctgattgcatctcgctgaccagaatctctgtacctgagcagcttaggggcaagaaagaacagctgttttcttgaagtgtggaaacattagtaatacttaggaaggggctggccattggagattatgtttgtggtaaaggcctgacctgctgcttgtgaacagccccagtgtggaagaggacacctaaccataaagagaagggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 937 - DNA, 2121 bp, Homo sapiens
  source 1..2121: mol_type = genomic DNA
acagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 938 - DNA, 2304 bp, Homo sapiens
  source 1..2304: mol_type = genomic DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 939 - DNA, 2435 bp, Homo sapiens
  source 1..2435: mol_type = genomic DNA
agagggcctgggaggaagtgggttttgcatacagtccctgttgactctagtgccccctgctggccccagacgcgagttccggcgaggcttcagggtacagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 940 - DNA, 2100 bp, Homo sapiens
  source 1..2100: mol_type = genomic DNA
acagctcccccgcagccagaagccgggcctgcagcgcctcagcaccgctccgggacaccccacccgcttcccaggcgtgacctgtcaacagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 941 - DNA, 2845 bp, Homo sapiens
  source 1..2845: mol_type = genomic DNA
agagggcctgggaggaagtgggttttgcatacagtccctgttgactctagtgccccctgctggccccagacgcgagttccggcgaggcttcagggtacagctcccccgcagccagaagccgggcctgcagcgcctcagcaccgctccgggacaccccacccgcttcccaggcgtgacctgtcaacagcaacttcgcggtgtggtgaactctctgaggaaaaacgtaagttcgagccctgattcctccgcttccccgcagggtgaccttgggcttgtgcccccagcaccacccctgtcccgggtccctgttttctctctggaaatgggttgaagaccaaagaaaataatgtgcgccacttgggtcaccccgggccgcctgccccggaaaattggccccagttgaggagttgtggctgtaaggatgccttgaaccgaggcggcggtgctcgtggttggagctctccagggtgggtgcgcatttgtaatgcggtggatgctctgggactcggcccctctgaaggtgctgggggttggggacggcccaggcagtggcgtaggcgtcctaggaaggcgggagcagaggcagaaatgtcgctgcaagaccgtagtcagggtccttgaccacaggggtcacttgtgaccaaccacatggtctgttgttcctcctgccccctggttcagcccaggaaacactggtgctcaggtttggagccagagatttgcactgaaagggcgggattgagtcgccagttgtcagtttcctcagcagtatttgcggaggttttcacaggaggccgttgcttcgtaaatattatacatgtattcttctttttggagcattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcacctggaaaaaaaaa

SEQ ID NO: 942 - protein, 493 aa, Homo sapiens
MSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 943 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 944 - protein, 509 aa, Homo sapiens; identical to SEQ ID NO: 943

SEQ ID NO: 945 - protein, 509 aa, Homo sapiens; identical to SEQ ID NO: 943

SEQ ID NO: 946 - protein, 509 aa, Homo sapiens; identical to SEQ ID NO: 943

SEQ ID NO: 947 - protein, 509 aa, Homo sapiens; identical to SEQ ID NO: 943

SEQ ID NO: 948 - protein, 509 aa, Homo sapiens; identical to SEQ ID NO: 943

SEQ ID NO: 949 - protein, 493 aa, Homo sapiens; identical to SEQ ID NO: 942

SEQ ID NO: 950 - protein, 493 aa, Homo sapiens; identical to SEQ ID NO: 942

SEQ ID NO: 951 - protein, 493 aa, Homo sapiens; identical to SEQ ID NO: 942

SEQ ID NO: 952 - protein, 509 aa, Homo sapiens; identical to SEQ ID NO: 943

SEQ ID NO: 953 - protein, 9 aa, Homo sapiens
SLLMWITQC

SEQ ID NO: 954 - protein, 6 aa, synthetic construct
  REGION 1..6: note = source = /note="Description of Artificial Sequence: Synthetic 6xHis tag"
HHHHHH